(19) United States
(12) Patent Application Publication
(10) Pub. No.: US 2025/0255985 A1
(43) Pub. Date: Aug. 14, 2025

(54) PARVOVIRUS ITR-BASED GENE DELIVERY VECTOR SYSTEM

(71) Applicant: ModeX Therapeutics, Inc., Weston, MA (US)

(72) Inventors: Xiangmudong KONG, Boston, MA (US); Nicholas Edward JONES, Northborough, MA (US); Leon BROWN, Spencer, MA (US); Gary J. NABEL, Delray Beach, FL (US); Zhi-yong YANG, Newton Center, MA (US)

(21) Appl. No.: 19/051,107

(22) Filed: Feb. 11, 2025

Related U.S. Application Data
(60) Provisional application No. 63/552,575, filed on Feb. 12, 2024.

(51) Int. Cl. A61K 48/00; A61K 47/68; A61K 47/69; C12N 15/86; C12N 15/88

(52) U.S. Cl. CPC A61K 48/0041; A61K 47/6879; A61K 47/6913; C12N 15/86; C12N 15/88; C12N 2750/14143; C12N 2830/50

(57) ABSTRACT

The present disclosure provides an AAV-ITR based gene delivery system comprising (i) an AAV-ITR vector comprising a double stranded polynucleotide encoding a gene of interest flanked by ITRs and (ii) a Rep vector comprising an mRNA encoding a Rep protein such as Rep78, wherein both vectors are encapsulated, e.g., in a liposome or LNP. Such a combination of ITR vector with the expression of Rep68/78 results in amplification of the AAV-ITR vector and enhanced episomal maintenance of the vector in the cells, which provides the base for sustained transgene expression. The liposomes containing the AAV-ITR vector and Rep vector components of the gene delivery system can be targeted to one or more surface proteins on a targeted cell or tissue. The gene of interest can be, for example, chimeric antigen receptors (CAR), or antibodies. Also provided are pharmaceutical compositions, vectors, lipid compositions, kits, and methods of treatment.

### CROSS-REFERENCE TO RELATED APPLICATIONS AND INCORPORATION BY REFERENCE

This application claims the priority benefit of U.S. Provisional Application No. 63/552,575, filed on Feb. 12, 2024, which is herein incorporated by reference in its entirety.

### REFERENCE TO SEQUENCE LISTING SUBMITTED ELECTRONICALLY

The content of the electronically submitted ST.26 sequence listing in XML format (Name 4850_0190002_SequenceListing_ST26.xml; Size: 2,104,957 bytes; and Date of Creation: Feb. 11, 2025) filed with the application is incorporated herein by reference in its entirety.

### FIELD

The present disclosure relates to gene transfer system utilizing Parvovirus ITR elements in combination with suitable delivery methods to administer payloads of a nucleic acid of interest that codes for therapeutic polypeptides to cells in eukaryotic subjects.

### BACKGROUND

Parvoviruses are a family of mammalian viruses including the human Adeno-associated viruses (AAV). Their single-stranded DNA (ssDNA) genomes is flanked by Inverted Terminal Repeat (ITR) at each end that form into hairpin loops that are important for viral life cycle including replication, viral packaging and gene expression. AAV vectors have been utilized for years in research and in development of the transfer of genetic material coding for genes of interest into subjects that need treatment thereof. Gene therapy strives to correct genes that are defective, and which are associated with or responsible for an underlying disease or condition. AAVs are comparatively small, single stranded DNA viruses which require helper virus for efficient replication. The genome of AAV is 4.7 kb and is characterized by two ITR and two open reading frames (ORF) which encode Rep proteins and Cap proteins. The ORF encoding the Rep protein encodes four proteins having molecular weights of 78 kD, 68 kD, 52 kD and 40 kD. The Rep proteins are responsible for regulating AAV replication and integration.

Gene transfer technology is the key component of transformative medical intervention such as gene therapy. Recombinant viral vectors based on AAVs and lentivirus are the main gene transfer technologies currently being used in gene therapies. Viral vector systems generally have intrinsic technical and biological hurdles such as insert capacity, industrial scale manufacturability, host immunogenicity against viral vector and complications from high dose delivery. Non-viral gene delivery is preferred, but current technologies lack robustness in gene transfer efficiency and only allow for short-term gene expression. Recent progress in mRNA/LNP delivery technology may provide a path forward, albeit a technology to provide sustainable gene expression is required.

To develop a non-viral vector system for long-term gene expression, it is essential to use host cellular machineries for replication/transcription/translation and avoid permanent presence of viral protein that can induce immuno-response resulting in the elimination of transduced cells by host immune system. The avoidance of an immune response in a gene transfer system remains an unmet medical need. The present inventors have discovered a system that maintains the long-term expression of genes and other proteins of interest to be used as successful therapeutics in humans.

U.S. Pat. No. 7,186,552 discloses the nucleic acid sequences of AAV serotype 1 and vectors and host cells containing these sequences and functional fragments thereof. This patent discloses a recombinant vector comprising an AAV-1 ITR and a selected transgene with the vector comprising both the 5′ and 3′ AAV-1 ITRs between which the transgene is located. This patent discloses host cells stably transduced with an AAV-1 P5 promoter. U.S. Pat. No. 9,598,703 discloses isolated, linear capsid-free nucleic acid molecules which comprise a first AAV ITR, a nucleotide sequence of interest (NSI or GOI) and a second AAV ITR wherein the nucleic acid molecule is devoid of AAV capsid protein coding sequences. This patent discloses that the nucleotide sequence of interest can be an expression cassette of an exogenous DNA. The isolated linear capsid-free nucleic acid molecule is described therein as single-stranded. WO 2016/134337 discloses site-specific integrating recombinant AAV vectors for gene therapy.

### BRIEF SUMMARY

The present disclosure provides an AAV-ITR based gene delivery system comprising (i) a linear double-stranded DNA (dsDNA) AAV-ITR vector comprising 5′ and 3′ AAV ITR flanking a polynucleotide sequence encoding a therapeutic polypeptide, a therapeutic polynucleotide, or a combination thereof, and, (ii) a mRNA Rep vector encoding a Rep protein. In some aspects, (i) is encapsulated in a first liposome or lipid nanoparticle (LNP) and (ii) encapsulated in a second liposome or LNP. In some aspects, the ITR comprises an ITR sequence selected from AAV1, AAV2, AAV3, AAV3B, AAV4, AAV5, AAV6, AAV7, AAV8, AAV9, AAV10, AAV11, AAV12, AAV13, it is an ortholog parvoviral ITR, it is a functional fragment of a parvoviral ITR, or it is a functional variant of a parvoviral ITR. In some aspects, the mRNA Rep vector comprises an mRNA encoding an AAV Rep protein. In some aspects, the AAV Rep protein comprises a Rep78 protein, a Rep68 protein, a variant thereof, or a functional fragment thereof. In some aspects, the AAV Rep78 protein is from AAV1, AAV2, AAV3, AAV3B, AAV4, AAV5, AAV6, AAV7, AAV8, AAV9, AAV10, AAV11, AAV12, or AAV13, it is a Rep78 variant, or it is a Rep78 functional fragment.

In some aspects, the AAV-ITR based gene delivery system of the present disclosure comprises an AAV-ITR vector having the topology of Schema I, shown below:

[ITRL]-[E]-[P]-[I]-[GOI]-[P(A) signal]-[ITRR]

wherein ITRL and ITRR are ITRs, and ITRR is the reverse complement if ITRL; E is an enhancer; P is a promoter; I is an intron; GOI is a polynucleotide sequence encoding a therapeutic polypeptide, a therapeutic polynucleotide, or a combination thereof; and, P(A) signal is a polyadenylation signal.

In some aspects, the AAV-ITR based gene delivery system of the present disclosure comprises a Rep vector having the topology of Schema II, shown below:

[P]-[5′ UTR]-[Rep78/68]-[3′ UTR]-[P(A) tail]

wherein P is a promoter; 5′ UTR is a 5′ untranslated region (UTR); 3′ UTR is a 3′ untranslated region; Rep78/68 is a polynucleotide sequence encoding a Rep78 or Rep68 protein; and, P(A) tail is a polyadenylation tail.

In some aspects, the polynucleotide sequence encodes an antibody, an enzyme, a receptor, an ion channel, a vaccine antigen, a chimeric antigen receptor (CAR), a hormone, a cytokine, a growth factor, or an apoptosis regulator. The liposome and/or LNP comprises (i) a cationic or ionizable lipid or lipidoid; (ii) a structural lipid; (iii) a helper lipid; (iv) a stabilizing lipid; or, (v) a combination thereof. In some aspects, the ionizable lipid or lipidoid is selected from the group consisting of cKK-E12, AIC-0315, SM-102, YK-009, DLin-MC3-DMA (MC3), DLin-KC2-DMA (KC2), A6, OF-02, A18-Iso5-2DC18, 98N12-5, 9A1p9, C12-200, 7C1, G0-C14, L319, 304O13, OF-Deg-Lin, 306-O12B, 306O110, FTT5, Lipid 10, and combinations thereof. In some aspects, the delivery system is targeted using at least one targeting molecule. In some aspects, the at least one targeting molecule is an antibody or a combination thereof. In some aspects, the antibody is an M-STAR antibody or a combination thereof.

The present disclosure also provides a method of replicating a polynucleotide encoding a therapeutic polypeptide, therapeutic polynucleotide or combination thereof in vivo in a patient in need of treatment for a disease or condition, comprising co-delivering the AAV-ITR based gene delivery system of the present disclosure to the patient to transiently express the Rep protein encoded by the Rep vector to replicate and amplify the polynucleotide encoding the therapeutic polypeptide, therapeutic polynucleotide or combination encoded by the AAV-ITR vector. Also provided is a method of delivering a therapeutic polypeptide, therapeutic polynucleotide or combination thereof in vivo to a patient in need of treatment for a disease or condition, comprising co-delivering the AAV-ITR based gene delivery system of the present disclosure to the patient, wherein the Rep protein encoded by the Rep vector is expressed transiently and replicates and amplifies the polynucleotide encoding the therapeutic polypeptide, therapeutic polynucleotide or combination encoded by the AAV-ITR vector, and wherein the expression of the therapeutic polypeptide, therapeutic polynucleotide or combination thereof treats the disease or condition in the patient.

The present disclosure also provides a therapeutic polypeptide, therapeutic polynucleotide or combination produced in vivo or ex vivo by co-delivering the AAV-ITR based gene delivery system of the present disclosure to a host cell, wherein the Rep protein encoded by the Rep vector is expressed transiently and replicates and amplifies the polynucleotide encoding the therapeutic polypeptide, therapeutic polynucleotide or combination encoded by the AAV-ITR vector, and wherein the cell expresses the therapeutic polypeptide, therapeutic polynucleotide or combination thereof. Also provided is a set of vectors comprising (i) a linear dsDNA AAV-ITR vector comprising 5′ and 3′ AAV ITR flanking a polynucleotide sequence encoding a therapeutic polypeptide, a therapeutic polynucleotide, or a combination thereof, and, (ii) a linear mRNA Rep vector encoding a Rep protein, wherein the vectors are co-encapsulated in a lipidic or polymeric delivery system, and wherein the lipidic or polymeric delivery system is targeted to a specific cell or tissue. Also provided is a host cell comprising (i) a linear dsDNA AAV-ITR vector comprising 5′ and 3′ AAV ITR flanking a polynucleotide sequence encoding a therapeutic polypeptide, a therapeutic polynucleotide, or a combination thereof; and, (ii) a linear mRNA Rep vector encoding a Rep protein. Also provided is a kit comprising (i) a linear dsDNA AAV-ITR vector comprising 5′ and 3′ AAV ITR flanking a polynucleotide sequence encoding a marker; and, (ii) a linear mRNA Rep vector encoding a Rep protein, and instructions to replace the marker with a polynucleotide encoding a therapeutic polypeptide, a therapeutic polynucleotide, or a combination thereof.

### BRIEF DESCRIPTION OF THE DRAWINGS/FIGURES

Some aspects of the invention are herein described, by way of example only, with reference to the accompanying drawings. With specific reference now to the drawings in detail, it is stressed that the particulars shown are by way of example and for purposes of illustrative discussion of aspects of the invention.

FIG. 1A shows a schematic of the replication of an AAV DNA.

FIG. 1B shows a Southern blot of replicated AAV-ITR DNA in the presence of different cellular replication proteins. Arrows indicate the proteins that are necessary for DNA replication (Pol d, RFC, PCNA, MCM complex, and Rep68/78).

FIG. 2A shows a schematic representation of a linear dsDNA AAV-ITR vector that comprises a polynucleotide encoding a gene of interest (GOI) and regulatory elements flanked by ITRs.

FIG. 2B shows a schematic representation of a mRNA Rep vector comprising a Rep78 mRNA encoding a Rep78 protein.

FIG. 3A depicts a schematic diagram indicating how the dsDNA of AAV-ITR vectors (AAV ITR-EGFP) are encapsulated in LIPOFECTAMINE™ 2000 liposomes and how the mRNA of Rep vectors (Rep78 vector) are encapsulated in LIPOFECTAMINE® MESSENGERMAX™ mRNA Transfection Reagent liposomes and mixed at different ratios of the two different vectors. The resulting liposomes encapsulating the AAV-ITR vector or Rep vector were transfected to C2C12 cells using the different ratios of AAV-ITR vector (AAV-GFP DNA) to Rep vector (Rep 78 RNA).

FIG. 3B depicts plots of the percentage of EGFP+ cells versus days post transfection for the various mixtures of AAV-ITR vector (AAV-EGFP DNA) to Rep vector (Rep 78 RNA). Data is presented as percentage of GFP+ cells (% GFP) over time and medium fluorescent intensity (MFI) over time, showing that ITR_EGFP dsDNA co-transfected with Rep78 mRNA results in sustained GFP protein expression in 3T3 embryonic fibroblast cells.

FIG. 4 shows the transient expression of Rep78 protein in C2C12 cells post co-transfection on the time points indicated (Day 3, Day 7).

FIG. 5 shows quantitative analysis of EGFP mRNA level and ITR-EGFP DNA level from C2C12 cells co-transfected with ITR-EGFP dsDNA and Rep78 mRNA measured at 1 week, 2 weeks, 3 weeks or 4 weeks after transfection. The level of EGFP RNA was measured using qPCR detecting EGFP mRNA.

FIG. 6 shows quantitative analysis of ITR-EGFP DNA level from C2C12 cells co-transfected with ITR-EGFP dsDNA and Rep78 mRNA. Genomic and Episomal DNA fractions were also quantitated using qPCR.

FIG. 7A shows the selection of clones for the analysis of ITR-EGFP DNA integration into host cell genome using whole genome sequencing.

FIG. 7B shows the percentage of EGFP located in the nucleus which is about 0.0 for each measurement.

FIG. 8A shows the experimental design of the in vivo expression experiment in mice. Luciferase expression was measured after preparation of liposomes made with in vivo-jetPEI® in vivo transfection reagent or in vivo-jetPEI® in vivo transfection reagent that contained an AAV-ITR vector (AAV ITR-Luc DNA) or a Rep78 vector (Rep78 mRNA), respectively. The liposomes were formulated in each case using an N/P ratio of 8.

FIG. 8B shows the relative Luciferase expression following administration of only the IG. 8B shows the luciferase expression in mice receiving AAV-ITR vector (AAV ITR-Luc DNA), or AAV-ITR vector (AAV ITR-Luc DNA) plus Rep78 vector (Rep78 mRNA).

FIG. 9 shows the exemplary structure of an AVV-ITR vector (A) comprising a polynucleotide encoding a CAR as payload. Diagrams (B) and (C) show the architectures of two CAR-encoding polynucleotides that can be integrated in the AAV-ITR vector (A). Diagram (B) corresponds to the architecture of a construct encoding a bispecific CAR with a CD19 and CD18 antigen binding moiety in MSTAR format, and with a CD8-derived spacer and transmembrane domain. A specific CAR construct with this architecture is set forth in SEQ ID NO: 18. Diagram (C) corresponds to the architecture of a construct encoding a bispecific CAR with a CD19 and CD18 antigen binding moiety in MSTAR format, and with a CD8-derived spacer and transmembrane domain. A specific CAR construct with this architecture is set forth in SEQ ID NO: 19.

FIG. 10 is a schematic representation of lipid delivery systems that can be used to encapsulate the AAV-ITR vector and/or Rep vector of the delivery systems of the present disclosure. For example the AAV-ITR vector and/or Rep vector can be independently or simultaneously delivered using liposomes (e.g., multilamellar vesicles, large unilamellare vesicles, or small unilamellar vesicles), lipid nanoparticles, or combinations thereof. The liposomes and/or lipid nanoparticles can be, e.g., untargeted, targeted, or stealth.

FIG. 11A shows the sequences and structures of the 3′ ITRs from AAV1, AAV2, AAV3, AAV4, AAV6 and AAV7. Bold letters denote non-conserved nucleotides between the ITR sequences.

FIG. 11B shows a consensus AAV 3′ ITR sequence. IUPAC nomenclature is used to designate variable nucleotides: Y is C or T; R is A or G; S is G or C; W is A or T; K is G or T; M is A or C; B is G or T or C; V is G or C or A; and N is any nucleotide. In some aspects, the invention disclosed herein can the practiced using ITRs comprising a consensus sequence that comprises one of more universal bases.

FIG. 12 shows a detailed secondary structure diagram of the AAV2 3′ ITR. The AAV2 3′ ITR serves as origin of replication and is composed of two arm palindromes (B-B′ and C-C′) embedded in a larger stem palindrome (A-A′). The ITR can acquire two configurations (flip and flop). The flip (depicted) and flop configurations have the B-B′ and the C-C′ palindrome closest to the 3′ end, respectively. The D sequence is present only once at each end of the genome thus remaining single-stranded. The boxed motif corresponds to the Rep-binding element (RBE) where the AAV Rep78 and Rep68 proteins bind. The RBE consists of a tetranucleotide repeat with the consensus sequence 5′-GNGC-3′. The ATP-dependent DNA helicase activities of Rep78 and Rep68 remodel the A-A′ region generating a stem-loop that locates at the summit of the terminal resolution site (trs) in a single-stranded form. In this configuration, the strand- and site-specific endonuclease catalytic domain of Rep78 and Rep68 introduces a nick at the trs. The shaded nucleotides at the apex of the T-shaped structure correspond to an additional RBE (RBE′) that stabilizes the association between the two largest Rep proteins and the ITR.

### DETAILED DESCRIPTION

The present invention provides an improved AAV-ITR based gene delivery system that delivers nucleic acids coding for therapeutic agents (e.g., therapeutic polypeptides, therapeutic polynucleotides, or combinations thereof) to a host or host cell in need of treatment with high efficiency and sustained gene expression. In some aspects, the AAV-ITR based gene delivery system of the present disclosure comprises a combination of a double stranded AAV-ITR vector comprising a polynucleotide encoding a therapeutic agent, and a Rep vector comprising an mRNA encoding a Rep protein (e.g., Rep 68 or Rep 78).

Linear dsDNA vectors containing ITRs from AAV or other parvovirus flanking a transcription unit (cassette) containing at least one polynucleotide encoding a GOI have been developed (AAV-ITR vectors). Advantageously, the amplification of a dsDNA AAV-ITR vector comprising a polynucleotide encoding a GOI in the presence of transiently expressed a Rep78 or a Rep68 protein encoded by a second linear vector (Rep vector) results in persistent gene expression through episomal. The AAV-ITR based gene delivery system of the present disclosure is optionally capsid free. As used herein, the term “capsid free” refers to AAV-ITR based gene delivery system that is not encapsulated in a native parvoviral capsid. In some aspects, an AAV-ITR based gene delivery system can be encapsulated in a virus-like particle, a chimeric capsid comprising one or more parvoviral capsid component or fragment thereof, or lipid-based structure (e.g., liposome or lipid nanoparticle) comprising one or more parvoviral capsid component or fragment thereof,

Such a combination of ITR vector with the expression of Rep68/78 results in amplification of ITR vector and enhanced episomal maintenance of the vector in the cells, which provides the base for sustained transgene expression.

The AAV-ITR vector and Rep vectors of the present disclosure can be encapsulated in lipidic delivery systems such as liposomes or LNP, or in polymeric delivery systems. Alternatively, the vectors can be transfected into host cells using other transfection methods and compositions known in the art, for example, electroporation, lipofection or any non-viral gene transfer method.

In some aspects, the lipidic delivery system used to encapsulate the AAV-ITR vector is a liposome or LNP optimized for dsDNA delivery. In some aspects, the lipidic delivery system used to encapsulate the Rep vector is a liposome or LNP optimized for mRNA delivery.

The present disclosure also provides methods of enhanced transfection, methods of treatment, vectors, cells, and kits comprising the AAV ITR-base vector for gene delivery system of the present disclosure. LNPs, lipids and other LNP and delivery system components, payloads (e.g., CAR), targeting moieties (e.g., antibodies), etc. disclosed in PCT application PCT/US2024/053954 and U.S. application Ser. No. 18/933,688, can be used in combination with the disclosures of the present application the practice the inventions disclosed herein without undue experimentation. For example, any one the ionizable cationic lipids disclosed herein can be replaced with one of the ionizable cationic lipids disclosed in PCT application PCT/US2024/053954 and U.S. application Ser. No. 18/933,688. Likewise, any of the payloads disclosed herein can be replaced with any of the payload disclosed in PCT application PCT/US2024/053954 and U.S. application Ser. No. 18/933,688; any of the MSTAR constructs disclosed herein can be replaced with any of the MSTAR constructs disclosed in the PCT application PCT/US2024/053954 and U.S. application Ser. No. 18/933,688. The PCT application PCT/US2024/053954 and U.S. application Ser. No. 18/933,688 are incorporated herein in their entireties.

The present disclosure also provides methods of gene delivery to any cell type where expression of gene of interest can provide therapeutic benefit to certain diseases. Various terms relating to aspects of disclosure are used throughout the specification and claims. Such terms are to be given their ordinary meaning in the art, unless otherwise indicated. Other specifically defined terms are to be construed in a manner consistent with the definitions provided herein.

### Definitions

In order that the present description can be more readily understood, certain terms are first defined. Additional definitions are set forth throughout the detailed description. It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone). It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary of Biochemistry and Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. Sequences identified by SEQ ID NO are included in the ST.26 sequence listing according to the requirements of the standard, which requires uracils (u) to the replaced by thymines (t) in RNA sequences. It is to be understood that in some cases mixed sequences such as RNAi compounds can comprise both RNA and DNA nucleotides in the same molecule. Mixed type molecules are identified by an * following the SEQ ID NO. A person of ordinary skill in the art can determine whether a ‘t’ corresponds to a ‘t’ or an ‘u’ in the actual drug based and whether a nucleotide is RNA or DNA based on additional information provided in the specification, e.g., the INN (International Nonproprietary Name) of the compound.

The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 15 percent, up or down (higher or lower). Thus, in some aspects, about is interchangeable with ±15%. When particular values or compositions are provided in the application and claims, unless otherwise stated, the meaning of “about” should be assumed to be within an acceptable error range for that particular value or composition.

As described herein, any numerical range, concentration range, percentage range, ratio range or integer range is to be understood to include the value of any integer within the recited range and, when appropriate, fractions thereof (such as one-tenth and one-hundredth of an integer), unless otherwise indicated.

As used herein, the term “antigen binding polypeptide” refers to a polypeptide having the ability to specifically bind to one or more substances that induce an immune response (i.e., one or more antigens or epitopes).

As used herein, the term “antigen binding polypeptide complex” refers to a group of two, three, four, or more associated polypeptides, wherein at least one polypeptide has the ability to specifically bind to one or more antigens. An antigen binding polypeptide complex, includes, but is not limited to, an antibody or antigen binding fragment thereof.

The term “antibody” includes, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. A heavy chain can have the C-terminal lysine or not. Unless specified otherwise herein, the amino acids in the variable regions are numbered using the Kabat numbering system and those in the constant regions are numbered using the EU system.

The term “monoclonal antibody,” as used herein, refers to an antibody that is produced by a single clone of B-cells and binds to the same epitope. In contrast, the term “polyclonal antibody” refers to a population of antibodies that are produced by different B-cells and bind to different epitopes of the same antigen. The term “antibody” includes, by way of example, monoclonal and polyclonal antibodies; chimeric and humanized antibodies; human or non-human antibodies; wholly synthetic antibodies; and single chain antibodies. A non-human antibody can be humanized by recombinant methods to reduce its immunogenicity in man.

The antibody can be an antibody that has been altered (e.g., by mutation, deletion, substitution, conjugation to a non-antibody moiety). For example, an antibody can include one or more variant amino acids (compared to a naturally occurring antibody) which change a property (e.g., a functional property) of the antibody. For example, several such alterations are known in the art which affect, e.g., half-life, effector function, and/or immune responses to the antibody in a patient. The term antibody also includes artificial polypeptide constructs which comprise at least one antibody-derived antigen-binding site.

An “antigen binding fragment” of an antibody refers to one or more fragments or portions of an antibody that retain the ability to bind specifically to the antigen bound by the whole antibody. It has been shown that the antigen binding function of an antibody can be performed by fragments or portions of a full-length antibody. An antigen-binding fragment can contain the antigenic determining regions of an intact antibody (e.g., the CDRs). Examples of antigen binding fragments of antibodies include, but are not limited to, Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody.

Antigen binding fragments are obtained using conventional techniques known to those with skill in the art, and the fragments screened for utility in the same manner as are intact antibodies. Antigen binding fragments can be produced by recombinant DNA techniques, or by enzymatic or chemical cleavage of intact immunoglobulins.

As used herein, the term “variable region” typically refers to a portion of an antibody, generally, a portion of a light or heavy chain, typically about the amino-terminal 110 to 120 amino acids, or 110 to 125 amino acids in the mature heavy chain and about 90 to 115 amino acids in the mature light chain, which differ extensively in sequence among antibodies and are used in the binding and specificity of a particular antibody for its particular antigen.

The terms “complementarity determining region” or “CDR”, as used herein, refer to each of the regions of an antibody variable domain which are hypervariable in sequence and/or form structurally defined loops (hypervariable loops) and/or contain the antigen-contacting residues. Antibodies can comprise six CDRs, e.g., three in the VH and three in the VL.

The terms “VL”, “VL region,” and “VL domain” are used herein interchangeably to refer to the light chain variable region of an antigen binding polypeptide, antigen binding polypeptide complex, antibody or antigen binding fragment thereof. In some aspects, a VL region is referred to herein as VL1 to denote a first light chain variable region, VL2 to denote a second light chain variable region, VL3 to denote a third light chain variable region, and VL4 to denote a fourth light chain variable region. An enumerated VL region (e.g., VL1) can have the same or different antigen binding properties and/or the same or different sequence as another enumerated VL region (e.g., VL2).

The terms “VH”, “VH region,” and “VH domain” are used herein interchangeably to refer to the heavy chain variable region of an antigen binding polypeptide, antigen binding polypeptide complex, antibody or antigen binding fragment thereof. In some aspects, a VH region is referred to herein as VH1 to denote a first heavy chain variable region, VH2 to denote a second heavy chain variable region, VH3 to denote a third heavy chain variable region, and VH4 to denote a fourth heavy chain variable region. An enumerated VH region (e.g., VH1) can have the same or different antigen binding properties and/or the same or different sequence as another enumerated VH region (e.g., VH2).

As used herein, “Kabat numbering” and like terms are recognized in the art and refer to a system of numbering amino acid residues in the heavy and light chain variable regions of an antibody or antigen binding fragment thereof. In some aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

As used herein, the terms “constant region” or “constant domain” are used interchangeably to refer to a portion of an antigen binding polypeptide, antigen binding polypeptide complex, antibody or antigen binding fragment thereof, e.g., a carboxyl terminal portion of a light and/or heavy chain which is not directly involved in binding of an antibody to antigen but which can exhibit various effector functions, such as interaction with the Fc region. The constant region generally has a more conserved amino acid sequence relative to a variable region. In some aspects, an antigen binding polypeptide, antigen binding polypeptide complex, antibody or antigen binding fragment thereof comprises a constant region or portion thereof that is sufficient for antibody-dependent cell-mediated cytotoxicity (ADCC), antibody-dependent cellular phagocytosis (ADCP), and complement-dependent cytotoxicity (CDC). A constant region includes, but is not limited to, a light chain constant region (CL) or heavy chain constant region (CH1, CH2, CH3).

As used herein, the terms “fragment crystallizable region,” “Fc region,” or “Fc domain” are used interchangeably herein to refer to the tail region of an antibody that interacts with cell surface receptors called Fc receptors and some proteins of the complement system. Fc regions typically comprise CH2 and CH3 regions, and, optionally, an immunoglobulin hinge.

As used herein, the terms “immunoglobulin hinge,” “hinge,” “hinge domain” or “hinge region” are used interchangeably to refer to a stretch of heavy chains between the Fab and Fc portions of an antigen binding polypeptide, antigen binding polypeptide complex, antibody or antigen binding fragment thereof. A hinge provides structure, position and flexibility, which assist with normal functioning of antibodies (e.g., for crosslinking two antigens or binding two antigenic determinants on the same antigen molecule). An immunoglobulin hinge is divided into upper, middle and lower hinge regions that can be separated based on structural and/or genetic components. An immunoglobulin hinge of the invention can contain one, two or all three of these regions. Structurally, the upper hinge region stretches from the C terminal end of CH1 to the first hinge disulfide bond. The middle hinge region stretches from the first cysteine to the last cysteine in the hinge. The lower hinge region extends from the last cysteine to the glycine of CH2. The cysteines present in the hinge form interchain disulfide bonds that link the immunoglobulin monomers.

As used herein, the term “Fab” refers to a region of an antibody that binds to an antigen. It is typically composed of one constant and one variable domain of each of the heavy and the light chain.

As used herein, the term “heavy chain” refers to a portion of an antigen binding polypeptide, antigen binding polypeptide complex, antibody or antigen binding fragment thereof typically composed of a heavy chain variable region (VH), a heavy chain constant region 1 (CH1), a heavy chain constant region 2 (CH2), and a heavy chain constant region 3 (CH3). A typical antibody is composed of two heavy chains and two light chains. When used in reference to an antibody, a heavy chain can refer to any distinct type, e.g., alpha (a), delta (6), epsilon (F), gamma (γ), and mu (p), based on the amino acid sequence of the constant region, which gives rise to IgA, IgD, IgE, IgG, and IgM classes of antibodies, respectively, including subclasses of IgG, e.g., IgG1, IgG2, IgG3, and IgG4. Heavy chain amino acid sequences are known in the art. In some aspects, the heavy chain is a human heavy chain.

As used herein, the term “light chain” refers to a portion of an antigen binding polypeptide, antigen binding polypeptide complex, antibody or antigen binding fragment thereof typically composed of a light chain variable region (VL) and a light chain constant region (CL). A typical antibody is composed of two light chains and two heavy chains. When used in reference to an antibody, a light chain can refer to any distinct type, e.g., kappa (κ) or lambda (λ), based on the amino acid sequence of the constant region. Light chain amino acid sequences are known in the art. In some aspects, the light chain is a human light chain.

The term “chimeric” antibody or antigen-binding fragment thereof refers to an antibody or antigen binding fragments thereof wherein the amino acid sequence is derived from two or more species. Typically, the variable region of both light and heavy chains corresponds to the variable region of antibodies or antigen binding fragments thereof derived from one species of mammals (e.g., mouse, rat, rabbit, etc.) with the desired specificity, affinity and capability, while the constant regions are homologous to the sequences in antibodies or antigen binding fragments thereof derived from another (usually human) to avoid eliciting an immune response in that species.

The term “humanized” antibody or antigen binding fragment thereof refers to forms of non-human (e.g., murine) antibodies or antigen binding fragments that are specific immunoglobulin chains, chimeric immunoglobulins, or fragments thereof that contain minimal non-human (e.g., murine) sequences. Typically, humanized antibodies or antigen binding fragments thereof are human immunoglobulins in which residues from a complementary determining region (CDR) are replaced by residues from a CDR of a non-human species (e.g., mouse, rat, rabbit, hamster) that have the desired specificity, affinity, and capability (Jones et al., Nature 321:522-525 (1986); Riechmann et al., Nature 332:323-327 (1988); Verhoeyen et al., Science 239:1534-1536 (1988)). In some aspects, the Fv framework region (FR) residues of a human immunoglobulin are replaced with the corresponding residues in an antibody or fragment from a non-human species that has the desired specificity, affinity, and capability. The humanized antibody or antigen binding fragment thereof can be further modified by the substitution of additional residues either in the Fv framework region and/or within the replaced non-human residues to refine and optimize antibody or antigen-binding fragment thereof specificity, affinity, and/or capability. In general, a humanized antibody or antigen binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. A humanized antibody or antigen binding fragment thereof can also comprise at least a portion of a constant region, typically that of a human immunoglobulin. Examples of methods used to generate humanized antibodies are known and described, for example, in U.S. Pat. No. 5,225,539; Roguska et al., Proc. Natl. Acad. Sci., USA, 91(3):969-973 (1994), and Roguska et al., Protein Eng. 9(10):895-904 (1996).

The term “human” antibody or antigen-binding fragment thereof, as used herein, means an antibody or antigen-binding fragment thereof having an amino acid sequence derived from a human immunoglobulin gene locus, where such antibody or antigen-binding fragment is made using recombinant techniques known in the art. This definition of a human antibody or antigen-binding fragment thereof includes intact or full-length antibodies and fragments thereof.

A polypeptide, polypeptide complex, antibody, antigen binding fragment thereof, polynucleotide, vector or host cell which is “isolated” is a polypeptide, polypeptide complex, antibody, antigen binding fragment thereof, polynucleotide, vector or host cell which is in a form not found in nature. Isolated polypeptides, polypeptide complexes, antibodies, antigen binding fragments thereof, polynucleotides, vectors or host cells include those which have been purified to a degree that they are no longer in a form in which they are found in nature. In some aspects, a polypeptide, polypeptide complex, antibody, antigen-binding fragment thereof, polynucleotide, vector or host cell which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., 70% sequence identity.”

Calculation of the percent identity of two polypeptide sequences, for example, can be performed by aligning the two sequences for optimal comparison purposes (e.g., gaps can be introduced in one or both of a first and a second polypeptide sequences for optimal alignment and non-identical sequences can be disregarded for comparison purposes). In certain aspects, the length of a sequence aligned for comparison purposes is at least about 30%, at least about 40%, at least about 50%, at least about 60%, at least about 70%, at least about 80%, at least about 90%, at least about 95%, or about 100% of the length of the reference sequence. The amino acids at corresponding amino acid positions are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some aspects of the present disclosure, the biologically active molecule attached to the EV, e.g., exosome, via a maleimide moiety is a polynucleotide, e.g., an antisense oligonucleotide. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects of the present disclosure, the biologically active molecule is a polynucleotide. In some aspects, a polynucleotide disclosed herein can be modified to introduce a thiol group that can be used to react with a maleimide moiety. In some aspects, a polynucleotide disclosed herein can be modified to introduce a maleimide moiety group that can be used to react with a thiol group.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; e.g., disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. In some aspects of the present disclosure, the biologically active molecule attached to the EV, e.g., exosome, via a maleimide moiety is a polypeptide, e.g., an antibody or a derivative thereof such as an ADC, a PROTAC, a toxin, a fusion protein, or an enzyme.

The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

In some aspects, a polypeptide disclosed herein can be modified to introduce a thiol group that can be used to react with a maleimide moiety. In some aspects, a polypeptide disclosed herein can be modified to introduce a maleimide moiety that can be used to react with a thiol group.

As used herein, the term “nanoparticle” refers to particles having a particle size on the nanometer scale, less than 1 micrometer. For example, the nanoparticle can have a particle size up to about 50 nm. In another example, the nanoparticle can have a particle size up to about 10 nm. In another example, the nanoparticle can have a particle size up to about 6 nm. As used herein, “nanoparticle” refers to a number of nanoparticles, including, but not limited to, nanoclusters, nanovesicles, micelles, lamaellae shaped particles, polymersomes, dendrimers, and other nano-size particles of various other small fabrications that are known to those in the art. The shapes and compositions of nanoparticles can be guided during condensation of atoms by selectively favoring growth of particular crystal facets to produce spheres, rods, wires, discs, cages, core-shell structures and many other shapes. The definitions and understandings of the entities falling within the scope of nanocapsule are known to those of skill in the art, and such definitions are incorporated herein by reference and for the purposes of understanding the general nature of the subject matter of the present application.

As used herein, the term “Chimeric Antigen Receptor” or alternatively a “CAR” refers to a recombinant polypeptide construct comprising at least an extracellular antigen binding domain, a transmembrane domain, and a cytoplasmic signaling domain comprising a functional signaling domain derived from a stimulatory molecule as defined below. In one aspect, the stimulatory molecule is the zeta chain associated with the T cell receptor complex. In one aspect, the cytoplasmic signaling domain further comprises one or more functional signaling domains derived from at least one costimulatory molecule as defined below. In one aspect, the costimulatory molecule is chosen from 4 1BB (i.e., CD137), CD3, and/or CD28. In one aspect, the CAR comprises a chimeric fusion protein comprising an extracellular antigen recognition domain, a transmembrane domain, and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the scFv domain during cellular processing and localization of the CAR to the cellular membrane.

The portion of the CAR composition comprising an antibody or antibody fragment thereof can exist in a variety of forms where the antigen binding domain is expressed as part of a contiguous polypeptide chain including, for example, a single domain antibody fragment (sdAb), a single chain antibody (scFv) and a humanized antibody. In one aspect, the antigen-binding domain of a CAR composition of the invention comprises an antibody fragment. In one embodiment, the CAR comprises an antibody fragment that comprises a scFv.

As used herein, the term “MSTAR” refers to an antibody format disclosed in U.S. Appl. Publ. Nos. US20230227553A1, US20230235092A1, US20230203199A1, and PCT Publ. Nos. WO2023056312, WO2023056313, WO2023056314, which are herein incorporated by reference in their entireties. In some aspects, the AAV-ITR based gene delivery system of the present disclosure can be targeted to a specific cell or tissue using, e.g., a monospecific, bispecific or trispecific MSTAR antibody. In some aspects, the AAV-ITR based gene delivery system of the present disclosure comprises an AAV-ITR vector wherein the polynucleotide encoding a therapeutic polypeptide, therapeutic polynucleotide, or combination thereof comprising ORFs encoding a MSTAR antibody. In some aspects, the polynucleotide encoding the MSTAR antibody is biscistronic. In some aspects, the polynucleotide comprises multiples cistrons, wherein each cistron encodes a polypeptide chain of an MSTAR antibody.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

An “adeno-associated virus” or “AAV” is a non-enveloped, single-stranded DNA a Dependoparvovirus (genus) within the Parvoviridae family of viruses. In contrast to most other members of the Parvoviridae family, AAV is replication defective and is only able to replicate efficiently in the presence of a helper virus such as adenovirus or herpes virus. AAV was first reported in the mid 1960's as a contaminant of viral preparations of adenovirus. See Atchison et al Science. (1965) 149:754-756. Since then, progressively safer and more effective methods to use AAV as a recombinant DNA vector have been developed. See, e.g., Hermonat et al. PNAS (1984) 81:6466-6470; Laughlin et al. (1983) Gene 23:65-73; Matsushita et al. (1998) Gene Ther. 5:938-945; Xiao et al. (1998) J. Virol. 72:2224-2232. It has been reported that low numbers of AAV genomes can integrate into the host chromosome. Cheung et al. (1980) J. Virol. 33:739-748.

As used herein, the term “adeno-associated virus” or “AAV” includes but is not limited to, serotypes AAV type 1, AAV type 2, AAV type 3 (including types 3 and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, primate AAV, non-primate AAV, and those AAV serotypes and clades disclosed by Gao et al. (2004) J. Virol. 78:6381 and Moris et al. (2004) Virol. 33:375 (2004), and any other AAV known in the art. “Primate AAV” refers to AAV that infect primates, “non-primate AAV” refers to AAV that infect non-primate mammals, “bovine AAV” refers to AAV that infect bovine mammals, etc. See Fields et al. Virology, volume 2, chapter 69 (4th ed., Lippincott-Raven Publishers).

The term “Parvovirus” as used herein refers to any member of the Parvoviridae family. They have linear ssDNA genomes about 4-6 kilobases (kb) in length that typically contain two genes encoding for a replication initiator protein, called NS1, and the protein the viral capsid is made of. NS1 contains an HUH superfamily endonuclease domain near its N-terminus, containing both site-specific binding activity and site-specific nicking activity, and a superfamily 3 (SF3) helicase domain toward the C-terminus. The coding portion of the genome is flanked by telomeres at each end that form into hairpin loops that are important during replication. During replication, the hairpins repeatedly unfold, are replicated, and refold to change the direction of replication to progress back and forth along the genome in a process called rolling hairpin replication that produces a molecule containing numerous copies of the genome. Progeny ssDNA genomes are excised from this concatemer.

The coding portion of the genome of a parvovirus is flanked at each end by terminal sequences about 116-550 nucleotides (nt) in length that consist of imperfect palindromes folded into hairpin loop structures. These hairpin loops contain most of the cis-acting information required for DNA replication and packaging and act as hinges during replication to change the direction of replication. When the genome is converted to double-stranded forms, replication origin sites are created involving sequences in and adjacent to the hairpins.

In some aspects, the compositions and methods of the present disclosure can comprise any parvoviral NS1/Rep protein in the role of the Rep68 or Rep78 disclosed herein, and any telomeric palindromic hairpin loop in the role of the ITR disclosed herein. Thus, in some aspects of the present disclosure the terms Rep, Rep68, or Rep78 can refer to an orthologous sequence of the AAV2 Rep68 protein or the AAV2 Rep78 protein or a nucleic acid sequence encoding said proteins from any of the parvovirus genomes disclosed herein. In some aspects of the present disclosure the terms Rep, Rep68, or Rep78 can refer to an orthologous sequence of the AAV2 Rep68 protein or AAV2 Rep78 protein or nucleic acid sequence encoding said proteins from any of the parvovirus genomes disclosed herein, wherein the Rep protein (e.g., Rep68 or Rep78) is a functional variant or functional fragment that contains a Rep_N Rep protein catalytic-like domain (Interpro IPR014835). In some aspects of the present disclosure the terms Rep, Rep68, or Rep78 can refer to a protein or nucleic acid sequence encoding said protein that contains a Rep_N Rep protein catalytic-like domain (Interpro IPR014835).

In some aspects of the present disclosure the term ITR can refer to orthologous sequences of the AAV2 ITR sequences from any of the parvovirus genomes disclosed herein. As used herein, the terms “orthologous,” “ortholog,” and grammatical variants thereof refer to refers to a gene from a different species that encodes a similar protein that performs the same biological function. The term also refers to the protein encoded by the gene. For example, the Rep78 genes from, for example, two different species of parvovirus are orthologs.

Within each ITR there are two sequences required for replication: a Rep binding sequence (RBS) consisting of tetranucleotide direct repeats, and a terminal resolution site (trs) where Rep cleaves the viral DNA. In the presence of ATP and a divalent metal ion, Rep78 and Rep68 bind to and nick linear double-stranded (ds) DNA substrates that contain an RBS and a trs, indicating that the entire ITR is not required for these Rep activities. See Nash et al. (2009) J. Virol. 83: 454-469; Hickman et al. (2002) Mol. Cell 10: 327-33; Chiorini et al. (1994) J. Virol. 68:7448-7457; and Chiorini et al. (1994) J. Virol. 68:797-804, which are herein incorporated by reference.

Thus, in some aspects of the present disclosure, the term ITR can refer to orthologous sequences of the AAV2 ITR sequences from any of the parvovirus genomes disclosed herein wherein the sequence is a functional variant or functional fragment comprising a Rep-binding element (RBE) and a terminal resolution site (trs).

As used herein, the term “fragment,” e.g., a fragment of a Rep78 or a fragment of an ITR disclosed herein, refers to a sequence (polypeptide or polynucleotide) that is shorter than a sequence disclosed herein (e.g., an mRNA encoding AAV2 Rep78, a Rep78 protein, or a naturally occurring ITR capable of binding to a Rep78 protein) by deleting a portion of the sequence disclosed herein (e.g., 5′-end and/or 3′-end and/or internal deletion of a polynucleotide disclosed herein, or C-terminus and/or N-terminus and/or internal deletion of a polypeptide disclosed herein). In some aspects, the fragment is a subsequence of a naturally occurring polypeptide or polynucleotide.

As used herein, the term “functional fragment” refers to a polynucleotide fragment or a protein fragment derived from a polynucleotide or polypeptide sequence disclosed herein that retains at least part of the functional characteristics of the original polynucleotide or polypeptide that are necessary for their use in the compositions and methods closed herein. Conversely, a “non-functional fragment” would lack one or more of the functional characteristics of the parent molecule and therefore it is not suitable for use in the compositions and methods closed herein.

Whether a fragment of a polynucleotide or polypeptide disclosed herein is a functional fragment can be assessed by any art known methods without undue experimentation. In some aspects, the functional fragment retains, e.g., at least about 20%, at least about 30%, at least about 40%, at least about 50%, at least about 60%, at least about 70%, at least about 80%, at least about 90% or at least about 100% of the ability of an ITR or Rep78/Rep68 polynucleotide or Rep78/Rep68 polypeptide to effectively deliver a gene of interest according to the methods disclosed herein and promote sustained transgene expression with respect to an unmodified ITR or Rep78/Rep68 polynucleotide or Rep78/Rep68 polypeptide.

As used herein, the term “functional variant” refers to a polynucleotide or polypeptide derived from a polynucleotide or polypeptide disclosed herein, e.g., via point mutation, insertion, deletion, etc., that retains at least part of the function of the original polynucleotide or polypeptide and can be used in the compositions and methods disclosed herein. For example, in some aspects, a functional variant of a Rep78 protein encoded by a functional variant disclosed herein, retains the ability to bind to an ITR and to deliver a gene of interest according to the methods disclosed herein and promote sustained transgene expression. Conversely, a “non-functional variant” would lack one or more of the functional characteristics of the parent molecule.

Whether a variant of a polynucleotide or polypeptide disclosed herein is a functional variant can be assessed by any art known methods without undue experimentation. In some aspects, the functional variant retains, e.g., at least about 20%, at least about 30%, at least about 40%, at least about 50%, at least about 60%, at least about 70%, at least about 80%, at least about 90% or at least about 100% of the ability of an ITR or Rep78/Rep68 (i.e., Rep78 or Rep68) polynucleotide or Rep78/Rep68 polypeptide to effectively deliver a gene of interest according to the methods disclosed herein with respect to an unmodified ITR or Rep78/Rep68 polynucleotide or Rep78/Rep68 polypeptide.

The term “Dependoparvovirus” as used herein is interchangeable with the terms Dependovirus or Adenoassociated virus group and refers to the genus in the subfamily Parvovirinae of the virus family Parvoviridae that comprises the AAV viruses disclosed herein. AAV viruses receive their name because they cannot replicate productively in their host cell without the cell being coinfected by a helper virus such as an adenovirus, a herpesvirus, or a vaccinia virus. The Dependoparvovirus genus comprises eleven recognized species: Adeno-associated dependoparvovirus A, Adeno-associated dependoparvovirus B, Anseriform dependoparvovirus 1, Avian dependoparvovirus 1, Carnivore dependoparvovirus 1, Chiropteran dependoparvovirus 1, Pinniped dependoparvovirus 1, Rodent dependoparvovirus 1, Rodent dependoparvovirus 2, Squamate dependoparvovirus 1, and Squamate dependoparvovirus 2.

Dependoparvovirus is not infectious enough to trigger an immune response; this makes it a good virus to use as a gene therapy tool. Since this virus does not stimulate an immune response it can be used multiple times effectively without being neutralized before infection. Another reason these viruses are reliable vectors is the known insertion point for the genome. This virus always inserts its contents into the same place on chromosome 19. This predictability can cut down on the chances of inserting into an important area that might disrupt normal gene function or increase the risk of developing cancer.

As used herein, an antigen binding polypeptide or antigen binding polypeptide complex (e.g., an antibody or antigen binding fragment thereof), or region or domain thereof that “specifically binds” refers to its association with an epitope by its antigen binding domain, and that the binding entails some complementarity between the antigen binding domain and the epitope. Specific binding to an epitope occurs where there is binding to that epitope via its antigen binding domain more readily than there would be binding to a random, unrelated epitope.

In some aspects, the compositions and methods of the present disclosure can comprise any dependoparvoviral NS1/Rep protein in the role of the Rep68 or Rep78 disclosed herein, and any telomeric palindromic hairpin loop from any devendoparvovirus in the role of the ITR disclosed herein.

As used herein, an “epitope” refers to a localized region of an antigen to which an antigen binding polypeptide or antigen binding polypeptide complex (e.g., antibody or antigen binding fragment thereof) can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). See, e.g., Giege R et al., (1994) Acta Crystallogr D Biol Crystallogr 50(Pt 4):339-350; McPherson (1990) Eur. J. Biochem. 189:1-23; Chayen (1997) Structure 5:1269-1274; McPherson (1976) J. Biol. Chem. 251:6300-6303; Meth. Enzymol. (1985) volumes 114 & 115, eds. Wyckoff et al., U.S. Pub. No. 2004/0014194, Bricogne G (1993) Acta Crystallogr D Biol Crystallogr 49(Pt 1):37-60, Bricogne G (1997) Meth. Enzymol. 276A:361-423, ed. Carter, Roversi et al. (2000) Acta Crystallogr D Biol Crystallogr 56(Pt 10):1316-1323 (X-ray diffraction crystallography studies); Champe et al. (1995) J. Biol. Chem. 270:1388-1394, and Cunningham & (1989) Science 244:1081-1085 (mutagenesis mapping).

Specific binding can be represented by a “binding affinity.” Binding affinity refers to an intrinsic binding affinity which reflects a 1:1 interaction between members of a binding pair (e.g., an antigen binding polypeptide complex and an antigen). Binding affinity can be measured and/or expressed in several ways known in the art, including, but not limited to, equilibrium dissociation constant (KD). KD is calculated from the quotient of koff/kon, where kon refers to the association rate constant of, e.g., an antigen binding polypeptide complex to an antigen, and koff refers to the dissociation of, e.g., an antigen binding polypeptide complex from an antigen. The kon and koff can be determined by techniques known to one of ordinary skill in the art, such as Octet® BLI, BIAcore® or KinExA. Accordingly, in some aspects, an antigen binding polypeptide complex provided herein is an antibody or antigen binding fragment thereof. In some aspects, an antigen binding polypeptide provided herein is part of an antibody or antigen-binding fragment thereof. In some aspects, the antibody or antigen binding fragment thereof specifically binds to an antigen with an equilibrium dissociation constant (KD) of from about 10 μM to about 1 pM.

A “capsid-free” or “capsid-less” (or variations thereof) vector or nucleic acid molecule refers to a nucleic acid molecule or vector construct that is free of a capsid, e.g., free of a viral capsid. In some aspects, “capsid-free” or “capsid-less” (or variations thereof) vector or nucleic acid molecule refers to a nucleic acid molecule or vector construct that is free of a capsid protein. For example, a nucleic acid molecule or vector can comprise a nucleic acid sequence of a virus genome while not containing any nucleic acid sequences encoding a viral capsid protein or fragment thereof. A composition can comprise a nucleic acid molecule or vector comprising a nucleic acid sequence of a viral genome while not containing any polypeptide sequences of a viral capsid protein or fragment thereof. E.g., the capsid-less vector or nucleic acid molecule does not contain sequences encoding an AAV Cap protein or AAV capsid protein amino acid sequences.

### AAV-ITR Based Gene Delivery System

In some aspects, the present disclosure provides an AAV-ITR based gene delivery system comprising (i) a first linear vector comprising a dsDNA comprising a genetic cassette comprising a polynucleotide encoding a therapeutic polypeptide (e.g., a CAR or antibody), a therapeutic polynucleotide, or a combination thereof (e.g., a gene editing system comprising a Cas protein and a gRNA) flanked by AAV ITRs, named “AAV-ITR vector” throughout this disclosure; and, (ii) a linear mRNA encoding an AAV Rep protein, e.g., Rep68 or Rep78, named “Rep vector” throughout this disclosure.

In other aspects, the present disclosure provides a capsid-free AAV-ITR based gene delivery system comprising (i) a first linear vector comprising a dsDNA comprising a genetic cassette comprising a polynucleotide encoding a therapeutic polypeptide (e.g., a CAR or antibody), a therapeutic polynucleotide, or a combination thereof (e.g., a gene editing system comprising a Cas protein and a gRNA) flanked by AAV ITRs, named “AAV-ITR vector” throughout this disclosure; and, (ii) a linear mRNA encoding an AAV Rep protein, e.g., Rep68 or Rep78, named “Rep vector” throughout this disclosure.

Accordingly, the terms “AAV-ITR based gene delivery system” and “AAV-ITR based gene delivery system of the present disclosure refers to the combination of a first linear vector comprising a dsDNA comprising a genetic cassette comprising a polynucleotide encoding a therapeutic polypeptide (e.g., a CAR or antibody), a therapeutic polynucleotide, or a combination thereof flanked by AAV ITRs, e.g., AAV2 ITRs, and a second linear mRNA vector encoding an AAV Rep protein, e.g., an AAV2 Rep78 or Rep68 protein.

The term “AAV-ITR vector,” referred to also using the terms “ITR_GOI vector” or “AAV ITR_GOI vector” refers, e.g., to an AAV genome in which all of the rep and cap genes have been replaced with heterologous sequences, and wherein the heterologous sequences are located between the AAV ITRs. In other words, the AAV-ITR vector of the present disclosure is a construct in which the only two AAV components are the ITRs. This reflects the fact that the only cis-element required for the production of AAV genomes are the AAV ITRs. Thus, in some aspects disclosed herein, an AAV-ITR vector contain only a polynucleotide encoding a therapeutic polypeptide (e.g., a CAR or antibody), a therapeutic polynucleotide, or a combination thereof flanked by the ITRs.

In some aspects the AAV-ITR vector can include one or more regulatory sequences. In some aspects, the regulatory sequences are flanked by the ITRs. In some aspects the AAV-ITR vector can include a non-coding regulatory DNA. In some aspects, the AAV-ITR vector comprises a genetic cassette comprising a regulatory sequence or regulatory element that controls expression of a therapeutic polypeptide, a therapeutic polynucleotide, or a combination thereof.

The terms “regulatory sequence” and “regulatory element” are used interchangeably and are intended to include promoters, enhancers, introns, and other expression control elements (e.g., polyadenylation signals) that control the transcription or translation of a polynucleotide sequence that encodes a therapeutic polypeptide, a therapeutic polynucleotide, or a combination thereof. Such regulatory sequences are described, for example, in Goeddel (Gene Expression Technology. Methods in Enzymology 185, Academic Press, San Diego, CA (1990)). It will be appreciated by those skilled in the art that the design of the AAV-ITR vector, including the selection of regulatory sequences, can depend on such factors, e.g., the choice of the host cell to be transformed, or the level of expression of protein desired.

In some aspects, the AAV-ITR vector comprises mRNA splice donor/splice acceptor sites. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter or AdMLP) and polyoma. Alternatively, nonviral regulatory sequences can be used, such as the ubiquitin promoter or β-globin promoter. Still further, regulatory elements composed of sequences from different sources, such as the SRa promoter system, which contains sequences from the SV40 early promoter and the long terminal repeat of human T cell leukemia virus type 1 (Takebe et al. (1988) Mol. Cell. Biol. 8:466-472), can be used.

In some aspects, the regulatory elements in the AAV-ITR vector can be constitutive. In some aspects, the regulatory elements in the AAV-ITR vector can be regulatable. In some aspects, the regulatory elements in the AAV-ITR vector can be tissue-specific. In certain aspects, the regulatory sequence comprises a tissue-specific promoter. In some aspects, the tissue specific promoter drives expression of the gene of interest in a tissue selected from the group consisting of heart, liver, lungs, eyes, nervous system, lymphatic system, muscle and stem cells.

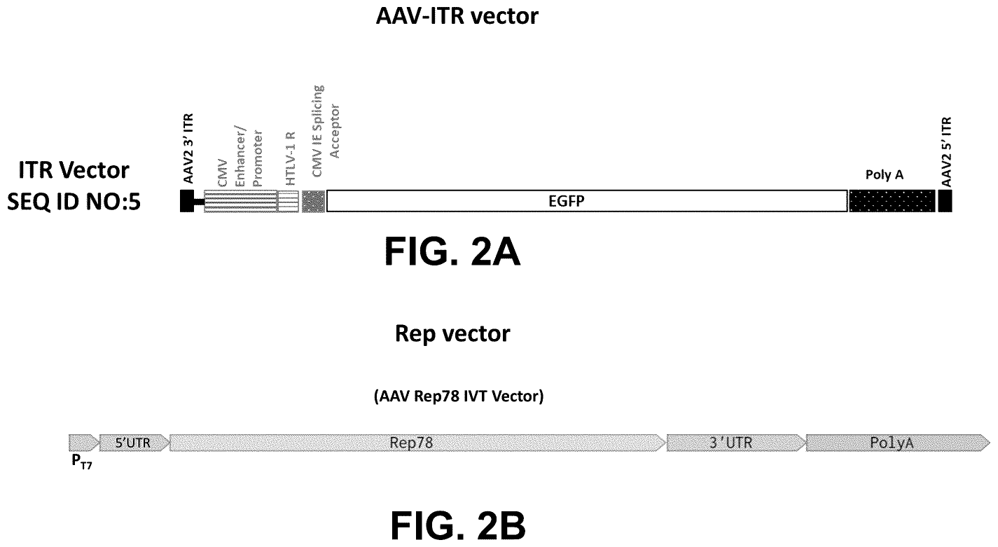

In some aspects, an AAV-ITR vector can include additional regulatory elements including an intron and/or a polyadenylation signal. In some aspects, the intron is located 5′ of the polynucleotide sequence encoding the therapeutic polypeptide, therapeutic polynucleotide, or combination thereof. In some aspects, the polyadenylation signal is located 3′ of the polynucleotide sequence encoding the therapeutic polypeptide, therapeutic polynucleotide, or combination thereof. A representative structure of an AAV-ITR vector is presented in FIG. 2A.

As used herein, the term “Rep vector” refers to a linear mRNA encoding an AAV Rep protein, e.g., AAV2 Rep78 or AAV2 Rep68. In some aspects the Rep vector can include regulatory elements such as expression control elements (e.g., promoters or enhancers) that drive the translation of the polynucleotide sequence encoding the Rep78 or Rep68 protein. In some aspects, a Rep vector can include a promoter, 5′ UTR, 3′ UTR, and a polyadenine tail. A representative structure of a Rep vector is presented in FIG. 2B.

In some aspects, the Rep vector comprises an mRNA encoding a Rep78 protein, i.e., the Rep vector is a Rep78 vector. In some aspects, the Rep vector comprises an mRNA encoding a Rep68 protein, i.e., the Rep vector is a Rep68 vector. In some aspects, the Rep vector comprises an mRNA encoding an AAV2 Rep78 protein, i.e., the Rep vector is an AAV2 Rep78 vector. In some aspects, the Rep vector comprises an mRNA encoding an AAV2 Rep68 protein, i.e., the Rep vector is an AAV2 Rep68 vector.

In some aspects, the topology of the AAV-ITR vector corresponds to Schema I

[ITRL]-[E]-[P]-[I]-[GOI]-[P(A) signal]-[ITRR] Schema I

wherein:
ITRL and ITRR are ITRs, and ITRR is the reverse complement if ITRL;
E is an enhancer;
P is a promoter;
I is an intron;
GOI is a polynucleotide sequence encoding a therapeutic polypeptide, a therapeutic
polynucleotide, or a combination thereof, and,
P(A) signal is a polyadenylation signal.

In some aspects, the ITR can be an ITR from AAV2. In other aspects, the ITR can be from an AAV serotype selected from the group consisting of AAV1, AAV2, AAV3, AAV3B, AAV4, AAV5, AAV6, AAV7, AAV8, AAV9, AAV10, AAV11, AAV12 and AAV13. ITR that can be used in the AAV-ITR based gene delivery systems of the present disclosure are discussed more in detail in a specific section below.

In some aspects, the enhancer/promoter is CMV. In some aspects, the enhancer/promoter is mouse CMV. In some aspects, the PolyA signal is a PolyA signal derived from bovine growth hormone mRNA. In some aspects, the Poly A is a rabbit beta-globin Poly A signal. In some aspects, the Poly A is an SV40 Poly A signal.

In some aspects, the AAV-ITR vector is an AAV2 ITR_CMVR_EGFP_Tbgh vector (SEQ ID NO:35), which comprises an ORF sequence encoding Enhanced Green Fluorescent Protein (EGFP) (SEQ ID NO:36). The ORF encoding EGPF in the AAV2 ITR_CMVR_EGFP_Tbgh vector can be replaced with a polynucleotide encoding a GOI.

The architecture of the AAV-ITR vector is shown in the table below.

TABLE 1
Architecture of the AAV-ITR vector of SEQ ID NO: 35.

| Component | Position (nt) |
|---|---|
| AAV2 3′ ITR | 10-139 |
| CMV enhancer | 244-696 |
| CMV promoter | 697-985 |
| HTLV R region | 986-1216 |
| CMV IE intron | 1217-1324 |
| EGFP | 1379-2098 |
| Poly A | 2111-2661 |
| AAV2 5′ ITR | 2713-2842 |

Thus, in some aspects, an AVV-ITR vector of the present disclosure comprises the sequence of the AAV-ITR disclosed above in which the coding sequence between positions 1379 and 2098 has been replaced with a coding sequence (CDS) encoding a payload such as an antibody (e.g., an MSTAR antibody) or a CAR. The polynucleotide sequence encoding EGFP in the AAV-ITR vector of SEQ ID NO:35 can be replaced with a polynucleotide encoding a therapeutic polypeptide, therapeutic polynucleotide, or combination using recombinant techniques known in the art. The ITR sequences in the AAV-ITR vector of SEQ ID NO:35 can be replaced with other parvoviral ITR sequences known in the art.

In some aspects, the topology of the Rep vector corresponds to Schema II

[P]-[5′ UTR]-[Rep78/68]-[3′ UTR]-[P(A) tail] Schema II

wherein:
P is a promoter;
5′ UTR is a 5′ untranslated region;
3′ UTR is a 3′ untranslated region;
Rep78/68 is a polynucleotide sequence encoding a Rep68 or Rep68 protein; and,
P(A) tail is a polyadenylation tail.

In some aspects, the promoter of the Rep vector is a T7 promoter. The T7 promoter is a sequence of DNA 18 base pairs long up to transcription start site at +1 having the sequence taatacgactcactatag (SEQ ID NO:37) that is recognized by T7 RNA polymerase. Only the full-length Rep78 protein and alternatively spliced variant Rep68 can replicate an AAV-ITR linear dsDNA vector. Any 5′ UTR or 3′ UTR from an eukaryotic mRNA can be used in the Rep vectors of the present disclosure. In some aspects, the Rep vector is an AAV2_REP78_IVT vector of SEQ ID NO: 38. In some aspects, the Rep78 sequence encoded by the AAV2_REP78_IVT vector comprises the sequence set forth in SEQ ID NO: 39. Thus, in some aspects, the Rep78 protein corresponds to the Genbank QDH44117.1. A detailed description of the architecture of the Rep vector AAV2_REP78_IVT (SEQ ID NO: 38) is presented in TABLE 2, below.

TABLE 2
Detailed structure of the Rep vector of SEQ ID NO: 38.

| Component | Position (nt) |
|---|---|
| TEV | 1-135 |
| AAV2 Rep78 | 136-2002 |
| hbGlobin 3′ UTR | 2011-2070 |
| PolyA | 2171-2270 |

The RNA sequence encoding the Rap78 protein in the Rep vector of SEQ ID NO: 38 can be replaced with an RNA encoding an AAV2 Rep68 protein or any homologous parvoviral Rep protein using recombinant techniques known in the art. In some aspects, the Rep protein can be a Rep78 from AAV2. In other aspects, the Rep protein can be a Rep78 from an AAV serotype selected from the group consisting of AAV1, AAV2, AAV3, AAV3B, AAV4, AAV5, AAV6, AAV7, AAV8, AAV9, AAV10, AAV11, AAV12 and AAV13. In some aspects, the Rep protein can be a Rep68 from AAV2. In other aspects, the Rep protein can be a Rep68 from an AAV serotype selected from the group consisting of AAV1, AAV2, AAV3, AAV3B, AAV4, AAV5, AAV6, AAV7, AAV8, AAV9, AAV10, AAV11, AAV12 and AAV13. Rep68 and Rep78 polynucleotides encoding Rep68 and Rep78 proteins that can be used in the AAV-ITR based gene delivery systems of the present disclosure are discussed more in detail in a specific section below.

In some aspects, the ITR of the AAV-ITR vector and the Rep78 mRNA of the Rep vector of an AAV-ITR based gene delivery system of the present disclosure are from AAV1. In some aspects, the ITR of the AAV-ITR vector and the Rep78 mRNA of the Rep vector of an AAV-ITR based gene delivery system of the present disclosure are from AAV2. In some aspects, the ITR of the AAV-ITR vector and the Rep78 mRNA of the Rep vector of an AAV-ITR based gene delivery system of the present disclosure are from AAV3. In some aspects, the ITR of the AAV-ITR vector and the Rep78 mRNA of the Rep vector of an AAV-ITR based gene delivery system of the present disclosure are from AAV3B. In some aspects, the ITR of the AAV-ITR vector and the Rep78 mRNA of the Rep vector of an AAV-ITR based gene delivery system of the present disclosure are from AAV4. In some aspects, the ITR of the AAV-ITR vector and the Rep78 mRNA of the Rep vector of an AAV-ITR based gene delivery system of the present disclosure are from AAV5. In some aspects, the ITR of the AAV-ITR vector and the Rep78 mRNA of the Rep vector of an AAV-ITR based gene delivery system of the present disclosure are from AAV6. In some aspects, the ITR of the AAV-ITR vector and the Rep78 mRNA of the Rep vector of an AAV-ITR based gene delivery system of the present disclosure are from AAV7. In some aspects, the ITR of the AAV-ITR vector and the Rep78 mRNA of the Rep vector of an AAV-ITR based gene delivery system of the present disclosure are from AAV8. In some aspects, the ITR of the AAV-ITR vector and the Rep78 mRNA of the Rep vector of an AAV-ITR based gene delivery system of the present disclosure are from AAV9. In some aspects, the ITR of the AAV-ITR vector and the Rep78 mRNA of the Rep vector of an AAV-ITR based gene delivery system of the present disclosure are from AAV10. In some aspects, the ITR of the AAV-ITR vector and the Rep78 mRNA of the Rep vector of an AAV-ITR based gene delivery system of the present disclosure are from AAV11. In some aspects, the ITR of the AAV-ITR vector and the Rep78 mRNA of the Rep vector of an AAV-ITR based gene delivery system of the present disclosure are from AAV12. In some aspects, the ITR of the AAV-ITR vector and the Rep78 mRNA of the Rep vector of an AAV-ITR based gene delivery system of the present disclosure are from AAV13.

In some aspects, the ITR of the AAV-ITR vector and the Rep68 mRNA of the Rep vector of an AAV-ITR based gene delivery system of the present disclosure are from AAV1. In some aspects, the ITR of the AAV-ITR vector and the Rep68 mRNA of the Rep vector of an AAV-ITR based gene delivery system of the present disclosure are from AAV2. In some aspects, the ITR of the AAV-ITR vector and the Rep68 mRNA of the Rep vector of an AAV-ITR based gene delivery system of the present disclosure are from AAV3. In some aspects, the ITR of the AAV-ITR vector and the Rep68 mRNA of the Rep vector of an AAV-ITR based gene delivery system of the present disclosure are from AAV3B. In some aspects, the ITR of the AAV-ITR vector and the Rep68 mRNA of the Rep vector of an AAV-ITR based gene delivery system of the present disclosure are from AAV4. In some aspects, the ITR of the AAV-ITR vector and the Rep68 mRNA of the Rep vector of an AAV-ITR based gene delivery system of the present disclosure are from AAV5. In some aspects, the ITR of the AAV-ITR vector and the Rep68 mRNA of the Rep vector of an AAV-ITR based gene delivery system of the present disclosure are from AAV6. In some aspects, the ITR of the AAV-ITR vector and the Rep68 mRNA of the Rep vector of an AAV-ITR based gene delivery system of the present disclosure are from AAV7. In some aspects, the ITR of the AAV-ITR vector and the Rep68 mRNA of the Rep vector of an AAV-ITR based gene delivery system of the present disclosure are from AAV8. In some aspects, the ITR of the AAV-ITR vector and the Rep68 mRNA of the Rep vector of an AAV-ITR based gene delivery system of the present disclosure are from AAV9. In some aspects, the ITR of the AAV-ITR vector and the Rep68 mRNA of the Rep vector of an AAV-ITR based gene delivery system of the present disclosure are from AAV10. In some aspects, the ITR of the AAV-ITR vector and the Rep68 mRNA of the Rep vector of an AAV-ITR based gene delivery system of the present disclosure are from AAV11. In some aspects, the ITR of the AAV-ITR vector and the Rep68 mRNA of the Rep vector of an AAV-ITR based gene delivery system of the present disclosure are from AAV12. In some aspects, the ITR of the AAV-ITR vector and the Rep68 mRNA of the Rep vector of an AAV-ITR based gene delivery system of the present disclosure are from AAV13.

In some aspects, the ITR of the AAV-ITR vector can be selected from an AAV serotype selected from the group consisting of AAV1, AAV2, AAV3, AAV3B, AAV4, AAV5, AAV6, AAV7, AAV8, AAV9, AAV10, AAV11, AAV12 and AAV13, and the Rep78 mRNA of the Rep vector can be selected from an AAV serotype selected from the group consisting of AAV1, AAV2, AAV3, AAV3B, AAV4, AAV5, AAV6, AAV7, AAV8, AAV9, AAV10, AAV11, AAV12 and AAV13. In some aspects, the ITR of the AAV-ITR vector can be selected from an AAV serotype selected from the group consisting of AAV1, AAV2, AAV3, AAV3B, AAV4, AAV5, AAV6, AAV7, AAV8, AAV9, AAV10, AAV11, AAV12 and AAV13, and the Rep68 mRNA of the Rep vector can be selected from an AAV serotype selected from the group consisting of AAV1, AAV2, AAV3, AAV3B, AAV4, AAV5, AAV6, AAV7, AAV8, AAV9, AAV10, AAV11, AAV12 and AAV13.

The AAV-ITR based gene delivery system of the present disclosure can be generally described as an AAV-ITR vector comprising a linear DNA fragment flanked by two Parvo virus ITR (e.g., AAV2 ITR) wherein the linear dsDNA fragment comprises a transcription unit for expression of a gene of interest, and wherein the AAV-ITR vector is co-delivered with a Rep vector comprising a Parvo viral replicase mRNA (e.g., Rep78 of AAV2) for transient transcription of the replicase. The replication of the linear AAV-ITR vector only requires Rep68/78 and host cell proteins such as Polymerase δ (Pol δ), Replication Factor C (RFC), Proliferating Cell Nuclear Antigen (PCNA) and MCM complex in dividing cells. In some aspects, the AAV-base gene delivery system of the present disclosure can express two or more therapeutic polypeptides, therapeutic polynucleotides, or combinations thereof using a bicistronic element or 2A peptide.

The linear parvovirus AAV-ITR vector can be temporarily amplified by co-delivery with the Rep vector encoding Rep68 or Rep78 mRNA, which would result in the transient expression of Rep68 or Rep78 protein, supporting the replication of the AAV-ITR vector in dividing cells. The increased copy number of the AAV-ITR DNA in transduced cells enhances the integration frequency of AAV-ITR DNA into host cell genome in the presence of Rep68 or Rep78, which results in higher percentage of persistent transgene expression in the target cell population.

In some aspects, the AAV-ITR vector and/or Rep vector are encapsulated in a delivery system. In some aspects, the delivery system is a lipidic delivery system, e.g., an LNP or liposome, or a polymeric delivery system, or combination thereof. In some aspects, the AAV-ITR based gene delivery system of the present disclosure can be directed to a specific cell (e.g., T cell) or tissue using the methods and targeting compositions disclose in U.S. Provisional Patent Application No. 63/594,875; filed Oct. 31, 2023, which is herein incorporated by reference in its entirety.

In some aspects, the AAV-ITR vector and/or Rep vector are encapsulated in a lipidic delivery system, in a polymeric delivery system, or in a combination thereof, e.g., one vector can be encapsulated in a lipidic delivery system and the other vector can be encapsulated in a polymeric delivery system, or each one can be encapsulated in a lipidic delivery system or polymeric delivery system, or both can be co-encapsulated in a single lipidic delivery system or polymeric delivery system.

As used herein, the term “lipidic delivery system” refers to a lipid-based delivery vehicle or set thereof such as liposomes, lipid nanoparticles, or combinations thereof that that encapsulates a nucleic acid (e.g., a dsDNA, an mRNA, or both) and mediates the transfection of the nucleic acid into the cytoplasm of a target cell. As used herein, the term “polymeric delivery system” refers to a synthetic or natural polymer or a combination thereof that is capable of interacting with a nucleic acid (e.g., a dsDNA, a mRNA, or both) and mediates the transfection of the nucleic acid into the cytoplasm of a target cell.

In some aspects, transfection of an AAV-ITR vector of the present disclosure into a target eukaryotic cell (e.g., a T cell) or tissue (e.g., muscle) by using a lipidic or polymeric delivery systems or other methods known in the art (e.g., electroporation or calcium phosphate-mediate transfection) results in the integration of the sequence flanked by the ITRs into the genome of the target cell. In some aspects, transfection of a Rep vector of the present disclosure into a target eukaryotic cell results in the transient expression of the Rep protein (e.g., AAV2 Rep78 or Rep68 or a functional fragment thereof). In some aspects, the duration of the transient expression of the Rep78 or Rep68 can be modulated by regulatory elements present in the Rep vector, e.g., promoters. In some aspects, the duration of the transient expression of the Rep78 or Rep68 can be modulated by the presence of chemical modifications (e.g., replacement of uridines with pseudouridines) in the sequence of the mRNA encoding the Rep78 or Rep68 protein, wherein the chemical modifications alter (e.g., improve) the resistance of the mRNA by endogenous nucleases in the target cell.

Modified mRNAs: In some aspects, the expression of the Rep protein encoded by the Rep vector can be modulated by incorporating nucleotides analogues that increase the resistance of the mRNA encoding the Rep protein to endonucleases. In some aspects, the mRNA encoding the Rep protein, e.g., a Rep78 or Rep68 protein, comprises non-naturally occurring nucleotide analogues, e.g., nucleotides which have modified sugar moieties, such as bicyclic nucleotides or 2′ modified nucleotides, such as 2′ substituted nucleotides. The replacement of naturally occurring nucleotides with non-naturally analogues can confer desirable characteristics or properties to the mRNA, for example, increased resistance to degradation or stability. In some aspects, the nucleotide analogues increase resistance to intracellular nucleases and/or increase ease of transport into the cell. Specific examples of nucleoside analogues are described by, e.g. Freier & Altmann (1997) Nucl. Acid Res. 25:4429-4443, and Uhlmann (2000) Curr. Opinion in Drug Development 3:293-213.

The present disclosure provides for modified nucleosides and nucleotides of a mRNA polynucleotide encoding a Rep78 or Rep68 protein. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides can by synthesized by any useful method, such as, for example, chemically, enzymatically, or recombinantly, to include one or more modified or non-natural nucleosides. Polynucleotides can comprise a region or regions of linked nucleosides. Such regions can have variable backbone linkages. The linkages can be standard phosphodiester linkages, in which case the polynucleotides would comprise regions of nucleotides.

The modified polynucleotides disclosed herein can comprise various distinct modifications. In some aspects, the modified polynucleotides can contain one, two, or more (optionally different) nucleoside or nucleotide modifications. In some aspects, a modified polynucleotide, introduced to a cell can exhibit one or more desirable properties, e.g., improved protein expression, reduced immunogenicity, or reduced degradation in the cell, as compared to an unmodified polynucleotide.

Structural Modifications: In some aspects, the mRNA polynucleotide encoding a Rep78 or Rep68 protein or a functional fragment or variant thereof is structurally modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” can be chemically modified to “AT-5meC-G”. The same polynucleotide can be structurally modified from “ATCG” to “ATCCCG”. Here, the dinucleotide “CC” has been inserted, resulting in a structural modification to the polynucleotide.

Chemical Modifications: In some aspects, the mRNA polynucleotide encoding a Rep78 or Rep68 protein or a functional fragment or variant thereof is chemically modified. As used herein in reference to a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population. Generally, herein, these terms are not intended to refer to the ribonucleotide modifications in naturally occurring 5′-terminal mRNA cap moieties.

In some aspects, the mRNA polynucleotide encoding a Rep78 or Rep68 protein or a functional fragment or variant thereof can have a uniform chemical modification of all or any of the same nucleoside type or a population of modifications produced by mere downward titration of the same starting modification in all or any of the same nucleoside type, or a measured percent of a chemical modification of all any of the same nucleoside type but with random incorporation, such as where all uridines are replaced by a uridine analog, e.g., 5-methoxyuridine. In another aspect, the polynucleotides can have a uniform chemical modification of two, three, or four of the same nucleoside type throughout the entire polynucleotide (such as all uridines and all cytosines, etc. are modified in the same way).

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s.

Base Modifications: Modifications of polynucleotides (e.g., RNA polynucleotides, such as mRNA polynucleotides) that are useful in the composition of the present disclosure are selected from the group consisting of pseudouridine (W), N1-methylpseudouridine (m1ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine), 5-methoxyuridine and 2′-O-methyl uridine. In some aspects, the polynucleotide (e.g., RNA polynucleotide, such as mRNA polynucleotide) includes a combination of at least two (e.g., 2, 3, 4 or more) of the aforementioned modified nucleobases.

In some aspects, modified nucleobases in the polynucleotide (e.g., RNA polynucleotide, such as mRNA polynucleotide) are selected from the group consisting of 5-methoxyuridine (mo5U), 1-methyl-pseudouridine (m1ψ), 5-methyl-cytidine (m5C), pseudouridine (ψ), α-thio-guanosine and α-thio-adenosine. In some embodiments, the polynucleotide includes a combination of at least two (e.g., 2, 3, 4 or more) of the aforementioned modified nucleobases.

In some aspects, at least 95% of a type of nucleobases (e.g., uridine) in an mRNA polynucleotide encoding a Rep78 or Rep68 protein or a functional fragment or variant thereof have been replaced by one of the aforementioned modified nucleobases. In some aspects, at least 95% of uridine in an mRNA polynucleotide encoding a Rep78 or Rep68 protein or a functional fragment or variant thereof have been replaced by 5-methoxyuridine.

In some aspects, the mRNA polynucleotide encoding a Rep78 or Rep68 protein or a functional fragment or variant thereof comprises pseudouridine (W) and 5-methyl-cytidine (m5C). In some aspects, the mRNA polynucleotide encoding a Rep78 or Rep68 protein or a functional fragment or variant thereof comprises 5-methoxyuridine (5mo5U) and 5-methyl-cytidine (m5C). In some embodiments, the mRNA polynucleotide encoding a Rep78 or Rep68 protein or a functional fragment or variant thereof comprises 1-methyl-pseudouridine (m1ψ). In some aspects, the mRNA polynucleotide encoding a Rep78 or Rep68 protein or a functional fragment or variant thereof comprises 1-methyl-pseudouridine (m1ψ) and 5-methyl-cytidine (m5C). In some aspects, the mRNA polynucleotide encoding a Rep78 or Rep68 protein or a functional fragment or variant thereof comprises 2-thiouridine (s2U). In some aspects, the mRNA polynucleotide encoding a Rep78 or Rep68 protein or a functional fragment or variant thereof comprises 2-thiouridine and 5-methyl-cytidine (m5C). In some aspects, the mRNA polynucleotide encoding a Rep78 or Rep68 protein or a functional fragment or variant thereof comprises methoxy-uridine (mo5U). In some aspects, the mRNA polynucleotide encoding a Rep78 or Rep68 protein or a functional fragment or variant thereof comprises 5-methoxy-uridine (mo5U) and 5-methyl-cytidine (m5C). In some aspects, the mRNA polynucleotide encoding a Rep78 or Rep68 protein or a functional fragment or variant thereof comprises 2′-O-methyl uridine. In some aspects, the mRNA polynucleotide encoding a Rep78 or Rep68 protein or a functional fragment or variant thereof comprises 2′-O-methyl uridine and 5-methyl-cytidine (m5C). In some aspects, the mRNA polynucleotide encoding a Rep78 or Rep68 protein or a functional fragment or variant thereof comprises N6-methyl-adenosine (m6A). In some aspects, the mRNA polynucleotide encoding a Rep78 or Rep68 protein or a functional fragment or variant thereof comprises N6-methyl-adenosine (m6A) and 5-methyl-cytidine (m5C.

In some aspects, the mRNA polynucleotide encoding a Rep78 or Rep68 protein or a functional fragment or variant thereof is uniformly modified (e.g., fully modified, modified throughout the entire sequence) for a particular modification. For example, a polynucleotide can be uniformly modified with 5-methyl-cytidine (m5C), meaning that all cytosine residues in the mRNA sequence are replaced with 5-methyl-cytidine (m5C). Similarly, a polynucleotide can be uniformly modified for any type of nucleoside residue present in the sequence by replacement with a modified residue such as any of those set forth above.

In some aspects, the modified nucleobase is a modified cytosine. Examples of nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine. In some aspects, a modified nucleobase is a modified uridine. Example nucleobases and nucleosides having a modified uridine include 5-cyano uridine or 4′-thio uridine. In some aspects, a modified nucleobase is a modified adenine. Example nucleobases and nucleosides having a modified adenine include 7-deaza-adenine, 1-methyl-adenosine (m1A), 2-methyl-adenine (m2A), N6-methyl-adenosine (m6A), and 2,6-diaminopurine. In some aspects, a modified nucleobase is a modified guanine. Example nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

In some aspects, the mRNA polynucleotide encoding a Rep78 or Rep68 protein or a functional fragment or variant thereof is chemically modified by at least about 10%, at least about 20%, at least about 30%, at least about 40%, at least about 50%, at least about 60%, at least about 70%, at least about 80%, at least about 90%, at least about 95%, at least about 99%, or about 100%. In some aspects, the uridine nucleosides in the open reading frame encoding a Rep78 or Rep68 protein or a functional fragment or variant thereof are chemically modified by at least 10%, at least about 20%, at least about 30%, at least about 40%, at least about 50%, at least about 60%, at least about 70%, at least about 80%, at least about 90%, at least about 95%, at least about 99%, or about 100%. In some aspects, the adenine nucleosides in the open reading frame encoding a Rep78 or Rep68 protein or a functional fragment or variant thereof are chemically modified by at least about 10%, at least about 20%, at least about 30%, at least about 40%, at least about 50%, at least about 60%, at least about 70%, at least about 80%, at least about 90%, at least about 95%, at least about 99%, or about 100%. In some aspects, the cytosine nucleosides in the open reading frame encoding a Rep78 or Rep68 protein or a functional fragment or variant thereof are chemically modified by at least at least about 10%, at least about 20%, at least about 30%, at least about 40%, at least about 50%, at least about 60%, at least about 70%, at least about 80%, at least about 90%, at least about 95%, at least about 99%, or about 100%. In some aspects, the guanine nucleosides in the open reading frame encoding a Rep78 or Rep68 protein or a functional fragment or variant thereof are chemically modified by at least at least about 10%, at least about 20%, at least about 30%, at least about 40%, at least about 50%, at least about 60%, at least about 70%, at least about 80%, at least about 90%, at least about 95%, at least about 99%, or about 100%.

In some embodiments, the polynucleotides can include any useful linker between the nucleosides. Such linkers, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, —CH2-O—N(CH3)-CH2-, -CH2-N(CH3)-N(CH3)-CH2-, —CH2-NH—CH2-, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH3)-CH2-CH2-, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

Sugar Modifications: The modified nucleosides and nucleotides (e.g., building block molecules), which can be incorporated into a mRNA polynucleotide encoding a Rep78 or Rep68 protein or a functional fragment or variant thereof, can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C1-6 alkyl; optionally substituted C1-6 alkoxy; optionally substituted C6-10 aryloxy; optionally substituted C3-8 cycloalkyl; optionally substituted C3-8 cycloalkoxy; optionally substituted C6-10 aryloxy; optionally substituted C6-10 aryl-C1-6 alkoxy, optionally substituted C1-12 (heterocyclyl)oxy; a sugar (e.g., ribose, pentose, or any described herein); a polyethyleneglycol (PEG), —O(CH2CH2O)nCH2CH2OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C1-6 alkylene or C1-6 heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar. Such sugar modifications are taught International Patent Publication Nos. WO2013052523 and WO2014093924, the contents of each of which are incorporated herein by reference in their entireties.

Combinations of Modifications: The mRNA polynucleotide encoding a Rep78 or Rep68 protein or a functional fragment or variant thereof can include a combination of modifications to the sugar, the nucleobase, and/or the internucleoside linkage. These combinations can include any one or more modifications described herein. Unless otherwise noted, the modified nucleotides can be completely substituted for the natural nucleotides of the polynucleotides of the invention. As a non-limiting example, the natural nucleotide uridine can be substituted with a modified nucleoside described herein. In another non-limiting example, the natural nucleotide uridine can be partially substituted (e.g., about 0.1%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 99.9%) with at least one of the modified nucleoside disclosed herein. Any combination of base/sugar or linker can be incorporated into the polynucleotides of the invention and such modifications are taught in WO2013052523, WO2014093924, US20130115272 and US20150307542, the contents of each of which are incorporated herein by reference in its entirety.

In some aspect, an AAV-ITR based gene delivery system of the present disclosure or a component thereof, such as an AAV-ITR vector or Rep vector, is encapsulated in a liposome or lipid nanoparticle. In some aspects, the AAV-ITR vector is encapsulated in a first lipidic delivery system (e.g., a first liposome or LNP). In some aspects, the AAV-ITR vector is encapsulated in a first lipidic delivery system (e.g., a first liposome or LNP) optimized to deliver (transfect) a double stranded DNA into a target cell. In some aspects, the Rep vector is encapsulated in a second lipidic delivery system (e.g., a second liposome or LNP). In some aspects, the Rep vector is encapsulated in a second lipidic delivery system (e.g., a second liposome or LNP) optimized to deliver (transfect) an mRNA into a target cell. In some aspects, the first lipidic delivery system (e.g., a first liposome or LNP) containing an AAV-ITR vector and the second lipidic delivery system (e.g., a second liposome or LNP) containing a Rep vector have the same lipidic composition. In some aspects, the first lipidic delivery system (e.g., a first liposome or LNP) containing an AAV-ITR vector and the second lipidic delivery system (e.g., a second liposome or LNP) containing a Rep vector have different lipidic compositions.

In some aspects, the AAV-ITR vector and Rep vector are coencapsulated in a single lipidic delivery system (e.g., a single liposome or LNP). In some aspects, the AAV-ITR vector and the Rep vector are coencapsulated in a single polymeric delivery system.

In some aspects, the AAV-ITR vector in encapsulated in a liposome. In some aspects, the Rep vector is encapsulated in a liposome. In some aspects, both the AAV-ITR vector and Rep vector are encapsulated in liposomes. In some aspects, the AAV-ITR vector is encapsulated in a first liposome and the Rep vector is encapsulated in a second liposome. In some aspects, the first and second liposome have the same lipidic composition. In some aspects, the first and second liposome have different lipidic compositions.

In some aspects, the AAV-ITR vector in encapsulated in a LNP. In some aspects, the Rep vector is encapsulated in a LNP. In some aspects, both the AAV-ITR vector and Rep vector are encapsulated in LNPs. In some aspects, the AAV-ITR vector is encapsulated in a first LNP and the Rep vector is encapsulated in a second LNP. In some aspects, the first and second LNP have the same lipidic composition. In some aspects, the first and second LNP have different lipidic compositions.

In some aspects, the AAV-ITR vector is encapsulated in a liposome and the Rep vector is encapsulated in a LNP. In some aspects, the AAV-ITR vector is encapsulated in a LNP and the Rep vector is encapsulated in a liposome.

The lipidic delivery systems used to encapsulate and deliver (transfect) the AAV-ITR based gene delivery systems of the disclosure can be selected from commercially available systems and/or can be specially designed. For example, in some aspects, the AAV-ITR vector can be encapsulated in a LIPOFECTAMINE™ 2000 liposome. In some aspects, the Rep vector can be encapsulated in a LIPOFECTAMINE™ MESSENGERMAX™ liposome. In some aspects, the AAV-ITR vector can be encapsulated in a LIPOFECTAMINE™ 2000 liposome and the Rep vector can be encapsulated in a LIPOFECTAMINE™ MESSENGERMAX™ liposome.

Other commercially available lipidic delivery systems that can be used to encapsulate and deliver the AAV-ITR based gene delivery system of the present disclosure are, e.g., LIPOFECTAMINE™ 3000, LIPOFECTAMINE™ RNAiMAX, LIPOFECTINE™ LIPOFECTAMINE™ LTX, LIPOFECTAMINE™ Stem Transfection Reagent, LIPOFECTAMINE™ CRISPRMAX™ Cas9 Transfection Reagent, VIAFECT™ (cationic system optimized for transient expression), TRANSTAST™, INVIVOFECTAMINE™ 3.0, MIRUS BIO™ TRANSIT™-2020, GIBCO™ EXPIFECTAMINE™.

In some aspects, an AAV ITR-based gene delivery system of the present disclosure can be transfected into a target cell using electroporation. In some aspects, an AAV-ITR based gene delivery system of the present disclosure can be transfected into a target cell using a nonliposomal formulation such as FUGENE™ 6 or FUGENE™ I-ID. In some aspects, an AAV-ITR based gene delivery system of the present disclosure can be transfected into a target cell using a calcium phosphate-mediated system such as PROFECTION™. See, e.g., Chong et al. (2021) Peer J. 9:e11165; and Fus-Kujawa et al. (2021) Front. Bioeng. Biotechnol. 9:701031, which are herein incorporated by reference in their entireties.

In general, the AAV ITR-based gene delivery system of the present disclosure encompasses any delivery system or combination thereof that can deliver an AAV-ITR vector disclosed herein and a corresponding Rep vector disclosed herein to a specific cell or tissue.

In some aspects, the AAV ITR-based gene delivery system of the present disclosure can be targeted to a specific cell type (e.g., T cells, B cells or NK cells) or tissue (e.g., muscle) via a targeting molecule (e.g., an antibody) that specifically binds to specific surface proteins (e.g., one, two, three, or more) on the surface of a particular cell type (e.g., a T cell) or tissue (e.g., muscle tissue) thereby directing the AAV-ITR based gene delivery system of the present disclosure to the targeted cell type or tissue. In some aspects that targeting molecule, e.g., a MSTAR antibody, can be covalently attached to the surface of the lipidic delivery system (e.g., a liposome or LNP) or to a polymeric delivery system.

In some aspects, the lipidic delivery system comprises a LNP or liposome comprising a surface anchored targeting molecule (e.g., an antibody) that specifically binds to at least one cell- (e.g., a T cell, B cell or NK cell) or tissue- (e.g., muscle tissue) specific surface protein. In some aspects, the lipidic delivery system comprises an LNP comprising at least two surface anchored targeting molecules (e.g., two antibodies), wherein each cell- or tissue-specific targeting molecule specifically binds to at least one cell- or tissue-specific target protein (e.g., a first antibody targets a first target protein and a second antibody targets a second target protein). In some aspects, the lipidic delivery system comprises a set of LNPs comprising at least two LNPs, wherein the first LNP comprises a first cell- or tissue-specific targeting molecule (e.g., an antibody) that specifically binds to a first cell or tissue specific surface protein, and the second LNP comprises a second cell- or tissue-specific targeting molecule that specifically binds to a second cell- or tissue-specific specific surface protein.

The present disclosure provides a targeted delivery system comprising a LNP, liposome, or combination thereof comprising a surface anchored targeting molecule (e.g., antibody) or combination thereof that specifically binds to one or more specific surface proteins in a target cell (e.g., a T cell, B cell, muscle cell, fibroblast, etc.) or tissue (e.g., a specific organ, a tumor, or the microenvironment of a tumor) and efficiently delivers an AAV ITR-based gene delivery system of the present disclosure encapsulated in the LNP, liposome, or combination thereof to the target cell or tissue.

Any combination of cells within the hematopoietic lineage can be targeted by the targeted delivery systems of the present disclosure. In some aspects, target cells selected from bone marrow cells, hematopoietic stem cells (HSCs), hematopoietic stem and progenitor cells (HSPCs), peripheral blood mononuclear cells (PBMCs), myeloid progenitor cells, lymphoid progenitor cells, T-cells, B-cells, NKT cells, NK cells, dendritic cells, monocytes, granulocytes, erythrocytes, megakaryocytes, mast cells, basophils, eosinophils, neutrophils, macrophages, erythroid progenitor cells (e.g., HUDEP cells), megakaryocyte-erythroid progenitor cells (MEPs), common myeloid progenitor cells (CMPs), multipotent progenitor cells (MPPs), hematopoietic stem cells (HSCs), short term HSCs (ST-HSCs), IT-HSCs, long term HSCs (LT-HSCs), endothelial cells, neurons, astrocytes, pancreatic cells, pancreatic β-islet cells, liver cells, muscle cells, skeletal muscle cells, cardiac muscle cells, hepatic cells, fat cells, intestinal cells, cells of the colon, and cells of the stomach.

Non-limiting examples of various applications of the targeted AAV ITR-based gene delivery systems of the present disclosure (e.g., for targeting neurons, cells of the pancreas, hematopoietic stem cells and multipotent progenitors, etc.) are described next. For example, hematopoietic stem cells and multipotent progenitors can be targeted for gene editing (e.g., insertion) in vivo or ex vivo. As another example, pancreatic cells (e.g., β islet cells) can be targeted, e.g., to treat pancreatic cancer, to treat diabetes, etc. As another example, somatic cells in the brain such as neurons can be targeted (e.g., to treat indications such as Huntington's disease, Parkinson's, e.g., due to LRRK2 mutations, and ALS, e.g., due to SOD1 mutations). As another example, endothelial cells and cells of the hematopoietic system (e.g., megakaryocytes and/or any progenitor cell upstream of a megakaryocyte such as a megakaryocyte-erythroid progenitor cell (MEP), a common myeloid progenitor cell (CMP), a multipotent progenitor cell (MPP), a hematopoietic stem cells (HSC), a short term HSC (ST-HSC), an IT-HSC, a long term HSC (LT-HSC) can be targeted with a targeted AAV-based gene delivery system of the present disclosure to treat Von Willebrand's disease. For example, a cell (e.g., an endothelial cell, a megakaryocyte and/or any progenitor cell upstream of a megakaryocyte such as an MEP, a CMP, an MPP, an HSC such as an ST-HSC, an IT-HSC, and/or an LT-HSC) harboring a mutation in the gene encoding von Willebrand factor (VWF) can be targeted (in vitro, ex vivo, or in vivo) in order to introduce an active protein (e.g., via delivery of a functional VWF protein and/or a nucleic acid encoding a functional VWF protein) and/or in order to edit the mutated gene, e.g., by introducing a replacement sequence (e.g., via delivery of a gene editing tool and delivery of a DNA donor template). In some of the above cases (e.g., related to treating Von Willebrand's disease, related to targeting a cell harboring a mutation in the gene encoding VWF), a targeted AAV-based gene delivery system of the present disclosure provides for targeted binding to E-selectin.

As another example, a cell of a stem cell lineage (e.g., a stem and/or progenitor cell of the hematopoietic lineage, e.g., a GMP, MEP, CMP, MLP, MPP, and/or an HSC) can be targeted with a targeted AAV-based gene delivery system of the present disclosure to increase expression of stem cell factor (SCF) in the cell, which can therefore drive proliferation of the targeted cell. For example, a targeted AAV-based gene delivery system of the present disclosure can be used to deliver SCF and/or a nucleic acid (DNA or mRNA) encoding SCF to the targeted cell.

Methods and compositions of this disclosure can be used to treat any number of diseases, including any disease that is linked to a known causative mutation, e.g., a mutation in the genome. For example, methods and compositions of this disclosure can be used to treat sickle cell disease, β thalassemia, HIV, myelodysplasia syndromes, JAK2-mediated polycythemia vera, JAK2-mediated primary myelofibrosis, JAK2-mediated leukemia, and various hematological disorders. As additional non-limiting examples, the methods and compositions of this disclosure can also be used for B-cell antibody generation, immunotherapies (e.g., delivery of a checkpoint blocking reagent), and stem cell differentiation applications.

In some aspects, the AAV-ITR vector comprises a polynucleotide encoding a multispecific antibody such as a tetravalent/trispecific or a tetravalent/tetraspecific antibody that neutralizes a COVID-19 infective agent such as any known variant of a SARS CoV-2 virus. In some aspects, the antibody is a MSTAR antibody. In some aspects, the AAV-ITR vector can be administered to a COVID-19 patient in the form of a delivery system comprising a combination of the AAV-ITR and a Rep vector comprising a Rep68/78 mRNA, e.g., encapsulated in a lipidic delivery system disclosed herein (e.g., a LNP, a liposome, or a combination thereof), or in a polymeric delivery system, or in a combination thereof.

In some aspects, the AAV-ITR vector comprises a polynucleotide encoding a multispecific antibody directed against influenza or against HIV. In some aspects, the antibody can be a MSTAR antibody. In some aspects, the AAV-ITR vector can be administered to an HIV/AIDS patient in the form of a delivery system comprising a combination of the AAV-ITR and a Rep vector comprising a Rep68/78 mRNA, e.g., encapsulated in a lipidic delivery system disclosed herein (e.g., a LNP, a liposome, or a combination thereof), or in a polymeric delivery system, or in a combination thereof.

In some aspects, the AAV-ITR vector comprises a polynucleotide encoding a GLP-1 or GIP agonist or a glucagon agonist. In some aspects, the AAV-ITR vector can be administered to a type 1 diabetes patient in the form of a delivery system comprising a combination of the AAV-ITR and a Rep vector comprising a Rep68/78 mRNA, e.g., encapsulated in a lipidic delivery system disclosed herein (e.g., a LNP, a liposome, or a combination thereof), or in a polymeric delivery system, or in a combination thereof.

In some aspects, a lipidic delivery system (i.e., a LNP, liposome, or a combination thereof) comprising a surface anchored targeting molecule (e.g., an anti-CD3 antibody, such as an MSAT anti CD3 antibody) can deliver an AAV ITR-based gene delivery system of the present disclosure to a T-cell. In some aspect, the surface anchored T-cell targeting molecule (e.g., an antibody) specifically binds to at least two T-cell specific surface proteins (e.g., CD3 and CD28).

In addition to the AAV-ITR vector and Rep vector components of an AAV-ITR based gene delivery system of the present disclosure, there is a wide range of therapeutic payloads that can be co-encapsulated or attached (e.g., via cleavable linkers) to the lipidic delivery systems of the present disclosure (e.g., LNP, liposomes and combinations thereof) including for example, small molecules, proteins, nucleic acids, or diagnostic agents.

In some aspects, the polynucleotide encoding a therapeutic polypeptide, a therapeutic polynucleotide, or a combination thereof in the AAV-ITR component of an AAV ITR-based gene delivery system of disclosed herein can comprise a dsDNA encoding, e.g., an enzyme (e.g., for gene replacement therapy), a component of a gene editing system, (e.g., a Cas enzyme and/or a gRNA), a vaccine (e.g., a SARS-CoV2 vaccine such as mRNA-1273 or BNR162b2), a CAR, a TCR, a hormone, a growth factor, or an anti-inflammatory protein (e.g., a cytokine).

In some aspects, an AAV-ITR vector of the present disclosure comprises a genetic cassette encoding more than one therapeutic protein or polynucleotide. Thus, in some aspects, the AAV-ITR component of an AAV-ITR based gene delivery system of the present disclosure comprises a bicistronic construct, i.e., a polynucleotide comprising at least two cistrons. The presence of a genetic cassette encoding more than one gene of interest (e.g., two therapeutic proteins, two polypeptide subunits of a therapeutic protein such as an antibody, or components of a gene editing system such as a Cas nuclease and a gRNA) and further including elements such as at least one IRES or at least one 2A element allows co-expression from a single promoter.

As used herein, a “bicistronic” polynucleotide refer to a polynucleotide that upon transcription produces a single mRNA which comprises two coding sequences (i.e., cistrons) and encodes more than one product, e.g., two proteins. As used herein, a “polycistronic” polynucleotide refer to a polynucleotide that upon transcription produces a single mRNA which comprises more than two coding sequences (i.e., cistrons) and encodes more than two products, e.g., two proteins.

A bicistronic or polycistronic mRNA can comprise any element known in the art to allow for the translation of two or more genes from the same mRNA molecule including, but not limited to, an Internal Ribosome Entry Site (IRES) element, a T2A element, a P2A element, an E2A element, and an F2A element.

In some aspects, the AAV-ITR vector comprises two coding regions (e.g., protein coding regions) separated by an IRES element. In some aspects, the AAV-ITR vector comprises two coding regions (e.g., protein coding regions) separated by a 2A element. In some aspects, the AAV-ITR vector comprises three coding regions (e.g., protein coding regions) separated by IRES elements between each coding region. In some aspects, the AAV-ITR vector comprises three coding regions (e.g., protein coding regions) separated by a 2A element between each coding region.

In some aspects, the AAV-ITR vector comprises two protein-coding regions separated by IRES elements between each coding region, wherein the two proteins are polypeptide components of an antibody. In some aspects, the AAV-ITR vector comprises two protein coding regions) separated by a 2A element between each coding region, wherein the two proteins are polypeptide components of an antibody. In some aspects, the AAV-ITR vector comprises two protein-coding regions separated by IRES elements between each coding region, wherein the two proteins are polypeptide components of an MSTAR antibody. In some aspects, the AAV-ITR vector comprises two protein coding regions) separated by a 2A element between each coding region, wherein the two proteins are polypeptide components of an MSTAR antibody.

In some aspects, two cistrons are connected by a 2A element (e.g., a P2A element). In some aspects, two cistrons are connected by an IRES element. IRES elements are RNA regions that recruit the 40S ribosomal subunit through cap-independent mechanisms. IRES elements often adopt complex RNA structures, which serve as the anchoring site for the ribosome guided by RNA-RNA and/or RNA-protein interactions. 2A self-cleaving peptides, or 2A peptides, are a class of 18-22 aa-long peptides, which can induce ribosomal skipping during translation of a protein in a cell. 2A elements are good candidates to replace IRES because of its small size and high cleavage efficiency between genes upstream and downstream of the 2A peptide. In some aspects, the 2A element is a P2A element (porcine teschovirus-1 2A). In other aspects, the 2A element is a F2A element (foot-and-mouth disease virus), E2A element (equine rhinitis A virus), or T2A element (thosea asigna virus 2A). In some aspects, the 2A element portion of a bicistronic polynucleotide comprises 2 or more 2A elements in tandem, wherein the 2A elements are selected from P2A, E2A, F2A, E2A, and T2A. In some aspects, the 2A element is P2A-T2A. In some aspects, the 2A element is 2A-T2A-E2A.

The AAV-ITR based gene delivery system of the present disclosure can be targeted to any cell, tissue, or cellular compartment as long as the appropriate targeting molecule or set thereof specifically binds to one or more target molecules (e.g., receptors) on the target cell, tissue, or cellular compartment. In some aspects, an AAV-ITR based gene delivery system of the present disclosure can be targeted to a human cell, e.g., a T cell. In some aspects, an AAV-ITR based gene delivery system of the present disclosure can be targeted to an organ, e.g., liver. In some aspects, an AAV-ITR based gene delivery system of the present disclosure can be targeted to a tissue, e.g., muscle tissue. In some aspects, an AAV-ITR based gene delivery system of the present disclosure can be targeted to a tumor.

In some aspects, the present disclosure provides an AAV-ITR based gene delivery system for targeted delivery comprising a single population of liposomes or LNP with targeting molecules (e.g., monospecific or bispecific antibodies) conjugated to their surface, wherein the single population of liposomes or LNP contains the AAV-ITR vector and Rep vector. In some aspects, the present disclosure an AAV-ITR based gene delivery system for targeted delivery comprising two populations of liposomes or LNP with targeting molecules (e.g., monospecific or bispecific antibodies) conjugated to their surfaces, wherein a first population of liposomes or LNP contains the AAV-ITR vector and a second population of liposomes or LNP contains the Rep vector.

In some aspects, the present disclosure an AAV-ITR based gene delivery system for targeted delivery comprising a single population of LNP with targeting molecules (e.g., monospecific or bispecific antibodies) conjugated to their surface, wherein the single population of LNP contains the AAV-ITR vector and Rep vector. In some aspects, the present disclosure an AAV-ITR based gene delivery system for targeted delivery comprising a single population of liposomes with targeting molecules (e.g., monospecific or bispecific antibodies) conjugated to their surface, wherein the single population of liposomes contains the AAV-ITR vector and Rep vector.

In some aspects, the disclosure provides an AAV-ITR based gene delivery system for targeted delivery comprising two populations of liposomes with targeting molecules (e.g., monospecific or bispecific antibodies) conjugated to their surfaces, wherein a first population of liposomes contains the AAV-ITR vector and a second population contains the Rep vector.

In some aspects, the present disclosure provides an AAV-ITR based gene delivery system for targeted delivery comprising two populations of LNP with targeting molecules (e.g., monospecific or bispecific antibodies) conjugated to their surfaces, wherein a first population of LNP contains the AAV-ITR vector and a second population of LNP contains the Rep vector.

In some aspects, the present disclosure provides an AAV-ITR based gene delivery system for targeted delivery comprising a population of liposomes with targeting molecules (e.g., monospecific or bispecific antibodies) conjugated to their surfaces which contains the AAV-ITR vector, and a population of LNP with targeting molecules (e.g., monospecific or bispecific antibodies) conjugated to their surfaces which contains the Rep vector.

In some aspects, the present disclosure provides an AAV-ITR based gene delivery system for targeted delivery comprising a population of LNP with targeting molecules (e.g., monospecific or bispecific antibodies) conjugated to their surfaces which contains the AAV-ITR vector, and a population of liposomes with targeting molecules (e.g., monospecific or bispecific antibodies) conjugated to their surfaces which contains the Rep vector.

In some aspects, targeting of a liposome, LNP, or other non-lipidic delivery system, e.g., a polymeric delivery system, comprising an AAV-ITR based gene delivery system of the present disclosure to a specific cell type (e.g., a T cell) or tissue (e.g., muscle) can be achieved, e.g., by a Fab fragment or an MSTAR antibody. In some aspects, the MSTAR antibody comprises a single chain CHCL. In some aspects, the MSTAR antibody does not comprise a single chain CHCL. In some aspects, the MSTAR antibody is monospecific. In some aspects, the MSTAR antibody is bispecific. In some aspects, the MSTAR antibody is trispecific. In some aspects, the MSTAR antibody is multispecific.

The lipidic delivery systems disclosed herein a not limited to liposomes or LNPs. In some aspects, the lipidic delivery system is a nanocarrier other than a LNP, e.g., a micelle. Micelles are colloidal dispersions with a particle size between 5-100 nm. In some aspects, the nanocarrier is an extracellular vesicle (EV). In some aspects, the extracellular vesicle is an exosome (size between 30-200 nm) or a microvesicle (size between 100-1000 nm).

In some aspects, the amount of Rep vector in an AAV-ITR based gene delivery system of the present disclosure is about 30%, about 10%, about 3.5%, about 1.5%, or about 0.5% of the amount of AAV-ITR vector. In some aspects, AAV-ITR vector and Rep vector are present in an AAV-ITR based gene delivery system of the present disclosure at an about 100:30 ratio, about 100:10 ratio, about 100:3.5 ratio, about 100:1.5 ratio, or about 100:0.5 ratio.

In some aspects, the amount of Rep vector in an AAV-ITR based gene delivery system of the present disclosure is about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4.5%, about 4%, about 3.5%, about 3%, about 2.5%, about 2%, about 1.5%, about 1%, about 0.5%, about 0.4%, about 0.3%, about 0.2% or about 0.1% of the amount of AAV-ITR vector.

In some aspects, the amount of Rep vector in an AAV-ITR based gene delivery system of the present disclosure is less than about 40%, less than about 39%, less than about 38%, less than about 37%, less than about 36%, less than about 35%, less than about 34%, less than about 33%, less than about 32%, less than about 31%, less than about 30%, less than about 29%, less than about 28%, less than about 27%, less than about 26%, less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about %, less than about 9%, less than about 8%, less than about 7%, less than about 6%, less than about 5%, less than about 4.5%, less than about 4%, less than about 3.5%, less than about 0.5%, less than about 2.5, less than about 2%, less than about 1.5%, less than about 1%, less than about 0.5%, less than about 0.4%, less than about 0.3%, less than about 0.2% or less than about 0.1% of the amount of AAV-ITR vector.

In some aspects, the amount of Rep vector in an AAV-ITR based gene delivery system of the present disclosure is between about 40% and about 35%, between about 35% and about 30%, between about 30% and about 25%, between about 25% and about 20%, between about 20% and about 15%, between about 15% and about 10%, between about 10% and about 5%, between about 5% and about 0.1%, between about 10% and about 8%, between about 9% and about 7%, between about 8% and about 6%, between about 7% and about 5%, between about 6% and about 4%, between about 5% and about 3%, between about 4% and about 2%, between about 3% and about 1%, between about 2% and about 0.1%, between about 5% and about 4%, between about 4% and about 3%, between about 3% and about 2%, between about 2% and about 1%, between about 1% and about 0.1%, between about 5% and about 4.5%, between about 4.5% and about 4%, between about 4% and about 3.5%, between about 3.5% and about 3%, between about 3% and about 2.5%, between about 2.5% and about 2%, between about 2% and about 1.5%, between about 1.5% and about 1%, between about 1% and about 0.5%, between about 0.5% and about 0.1%, between about 1% and about 0.9%, between about 0.9% and about 0.8%, between about 0.8% and about 0.7%, between about 0.7% and about 0.6%, between about 0.6% and about 0.5%, between about 0.5% and about 0.4%, between about 0.4% and about 0.3%, between about 0.3% and about 0.2%, or between about 0.2% and about 0.1% of the amount of AAV-ITR vector.

The disclosure provides methods of treatment achieving sustained gene expression by transfecting target cells with a linear dsDNA vector comprising a polynucleotide encoding a therapeutic polypeptide, polynucleotide, or combination thereof flanked by parvoviral ITR (AAV-ITR vector), wherein the dsDNA vector is co-transfected with a mRNA vector encoding a parvoviral Rep78 or Rep68 protein which is transiently co-expressed in the target cell (Rep vector).

The methods of the present disclosure comprise the administration of a combination of vectors, an AAV-ITR vector (e.g., a linear dsDNA vector encoding a therapeutic polypeptide, polynucleotide, or combination thereof) and a Rep vector, wherein the Rep vector (e.g., a Rep78 vector) is in a sufficient molar amount (ratio) relative to the amount of AAV-ITR vector to induce the transient expression of Rep protein (e.g., Rep78) encoded by the Rep vector. The linear parvovirus ITR-DNA vector is thus temporarily amplified which results in persistent gene expression of the therapeutic polypeptide, polynucleotide, or combination thereof encoded by the AAV-ITR vector through episomal maintenance and/or thorough chromosomal integration. The Rep protein (e.g., Rep78), introduced transiently, increases the frequency of linear AAV-ITR DNA integration in dividing cells.

The AAV-ITR based gene delivery systems of the present disclosure result in an increased copy number of the AAV-ITR vector transduced into cells, which enhances the integration frequency of the AAV-ITR DNA into host cell genome, e.g., at AAVS1, resulting in higher percentages of persistent transgene expressing cell populations. In vivo, this results in long-term, high-level gene expression, providing therapeutic opportunities to treat various conditions and/or diseases including infectious diseases, cancers, autoimmune diseases, and genetic diseases. Furthermore, the co-administration (co-transfection) of a Rep vector comprising a Rep78 or Rep68 mRNA together with the AAV-ITR vector comprising a polynucleotide encoding a gene on interest results in a short-time (transient) expression of the Rep78 or Rep68 protein that can avoid generating an immune response by the host which can result in the elimination of transgene expressing cells. Therefore, increased AAV-ITR copy number and increased AAV-ITR genomic integration in transduced cells and avoidance of immune-mediated cell elimination, together support sustained therapeutic transgene protein expression. This allows the AAV-ITR based gene delivery systems of the present disclosure to be used to treat, e.g., systemic infections wherein the infectious agent such as a virus (e.g., SARS-CoV2, HIV, influenza, etc.) infects multiple cell types and organs. Furthermore, this also allows that methods, nucleic acids, compositions and delivery systems described herein can be used to target specific cell types, such as T cells, B cells, liver cells, lung cells, and different cancer cells to treat localized diseases, infections or other conditions.

The present disclosure provides diagnostic methods and methods of treatment comprising the use the AAV-ITR based gene delivery systems of the present disclosure, which can be encapsulated as disclosed about and elsewhere is this disclosure, e.g., in lipidic delivery systems (e.g., liposomes, lipid nanoparticles, micelles, or combination thereof), polymeric delivery systems, extracellular vesicles, exosomes (e.g., natural, synthetic, semisynthetic, engineered, nanovesicles, hybrid exosomes, or exosome-mimetics, e.g., biomimetic exosomes).

The present disclosure provides a method of delivering a therapeutic polypeptide, polynucleotide, or a combination thereof in vivo or ex vivo to a subject in need of treatment of a disease or condition, wherein the method comprises delivering an AAV-ITR based gene delivery system of the present disclosure to the subject. In some aspects, the method comprises delivering an AAV-ITR disclosed herein and a Rep vector disclosed herein. In some aspects, the AAV-ITR vector comprises a polynucleotide encoding a therapeutic polypeptide, polynucleotide, or a combination thereof, e.g., in a monocistronic, bicistronic, or multicistronic construct. In some aspects, the method comprises co-delivery a Rep vector disclosed herein with the AAV-ITR vector, wherein the Rep vector comprises an mRNA encoding an AAV Rep78 or Rep78 protein. In some aspects, transfection of a patient's cell or tissue with the Rep vector results in transient expression of the Rep protein, wherein the Rep protein causes the replication and amplification of the polynucleotide encoding the therapeutic polypeptide, polynucleotide, or combination thereof and expression of the therapeutic polypeptide to treat a disease or condition in the subject. In some aspects, the AAV-ITR vector is a dsDNA. In some aspects, the AAV-ITR vector and the Rep vector can be in separate delivery vehicles, e.g., a lipidic delivery system comprising a liposome and/or a lipid nanoparticle, a polymeric delivery system, or a combination thereof. In some aspects, the AAV-ITR vector is in a first liposome and the Rep vector is in a second liposome. In some aspects, the compositions of the first and second liposome are different. In some aspects, the compositions of the first and second liposome are the same. In some aspects, the AAV-ITR vector is in a first LNP and the Rep vector is in a second LNP. In some aspects, the compositions of the first and second LNP are different. In some aspects, the compositions of the first and second LNP are the same. In some aspects, the AAV-ITR vector is in a liposome and the Rep vector is in an LNP. In some aspects, the AAV-ITR vector is in an LNP and the Rep vector is in a liposome. In some aspects, the AAV-ITR vector and the Rep vector are in one delivery vehicle, i.e., in the same lipidic delivery system (e.g., a liposome, LNP, micelle, extracellular vesicle, emulsion, or exosome) o polymeric delivery system (e.g., a dendrimer).

In some aspects, the target cell contains at least one component selected from the group consisting of DNA polymerase δ (DNA Pol δ), Replication Factor C (RFC), Proliferating Cell Nuclear Antigen (PCNA), and minichromosome maintenance protein complex (MCM complex). See, e.g., Nash et al. (2007) J. Virol. 81: 5777-5787. DNA Pol δ is an enzyme complex found in eukaryotes that is involved in DNA replication and repair. The DNA Pol δ complex consists of 4 subunits: POLD1, POLD2, POLD3, and POLD4. DNA Pol δ is an enzyme used for both leading and lagging strand synthesis. It exhibits increased processivity when interacting with the proliferating cell nuclear antigen (PCNA). As well, the multisubunit protein Replication Factor C, through its role as the clamp loader for PCNA (which involves catalyzing the loading of PCNA on to DNA) is important for DNA Pol δ function. Replication Factor C is a five-subunit protein complex that is required for DNA replication. RFC is used in eukaryotic replication as a clamp loader, similar to the γ Complex in Escherichia coli. Its role as a clamp loader involves catalyzing the loading of PCNA onto DNA. It binds to the 3′ end of the DNA and uses ATP to open the ring of PCNA so that it can encircle the DNA. ATP hydrolysis causes the release of RFC, with concomitant clamp loading onto DNA. For DNA polymerase, RFC serves as primer identification. PCNA is a DNA clamp that acts as a processivity factor for DNA polymerase δ in eukaryotic cells and is essential for replication. PCNA is a homotrimer and achieves its processivity by encircling the DNA, where it acts as a scaffold to recruit proteins involved in DNA replication, DNA repair, chromatin remodeling and epigenetics. Many proteins interact with PCNA via the two known PCNA-interacting motifs PCNA-interacting peptide (PIP) box and AlkB homologue 2 PCNA interacting motif (APIM). Proteins binding to PCNA via the PIP-box are mainly involved in DNA replication whereas proteins binding to PCNA via APIM are mainly important in the context of genotoxic stress. The minichromosome maintenance protein complex (MCM complex) is a DNA helicase essential for genomic DNA replication. Eukaryotic MCM consists of six gene products, MCM2 to MCM7, which form a heterohexamer. As a critical protein for cell division, MCM is also the target of various checkpoint pathways, such as the S-phase entry and S-phase arrest checkpoints. Both the loading and activation of MCM helicase are strictly regulated and are coupled to cell growth cycles. The MCM2-7 heterohexamer is required for both DNA replication initiation and elongation; its regulation at each stage is a central feature of eukaryotic DNA replication.

In some aspects, the present disclosure provides a therapeutic polypeptide, polynucleotide, or combination thereof produced in vivo or ex vivo according to the methods disclosed herein and using the AAV-ITR based gene delivery system disclosed herein, which comprises a combination of AAV-ITR and Rep vectors. The present disclosure provides a set of vectors comprising an AAV-ITR vector disclosed herein and a Rep vector disclosed herein. In some aspects, the vectors are encapsulated in a lipidic or polymeric delivery system or a combination thereof.

The cellular targets for specific delivery of the combination of (i) an AAV-ITR vector comprising a polynucleotide encoding a therapeutic polypeptide, polynucleotide, or combination thereof and (ii) a Rep vector comprising the Rep78 or Rep68 mRNA include any cell type depending upon the therapeutic polypeptide, polynucleotide, or combination thereof encoded by the AAV-ITR vector and the particular disease or condition to be treated.

In theory and in practice, essentially any biological moiety such as a therapeutic polypeptide can be delivered to a patient in need of treatment thereof. The diseases or conditions that can be treated using the compositions and methods of the present disclosure are essentially any disease or infection, but infectious diseases and/or oncological conditions or diseases are particularly well suited for treatment using an AAV-base gene delivery system of the present disclosure. In order to achieve effective delivery to such patients, and depending upon the target cell or organ, a suitable delivery or packaging system (e.g., a lipidic delivery system, a polymeric delivery system, or a combination thereof) must be selected.

In some aspects, a therapeutic polypeptide, polynucleotide, or combination thereof disclosed in detail below (e.g., antibodies, CARs, components of a gene editing systems, peptides or enzymes for replacement therapy, among others) are produced in vivo in a patient in need of treatment thereof by administration of the combination of an AAV-ITR vector having a polynucleotide encoding the therapeutic polypeptide, polynucleotide, or combination thereof and a Rep vector, e.g., a Rep 78 vector of SEQ ID NO:38.

In some aspects, the AAV-ITR comprises a polynucleotide sequence presented in the table below.

TABLE 3
Exemplary AAV-ITR vectors.

| | | | | Nucleic |
|---|---|---|---|---|
| | | | Protein | acid |
| Gene or plasmid | MX | | SEQ ID | SEQ ID |
| background | Code | Molecule Designation | NO | NO |
| Bispecific CD19 × CD20 | MX1954 | STAR_ahCD19_hB43xahCD20_ofa_{—} | 40 | 41 |
| CAR | | CD8BBZ | | |
| Bispecific CD19 × CD20 | MX1955 | STAR_ahCD19_hB43xahCD20_ofa_{—} | 42 | 43 |
| CAR | | CD4TM_BBZ | | |
| Bispecific CD19 × CD20 | MX1956 | STAR_ahCD19_hB43xahCD20_ofa(altL1)_{—} | 44 | 45 |
| CAR | | CD8TM_BBZ | | |
| Bispecific CD19 × CD20 | MX1957 | STAR_ahCD19_hB43xahCD20_ofa(altL2)_{—} | 46 | 47 |
| CAR | | CD8TM_BBZ | | |
| hEPO gene in AAV2 | MX1959 | hEPO | 48 | 49 |
| ITR-hEPO vector | | | | |
| mCherry gene in AAV2 | MX1960 | mCherry | 50 | 51 |
| ITR-mCherry vector | | | | |
| mEPO gene in AAV2 | MX1961 | mEPO | 52 | 53 |
| ITR-mEPO vector | | | | |
| Luciferase gene in AAV2 | | Luciferase | 54 | 55 |
| ITR-Luc vector | | | | |
| Anti-COVID | MX1448 | Anti-COVID E12 × E8 | 56 | 57 |
| E12 × E8 bispecific Ab in | | bispecific Ab | | |
| AAV2 ITR vector | | | | |

In some aspects, the AAV-ITR vector comprises a polynucleotide sequence set forth the table above. In some aspects, the AAV-ITR vector is the MX1954 bispecific CD19xCD20 CAR AAV-ITR vector presented in the table above. In some aspects, the AAV-ITR vector is the MX1955 bispecific CD19xCD20 CAR AVV-ITR vector presented in the table above. In some aspects, the AAV-ITR vector is the MX1956 bispecific CD19xCD20 CAR AVV-ITR vector presented in the table above. In some aspects, the AAV-ITR vector is the MX1957 bispecific CD19xCD20 CAR AVV-ITR vector presented in the table above. In some aspects, the AAV-ITR vector is the MX1959 hEPO gene in AAV2 ITR-hEPO AVV-ITR vector presented in the table above. In some aspects, the AAV-ITR vector is the MX1960 mCherry gene in AAV2 ITR-mCherry AVV-ITR vector presented in the table above. In some aspects, the AAV-ITR vector is the MX1961 mEPO gene in AAV2 ITR-mEPO AVV-ITR vector presented in the table above. In some aspects, the AAV-ITR vector is Luciferase gene in AAV2 ITR-Luc AVV-ITR vector presented in the table above. In some aspects, the AAV-ITR vector is the MX1448 Anti-COVID E12xE8 bispecific Ab in AAV2 ITR vector presented in the table above.

In some aspects, the AAV-ITR vector is derived from the MX1954 bispecific CD19xCD20 CAR AAV-ITR vector presented in the table above, wherein the subsequence (CDS) encoding the corresponding protein sequence presented in the table above has been replaced with a polynucleotide encoding a therapeutic polypeptide, polynucleotide, or combination thereof. In some aspects, the AAV-ITR vector is derived from the MX1955 bispecific CD19xCD20 CAR AVV-ITR vector presented in the table above, wherein the subsequence (CDS) encoding the corresponding protein sequence presented in the table above has been replaced with a polynucleotide encoding a therapeutic polypeptide, polynucleotide, or combination thereof. In some aspects, the AAV-ITR vector is derived from the MX1956 bispecific CD19xCD20 CAR AVV-ITR vector presented in the table above, wherein the subsequence (CDS) encoding the corresponding protein sequence presented in the table above has been replaced with a polynucleotide encoding a therapeutic polypeptide, polynucleotide, or combination thereof. In some aspects, the AAV-ITR vector is derived from the MX1957 bispecific CD19xCD20 CAR AVV-ITR vector presented in the table above, wherein the subsequence (CDS) encoding the corresponding protein sequence presented in the table above has been replaced with a polynucleotide encoding a therapeutic polypeptide, polynucleotide, or combination thereof. In some aspects, the AAV-ITR vector is derived from the MX1959 hEPO gene in AAV2 ITR-hEPO AVV-ITR vector presented in the table above, wherein the subsequence (CDS) encoding the corresponding protein sequence presented in the table above has been replaced with a polynucleotide encoding a therapeutic polypeptide, polynucleotide, or combination thereof. In some aspects, the AAV-ITR vector is derived from the MX1960 mCherry gene in AAV2 ITR-mCherry AVV-ITR vector presented in the table above, wherein the subsequence (CDS) encoding the corresponding protein sequence presented in the table above has been replaced with a polynucleotide encoding a therapeutic polypeptide, polynucleotide, or combination thereof. In some aspects, the AAV-ITR vector is derived from the MX1961 mEPO gene in AAV2 ITR-mEPO AVV-ITR vector presented in the table above, wherein the subsequence (CDS) encoding the corresponding protein sequence presented in the table above has been replaced with a polynucleotide encoding a therapeutic polypeptide, polynucleotide, or combination thereof. In some aspects, the AAV-ITR vector is derived from the Luciferase gene in AAV2 ITR-Luc AVV-ITR vector presented in the table above, wherein the subsequence (CDS) encoding the corresponding protein sequence presented in the table above has been replaced with a polynucleotide encoding a therapeutic polypeptide, polynucleotide, or combination thereof. In some aspects, the AAV-ITR vector is derived from the MX1448 Anti-COVID E12xE8 bispecific Ab in AAV2 ITR vector presented in the table above, wherein the subsequence (CDS) encoding the corresponding protein sequence presented in the table above has been replaced with a polynucleotide encoding a therapeutic polypeptide, polynucleotide, or combination thereof.

In some aspects, the present disclosure provides the use of an AAV-ITR based gene delivery system (e.g., an AAV-ITR vector disclosed herein in combination with a Rep78 vector or Rep68 vector disclosed herein) as a medicament. In some aspects, the present disclosure provides the use of an AAV-ITR based gene delivery system (e.g., a combination of an AAV-ITR vector disclosed herein in combination with a Rep78 vector or Rep68 vector disclosed herein) for the treatment of a disease or conditions selected, e.g., from the group consisting of cancer, an infectious disease, or a genetic disease. In some aspects, the present disclosure provides the use of an AAV-ITR based gene delivery system (e.g., an AAV-ITR vector disclosed herein in combination with a Rep78 vector or Rep68 vector disclosed herein) in the manufacture of a medicament to treat a subject in need of treatment thereof.

In a specific aspect, the combination comprises an AAV-ITR vector of SEQ ID NO:35 in which the ORF has been replaced with polynucleotide encoding a therapeutic polypeptide, polynucleotide, or combination thereof, and a Rep78 vector having SEQ ID NO:38.

In some aspects, both vectors are co-encapsulated in a single lipidic or polymeric delivery system disclosed herein. In some aspects, each vector is encapsulated in lipidic or polymeric delivery system disclosed herein. For example, in some aspects, the AAV-ITR vector in encapsulated in a first LNP, and the Rep78 vector is encapsulated in a second LNP.

In some aspects, the present disclosure provides a method of delivering a therapeutic polypeptide, polynucleotide, or combination thereof in vivo comprising administering an AAV-ITR based gene delivery system of the present disclosure comprising an AAV-ITR vector disclosed herein and an Rep78 vector disclosed herein in a weight ratio (ngs/ngs) of AAV-ITR vector to Rep78 vector of about 1:1 to a patient in need of treatment thereof.

The present disclosure provides a method of treating a disease or condition in a subject in need of treatment comprising administering an AAV-ITR based gene delivery system of the present disclosure to the subject. The present disclosure provides a method of treating a disease or condition comprising co-administering an AAV-ITR vector of the present disclosure and a Rep vector of the present disclosure to a subject in need thereof. In some aspects, the disease or condition is selected, e.g., from the group consisting of cancer, an immune disease, an inflammatory disease, a neurodegenerative disorder, a central nervous disease, a metabolic disease, an autoimmune disease, an infectious disease, or a fibrotic disease. Diseases and conditions that can be treated using the compositions and methods disclosed herein are discussed in detail below.

The disclosure provides a cell comprising an AAV-ITR based gene delivery system of the present disclosure, i.e., an AAV-ITR vector and a Rep vector. In some aspects, the AAV-ITR vector is integrated in the cell's genome. Also provided is a cell genetically modified by using an AAV-ITR based gene delivery system of the present disclosure. E.g., the cell can be a cell in which a defective gene has been replaced by a healthy version of the gene delivered via the AAV-ITR vector of an AAV-ITR based gene delivery system of the present disclosure. E.g., the cell can be a cell in which a polynucleotide or polynucleotides encoding an antibody has been inserted in the genome of the cell by using an AAV-ITR based gene delivery system of the present disclosure. In some aspects, the cell can be a cell in which a polynucleotide encoding CAR has been inserted in the genome of the cell by using an AAV-ITR based gene delivery system of the disclosure.

The present disclosure also provides a pharmaceutical composition comprising an AAV-ITR based delivery system of the present disclosure and a pharmaceutically acceptable excipient. The present disclosure provides a pharmaceutical composition comprising an AAV-ITR vector of the present disclosure and a pharmaceutically acceptable excipient. The present disclosure provides a pharmaceutical composition comprising a Rep vector of the present disclosure and a pharmaceutically acceptable excipient. The present disclosure provides a pharmaceutical composition comprising an AAV-ITR vector of the present disclosure and a Rep vector of the present disclosure and a pharmaceutically acceptable excipient. The present disclosure also provides a pharmaceutical composition comprising a cell disclosed above.

In some aspects, the administration of the AAV-ITR based gene delivery system of the present disclosure, a cell disclosed herein, or a pharmaceutical composition disclosed herein is, for example, parenteral, intramuscular, intrapulmonary, intranasal, intravenous, intraarterial, intraperitoneal, subcutaneous, intraocular, intrathecal, intratumoral, or peritumoral. In some specific aspects, the AAV-ITR vector and Rep vector of the present disclosure are co-administered intramuscularly, e.g., via intramuscular injection, to a subject in need thereof. In some aspects, the AAV-ITR based gene delivery system of the present disclosure or cells (autologous or nonautologous) modified using the AAV-ITR based gene delivery system of the present disclosure can be administered to a subject in need thereof. In some aspects, the AAV-ITR based gene delivery system of the present disclosure or cells (autologous or heterologous) modified using the AAV-ITR based gene delivery system of the present disclosure can be administered to a subject in need thereof using an implantable device. See, e.g., Chang (1997) IEEE Eng. Med. Biol. Mag. 16:145-150. In some aspects, an AAV-ITR based gene delivery system of the present disclosure can be used to genetically modify a patient's cells for autologous CAR T-cell therapy. In some aspects, an AAV-ITR based gene delivery system of the present disclosure can be used to genetically modify T cells for allogeneic CAR T-cell therapy.

In some aspects, the present disclosure provides a method of sustained polypeptide expression in a host cell comprising transfecting the host cell with an AAV-ITR based delivery system of the present disclosure (i.e., a combination comprising an AAV-ITR vector and a Rep vector) without inducing a vector associated immune response.

The present disclosure provides compositions and methods as disclosed above wherein the therapeutic polypeptide encoded by a polynucleotide or polynucleotides in the AAV-ITR vector is selected from the group consisting of SARS-CoV2 (COVID) neutralizing antibodies, influenza neutralizing antibodies, HIV neutralizing antibodies, gastric inhibitory polypeptide (GIP) agonists, glucagon-like peptide-1 (GLP-1) agonists, CAR, anticancer antibodies, cytokines, enzymes, or ionic channels. The present disclosure provides compositions and methods as disclosed above wherein the therapeutic polynucleotide in the AAV-ITR vector is a functional gene to treat a disease caused by the present of a defective non-functional gene in a patient in need thereof. In a specific aspect, this disclosure provides compositions and methods as disclosed above wherein the therapeutic polynucleotide in the AAV-vector encodes human erythropoietin (EPO).

The present disclosure provides compositions and methods as disclosed above wherein the therapeutic polynucleotide in the AAV-ITR vector encodes components of a gene editing system to treat a genetic disease caused by a defective gene, thereby correcting the genetic defect in the subject. In some aspects, the therapeutic polynucleotide in the AAV-ITR vector encodes a gene editing nuclease (e.g., Cas) and a gRNA capable of correcting an error in a defective gene in a patient in need thereof. In some aspects, the therapeutic polynucleotide in the AAV-ITR vector encodes a gene editing nuclease (e.g., Cas) and a gRNA capable of correcting an error in a defective gene in a patient having a triplet repeat disease. Triplet repeat diseases (TRDs) are caused by pathogenic expansions of trinucleotide sequence repeats within coding and non-coding regions of different genes. They are typically progressive, very disabling and frequently involve the nervous system. In some aspects, the triplet repeat disease is selected from the group consisting of Huntington's disease, Spinocerebellar ataxia type 1 (SCA1), SCA2, SCA3, SCA6, SCA7, SCA8, SCA12, SCA17, spinal and bulbar muscular atrophy, dentatorubropallidoluysian atrophy, myotonic dystrophy type 1, oculopharingeal muscular dystrophy, Friedreich's ataxia, fragile X syndrome, fragile X-associated tremor/ataxia syndrome, fragile XE mental retardation, Huntington's disease-like 2, and Fuchs corneal dystrophy.

In some aspects, the present disclosure provides an AAV-ITR based delivery system comprising (i) an AAV-ITR vector comprising a polynucleotide encoding a CRISPR/Cas gene editing system and (ii) a Rep78 vector, wherein the CRISPR/Cas gene editing system comprises a polynucleotide encoding a Cas9 nuclease and a polynucleotide encoding a gRNA.

In some aspects, the present disclosure provides an AAV-ITR based delivery system comprising (i) an AAV-ITR vector comprising a polynucleotide encoding an antibody, e.g., an MSTAR antibody, and (ii) a Rep78 vector, wherein the polynucleotide encoding the antibody is bicistronic. In some aspects, one cistron encodes an antibody heavy chain and another cistron encodes an antibody light chain.

In some aspects, the present disclosure provides an AAV-ITR based delivery system comprising (i) an AAV-ITR vector comprising a polynucleotide encoding an anti-cMet/Trop2 bispecific antibody, and (ii) a Rep78 vector. In some aspects, the present disclosure provides an AAV-ITR based delivery system comprising (i) an AAV-ITR vector comprising a polynucleotide encoding an anti-COVID-19 bispecific antibody and (ii) a Rep78 vector. In some aspects, the present disclosure provides an AAV-ITR based delivery system comprising (i) an AAV-ITR vector comprising a polynucleotide encoding a CAR and (ii) a Rep78 vector. In some aspects, the present disclosure provides an AAV-ITR based delivery system comprising (i) an AAV-ITR vector comprising a polynucleotide encoding GLP-1 and/or a GLP-1 agonist and (ii) a Rep78 vector. In some aspects, the present disclosure provides an AAV-ITR based delivery system comprising (i) an AAV-ITR vector comprising a polynucleotide encoding a COVID neutralizing antibody and (ii) a Rep78 vector. In some aspects, the present disclosure provides an AAV-ITR based delivery system comprising (i) an AAV-ITR vector comprising a polynucleotide encoding an HIV neutralizing antibody and (ii) a Rep78 vector. In some aspects, the present disclosure provides an AAV-ITR based delivery system comprising (i) an AAV-ITR vector comprising a polynucleotide encoding an influenza-neutralizing antibody and (ii) a Rep78 vector.

Also provided is a kit comprising an AAV-ITR vector and a Rep vector, wherein the AAV-ITR vector comprises a polynucleotide encoding a therapeutic polypeptide, polynucleotide, or combination thereof. Also provided is a kit comprising an AAV-ITR vector and a Rep vector, wherein the AAV-ITR comprises a polynucleotide encoding a marker gene to be substituted by a polynucleotide encoding a therapeutic polypeptide, polynucleotide, or a combination thereof. In some aspects, the kit comprises reagents to prepare a lipidic delivery system or polymeric delivery system or a combination thereof to encapsulate the AAV-ITR vector and/or the Rep vector. The kits provided herein optionally include instructions for use.

### Inverted Terminal Repeats (ITR)

The AAV-ITR vectors of the AAV-ITR based gene delivery systems of the present disclosure contain 5′ and 3′ ITRs. As used herein, an “inverted terminal repeat” or “ITR” refers to a nucleic acid subsequence located at either the 5′ or 3′ end of the sequence, which comprises a set of nucleotides (initial sequence) followed downstream by its reverse complement, i.e., palindromic sequence. The intervening sequence of nucleotides between the initial sequence and the reverse complement can be any length and can contain a sequence of a gene of interest (GOI). ITRs are reported to be the minimum sequences required for AAV proviral integration. In some aspects, the ITR comprises a naturally occurring ITR, e.g., the ITR comprises all or a portion of a Parvovirus ITR. In some aspects, the ITR comprises a synthetic sequence. In one aspect, the first ITR or the second ITR comprises a synthetic sequence. In another aspect, each of the first ITR and the second ITR comprises a synthetic sequence. In some aspects, the first ITR or the second ITR comprises a naturally occurring sequence. In another aspect, each of the first ITR and the second ITR comprises a naturally occurring sequence.

The sequence from the 5′ ITR sequence from AAV serotype 1 corresponds to nucleotides 1 to 143 of the AAV2 complete genome available at Genbank NC_002077.1. The sequence of the 5′ ITR sequence from AAV serotype 2 corresponds to nucleotides 1 to 145 of the AAV1 complete genome available at Genbank NC_001401.2. In some aspects, the 5′ ITR comprises, consists, or consists essentially of the sequence set forth in SEQ ID NO: 58. In some aspects, the 3′ ITR comprises, consists, or consists essentially of the sequence set forth in SEQ ID NO: 59. The sequence of the 5′ ITR sequence from AAV serotype 3A corresponds to nucleotides 1 to 143 of the AAV3A complete genome available at Genbank JB292182.1. The sequence of the 5′ ITR sequence from AAV serotype 3B corresponds to nucleotides 1 to 143 of the AAV3B complete genome available at Genbank AF028705.1. The sequence of the 5′ ITR sequence from AAV serotype 4 corresponds to nucleotides 1 to 46 of the AAV4 complete genome available at Genbank NC_001829.1. The sequence of the 5′ ITR sequence from AAV serotype 7 corresponds to nucleotides 1 to 145 of the AAV7 complete genome available at Genbank ITR7 NC_006260.1. In some aspects, the ITR sequence is an ITR sequence from AAV serotype 5 (complete genome available at Genbank NC_006152.1), AAV serotype 6 (complete genome available at Genbank AF028704.1), AAV serotype 8 (complete genome available at Genbank NC_006261.1), AAV serotype 9 (complete genome available at Genbank AX753250.1), AAV serotype 10 (complete genome available at Genbank AY631965.1), AAV serotype 11 (complete genome available at Genbank AY631966.1), AAV serotype 12 (complete genome available at Genbank DQ813647.1), or AAV serotype 13 (complete genome available at Genbank EU285562.1).

In some aspects, 3′ ITR sequences are the reverse complement of the 5′ sequences. In some aspects, the ITR comprises an ITR from an AAV genome. In some aspects, the ITR is an ITR of an AAV genome selected from AAV1, AAV2, AAV3, AAV3B, AAV4, AAV5, AAV6, AAV7, AAV8, AAV9, AAV10, AAV11, AAV12, AAV13, and any combination thereof. In a particular aspect, the ITR is an ITR of the AAV2 genome. In some aspects, the ITR is a synthetic sequence genetically engineered to include at its 5′ and 3′ ends ITRs derived from one or more of AAV genomes. In some aspects, the ITRs are derived from the same genome, e.g., from the genome of the same virus, or from different genomes, e.g., from the genomes of two or more different AAV genomes. In some aspects, the ITRs are derived from the same AAV genome. In a specific aspect, the two ITRs present in the nucleic acid molecule of the disclosure are the same, and can in particular be AAV2 ITRs, AAV5 ITRs or AAV9 ITRs. In one particular aspect, the first ITR and the second ITR are identical.

In some aspects, the ITRs form hairpin loop structures. In one aspect, the first ITR forms a hairpin structure. In another aspect, the second ITR forms a hairpin structure. Still in another aspect, both the first ITR and the second ITR form hairpin structures.

In some aspects, an ITR in a polynucleotide described herein is a transcriptionally activated ITR. A transcriptionally-activated ITR can comprise all or a portion of a wild-type ITR that has been transcriptionally activated by inclusion of at least one transcriptionally active element. Various types of transcriptionally active elements are suitable for use in this context. In some aspects, the transcriptionally active element is a constitutive transcriptionally active element. Constitutive transcriptionally active elements provide an ongoing level of gene transcription, and are preferred when it is desired that the transgene be expressed on an ongoing basis. In other aspects, the transcriptionally active element is an inducible transcriptionally active element. Inducible transcriptionally active elements generally exhibit low activity in the absence of an inducer (or inducing condition), and are upregulated in the presence of the inducer (or switch to an inducing condition). Inducible transcriptionally active elements can be preferred when expression is desired only at certain times or at certain locations, or when it is desirable to titrate the level of expression using an inducing agent. Transcriptionally active elements can also be tissue-specific; that is, they exhibit activity only in certain tissues or cell types.

Transcriptionally active elements can be incorporated into an ITR in a variety of ways. In some aspects, a transcriptionally active element is incorporated 5′ to any portion of an ITR or 3′ to any portion of an ITR. In other aspects, a transcriptionally active element of a transcriptionally-activated ITR lies between two ITR sequences. If the transcriptionally active element comprises two or more elements, which must be spaced apart, those elements can alternate with portions of the ITR. In some aspects, a hairpin structure of an ITR is deleted and replaced with inverted repeats of a transcriptional element. This latter arrangement would create a hairpin mimicking the deleted portion in structure. Multiple tandem transcriptionally active elements can also be present in a transcriptionally-activated ITR, and these can be adjacent or spaced apart. In addition, protein-binding sites (e.g., Rep binding sites) can be introduced into transcriptionally active elements of the transcriptionally-activated ITRs. A transcriptionally active element can comprise any sequence enabling the controlled transcription of DNA by RNA polymerase to form RNA, and can comprise, for example, a transcriptionally active element, as defined below.

Transcriptionally-activated ITRs provide both transcriptional activation and ITR functions to the nucleic acid molecule in a relatively limited nucleotide sequence length which effectively maximizes the length of a transgene encoding a GOI which can be carried and expressed from the nucleic acid molecule. Incorporation of a transcriptionally active element into an ITR can be accomplished in a variety of ways. A comparison of the ITR sequence and the sequence requirements of the transcriptionally active element can provide insight into ways to encode the element within an ITR. For example, transcriptional activity can be added to an ITR through the introduction of specific changes in the ITR sequence that replicates the functional elements of the transcriptionally active element. A number of techniques exist in the art to efficiently add, delete, and/or change particular nucleotide sequences at specific sites (see, for example, Deng and Nickoloff (1992) Anal. Biochem. 200:81-88). Another way to create transcriptionally-activated ITRs involves the introduction of a restriction site at a desired location in the ITR. In addition, multiple transcriptionally activate elements can be incorporated into a transcriptionally-activated ITR, using methods known in the art.

By way of illustration, transcriptionally-activated ITRs can be generated by inclusion of one or more transcriptionally active elements such as: TATA box, GC box, CCAAT box, Spl site, Inr region, CRE (cAMP regulatory element) site, ATF-1/CRE site, APBβ box, APBα box, CArG box, CCAC box, or any other element involved in transcription as known in the art.

Any AAV ITR known in the art can be used in the compositions of the present disclosure. In some aspects, the AAV ITR is from an AAV serotype selected from the group consisting of AAV type 1, AAV type 2, AAV type 3, AAV type 3B, AAV type 4, AAV type 5, AAV type 6, AAV type 7, AAV type 8, AAV type 9, AAV type 10, AAV type 11, AAV type 12, AAV type 13, Rh10, Rh74, AAV-2i8, snake AAV, avian AAV, bovine AAV, canine AAV, equine AAV, ovine AAV, goat AAV, shrimp AAV, a synthetic AAV, an any combination thereof. In certain aspects, the AAV ITR is from an AAV type 2, e.g., AAV2.

For historical reasons and the sake of convenience, most of the AAV vectors contain the ITR of AAV serotype 2, the sole viral sequences required for the replication and packaging of the recombinant genome in AAV capsids.

TABLE 4
AAV ITR sequences

| AAV | 5′ ITR sequence SEQ | 3′ ITR sequence SEQ ID |
|---|---|---|
| Serotype | ID NO | NO |
| AAV1 | 60 | 61 |
| AAV2 | 62 | 63 |
| AAV3A | 64 | 65 |
| AAV4 | 66 | 67 |
| AAV5 | 68 | 69 |
| AAV6 | 70 | 71 |
| AAV7 | 72 | 73 |
| AAV8 | 74 | 75 |
| AAV9 | 76 | 77 |
| AAV10 | 78 | 79 |
| AAV11 | 80 | 81 |
| AAV12 | 82 | 83 |
| AAV13 | 84 | 85 |

ITR of AAV1 to 9 present a high degree of homology (82-90%), except for AAV5 (58%), resulting in differences in DNA cleavage specificity and nuclear factors involved in viral replication. Vector genomes carrying AAV ITR5 have been shown to be approximately 5 times more active than those based on ITR2. Similar experiments in mouse muscle with AAV1 and AAV6 capsid pseudotyped vectors also confirmed a higher efficiency of vectors based on AAV5 ITR compared to those based on the conventional AAV2 ITR, indicating that AAV5 ITR based AAV systems are more efficient systems to mediate gene expression than the standard AAV2 ITR based vector in particular for skeletal muscle targeting. See Le Bec et al. (2008) Musculoskeletal Gene and Cell Therapy: Bone, Joint, Tendon and Muscle Therapy, vol. 16, suppl. 1, S346.

ITRs contains three palindromic sequences (A-A′, B-B′, and C-C′ regions) and form a T-shaped structure. Deletion of the B-B′ and C-C′ regions of ITRs reduces rAAV productivity but increases transgene expression. See Zhou et al. (2017) Sci. Rep. 7, 5432. Accordingly, in some aspects of the present disclosure, the ITR does not contain a B-B′ and/or C-C′ region. In some aspects, the ITR does not contain a B-B′ and/or C-C′ region and includes only one palindromic sequence (A-A′ region). In some aspects, the ITR includes only one palindromic sequence (A-A′ region) and forms a U-shaped structure. In some aspects, the ITR comprises a sequence having 1, 2, 3, 4, 5, 6, 7, 8, 9, or 10 additional nucleotides at the 5′ end of an ITR sequence disclosed herein, wherein the ITR is capable of binding to a Rep protein (e.g., a Rep78 or Rep68 protein). In some aspects, the ITR comprises a sequence having 1, 2, 3, 4, 5, 6, 7, 8, 9, or 10 additional nucleotides at the 3′ end of an ITR sequence disclosed herein, wherein the ITR is capable of binding to a Rep protein (e.g., a Rep78 or Rep68 protein). In some aspects, the ITR comprises a sequence having 1, 2, 3, 4, 5, 6, 7, 8, 9, or 10 less nucleotides at the 5′ end of an ITR sequence disclosed herein, wherein the ITR is capable of binding to a Rep protein (e.g., a Rep78 or Rep68 protein). In some aspects, the ITR comprises a sequence having 1, 2, 3, 4, 5, 6, 7, 8, 9, or 10 less nucleotides at the 3′ end of an ITR sequence disclosed herein, wherein the ITR is capable of binding to a Rep protein (e.g., a Rep78 or Rep68 protein). In some aspects, the ITR comprises a sequence having 1, 2, 3, 4, 5, 6, 7, 8, 9, or 10 more nucleotides at the 5′ end and 1, 2, 3, 4, 5, 6, 7, 8, 9, or 10 more nucleotides at the 3′ end of an ITR sequence disclosed herein, wherein the ITR is capable of binding to a Rep protein (e.g., a Rep78 or Rep68 protein). In some aspects, the ITR comprises a sequence having 1, 2, 3, 4, 5, 6, 7, 8, 9, or 10 less nucleotides at the 5′ end and 1, 2, 3, 4, 5, 6, 7, 8, 9, or 10 less nucleotides at the 3′ end of an ITR sequence disclosed herein, wherein the ITR is capable of binding to a Rep protein (e.g., a Rep78 or Rep68 protein). In some aspects, the ITR comprises a sequence having 1, 2, 3, 4, 5, 6, 7, 8, 9, or 10 more nucleotides at the 5′ end and 1, 2, 3, 4, 5, 6, 7, 8, 9, or 10 less nucleotides at the 3′ end of an ITR sequence disclosed herein, wherein the ITR is capable of binding to a Rep protein (e.g., a Rep78 or Rep68 protein). In some aspects, the ITR comprises a sequence having 1, 2, 3, 4, 5, 6, 7, 8, 9, or 10 less nucleotides at the 5′ end and 1, 2, 3, 4, 5, 6, 7, 8, 9, or 10 more nucleotides at the 3′ end of an ITR sequence disclosed herein, wherein the ITR is capable of binding to a Rep protein (e.g., a Rep78 or Rep68 protein).

In some aspects, the ITR comprises, consists, or consists essentially of an ITR sequence disclosed herein except for 1, 2, 3, 4, 5, 6, 7, 8, 9, 10, 11, 12, 13, 14, 15, 16, 17, 18, 19, or 20 mutations, wherein the ITR is capable of binding to a Rep protein (e.g., a Rep78 or Rep68 protein). In some aspects, the ITR comprises, consists, or consists essentially of an ITR sequence having about 60%, about 65%, about 70%, about 75%, about 80%, about 85%, about 90%, about 95%, about 96%, about 97%, about 98%, or about 99% sequence identity to an ITR sequence disclosed herein, wherein the ITR is capable of binding to a Rep protein (e.g., a Rep78 or Rep68 protein). In general, the ITR of the present disclosure can be a functional variant or functional fragment of a naturally occurring ITR that has been engineered while preserving the Rep Binding Element (RBE) of the ITR.

### Rep Proteins

The Rep vector component of the AAV-ITR based gene delivery system of the present disclosure comprises an mRNA encoding a Parvovirus Rep protein, e.g., a Rep68 protein or a Rep78 protein.

The AAV rep gene, by using two promoters and alternative splicing, encodes four regulatory proteins that are dubbed Rep78, Rep68, Rep52 and Rep40. Rep proteins are required for viral genome replication and packaging. The Rep vectors of the present disclosure comprise an mRNA encoding a Rep protein, which can be naturally occurring or comprise a mutant or synthetic sequence. As used herein, the terms “Rep RNA” or “Rep mRNA” are interchangeable and refer to an RNA sequence that encodes an AAV Rep protein, a Rep78 protein or a Rep68 protein. The terms “Rep78 RNA” or “Rep78 mRNA” or “Rep78 RNA vector” or “Rep78 mRNA vector” or “Rep68 RNA” or Rep68 mRNA” or “Rep68 RNA vector” or “Rep68 mRNA vector” refer to an RNA sequence that respectively encodes an AAV Rep78 or AAV Rep68 protein. In some aspects, the Rep RNA encoding the Rep protein is produced in vitro, e.g., through in vitro transcription.

The present disclosure provides nucleic acids that encode an AAV Rep protein. In some aspects, the nucleic acids encode a Rep78 protein. In some aspects, the nucleic acids encode a Rep68 protein. In some aspects, the nucleic acid encoding a Rep78 protein is a ribonucleic acid (Rep78 RNA). In some aspects, the nucleic acid encoding a Rep68 protein is a ribonucleic acid (Rep68 RNA). In some aspects, the Rep78 RNA is an in vitro transcribed Rep78 mRNA. In some aspects, the Rep68 RNA is an in vitro transcribed Rep68 mRNA.

In some aspects, the Rep encoding nucleic acid comprises a synthetic sequence. In some aspects, the Rep78 encoding nucleic acid comprises a synthetic sequence. In some aspects, the Rep68 encoding nucleic acid comprises a synthetic sequence.

In some aspects, a Rep encoding nucleic acid is from an AAV genome selected from an AAV serotype AAV1, AAV2, AAV3, AAV3B, AAV4, AAV5, AAV6, AAV7, AAV8, AAV9, AAV10, AAV11, AAV12, AAV13 and any combination thereof. In some aspects, Rep78 encoding nucleic acid is from an AAV genome selected from AAV1, AAV2, AAV3, AAV3B, AAV4, AAV5, AAV6, AAV7, AAV8, AAV9, AAV10, AAV11, AAV12, AAV13 and any combination thereof. In some aspects, a Rep68 encoding nucleic acid is from an AAV genome selected from AAV1, AAV2, AAV3, AAV3B, AAV4, AAV5, AAV6, AAV7, AAV8, AAV9, AAV10, AAV11, AAV12, AAV13 and any combination thereof.

In some aspects, the Rep encoding RNA sequence is from the AAV1 genome. In some aspects, the Rep encoding RNA sequence is from the AAV2 genome. In some aspects, the Rep encoding RNA sequence is from the AAV3 genome. In some aspects, the Rep encoding RNA sequence is from the AAV3B genome. In some aspects, the Rep encoding RNA sequence is from the AAV4 genome. In some aspects, the Rep encoding RNA sequence is from the AAV5 genome. In some aspects, the Rep encoding RNA sequence is from the AAV6 genome. In some aspects, the Rep encoding RNA sequence is from the AAV7 genome. In some aspects, the Rep encoding RNA sequence is from the AAV8 genome. In some aspects, the Rep encoding RNA sequence is from the AAV9 genome. In some aspects, the Rep encoding RNA sequence is from the AAV10 genome. In some aspects, the Rep encoding RNA sequence is from the AAV11 genome. In some aspects, the Rep encoding RNA sequence is from the AAV12 genome. In some aspects, the Rep encoding RNA sequence is from the AAV13 genome.

In some aspects, the Rep protein encoded by a Rep vector of the present disclosure is a Rep protein variant that has about 80%, about 85%, about 90%, about 95%, about 96%, about 97% about 98%, or about 99% sequence identity to the sequence of AAV2Rep78. In some aspects, the Rep protein encoded by a Rep vector of the present disclosure is a Rep protein variant has about 80%, about 85%, about 90%, about 95%, about 96%, about 97%, about 98%, or about 99% sequence identity to the sequence of AAV2 Rep76. In some aspects, the Rep protein is a functional variant or functional fragment of a Rep78 or Rep68 protein that can bind to the Rep Binding Element (RBE) of the ITR.

TABLE 5
Human AAV Rep78 and Rep68 sequences

| | | Protein SEQ ID | Nucleic acid SEQ ID |
|---|---|---|---|
| Serotype | Rep type | NO | NO |
| AAV1 | 78 | 86 | 87 |
| | 68 | 88 | 89 |
| AAV2 | 78 | 90 | 91 |
| | 68 | 92 | 93 |
| AAV3 | 78 | 94 | 95 |
| | 68 | 96 | 97 |
| AAV3B | 78 | 98 | 99 |
| | 68 | 100 | 101 |
| AAV4 | 78 | 102 | 103 |
| | 68 | 104 | 105 |
| AAV5 | 78 | 106 | 107 |
| | 68 | 108 | 109 |
| AAV6 | 78 | 110 | 111 |
| | 68 | 112 | 113 |
| AAV7 | 78 | 114 | 115 |
| | 68 | 116 | 117 |
| AAV8 | 78 | 118 | 119 |
| | 68 | 120 | 121 |
| AAV9 | 78 | 122 | 123 |
| | 68 | 124 | 125 |
| AAV10 | 78 | 126 | 127 |
| | 68 | 128 | 129 |
| AAV11 | 78 | 130 | 131 |
| | 68 | 132 | 133 |
| AAV12 | 78 | 134 | 135 |
| | 68 | 136 | 137 |
| AAV13 | 78 | 138 | 139 |
| | 68 | 140 | 141 |

In some aspects, the Rep encoding RNA sequence is a Rep78 RNA sequence of an AAV serotype selected the group consisting of AAV1, AAV2, AAV3, AAV3B, AAV4, AAV5, AAV6, AAV7, AAV8, AAV9, AAV10, AAV11, AAV12, or AAV13. In some aspects, the Rep RNA sequence is a Rep78 RNA sequence of an AAV1. In some aspects, the Rep RNA sequence is a Rep78 RNA sequence of an AAV2. In some aspects, the Rep RNA sequence is a Rep78 RNA sequence of an AAV3. In some aspects, the Rep RNA sequence is a Rep78 RNA sequence of an AAV3B. In some aspects, the Rep RNA sequence is a Rep78 RNA sequence of an AAV4. In some aspects, the Rep RNA sequence is a Rep78 RNA sequence of an AAV5. In some aspects, the Rep RNA sequence is a Rep78 RNA sequence of an AAV6. In some aspects, the Rep RNA sequence is a Rep78 RNA sequence of an AAV7. In some aspects, the Rep RNA sequence is a Rep78 RNA sequence of an AAV8. In some aspects, the Rep RNA sequence is a Rep78 RNA sequence of an AAV9. In some aspects, the Rep RNA sequence is a Rep78 RNA sequence of an AAV10. In some aspects, the Rep RNA sequence is a Rep78 RNA sequence of an AAV11. In some aspects, the Rep RNA sequence is a Rep78 RNA sequence of an AAV12. In some aspects, the Rep RNA sequence is a Rep78 RNA sequence of an AAV13.

In some aspects, the Rep RNA sequence is a Rep68 RNA sequence of an AAV1. In some aspects, the Rep RNA sequence is a Rep68 RNA sequence of an AAV2. In some aspects, the Rep RNA sequence is a Rep68 RNA sequence of an AAV3. In some aspects, the Rep RNA sequence is a Rep68 RNA sequence of an AAV3B. In some aspects, the Rep RNA sequence is a Rep68 RNA sequence of an AAV4. In some aspects, the Rep RNA sequence is a Rep68 RNA sequence of an AAV5. In some aspects, the Rep RNA sequence is a Rep68 RNA sequence of an AAV6. In some aspects, the Rep RNA sequence is a Rep68 RNA sequence of an AAV7. In some aspects, the Rep RNA sequence is a Rep68 RNA sequence of an AAV8. In some aspects, the Rep RNA sequence is a Rep68 RNA sequence of an AAV9. In some aspects, the Rep RNA sequence is a Rep68 RNA sequence of an AAV10. In some aspects, the Rep RNA sequence is a Rep68 RNA sequence of an AAV11. In some aspects, the Rep RNA sequence is a Rep68 RNA sequence of an AAV12. In some aspects, the Rep RNA sequence is a Rep68 RNA sequence of an AAV13.

In some aspects, the Rep protein is a Rep78 or Rep68 protein selected from Genbak protein accession codes YP_680423.1, QDH44219.1, QDH44159.1, QDH44253.1, QDH44302.1, ASW20943.1, QDH44103.1, QDH44232.1, QDH44454.1, QDH44260.1, QDH44086.1, AAU05365.1, QDH44173.1, AAU05359.1, QDH44096.1, AAU05369.1, QDH44180.1, UM075523.1, QDH44214.1, QDH44461.1, UXD78474.1, UXD78506.1, QDH44187.1, QJS95374.1, QDH44267.1, UGV24180.1, UXD78434.1, QDH44344.1, UXD78466.1, UXD78578.1, QDH44330.1, UGV24174.1, UXD78586.1, QDH44440.1, QDH44398.1, QDH44384.1, QDH44110.1, QDH44152.1, AAU05367.1, UXD78562.1, QDH44117.1, UGV24183.1, UXD78546.1, UXD78450.1, UGV24172.1, UXD78530.1, UGV24181.1, AAU05363.1, QDH44405.1, QDH44433.1, QDH44370.1, QDH44412.1, QDH44138.1, QDH44309.1, QDH44079.1, QDH44323.1, QDH44377.1, QDH44145.1, QDH44419.1, QPP19815.1, QDH44447.1, QPP19823.1, QDH44426.1, QDH44337.1, UGV24184.1, UGV24168.1, QDH44288.1, QDH44316.1, UXD78538.1, QDH44239.1, UGV24188.1, ABZ10811.1, NP_044926.1, AAB95451.1, NP_043940.1, YP_077177.1, AAT46336.1, ABA71700.1, ABA71698.1, AAT46338.1, AB116638.1, APD78413.1, NP_049541.1, AAB95449.1, YP_077179.1, UGV24186.1, P03132.1, YP_680422.1, QDH44158.1, ASW20942.1, QDH44301.1, ABW34382.1, QDH44231.1, QDH44252.1, QDH44259.1, QDH44172.1, UXD78473.1, UXD78505.1, QDH44085.1, QDH44095.1, QDH44123.1, QDH44179.1, UXD78585.1, QDH44266.1, QDH44460.1, ACV74419.1, QDH44343.1, QDH44439.1, QDH44186.1, QDH44329.1, UXD78561.1, QDH44383.1, QDH44397.1, QDH44116.1, QDH44109.1, ACV74421.1, QDH44238.1, UXD78449.1, QDH44432.1, UXD78545.1, QDH44446.1, UXD78529.1, UXD78537.1, QDH44404.1, QDH44137.1, QDH44369.1, QDH44411.1, QDH44308.1, QDH44078.1, QDH44322.1, QDH44376.1, QDH44418.1, QDH44144.1, QDH44425.1, or QDH44315.1.

In some aspects, the Rep protein is a protein comprising a Rep protein catalytic-like domain (Interpro Accession No. IPR014835), i.e., a Rep_N domain. The Rep_N domain of the AAV Replication (Rep) protein is the catalytic domain of the Rep protein and has DNA binding and endonuclease activity. In some aspects, the Rep protein comprises a Rep_N domain form a parvovirus. In some aspects, the sequence encoding the Rep protein (i.e., a Rep_N domain-containing protein such as a Rep78 or Rep68) and the ITR sequences are selected, e.g., from the AAV2 Rep78, AAV2 Rep68, and AVV2 ITR homologous sequences in the genome of a parvovirus selected from the group consisting of Acheta domestica densovirus (Genbank NC_004290; SEQ ID NO: 142), Acheta domestica mini ambidensovirus (Genbank: NC_022564; SEQ ID NO: 143), Ambidensovirus_Croatia 17 S17 (Genbank: NC_077029; SEQ ID NO:144), Blattella germanica densovirus 1 (Genbank: NC_005041; SEQ ID NO:145), Bombus cryptarum densovirus (Genbank: NC_040626; SEQ ID NO:146), Bombyx mori densovirus 1 (Genbank: NC_003346; SEQ ID NO:147), Bombyx mori densovirus 5 (Genbank: NC_004287; SEQ ID NO: 148), Casphalia extranea densovirus (Genbank: NC_004288; SEQ ID NO:149), Sibine fusca densovirus (Genbank: NC_018399; SEQ ID NO:150), Cherax quadricarinatus densovirus (Genbank: NC_026943; SEQ ID NO:151), Culex pipiens densovirus (Genbank: NC_012685; SEQ ID NO:152), Danaus plexippus plexippus iteravirus (Genbank: NC_023842; SEQ ID NO:153), Decapod penstyldensovirus 1 (Genbank: NC_002190; SEQ ID NO:154), Dendrolimus punctatus densovirus (Genbank: NC_006555; SEQ ID NO:155), Diaphorina citri densovirus (Genbank: NC_030296; SEQ ID NO:156), Diatraea saccharalis densovirus (Genbank: NC_001899; SEQ ID NO:157), Dysaphis plantaginea densovirus (Genbank: NC_034532; SEQ ID NO:158), Galleria mellonella densovirus (Genbank: NC_004286; SEQ ID NO:159), Helicoverpa armigera densovirus (Genbank: NC_015718; SEQ ID NO:160), Junonia coenia densovirus (Genbank: NC_004284; SEQ ID NO:161), Mythimna loreyi densovirus (Genbank: NC_005341; SEQ ID NO:162), Pseudoplusia includens densovirus (Genbank: NC_019492; SEQ ID NO:163), Mosquito densovirus BR/07 (Genbank: NC_015115; SEQ ID NO:164), Myzus persicae densovirus 1 (Genbank: NC_005040; SEQ ID NO:165), Papilio polyxenes densovirus (Genbank: NC_018450; SEQ ID NO:166), Parus major densovirus (Genbank: NC_031450; SEQ ID NO:167), Penaeus monodon hepandensovirus 1 (Genbank: NC_007218; SEQ ID NO:168), Penaeus monodon hepandensovirus 4 (Genbank: NC_011545; SEQ ID NO:169), Periplaneta fuliginosa densovirus (NC_000936; SEQ ID NO:170), Planococcus citri densovirus (Genbank: NC_004289; SEQ ID NO:171), Sea star-associated densovirus (Genbank: NC_038532; SEQ ID NO:172), Solenopsis invicta densovirus (Genbank: NC_022748; SEQ ID NO:173), Aedes aegypti densovirus 2 (Genbank: NC_012636; SEQ ID NO:174), Anopheles gambiae densovirus (Genbank: NC_011317; SEQ ID NO:175), Aedes albopictus densovirus 2 (Genbank: NC_004285; SEQ ID NO:176), Fenneropenaeus chinensis hepandensovirus (Genbank: NC_014357; SEQ ID NO:177), Mouse kidney parvovirus (Genbank: NC_040843; SEQ ID NO:178), Penaeus stylirostris penstyldensovirus 1 (Genbank: NC_039043; SEQ ID NO:179), Porcine parvovirus 7 (Genbank: NC_040562; SEQ ID NO:180), Rat parvovirus 2 (Genbank: NC_055465; SEQ ID NO:181), Syngnathus scovelli chapparvovirus (Genbank: NC_055527; SEQ ID NO:182), Ursus americanus parvovirus (Genbank: NC_077031; SEQ ID NO:183), Cachavirus 1A (Genbank: NC_076028; SEQ ID NO:184), Chicken chapparvovirus HK (Genbank: NC_075989; SEQ ID NO:185), Turkey parvovirus 2 (Genbank: NC_075985; SEQ ID NO:186), Adeno-associated virus (Genbank: NC_040671; SEQ ID NO:187), Adeno-associated virus 1/Dependoparvovirus primate 1 (Genbank: NC_002077; SEQ ID NO:188), Adeno-associated virus 3/Dependoparvovirus primate 1 (Genbank: NC_001729; SEQ ID NO:189), Adeno-associated virus 4/Dependoparvovirus primate 1 (Genbank: NC_001829; SEQ ID NO:190), Adeno-associated virus 7 Dependoparvovirus primate 1 (Genbank: NC_006260; SEQ ID NO:191), Adeno-associated virus 8/Dependoparvovirus primate 1 (Genbank: NC_006261; SEQ ID NO:192), Adeno-associated virus 2/Dependoparvovirus primate 1 (Genbank: NC_001401; SEQ ID NO:193), Aleutian mink disease virus/Amdoparvovirus carnivoran 1 (Genbank: NC_001662; SEQ ID NO:194), Amdoparvovirus sp. (Genbank: NC_031751; SEQ ID NO:195), Artibeus jamaicensis parvovirus 1/Artiparvovirus chiropteran 1 (Genbank: NC_016752; SEQ ID NO:196), Avian adeno-associated virus ATCC VR-865/Dependoparvovirus avian 1 (Genbank: NC_004828; SEQ ID NO:197), Avian adeno-associated virus strain DA-1/Dependoparvovirus avian 1 (Genbank: NC_006263; SEQ ID NO:198), Bat adeno-associated virus YNM Dependoparvovirus chiropteran 1 (Genbank: NC_014468; SEQ ID NO:199), Bat bocavirus (Genbank: NC_029300; SEQ ID NO:200), Bat bocavirus WM40/Bocaparvovirus chiropteran 2 (Genbank: NC_039046; SEQ ID NO:201), Bat bocavirus AM30/Bocaparvovirus chiropteran 3 (Genbank: NC_039047; SEQ ID NO:202), Bearded dragon parvovirus/Dependoparvovirus squamate 2 (Genbank: NC_027429; SEQ ID NO:203), Bocaparvovirus lagomorph 1 (Genbank: NC_028973; SEQ ID NO:204), Bocaparvovirus primate 1 (Genbank: NC_007455; SEQ ID NO:205), Bocaparvovirus primate 1 (Genbank: NC_075120; SEQ ID NO:206), Bocaparvovirus primate 3 (Genbank: NC_055583; SEQ ID NO:207), Bocavirus gorilla/GBoV1/2009/Bocaparvovirus primate 1 (Genbank: NC_014358; SEQ ID NO:208), Human bocavirus 3/Bocaparvovirus primate 1 (Genbank: NC_012564; SEQ ID NO:209), Bocavirus pig/SX/China/2010/Bocaparvovirus ungulate 3 (Genbank: NC_038537; SEQ ID NO:210), Bosavirus MS-2016a/Copiparvovirus ungulate 5 (Genbank: NC_031959; SEQ ID NO:211), Bovine adeno-associated virus Dependoparvovirus mammalian 1 (Genbank NC_005889; SEQ ID NO:212), Adeno-associated virus 5/Dependoparvovirus mammalian 1 (Genbank: NC_006152; SEQ ID NO:213), Bovine hokovirus 1/Tetraparvovirus ungulate 1 (Genbank: NC_038898; SEQ ID NO:214), Bovine parvovirus/Bocaparvovirus ungulate 1 (Genbank: NC_001540; SEQ ID NO:215), Bovine parvovirus 1/Bocaparvovirus ungulate 1 (Genbank: NC_038895; SEQ ID NO:216), Bovine parvovirus 2/Copiparvovirus ungulate 1 (Genbank: NC_006259; SEQ ID NO:217), Bovine parvovirus 3/Erythroparvovirus ungulate 1 (Genbank: NC_074970; SEQ ID N02:218), Bovine parvovirus 3/Erythroparvovirus ungulate 1 (Genbank: NC_037053; SEQ ID NO:219), Bufavirus 3 (Genbank: NC_024888; SEQ ID NO:220), California sea lion adeno-associated virus 1 (Genbank: NC_038539; SEQ ID NO:221), California sea lion bocavirus 1/Bocaparvovirus pinniped 1 (Genbank: NC_038535; SEQ ID NO:222), California sea lion bocavirus 3/Bocaparvovirus pinniped 2 (Genbank: NC_038536; SEQ ID NO:223), Canine bocavirus 1/Bocaparvovirus carnivoran 2 (Genbank: NC_020499; SEQ ID NO:224), Canine minute virus/Bocaparvovirus carnivoran 1 (Genbank: NC_004442; SEQ ID NO:225), Canine parvovirus/Protoparvovirus carnivoran 1 (Genbank: NC_001539; SEQ ID NO:226), Chicken parvovirus ABU-P1/Aveparvovirus galliform 1 (Genbank: NC_024452; SEQ ID NO:227), Turkey parvovirus 1078/Aveparvovirus galliform 1 (Genbank: NC_024454; SEQ ID NO:228), Turkey parvovirus 260/Aveparvovirus galliform 1 (Genbank: NC_038534; SEQ ID NO:229), Chipmunk parvovirus Erythroparvovirus rodent 1 (Genbank: NC_038543; SEQ ID NO:230), Cutavirus Protoparvovirus primate 3 (Genbank: NC_039050; SEQ ID NO:231), Dromedary camel bocaparvovirus 1/Bocaparvovirus ungulate 7 (Genbank: NC_035186; SEQ ID NO:232), Dromedary camel bocaparvovirus 2/Bocaparvovirus ungulate 8 (Genbank: NC_035185; SEQ ID NO:233), Eidolon helvum (bat) parvovirus/Tetraparvovirus chiropteran 1 (Genbank: NC_016744; SEQ ID NO:234), Equine parvovirus H (Genbank: NC_040652; SEQ ID NO:235), Feline bocaparvovirus 2/Bocaparvovirus carnivoran 4 (Genbank: NC_022800; SEQ ID NO:236), Feline bocaparvovirus 3/Bocaparvovirus carnivoran 5 (Genbank: NC_039044; SEQ ID NO:237), Feline bocavirus/Bocaparvovirus carnivoran 3 (Genbank: NC_017823; SEQ ID NO:238), Goose parvovirus/Dependoparvovirus anseriform 1 (Genbank: NC_001701; SEQ ID NO:239), Muscovy duck parvovirus/Dependoparvovirus anseriform 1 (Genbank: NC_006147; SEQ ID NO:240), Gray fox amdovirus/Amdoparvovirus carnivoran 2 (Genbank: NC_038533; SEQ ID NO:241), Human bocavirus 2c PK/Bocaparvovirus primate 2 (Genbank: NC_012042; SEQ ID NO:242), Human bocavirus 4 NI/Bocaparvovirus primate 2 (Genbank: NC_012729; SEQ ID NO:243), Human erythrovirus V9 (Genbank: NC_004295; SEQ ID NO:244), Human parvovirus 4 GI/Tetraparvovirus primate 1 (Genbank: NC_007018; SEQ ID NO:245), Human parvovirus B19/Erythroparvovirus primate 1 (Genbank: NC_000883; SEQ ID NO:246), Lupine bocavirus (Genbank: NC_040533; SEQ ID NO:247), Megabat bufavirus 1/Protoparvovirus chiropteran 1 (Genbank: NC_029797; SEQ ID NO:248), Miniopterus schreibersii bat bocavirus/Bocaparvovirus chiropteran 4 (Genbank: NC_039048; SEQ ID NO:249), Mink bocavirus 1/Bocaparvovirus carnivoran 6 (Genbank: NC_030873; SEQ ID NO:250), Minute virus of mice/Protoparvovirus rodent 1 (Genbank: NC_001510; SEQ ID NO:251), Murine adeno-associated virus 1/Dependoparvovirus rodent 1 (Genbank: NC_055485; SEQ ID NO:252), Murine adeno-associated virus 2/Dependoparvovirus rodent 2 (Genbank: NC_055486; SEQ ID NO:253), Murine bocavirus/Bocaparvovirus rodent 2 (Genbank: NC_055487; SEQ ID NO:254), Myotis myotis bocavirus 1/Bocaparvovirus chiropteran 1 (Genbank: NC_039045; SEQ ID NO:255), Ovine hokovirus/Tetraparvovirus ungulate 4 (Genbank: NC_038547; SEQ ID NO:256), Parvovirus NIH-CQV (Genbank: NC_022089; SEQ ID NO:257), Parvovirus YX-2010/CHN/Tetraparvovirus ungulate 3 (Genbank: NC_038883; SEQ ID NO:258), Porcine parvovirus 2/Tetraparvovirus ungulate 3 (Genbank: NC_025965; SEQ ID NO:259), Pig-tailed macaque parvovirus/Erythroparvovirus primate 4 (Genbank: NC_038542; SEQ ID NO:260), Porcine bocavirus (Genbank: NC_023673; SEQ ID NO:261), Porcine bocavirus 1 pig/ZJD/China/2006/Bocaparvovirus ungulate 2 (Genbank: NC_024453; SEQ ID NO:262), Porcine bocavirus 3/Bocaparvovirus ungulate 5 (Genbank: NC_016031; SEQ ID NO:263), Porcine bocavirus 4-1/Bocaparvovirus ungulate 5 (Genbank: NC_016032; SEQ ID NO:264), Porcine bocavirus 5/JS677 (Genbank: NC_016647; SEQ ID NO:265), Porcine bocavirus H18/Bocaparvovirus ungulate 4 (Genbank: NC_038538; SEQ ID NO:266), Porcine hokovirus/Tetraparvovirus ungulate 2 (Genbank: NC_038546; SEQ ID NO:267), Porcine partetravirus (Genbank: NC_022104; SEQ ID NO:268), Porcine parvovirus/Protoparvovirus ungulate 1 (Genbank: NC_001718; SEQ ID NO:269), Porcine parvovirus 4/Copiparvovirus ungulate 2 (Genbank: NC_014665; SEQ ID NO:270), Porcine parvovirus 5 (Genbank: NC_023020; SEQ ID NO:271), Porcine parvovirus 6/Copiparvovirus ungulate 4 (Genbank: NC_023860; SEQ ID NO:272), Protoparvovirus Zsana/2013/HUN/Protoparvovirus ungulate 2 (Genbank: NC_043446; SEQ ID NO:273), Protoparvovirus eulipotyphla 1 (Genbank: NC_026815; SEQ ID NO:274), Protoparvovirus primate 1 (Genbank: NC_038544; SEQ ID NO:275), Raccoon dog amdovirus/Amdoparvovirus carnivoran 3 (Genbank: NC_025825; SEQ ID NO:276), Rat bocavirus/Bocaparvovirus rodent 1 (Genbank: NC_029133; SEQ ID NO:277), Rat bufavirus SY-2015/Protoparvovirus rodent 3 (Genbank: NC_028650; SEQ ID NO:278), Ratparvovirus 1/Protoparvovirus rodent 2 (Genbank: NC_038545; SEQ ID NO:279), Red-crowned crane parvovirus/Aveparvovirus gruiform 1 (Genbank: NC_040672; SEQ ID NO:280), Red-crowned crane parvovirus/Aveparvovirus gruiform 1 (Genbank: NC_040603; SEQ ID NO:281), Rhesus macaque parvovirus Erythroparvovirus primate 3 (Genbank: NC_038541; SEQ ID NO:282), Rhinolophus pusillus bocaparvovirus 1 (Genbank: NC_040623; SEQ ID NO:283), Rhinolophus pusillus bocaparvovirus 2 (Genbank: NC_040694; SEQ ID NO:284), Rhinolophus sinicus bocaparvovirus (Genbank: NC_031695; SEQ ID NO:285), Roe deer copiparvovirus/Copiparvovirus ungulate 3 (Genbank: NC_055518; SEQ ID NO:286), Rousettus leschenaultii bocaparvovirus 1/Bocaparvovirus chiropteran 5 (Genbank: NC_040695; SEQ ID NO:287), Seal parvovirus/Erythroparvovirus pinniped 1 (Genbank: NC_055458; SEQ ID NO:288), Simian parvovirus/Erythroparvovirus primate 2 (Genbank: NC_038540; SEQ ID NO:289), Skunk amdoparvovirus/Amdoparvovirus carnivoran 4 (Genbank: NC_034445; SEQ ID NO:290), Snake adeno-associated virus/Dependoparvovirus squamate 1 (Genbank: NC_006148; SEQ ID NO:291), Tetraparvovirus sp. (Genbank: NC_031670; SEQ ID NO:292), Tetraparvovirus ungulate 1 (Genbank: NC_028136; SEQ ID NO:293), Tetraparvovirus ungulate 3 (Genbank: NC_035180; SEQ ID NO:294), Ungulate bocaparvovirus 6/Bocaparvovirus ungulate 6 (Genbank: NC_030402; SEQ ID NO:295), Wuhary parvovirus 1/Protoparvovirus primate 2 (Genbank: NC_039049; SEQ ID NO:296), Red panda amdoparvovirus/Amdoparvovirus carnivoran 5 (Genbank: NC_077138; SEQ ID NO:297), Desmodus rotundus parvovirus (Genbank: NC_032097; SEQ ID NO:298), Sea otter parvovirus 1 (Genbank: NC_030837; SEQ ID NO:299), Sesavirus CSL10538 (Genbank: NC_026251; SEQ ID NO:300), Slow loris parvovirus 1 (Genbank: NC_025891; SEQ ID NO:301), Canine minute virus/Bocaparvovirus carnivoran 1 (Genbank: NC_075119; SEQ ID NO:302), Porcine parvovirus 6/Copiparvovirus ungulate 4 (Genbank: NC_075986; SEQ ID NO:303), Tusavirus 1/Protoparvovirus incertum 1 (Genbank: NC_075988; SEQ ID NO:304), Dromedary camel bocaparvovirus 1/Bocaparvovirus ungulate 7 (Genbank: NC_075998; SEQ ID NO:305), Equine parvovirus H (Genbank: NC_076001; SEQ ID NO:306), Fox parvovirus/Protoparvovirus carnivoran 4 (Genbank: NC_076133; SEQ ID NO:307), Canine protoparvovirus (Genbank: NC_076185; SEQ ID NO:308), Vicugna pacos bocaparvovirus/Bocaparvovirus ungulate 9 (Genbank: NC_076293; SEQ ID NO:309), Feline Dependoparvovirus/Dependoparvovirus carnivoran 1 (Genbank: NC_076473; SEQ ID NO:310), Canine bocavirus 3 (Genbank: NC_076995; SEQ ID NO:311), Rodent tetraparvovirus (Genbank: NC_077009; SEQ ID NO:312), Opossum tetraparvovirus (Genbank: NC_077010; SEQ ID NO:313), Pileated finch aveparvovirus (Genbank: NC_077011; SEQ ID NO:314), Rodent bocavirus (Genbank: NC_077012; SEQ ID NO:315), Desmodus rotundus dependoparvovirus (Genbank: NC_077013; SEQ ID NO:316), Avian chapparvovirus (Genbank: NC_077032; SEQ ID NO:317), Avian adeno-associated virus/Dependoparvovirus avian 1 (Genbank: NC_077033; SEQ ID NO:318), Macaca fascicularis chapparvovirus (Genbank: NC_077037; SEQ ID NO:319), Duck-associated chapparvovirus 1 (Genbank: NC_077098: SEQ ID NO:321), Duck-associated chapparvovirus 2 (Genbank: NC_077099; SEQ ID NO:322), Equine protoparvovirus (Genbank: NC_077112; SEQ ID NO:323), Newlavirus (Genbank: NC_077148; SEQ ID NO:324), Parvovirinae sp. (Genbank: NC_077156; SEQ ID NO:325), Feline panleukopenia virus FPV/INDIA/MZ26/Protoparvovirus carnivoran 1 (Genbank: PP035815; SEQ ID NO:326), Feline panleukopenia virus FPV/INDIA/MZ33/Protoparvovirus carnivoran 1 (Genbank: PP035816; SEQ ID NO:327), Feline panleukopenia virus FPV/INDIA/MZ35/Protoparvovirus carnivoran 1 (Genbank: PP035817; SEQ ID NO:328), Protoparvovirus carnivoran1/Protoparvovirus carnivoran 1 (Genbank: PP049248; SEQ ID NO:329), Goose parvovirus/Dependoparvovirus anseriform 1 (Genbank: PP058119; SEQ ID NO:330), Canine parvovirus 2b Arg22/Protoparvovirus carnivoran 1 (Genbank: OK888554; SEQ ID NO:331), Canine parvovirus 2c Arg35/Protoparvovirus carnivoran 1 (Genbank: OK888555; SEQ ID NO:332), Canine parvovirus 2a Arg41/Protoparvovirus carnivoran 1 (Genbank: OK888556; SEQ ID NO:333), Canine parvovirus 2c Arg 43/Protoparvovirus carnivoran 1 (Genbank: OK888557; SEQ ID NO:334), Canine parvovirus 2c Arg45/Protoparvovirus carnivoran 1 (Genbank: OK888558; SEQ ID NO:335), Canine parvovirus 2c UY382/Protoparvovirus carnivoran 1 (Genbank: OK888559; SEQ ID NO:336), Canine parvovirus 2a UY386/Protoparvovirus carnivoran 1 (Genbank: OK888560; SEQ ID NO:337), Canine parvovirus 2c UY391/Protoparvovirus carnivoran 1 (Genbank: OK888561; SEQ ID NO:338), Canine parvovirus 2a UY397/Protoparvovirus carnivoran 1 (Genbank: OK888562; SEQ ID NO:339), Canine parvovirus 2a UY406/Protoparvovirus carnivoran 1 (Genbank: OK888563; SEQ ID NO:340), Canine parvovirus 2c UY408/Protoparvovirus carnivoran 1 (Genbank: OK888564; SEQ ID NO:341), Canine parvovirus 2c UY410/Protoparvovirus carnivoran 1 (Genbank: OK888565; SEQ ID NO:342), Canine parvovirus 2c UY423/Protoparvovirus carnivoran 1 (Genbank: OK888566; SEQ ID NO:343), Canine parvovirus 2c UY426/Protoparvovirus carnivoran 1 (Genbank: OK888567; SEQ ID NO:344), Canine parvovirus 2a UY427/Protoparvovirus carnivoran 1 (Genbank: OK888568; SEQ ID NO:345), Canine parvovirus 2c UY430/Protoparvovirus carnivoran 1 (Genbank: OK888569; SEQ ID NO:346), Canine parvovirus 2a UY443/Protoparvovirus carnivoran 1 (Genbank: OK888570; SEQ ID NO:347), Canine parvovirus 2a UY445/Protoparvovirus carnivoran 1 (Genbank: OK888571; SEQ ID NO:348), Canine parvovirus 2a UY443/Protoparvovirus carnivoran 1 (Genbank: OK888572; SEQ ID NO:349), Canine parvovirus 2a UY459/Protoparvovirus carnivoran 1 (Genbank: OK888573; SEQ ID NO:350), Canine parvovirus 2a UY463/Protoparvovirus carnivoran 1 (Genbank: OK888574; SEQ ID NO:351), Canine parvovirus 2c UY467/Protoparvovirus carnivoran 1 (Genbank: OK888575; SEQ ID NO:352), Canine parvovirus 2a UY470/Protoparvovirus carnivoran 1 (Genbank: OK888576; SEQ ID NO:353), Canine parvovirus 2a UY476A/Protoparvovirus carnivoran 1 (Genbank: OK888577; SEQ ID NO:354), Canine parvovirus 2a UY486/Protoparvovirus carnivoran 1 (Genbank: OK888578; SEQ ID NO:355), Canine parvovirus 2a UY492/Protoparvovirus carnivoran 1 (Genbank: OK888579; SEQ ID NO:356), Canine parvovirus 2a UY503/Protoparvovirus carnivoran 1 (Genbank: OK888580; SEQ ID NO:357), Canine parvovirus 2a UY520/Protoparvovirus carnivoran 1 (Genbank: OK888581; SEQ ID NO:358), Canine parvovirus 2a UY530/Protoparvovirus carnivoran 1 (Genbank: OK888582; SEQ ID NO:359), Canine parvovirus 2a UY540/Protoparvovirus carnivoran 1 (Genbank: OK888583; SEQ ID NO:360), Canine parvovirus 2a UY568/Protoparvovirus carnivoran 1 (Genbank: OK888584; SEQ ID NO:361), Canine parvovirus 2a UY579/Protoparvovirus carnivoran 1 (Genbank: OK888585; SEQ ID NO:362), Canine parvovirus 2a UY593/Protoparvovirus carnivoran 1 (Genbank: OK888586; SEQ ID NO:363), Canine parvovirus 2a UY607/Protoparvovirus carnivoran 1 (Genbank: OK888587; SEQ ID NO:364), Canine parvovirus 2a UY618/Protoparvovirus carnivoran 1 (Genbank: OK888588; SEQ ID NO:365), Canine parvovirus 2a UY621/Protoparvovirus carnivoran 1 (Genbank: OK888589; SEQ ID NO:366), Canine parvovirus 2/Protoparvovirus carnivoran 1 (Genbank: OR037209; SEQ ID NO:367), Canine parvovirus 2/Protoparvovirus carnivoran 1 (Genbank: OR051684; SEQ ID NO:368), Canine parvovirus 2/Protoparvovirus carnivoran 1 (Genbank: OR051685; SEQ ID NO:369), Canine parvovirus 2/Protoparvovirus carnivoran 1 (Genbank: OR051686; SEQ ID NO:370), Human parvovirus B19/Erythroparvovirus primate 1 (Genbank: OR999319; SEQ ID NO:371), Human parvovirus 4/Tetraparvovirus primate 1 (Genbank: OR999320; SEQ ID NO:372), Cutavirus/Protoparvovirus primate 3 (Genbank: PP001440; SEQ ID NO:373), Cutavirus/Protoparvovirus primate 3 (Genbank: PP001441; SEQ ID NO:374), Cutavirus/Protoparvovirus primate 3 (Genbank: PP001442; SEQ ID NO:375), Cutavirus/Protoparvovirus primate 3 (Genbank: PP001443; SEQ ID NO:376), Cutavirus/Protoparvovirus primate 3 (Genbank: PP001444; SEQ ID NO:377), Cutavirus/Protoparvovirus primate 3 (Genbank: PP001445; SEQ ID NO:378), Cutavirus/Protoparvovirus primate 3 (Genbank: PP001446; SEQ ID NO:379), Canine parvovirus/Protoparvovirus carnivoran 1 (Genbank: OR992670; SEQ ID NO:380), Canine parvovirus/Protoparvovirus carnivoran 1 (Genbank: OR992671; SEQ ID NO:381), Canine parvovirus/Protoparvovirus carnivoran 1 (Genbank: OR992672; SEQ ID NO:382), Canine parvovirus/Protoparvovirus carnivoran 1 (Genbank: OR992673; SEQ ID NO:383), Canine parvovirus/Protoparvovirus carnivoran 1 (Genbank: OR992674; SEQ ID NO:384), Canine parvovirus/Protoparvovirus carnivoran 1 (Genbank: OR992675; SEQ ID NO:385), Feline panleukopenia virus Protoparvovirus carnivoran 1 (Genbank: OP985508; SEQ ID NO:386), and Feline panleukopenia virus Protoparvovirus carnivoran 1 (Genbank: OP985509; SEQ ID NO:387).

In the list above, when two names are separated by “/” the first name corresponds to the serotype of the virus and the second name corresponds to the species of the virus. In some aspects, the serotype of a virus can be followed by the isolate name. For example, in the entry “Canine parvovirus 2b Arg22/Protoparvovirus carnivoran 1,” the species name is “Protoparvovirus carnivoran 1,” the serotype is “Canine parvovirus 2b,” and the isolate is “Arg22.”

In some aspects, the mRNA sequence encoding the Rep78 protein, e.g., an AAV2 Rep78 protein or an ortholog thereof, is derived from the genome of a parvovirus having a genomic sequence selected from the group consisting of Genbank Acc. No. NC_004290, NC_022564, NC_077029, NC_005041, NC_040626, NC_003346, NC_004287, NC_004288, NC_018399, NC_026943, NC_012685, NC_023842, NC_002190, NC_006555, NC_030296, NC_001899, NC_034532, NC_004286, NC_015718, NC_004284, NC_005341, NC_019492, NC_015115, NC_005040, NC_018450, NC_031450, NC_007218, NC_011545, NC_000936, NC_004289, NC_038532, NC_022748, NC_012636, NC_011317, NC_004285, NC_014357, NC_040843, NC_039043, NC_040562, NC_055465, NC_055527, NC_077031, NC_076028, NC_075989, NC_075985, NC_040671, NC_002077, NC_001729, NC_001829, NC_006260, NC_006261, NC_001401, NC_001662, NC_031751, NC_016752, NC_004828, NC_006263, NC_014468, NC_029300, NC_03904, NC_039047, NC_027429, NC_028973, NC_007455, NC_075120, NC_055583, NC_014358, NC_012564, NC_038537, NC_031959, NC_005889, NC_006152, NC_038898, NC_001540, NC_038895, NC_006259, NC_074970, NC_037053, NC_024888, NC_038539, NC_038535, NC_038536, NC_020499, NC_004442, NC_001539, NC_024452, NC_024454, NC_038534, NC_038543, NC_039050, NC_035186, NC_035185, NC_016744, NC_040652, NC_022800, NC_039044, NC_017823, NC_001701, NC_006147, NC_038533, NC_012042, NC_012729, NC_004295, NC_007018, NC_000883, NC_040533, NC_029797, NC_039048, NC_030873, NC_001510, NC_055485, NC_055486, NC_055487, NC_039045, NC_038547, NC_022089, NC_038883, NC_025965, NC_038542, NC_023673, NC_024453, NC_016031, NC_016032, NC_016647, NC_038538, NC_038546, NC_022104, NC_001718, NC_014665, NC_023020, NC_023860, NC_043446, NC_026815, NC_038544, NC_025825, NC_029133, NC_028650, NC_038545, NC_040672, NC_040603, NC_038541, NC_040623, NC_040694, NC_031695, NC_055518, NC_040695, NC_055458, NC_038540, NC_034445, NC_006148, NC_031670, NC_028136, NC_035180, NC_030402, NC_039049, NC_077138, NC_032097, NC_030837, NC_026251, NC_025891, NC_075119, NC_075986, NC_075988, NC_075998, NC_076001, NC_076133, NC_076185, NC_076293, NC_076473, NC_076995, NC_077009, NC_077010, NC_077011, NC_077012, NC_077013, NC_077032, NC_077033, NC_077037, NC_077098, NC_077099, NC_077112, NC_077148, NC_077156, PP035815, PP035816, PP035817, PP049248, PP058119, OK888554, OK888555, OK888556, OK888557, OK888558, OK888559, OK888560, OK888561, OK888562, OK888563, OK888564, OK888565, OK888566, OK888567, OK888568, OK888569, OK888570, OK888571, OK888572, OK888573, OK888574, OK888575, OK888576, OK888577, OK888578, OK888579, OK888580, OK888581, OK888582, OK888583, OK888584, OK888585, OK888586, OK888587, OK888588, OK888589, OR037209, OR051684, OR051685, OR051686, OR999319, OR999320, PP001440, PP001441, PP001442, PP001443, PP001444, PP001445, PP001446, OR992670, OR992671, OR992672, OR992673, OR992674, OR992675, OP985508, and OP985509.

In some aspects, the mRNA sequence encoding the Rep68 protein, e.g., an AAV2 Rep78 protein or an ortholog thereof, is derived from the genome of a parvovirus having a genomic sequence selected from the group consisting of Genbank Acc. No. NC_004290, NC_022564, NC_077029, NC_005041, NC_040626, NC_003346, NC_004287, NC_004288, NC_018399, NC_026943, NC_012685, NC_023842, NC_002190, NC_006555, NC_030296, NC_001899, NC_034532, NC_004286, NC_015718, NC_004284, NC_005341, NC_019492, NC_015115, NC_005040, NC_018450, NC_031450, NC_007218, NC_011545, NC_000936, NC_004289, NC_038532, NC_022748, NC_012636, NC_011317, NC_004285, NC_014357, NC_040843, NC_039043, NC_040562, NC_055465, NC_055527, NC_077031, NC_076028, NC_075989, NC_075985, NC_040671, NC_002077, NC_001729, NC_001829, NC_006260, NC_006261, NC_001401, NC_001662, NC_031751, NC_016752, NC_004828, NC_006263, NC_014468, NC_029300, NC_03904, NC_039047, NC_027429, NC_028973, NC_007455, NC_075120, NC_055583, NC_014358, NC_012564, NC_038537, NC_031959, NC_005889, NC_006152, NC_038898, NC_001540, NC_038895, NC_006259, NC_074970, NC_037053, NC_024888, NC_038539, NC_038535, NC_038536, NC_020499, NC_004442, NC_001539, NC_024452, NC_024454, NC_038534, NC_038543, NC_039050, NC_035186, NC_035185, NC_016744, NC_040652, NC_022800, NC_039044, NC_017823, NC_001701, NC_006147, NC_038533, NC_012042, NC_012729, NC_004295, NC_007018, NC_000883, NC_040533, NC_029797, NC_039048, NC_030873, NC_001510, NC_055485, NC_055486, NC_055487, NC_039045, NC_038547, NC_022089, NC_038883, NC_025965, NC_038542, NC_023673, NC_024453, NC_016031, NC_016032, NC_016647, NC_038538, NC_038546, NC_022104, NC_001718, NC_014665, NC_023020, NC_023860, NC_043446, NC_026815, NC_038544, NC_025825, NC_029133, NC_028650, NC_038545, NC_040672, NC_040603, NC_038541, NC_040623, NC_040694, NC_031695, NC_055518, NC_040695, NC_055458, NC_038540, NC_034445, NC_006148, NC_031670, NC_028136, NC_035180, NC_030402, NC_039049, NC_077138, NC_032097, NC_030837, NC_026251, NC_025891, NC_075119, NC_075986, NC_075988, NC_075998, NC_076001, NC_076133, NC_076185, NC_076293, NC_076473, NC_076995, NC_077009, NC_077010, NC_077011, NC_077012, NC_077013, NC_077032, NC_077033, NC_077037, NC_077098, NC_077099, NC_077112, NC_077148, NC_077156, PP035815, PP035816, PP035817, PP049248, PP058119, OK888554, OK888555, OK888556, OK888557, OK888558, OK888559, OK888560, OK888561, OK888562, OK888563, OK888564, OK888565, OK888566, OK888567, OK888568, OK888569, OK888570, OK888571, OK888572, OK888573, OK888574, OK888575, OK888576, OK888577, OK888578, OK888579, OK888580, OK888581, OK888582, OK888583, OK888584, OK888585, OK888586, OK888587, OK888588, OK888589, OR037209, OR051684, OR051685, OR051686, OR999319, OR999320, PP001440, PP001441, PP001442, PP001443, PP001444, PP001445, PP001446, OR992670, OR992671, OR992672, OR992673, OR992674, OR992675, OP985508, and OP985509.

In some aspects, the ITR sequences, e.g., AAV2 ITR sequences or orthologs thereof, are derived from the genome of a parvovirus having a genomic sequence selected from the group consisting of Genbank Acc. No. NC_004290, NC_022564, NC_077029, NC_005041, NC_040626, NC_003346, NC_004287, NC_004288, NC_018399, NC_026943, NC_012685, NC_023842, NC_002190, NC_006555, NC_030296, NC_001899, NC_034532, NC_004286, NC_015718, NC_004284, NC_005341, NC_019492, NC_015115, NC_005040, NC_018450, NC_031450, NC_007218, NC_011545, NC_000936, NC_004289, NC_038532, NC_022748, NC_012636, NC_011317, NC_004285, NC_014357, NC_040843, NC_039043, NC_040562, NC_055465, NC_055527, NC_077031, NC_076028, NC_075989, NC_075985, NC_040671, NC_002077, NC_001729, NC_001829, NC_006260, NC_006261, NC_001401, NC_001662, NC_031751, NC_016752, NC_004828, NC_006263, NC_014468, NC_029300, NC_03904, NC_039047, NC_027429, NC_028973, NC_007455, NC_075120, NC_055583, NC_014358, NC_012564, NC_038537, NC_031959, NC_005889, NC_006152, NC_038898, NC_001540, NC_038895, NC_006259, NC_074970, NC_037053, NC_024888, NC_038539, NC_038535, NC_038536, NC_020499, NC_004442, NC_001539, NC_024452, NC_024454, NC_038534, NC_038543, NC_039050, NC_035186, NC_035185, NC_016744, NC_040652, NC_022800, NC_039044, NC_017823, NC_001701, NC_006147, NC_038533, NC_012042, NC_012729, NC_004295, NC_007018, NC_000883, NC_040533, NC_029797, NC_039048, NC_030873, NC_001510, NC_055485, NC_055486, NC_055487, NC_039045, NC_038547, NC_022089, NC_038883, NC_025965, NC_038542, NC_023673, NC_024453, NC_016031, NC_016032, NC_016647, NC_038538, NC_038546, NC_022104, NC_001718, NC_014665, NC_023020, NC_023860, NC_043446, NC_026815, NC_038544, NC_025825, NC_029133, NC_028650, NC_038545, NC_040672, NC_040603, NC_038541, NC_040623, NC_040694, NC_031695, NC_055518, NC_040695, NC_055458, NC_038540, NC_034445, NC_006148, NC_031670, NC_028136, NC_035180, NC_030402, NC_039049, NC_077138, NC_032097, NC_030837, NC_026251, NC_025891, NC_075119, NC_075986, NC_075988, NC_075998, NC_076001, NC_076133, NC_076185, NC_076293, NC_076473, NC_076995, NC_077009, NC_077010, NC_077011, NC_077012, NC_077013, NC_077032, NC_077033, NC_077037, NC_077098, NC_077099, NC_077112, NC_077148, NC_077156, PP035815, PP035816, PP035817, PP049248, PP058119, OK888554, OK888555, OK888556, OK888557, OK888558, OK888559, OK888560, OK888561, OK888562, OK888563, OK888564, OK888565, OK888566, OK888567, OK888568, OK888569, OK888570, OK888571, OK888572, OK888573, OK888574, OK888575, OK888576, OK888577, OK888578, OK888579, OK888580, OK888581, OK888582, OK888583, OK888584, OK888585, OK888586, OK888587, OK888588, OK888589, OR037209, OR051684, OR051685, OR051686, OR999319, OR999320, PP001440, PP001441, PP001442, PP001443, PP001444, PP001445, PP001446, OR992670, OR992671, OR992672, OR992673, OR992674, OR992675, OP985508, and OP985509.

The nucleotide sequence encoding a Rep78 or Rep68 protein or ortholog thereof in a genomic sequence disclosed herein can be determined by a person of ordinary skill in the art without undue experimentation. For example, the potential open reading frames identified via a 3-frame translation of the genomic sequence can be scanned for the presence of a Rep_N domain (Interpro IPR014835) alone or combined with a parvovirus NS1 helicase domain (Interpro IPR001257) using the InterproScan tool available at www.ebi.ac.uk/interpro/search/sequence/. Alternatively, the open reading frames can be identified an ORF-calling program such as Prodigal (e.g., version 2.6.3) (Hyatt et al. (2010) BMC Bioinformatics, 11, 119), GLIMMER (e.g., version 3.02) (Delcher et al. (2007) Bioinformatics 23, 673-679), GeneMarkS (e.g., version 4.32) (Besemer et al. (2001) Nucleic Acids Research 27, 3911-3920), PHANOTATE (e.g., version 1.5.0) (McNair et al. (2019) Bioinformatics 35, 4537-4542), Metaprodigal (e.g., version 2.6.3) (Hyatt et al. (2012) Bioinformatics 28, 2223-2230), FragGeneScan (e.g., version 1.31), (Rho et al. (2010) Nucleic Acids Research 38, e191-e191), MetaGeneAnnotator (MGA) (Noguchi et al. (2008) DNA Research 15, 387-396), or AUGUSTUS (e.g., version 3.4.0) (Stanke et al. (2008) Bioinformatics 24, 637-644). The nucleotide sequence of the ITRs in a genomic sequence disclosed herein can be determined by a person of ordinary skill in the art without undue experimentation. For example, since the 3′ ITR is the reverse complement of the 5′ ITR, a subsequence longer than the length of a typical ITR taken from the 5′ end of the parvovirus genome can be aligned with the reverse complement of a subsequence with the same length taken from the 3′ end of the parvovirus genome, and the aligned regions identical or almost identical would identify the position and sequences of the ITRs.

### Other AAV-ITR Vector Components

Enhancer: In some aspects, the AAV-ITR vector can comprise an enhancer region. In some aspects, the AAV-ITR vector can comprise a CMV enhancer region.

Promoter: The promoter drives the expression of the AAV transgene, i.e., the polynucleotide encoding a GOI in the AAV-ITR vector. Promoters are upstream or 5′ of the gene they control. Promoters can be organism or tissue type-specific, which can help restrict the expression of the transgene to a specific cell, tissue, or location. Promoters can also drive different levels of expression. For example, CAG is a strong promoter that drives very high, ubiquitous expression, while the human synapsin 1 promoter is neuronal specific (Haery et al., 2019).

In some aspects, the AAV-ITR vector can comprise a promoter such as, but not limited to, cytomegalovirus (CMV), U6, chicken β-actin (CBA), CAG, ubiquitin C (UBC), β glucuronidase (GUSB), NSE, Synapsin, MeCP2, human elongation factor 1a-subunit (EF 1a), or GFAP promoter. In some aspects, the promoter for an AAV-ITR vector of the present disclosure is a CMV promoter. As another non-limiting example, the promoter for an AAV-ITR vector of the present disclosure is a U6 promoter. In some aspects, the AAV-ITR vector can comprise a CMV and a U6 promoter. In some aspects, the AAV-ITR vector can comprise a HI promoter. In some aspects, the AAV-ITR vector can comprise a CBA promoter. In some aspects, the AAV-ITR vector can comprise a UBC promoter. In some aspects, the AAV-ITR vector can comprise a GUSB promoter. In some aspects, the AAV-ITR vector can comprise a NSE promoter. In some aspects, the AAV-ITR vector can comprise a Synapsin promoter. In some aspects, the AAV-ITR vector can comprise a MeCP2 promoter. In some aspects, the AAV-ITR vector can comprise a GFAP promoter.

Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (MCK) promoter, mammalian desmin (DES) promoter, mammalian troponin I (TNNI2) promoter, and mammalian skeletal alpha-actin (ASKA) promoter (see, e.g. U.S. Patent Publication US 20110212529, the contents of which are herein incorporated by reference in their entirety). Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene flP2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In some aspects, the AAV vector comprises a ubiquitous promoter. Non-limiting examples of ubiquitous promoters include, e.g., CMV, CBA (including derivatives CAG, CBh, etc.), EF-la, PGK, UBC, GUSB (hGBp), and UCOE (promoter of HNRPA2B1-CBX3).

Yu et al. (2011) Molecular Pain 7:63, evaluated the expression of EGFP under the CAG, EFIa, PGK and UBC promoters in rat DRG cells and primary DRG cells using lentiviral vectors and found that UBC showed weaker expression than the other 3 promoters and only 10-12% glial expression was seen for all promoters. Soderblom et al. (2015) E. Neuro 2(2), evaluated the expression of eGFP in AAV 8 with CMV and UBC promoters and AAV2 with the CMV promoter after injection in the motor cortex.

Intranasal administration of a plasmid containing a UBC or EFIa promoter showed a sustained airway expression greater than the expression with the CMV promoter. See, e.g., Gill et al. (2001) Gene Therapy 8:1539-1546. Husain et al. (2009) Gene Therapy 16(7):927-932, evaluated a HOH construct with a hGUSB promoter, an HSV-ILAT promoter and an NSE promoter and found that the HOH construct showed weaker expression than NSE in mouse brain. Passini and Wolfe (2001) J. Virol. 75:12382-12392, evaluated the long-term effects of the HOH vector following an intraventricular injection in neonatal mice and found that there was sustained expression for at least 1 year. Low expression in all brain regions was found by Xu et al. (2001) Gene Therapy 8:1323-1332, when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920 nucleotide promoter which are both absent in the liver but NFH is abundant in the sensory proprioceptive neurons, brain and spinal cord and NFH is present in the heart. Scn8a is a 470 nucleotide promoter which expresses throughout the DRG, spinal cord and brain with particularly high expression seen in the hippocampal neurons and cerebellar Purkinje cells, cortex, thalamus and hypothalamus. See, e.g., Drews et al. (0027) Mamm. Genome 18:723-731; and Raymond et al. (2004) J. Biol. Chem. 279(44) 46234-46241. Any of the promoters taught by the aforementioned Yu, Soderblom, Gill, Husain, Passini, Xu, Drews or Raymond can be used in the present disclosure.

In some aspects, the promoter is not cell specific. In some aspects, the promoter is an ubiquitin c (UBC) promoter. The UBC promoter can have a size of 300-350 nucleotides. In some aspects, the UBC promoter is 332 nucleotides. In some aspects, the promoter is a 0-glucuronidase (GUSB) promoter. The GUSB promoter can have a size of 350-400 nucleotides. In some aspects, the GUSB promoter is 378 nucleotides. In some aspects, the promoter is a neurofilament light (NFL) promoter. The NFL promoter can have a size of 600-700 nucleotides. In some aspects, the NFL promoter is 650 nucleotides. In some aspects, the promoter is a neurofilament heavy (NFH) promoter. The NFH promoter can have a size of 900-950 nucleotides. In some aspects, the promoter is a scn8a promoter.

In some aspects, the AAV-ITR vector comprises a Pol III promoter. In some aspects, the AAV vector comprises a PI promoter. In some aspects, the AAV-ITR vector comprises a FXN promoter. In some aspects, the promoter is a phosphoglycerate kinase 1 (PGK) promoter. In some aspects, the promoter is a chicken β-actin (CBA) promoter. In some aspects, the promoter is a CAG promoter which is a construct comprising the cytomegalovirus (CMV) enhancer fused to the chicken beta-actin (CBA) promoter. In some aspects, the promoter is a cytomegalovirus (CMV) promoter. In some aspects, the AAV-ITR vector comprises a HI promoter. In some aspects, the AAV-ITR vector comprises a U6 promoter. In some aspects, the AAV-ITR vector comprises a SP6 promoter.

In some aspects, the promoter is a liver or a skeletal muscle promoter. Non-limiting examples of liver promoters include human a-1-antitrypsin (hAAT) and thyroxine binding globulin (TBG). Non-limiting examples of skeletal muscle promoters include Desmin, MCK or synthetic C5-12. In some aspects, the promoter is an RNA pol III promoter. In some aspects, the RNA pol III promoter is U6. In some aspects, the RNA pol III promoter is HI. In some aspects, the AAV-ITR vector comprises two promoters. In some aspects, the promoters are an EFla promoter and a CMV promoter.

Intron: In some aspects, the payload region of an AAV-ITR vector of the present disclosure comprises at least one element to enhance the expression such as one or more introns or portions thereof. Non-limiting examples of introns include, MVM (67-97 bps), FIX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In some aspects, the intron or intron portion can be between about 100 nucleotides and about 500 nucleotides in length. The intron can have a length of about 80 nucleotides, about 90 nucleotides, about 100 nucleotides, about 110 nucleotides, about 120 nucleotides, about 130 nucleotides, about 140 nucleotides, about 150 nucleotides, about 160 nucleotides, about 170 nucleotides, about 171 nucleotides, about 172 nucleotides, about 173 nucleotides, about 174 nucleotides, about 175 nucleotides, about 176 nucleotides, about 177 nucleotides, about 178 nucleotides, about 179 nucleotides, about 180 nucleotides, about 190 nucleotides, about 200 nucleotides, about 210 nucleotides, about 220 nucleotides, about 230 nucleotides, about 240 nucleotides, about 250 nucleotides, about 260 nucleotides, about 270 nucleotides, about 280 nucleotides, about 290 nucleotides, about 300 nucleotides, about 310 nucleotides, about 320 nucleotides, about 330 nucleotides, about 340 nucleotides, about 350 nucleotides, about 360 nucleotides, about 370 nucleotides, about 380 nucleotides, about 390 nucleotides, about 400 nucleotides, about 410 nucleotides, about 420 nucleotides, about 430 nucleotides, about 440 nucleotides, about 450 nucleotides, about 460 nucleotides, about 470 nucleotides, about 480 nucleotides, about 490 nucleotides, or about 500 nucleotides. The intron can have a length between about 80 and about 100 nucleotides, between about 80 and about 120 nucleotides, between about 80 and about 140 nucleotides, between about 80 and about 160 nucleotides, between about 80 and about 180 nucleotides, between about 80 and about 200 nucleotides, between about 80 and about 250 nucleotides, between about 80 and about 300 nucleotides, between about 80 and about 350 nucleotides, between about 80 and about 400 nucleotides, between about 80 and about 450 nucleotides, between about 80 and about 500 nucleotides, between about 200 and about 300 nucleotides, between about 200 and about 400 nucleotides, between about 200 and about 500 nucleotides, between about 300 and about 400 nucleotides, between about 300 and about 500 nucleotides, or between about 400 and about 500 nucleotides.

Poly A Signal: In some aspects, the AAV-ITR vectors of the present disclosure comprise at least one polyadenylation signal. The polyadenylation or Poly(A) is the process required for the synthesis of messenger RNA (mRNA) in which an endonucleolytic RNA cleavage is coupled with synthesis of polyadenosine monophosphate (adenine base) on the newly formed 3′ end. The sequence elements for polyadenylation include the polyadenylation signal and the polyadenylation site. In mRNA or cDNA the added stretch of polyadenosine monophosphate is the polyadenylation tail. Most human polyadenylation signals (PolyA signal) contain the conserved motif AATAAA. The polyadenylation signals are located downstream of the 3′ exons. Polyadenylation sites (PolyA sites) are the sites of cleavage at which a polyA tail is added in mRNA. It is localized downstream of the PolyA signal. The sequence at/or immediately 5′ to the site of RNA cleavage is frequently (but not always) CA. The PolyA tail is a stretch of adenosine monophosphate (with only adenine bases) at the 3′ end of mRNA or cDNA. See Wahle et al. (1992) Ann. Rev. Biochem. 61:419-440; and Manley et al. (1996) Science 274:1481-1482, which are herein incorporated by reference in their entireties.

The terms “polyadenylation sequence” or “polyA sequence” is used interchangeable with the terms polyadenylation tail, and polyA tail. The polyadenylation or polyA tail aids in the nuclear export of RNA and RNA translation, and promotes RNA transcript longevity. Many protein-coding genes have more than one polyadenylation site. See Proudfoot (2011) Genes Dev. 25(17):1770-1782. The AAV vectors of the present disclosure can comprise a polyadenylation sequence between the 3′ end of the payload coding sequence and the 5′ end of the 3′ ITR. In some aspects, the polyA sequence can range from absent to about 500 nucleotides in length. In some aspects, the polyadenylation sequence is between about 50 and about 100 nucleotides, between about 50 and about 150 nucleotides, between about 50 and about 160 nucleotides, between about 50 and about 200 nucleotides, between about 60 and about 100 nucleotides, between about 60 and about 150 nucleotides, between about 60 and about 160 nucleotides, between about 60 and about 200 nucleotides, between about 70 and about 100 nucleotides, between about 70 and about 150 nucleotides, between about 70 and about 160 nucleotides, between about 70 and about 200 nucleotides, between about 80 and about 100 nucleotides, between about 80 and about 150 nucleotides, between about 80 and about 160 nucleotides, between about 80 and about 200 nucleotides, between about 90 and about 100 nucleotides, between about 90 and about 150 nucleotides, between about 90 and about 160 nucleotides, or between about 90 and about 200 nucleotides in length. In some aspects. The AAV-ITR vector of the present disclosure comprises a Tbgh polyA signal, i.e., a bovine growth hormone polyadenylation signal.

### Other Rep Vector Components

Promoter: In some aspects, the Rep vectors of the present disclosure comprise a promoter. In some aspects, the Rep vectors of the present disclosure comprise a T7 promoter (PT7). The T7 promoter is a sequence of DNA 18 base pairs long up to transcription start site at +1 (5′-taatacgactcactatag-3′) that is recognized by T7 RNA polymerase. The T7 promoter is commonly used to regulate gene expression of recombinant proteins.

5′ UTR: By definition, wild-type untranslated regions (UTRs) of a gene are transcribed but not translated. Generally, the 5′ UTR starts at the transcription start site and ends at the start codon and the 3′ UTR starts immediately following the stop codon and continues until the termination signal for transcription. Features typically found in abundantly expressed genes of specific target organs can be engineered into UTRs to enhance transcribed product stability and production. In some aspects, a 5′ UTR from mRNA normally expressed in the liver (e.g., albumin, serum amyloid A, apolipoprotein A/B/E, transferrin, alpha fetoprotein, erythropoietin, or Factor VIII) can be used in AAV vector of the disclosure to enhance expression, e.g., in brain tissue, and specifically in neuronal cells. Wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus ccr(a/g)ccaugg, where R is a purine (adenine or guanine) three bases upstream of the start codon (ATG), which is followed by another ‘G. In some aspects, the 5′UTR in an AAV vector of the present disclosure includes a Kozak sequence. In some aspects, the 5′UTR in an AAV vector of the present disclosure does not include a Kozak sequence.

Exemplary 5′-untranslated regions that can be used in the vectors disclosed herein include the 5′ UTR of SEQ ID NOS: 388 to 405. Variants of 5′ UTRs of SEQ ID NOS: 388 to 405 can be utilized wherein one or more nucleotides are added or removed to the termini, including A, T, C or G.

3′ UTR: Wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety). Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping uuauuua(u/a)(u/a) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Shown in the table below is a listing of 3′-untranslated regions that can be used in the vectors disclosed herein. Variants of 3′ UTRs can be utilized wherein one or more nucleotides are added or removed to the termini, including A, T, C or G.

TABLE 6
Exemplary 3′-Untranslated Regions

| Name/ Description | SEQ ID NO. |
|---|---|
| Creatine Kinase | 406 |
| Myoglobin | 407 |
| α-actin | 408 |
| Albumin | 409 |
| α-globin | 410 |
| G-CSF | 411 |
| Col1a2; collagen, type I, alpha 2 | 412 |
| Col6a2; collagen, type VI, alpha 2 | 413 |
| RPN1; ribophorin I | 414 |
| LRP1; low density lipoprotein receptor-related protein 1 | 415 |
| Nnt1; cardiotrophin-like cytokine factor 1 | 416 |
| Col6a1; collagen, type VI, alpha 1 | 417 |
| Calr; calreticulin | 418 |
| Col1a1; collagen, type I, alpha 1 | 419 |
| Plod1; procollagen-lysine, 2-oxoglutarate 5-dioxygenase | 420 |
| 1 | |
| Nucb1; nucleobindin 1 | 421 |
| α-globin (short) | 422 |
| α-globin | 423 |
| (long) | |

Poly A Tail: In some aspects, a Rep vector comprises a poly A tail. During RNA processing, a long chain of adenine nucleotides (poly-A tail) can be added to a polynucleotide such as an mRNA molecule in order to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, about 80 to about 250 nucleotides long, including about 80, about 90, about 100, about 110, about 120, about 130, about 140, about 150, about 160, about 170, about 180, about 190, about 200, about 210, about 220, about 230, about 240 or about 250 nucleotides long. In some aspects, the poly A tail includes terminal groups incorporated for stabilization. In some aspects, the poly A can include a des-3′ hydroxyl tail. The poly A tail can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present invention can be designed to encode transcripts with alternative poly A tail structures including a histone mRNA tail. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths can provide certain advantages to the vector of the present disclosure. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some aspects, the poly-A tail is designed relative to the length of the overall vector or the length of a particular region of the vector, e.g., an ORF encoding a gene of interest. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression. Additionally, multiple distinct polynucleotides can be linked together via the PABP (Poly-A binding protein) through the 3′-end using modified nucleotides at the 3′-terminus of the poly-A tail.

In some aspects, the polynucleotides of the present invention are designed to include a polyA-G Quartet region. The G-quartet is a cyclic hydrogen bonded array of four guanine nucleotides that can be formed by G-rich sequences in both DNA and RNA. In this aspect, the G-quartet is incorporated at the end of the poly-A tail. The resultant polynucleotide is assayed for stability, protein production and other parameters including half-life at various time points. The polyA-G quartet results in protein production from an mRNA equivalent to at least 75% of that seen using a poly-A tail of 120 nucleotides alone.

### Lipidic Delivery Systems and Polymeric Delivery Systems

In some aspects, the transfection and/or targeting of the AAV-ITR vector and/or Rep vector components of an AAV-ITR based gene delivery system of the present disclosure is mediated by a delivery system such as a lipidic delivery system (e.g., a liposome, a lipid nanoparticle, or a combination thereof) or a polymeric delivery system, or a combination thereof.

In a specific aspect, the AAV-ITR vector and Rep vector components of an AAV-ITR based gene delivery system of the present disclosure are encapsulated in liposomes. In some aspects, the AAV-ITR vector is encapsulated in a liposome optimized for delivery of dsDNA. In some aspects, the Rep vector is encapsulated in a liposome optimized for delivery of mRNA.

### Lipidic Delivery Systems: Liposomes and Lipid Nanoparticles

Physicochemical properties of lipids such as biocompatibility, low susceptibility to erosion phenomena, and slow water uptake make lipids an ideal nanocarrier system to improve active pharmaceutical ingredient aqueous solubility, bioavailability, and effective therapy. In addition, lipid-based systems improve storage and delivery while inhibiting oxidation, degradation, and decomposition. See Rawat et al. (2006) Biol. Pharm. Bull. 29: 1790-1798. Unlike other delivery systems, lipid-based drug delivery systems exhibit a major advantage over other methods due to their ability to cross the gut, gastro-intestinal tract (GIT), blood vessels, and blood brain barrier. See Patel et al. (2021) Eur. J. Lipid Sci. Technol. 2021, 123, 2000264, and Muller et al. (2002) Adv. Drug Deliv. Rev. 54: S131-S155. Some lipid-based drug delivery systems include self-emulsifying systems, LNPs, nanostructured lipid carriers (NLCs), and liposomes. See Salawi (2022) Drug Deliv. 29, 1811-1823; Mishra et al. (2021) Expert Opin. Drug Deliv. 18, 315-332; Khairnar et al. (2022) Pharmaceutics 14, 1886; Mirchandani et al. (2021) J. Control. Release 335, 457-464; Izza et al. (2022) ACS Appl. Nano Mater. 5, 9958-9969; Wang et al. (2022) AAPS PharmSciTech 23, 27; Sharma et al. (2021) Mater. Today Proc. 45, 2963-2966; and Hwang et al. (2016) RSC Adv. 6, 70592-70615, which are herein incorporated by reference in their entireties.

As used herein, the term “lipid” refers to a group of organic compounds that include, but are not limited to, esters of fatty acids and are characterized by being insoluble in water, but soluble in many organic solvents. Lipids are usually divided into at least three classes: (1) “simple lipids,” which include fats and oils as well as waxes; (2) “compound lipids,” which include phospholipids and glycolipids; and (3) “derived lipids” such as steroids. The selection of the individual lipid components of the lipid formulation is made to optimize delivery of a payload (e.g., a nucleic acid) to a target cell. As used herein, the phrase “lipid formulation” refers to a formulation comprising one or more lipids (e.g., cationic lipids, non-cationic lipids, lipid conjugates, and the like).

Liposomes: In some aspects, the AAV-ITR vector and/or the Rep vector are encapsulated in a liposome or combination thereof. The term “liposome” as used herein refers to a vesicle comprising a lipid bilayer, for example, a closed vesicle formed when amphipathic lipids (e.g., phospholipids or their derivatives) are dispersed in water. The liposomes of the present invention typically comprise one or more phospholipids, and can also contain mixed lipid chains with surfactant properties (e.g., egg phosphatidylethanolamine). Liposome can employ surface ligands to target binding to a specific tissue (e.g., healthy or diseased tissue such tumors or neoplastic cells) or cell. Liposomes typically have an aqueous core. Liposomes are spherical vesicles, typically comprising phospholipids, that have an internal aqueous volume that is enclosed by one or more concentric lipid bilayers with the polar head groups oriented towards the interior and exterior aqueous phases. Natural phospholipids are biocompatible and biodegradable as they are naturally occurring in the body and are a major constituent of cell membranes. Liposomes can act as carriers by entrapping biologically active molecules (e.g., small molecule drugs, polypeptides or polynucleotides) in the aqueous core and/or within the lipid bilayers. Liposomes range in size and can exist as unilamellar or multilamellar vesicles.

The term “bilayer” as used herein refers to a structure composed of amphiphilic lipid molecules (often phospholipids) arranged in two molecular layers, with the hydrophobic tails on the interior and the polar head groups on the exterior surfaces.

The terms “liposomal composition” and “liposome-containing composition” are used interchangeably herein and refer to liposome formulations or mixtures comprising lipids (e.g., phospholipids, hydrophilic polymer-derivatized lipids, sterol components such as cholesterols, and combinations thereof). A liposomal composition typically comprises an aqueous solution comprising the liposomes. Encapsulated aqueous solution is aqueous solution in the aqueous core of the liposomes. Non-encapsulated aqueous solution is aqueous solution in which the liposomes are dispersed.

Liposomes are categorized based on diameter range, number of internalized vesicles, and number of lamellas. Lipid vesicles can be prepared as a small unilammelar vesicles (SUVs) which are created by a bilayer membrane. Large unilammelar vesicles (LUVs) with approximately 20-100 nm diameter range benefit from a bilayer membrane, while double bilayer vesicles (DBVs) contain two bilayer membranes with above 300 nm diameter range. Oligolammelar vesicles (OLVs) contain 3-5 bilayers, while multilammelar vesicles (MLVs) contain more than five concentric bilayer vesicles. There are giant unilammelar vesicles (GUVs) which are generated by a bilayer, as well. Multivesicular vesicles (MVVs) include a bilayer liposome which encapsulates numerous small non-concentric vesicles. Nanoliposomes or sub-micron lipid vesicles are found as SUV, LUV, DBV, and OLV, while MVV, GUV, MLV, OLV, DBV, and LUV are regarded as liposomes

There are numerous methods to create liposomes including reverse-phase evaporation (RPE), thin film dispersion (TFD), spray-freeze-drying, and alcohol injection. Other methods involve pro-liposomal formulation applying a cryoprotectant and high-temperature procedure, as well as supercritical fluid of carbon dioxide (SCF-CO2) techniques to adjust the experimental pressure and temperature as a method to control the shape and size of the particles.

Generally, liposomes include closed phospholipid double layers and benefit from a hydrophilic core unlike micelles. Liposomes can carry amphiphilic, hydrophobic, and hydrophilic compounds. In other words, the polar substance is loaded into the hydrophilic core, while the hydrophobic compounds can be placed inside the bilayers of lipid domains.

The formation of liposomes can depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles can be prepared by the methods, apparatus and devices described in U.S. Patent Publication Nos. US20130177638, US20130177637, US20130177636, US20130177635, US20130177634, US20130177633, US20130183375, US20130183373, and US20130183372, the contents of which are herein incorporated by reference in their entireties.

In one aspect, liposomes disclosed herein can comprise 1,2-dioleyloxy-N,N-dimethylaminopropane (DODMA), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), MC3 (see US20100324120, which is herein incorporated by reference in its entirety), DOTMA, 1,2-dioleoyl-sn-3-phosphoethanolamine (DOPE), N-(14(2,3-dioleyloxy)propyl)-N-(2-(sperminecarboxamido)ethyl)-N,N-dimethylammonium trifluoroacetate (DOSPA), dioctadecylamidoglycyl carboxyspermine (DOGS), and combinations thereof.

In one aspects, liposomes disclosed herein include those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties).

The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations can contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some aspects, liposome formulations can comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. For example, formulations can comprise a percentage of cholesterol selected from the group consisting of 28.5%, 31.5%, 33.5%, 36.5%, 37.0%, 38.5%, 39.0% and 43.5%. In some embodiments, formulations can comprise from about 5.0% to about 10.0% DSPC and/or from about 7.0% to about 15.0% DSPC.

In one aspect, polynucleotides such as the AAV-ITR vector and/or Rep vector disclosed herein can be encapsulated by the liposome and/or it can be contained in an aqueous core that can then be encapsulated by the liposome (see International Pub. Nos. WO2012031046, WO2012031043, WO2012030901 and WO2012006378 and US Patent Publication Nos. US20130189351, US20130195969 and US20130202684; the contents of each of which are herein incorporated by reference in their entirety).

In another aspect, liposomes can be formulated for targeted delivery. As a non-limiting example, the liposome can be formulated for targeted delivery to the liver or to the muscle. The liposome used for targeted delivery can include, but is not limited to, the liposomes described in and methods of making liposomes described in US Patent Publication No. US20130195967, the contents of which are herein incorporated by reference in its entirety.

In another aspect, the polynucleotides (e.g., an AAV-ITR vector and/or Rep vector disclosed herein) can be formulated in a cationic oil-in-water emulsion where the emulsion particle comprises an oil core and a cationic lipid that can interact with the polynucleotide anchoring the molecule to the emulsion particle (see International Pub. No. WO2012006380; herein incorporated by reference in its entirety).

In one aspects, the polynucleotides (e.g., an AAV-ITR vector and/or Rep vector disclosed herein) can formulated in a water-in-oil emulsion comprising a continuous hydrophobic phase in which the hydrophilic phase is dispersed. As a non-limiting example, the emulsion can be made by the methods described in International Publication No. WO201087791, the contents of which are herein incorporated by reference in its entirety.

In another aspect, the liposome can include at least cationic lipid, a lipid that can enhance transfection and a least one lipid that contains a hydrophilic head group linked to a lipid moiety (International Pub. No. WO2011076807 and U.S. Pub. No. 20110200582; the contents of each of which is herein incorporated by reference in their entirety). In another aspect, the liposome can have crosslinks between functionalized lipid bilayers (see U.S. Pub. No. 20120177724, the contents of which is herein incorporated by reference in its entirety).

In one aspect, the liposome is a liposome described in International Patent Publication No. WO2013086526, the contents of which is herein incorporated by reference in its entirety.

In some aspects, the AAV-ITR and/or Rep vector be encapsulated in a liposome using reverse pH gradients and/or optimized internal buffer compositions as described in International Patent Publication No. WO2013086526, the contents of which is herein incorporated by reference in its entirety. In some aspects embodiment, the liposome comprises a cationic lipid. In some aspects, the liposome can have a molar ratio of nitrogen atoms in the cationic lipid to the phosphates in the RNA (N:P ratio) of between 1:1 and 20:1 as described in International Publication No. WO2013006825, herein incorporated by reference in its entirety. In another embodiment, the liposome can have a N:P ratio of greater than 20:1 or less than 1:1.

The liposome formulation can be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28:172-176; herein incorporated by reference in its entirety), the liposome formulation was composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen-presenting cells (Basha et al. Mol Ther. 2011 19:2186-2200; herein incorporated by reference in its entirety). In some aspects, liposome formulations can comprise from about 35 to about 45% cationic lipid, from about 40% to about 50% cationic lipid, from about 50% to about 60% cationic lipid and/or from about 55% to about 65% cationic lipid. In some aspects, the ratio of lipid to dsDNA or mRNA in liposomes can be from about 5:1 to about 20:1, from about 10:1 to about 25:1, from about 15:1 to about 30:1 and/or at least 30:1.

Lipid Nanoparticles (LNP): In some aspects, the AAV-ITR vector and/or the Rep vector are encapsulated in a LNP or combination thereof. As used herein, the term “lipid nanoparticle” is used interchangeably with the abbreviation “LNP” and refers to a microscopic lipid formulation that can be used to deliver an active agent or therapeutic agent, such as a nucleic acid (e.g., an mRNA, dsDNA), to a target site of interest (e.g., an immune cell). Lipid nanoparticles typically have a size of less than about 1000 nm in at least one dimension. In various aspects, the LNPs of the present disclosure have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

Components suitable for the generation of lipidic delivery systems such as liposomes or lipid nanoparticles are discussed in detail below.

### Components of Lipidic Delivery Systems: Cationic or Ionizable Lipids or Lipidoids

As used herein, the term “ionizable lipid” refers to any of a number of lipid species that carry a net positive charge at a selected pH, such as physiological pH 4 and a neutral charge at other pHs such as physiological pH 7. Ionizable lipids can be used as a component of a lipidic delivery systems of the present disclosure (ego, a liposome or a LNP) to facilitate or enhance the delivery and release of a nucleic acid, e.g., a dsDNA in the case of an AAV-ITR vector or an mRNA in the case of a Rep vector, to one or more target cells (e.g., by permeating or fusing with the lipid membranes of such target cells).

In some specific aspects, the ionizable lipid or lipidoid is cKK-E12 (3,6-bis(4-(bis(2-hydroxydodecyl)amino)butyl)piperazine-2,5-dione). In some specific aspects, the ionizable lipid or lipidoid is Lipid 10, disclosed in WO2018087753, which is herein incorporated by reference in its entirety. In some aspects, the ionizable lipid or lipidoid is selected from the group consisting of cKK-E12, ALC-0315, SM-102, YK-009, DLin-MC3-DMA, KC2, A6, OF-02, A18-Iso5-2DC18, 98N12-5, 9A1p9, C12-200, 7C1, G0-C14, L319, 304O13, OF-Deg-Lin, 306-O12B, 3060i10, FTT5, Lipid 10, and combinations thereof.

cKK-E12 is an ionizable cationic lipomer that has been used in combination with other lipids in the formation of LNPs for the delivery of mRNA. ALC-0315 is an ionizable lipid that has been used to form lipid nanoparticles for delivery of RNA. ALC-0315 is one of the components in the BNT162b2 vaccine against SARS-CoV-2 in addition to ALC-0159, DSPC, and cholesterol. SM-102 is a synthetic amino lipid that is generally used in combination with other lipids to form LNPs. cKK-E12, ALC-0315, and SM-102 are used for the delivery of mRNA-based vaccines, and in particular SM-102 forms part of the drug delivery system for the Moderna COVID-19 vaccine. In the context of the present disclosure, cKK-E12, ALC-0315, SM-102, and Lipid 10 can be used as suitable lipids for the delivery of dsDNAS (e.g., an AAV-ITR vector) or an mRNA (e.g., a Rep vector) to one or more target cells.

In certain aspects, an ionizable lipid comprises one or more cleavable functional groups (e.g., a disulfide) that allow, for example, a hydrophilic functional head-group to dissociate from a lipophilic functional tail-group of the compound (e.g., upon exposure to oxidative, reducing or acidic conditions), thereby facilitating a phase transition in the lipid bilayer of the one or more target cells. In some embodiments, an ionizable lipid is a lipid as represented by formula 1 or as listed in Tables 1 or 2 of U.S. Pat. No. 9,708,628, the content of which is herein incorporated by reference in its entirety. In some embodiments, an ionizable lipid is as described in pages 7-13 of U.S. Pat. No. 9,765,022 or as represented by formula 1 of U.S. Pat. No. 9,765,022, the content of which is herein incorporated by reference in its entirety. In some embodiments, an ionizable lipid is described in pages 12-24 of WO2019152848A1 or as represented by formula 1 of International Patent Application WO2019152848A1, the contents of which are herein incorporated by reference in their entirety.

As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In some aspects, the cationic lipid is DOTAP or DOTMA. In some aspects, the cationic lipid comprises any of a number of lipid species that carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE).

Additionally, a number of commercial preparations of cationic lipids are available which can be used in the LNP of the present disclosure. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(14(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.).

The following lipids are cationic and have a positive charge at below physiological pH: DODAP, DODMA, DMDMA, 1,2-dilinoleyloxy-N,N-dimethylaminopropane (DLinDMA), and 1,2-dilinolenyloxy-N,N-dimethylaminopropane (DLenDMA).

In some aspects, the cationic lipid is an amino lipid. Suitable amino lipids useful in the lipidic delivery systems of the present disclosure (e.g., a LNP, a liposome, or a combination thereof) include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), I-linoleoyl-2-linoleyloxy-3-dimethylaminopropane (DLin-2-DMAP), 1,2-dilinoleyloxy-3-trimethylaminopropane chloride salt (DLin-TMA·Cl), 1,2-dilinoleoyl-3-trimethylaminopropane chloride salt 30 (DLin-TAP·Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In some aspects, the cationic or ionizable lipid or lipidoid component in a lipidic delivery system of the present disclosure (e.g., a LNP, a liposome, or a combination thereof) can comprise an ionizable cationic lipid, an ionizable cationic amino lipid, an ionizable cationic lipidoid, a polyamine branched-chain lipidoid, a lipid catechol, an ionizable dendrimer, a branched-chain ionizable cationic lipidoid, an ionizable cationic trialkyl lipid, a biodegradable alkyne lipid, an ionizable cationic SSPalm, an ionizable cationic self-degradable SSPalm, a multi-tail ionizable cationic phospholipid or a combination thereof.

Cationic ionizable that can be use in the lipidic delivery systems of the present disclosure comprise 1,2(R)-Dioleyloxy-3-dimethylamino-propane (Cayman Chemical Item 8004302; CAS No. 666234-78-2, also known as R-DODMA), Lipid R6 (Cayman Chemical Item No. 39130), 3060i9-cis2 (Cayman Chemical Item No. 39557), Lipid 16 (Cayman Chemical Item No. 38861), Lipid AX4 (Cayman Chemical Item No. 39070, CAS No. 2735814-23-8), RM 137-15 (Cayman Chemical Item No. 38918), C12-113 (Cayman Chemical Item No. 39335; CAS No. 1220890-27-6), C12-SPM (Cayman Chemical Item No. 38784, CAS No. 2055647-81-7, also known as C12-spermine), Lipid 10 (Cayman Chemical Item No. 38705, CAS No. 2430034-02-7, also known as EA-PIP), RM 133-3 (Cayman Chemical Item No. 38917, CAS No. 2941228-90-4), AA-T3A-C12 (Cayman Chemical Item No. 38648), Lipid 23 (Cayman Chemical Item No. 38862), OC2-K3-E10 (Cayman Chemical Item No. 38243, also known as I-28), CL4F8-6 (Cayman Chemical Item No. 38802, CAS No. 2766493-12-1), Lipid Catechol (Cayman Chemical Item No. 38665), 1014 (Cayman Chemical Item No. 38150), Lipid III-45 (Cayman Chemical Item No. 39243, CAS No. 2096984-25-5, also known as Cationic Lipid A), Lipid 8 (Cayman Chemical Item No. 38746, CAS No. 2226547-25-5), RCB-4-8 (Cayman Chemical Item No. 38803, CAS No. 2941228-91-5), YK-009 (Cayman Chemical Item No. 38282, CAS No. 2761458-86-8), 4A3-SC8 (Cayman Chemical Item No. 38155, CAS No. 1857340-78-3), Lipid 2,2(8,8) 4C CH3 (Cayman Chemical Item No. 37910, CAS No. 2230647-30-8, also known as ATX-0114), ATX-001 (Cayman Chemical Item No. 39037, CAS No. 1777792-33-2), ALC-0315 analogous-1 (Cayman Chemical Item No. 38591, CAS No. 2430034-17-4), C14-SPM (Cayman Chemical Item No. 38785, CAS No. 2241864-59-3, also known as C14-spermine), Lipid 222 (Cayman Chemical Item No. 38338), Lipid A4 (Cayman Chemical Item No. 38351, CAS No. 2639634-71-0), C14-4 (Cayman Chemical Item No. 38942, CAS No. 2639634-80-1), A12-Iso5-2DC18 (Cayman Chemical Item No. 38586, CAS No. 2412492-06-7), 1,2-Dipalmitoyl-3-dimethylammonium-propane (Cayman Chemical Item No. 38311, CAS No. 96326-74-8, also known as 16:0 DAP or DPDAP), 1,2-Dimyristoyl-3-dimethylammonium-propane (Cayman Chemical Item No. 38310, CAS No. 72719-84-7, also known as 14:0 DAP or DMDAP), Lipid CL1 (Cayman Chemical Item No. 38320, CAS No. 1450888-71-7), L202 (Cayman Chemical Item No. 37841, CAS No. 2170488-92-1), C13-112-tetra-tail (Cayman Chemical Item No. 38329, CAS No. 1381861-92-2), Lipid 14 (Cayman Chemical Item No. 38589, CAS No. 2430034-05-0), OF-Deg-Lin (Cayman Chemical Item No. 37853, CAS No. 1853202-95-5), DOG-IM4 (Cayman Chemical Item No. 37441, CAS No. 2758097-38-8), AL-A12 (Cayman Chemical Item No. 38001), 98N12-5 (Cayman Chemical Item No. 37651, CAS No. 917572-74-8, also known as ND98), TT3 (Cayman Chemical Item No. 37909, CAS No. 1821214-50-9), 246C10 (Cayman Chemical Item No. 37907, CAS No. 2635329-26-7), OF-C4-Deg-Lin (Cayman Chemical Item No. 37856, CAS No. 1853203-01-6), Lipid A6 (Cayman Chemical Item No. 35052), IC8 (Cayman Chemical Item No. 37986, CAS No. 2349307-32-8), PPZ-A10 (Cayman Chemical Item No. 9004144), AA3-DLin (Cayman Chemical Item No. 37903), BAMEA-016B (Cayman Chemical Item No. 37439, CAS No. 2490668-30-7, also known as BAMPA-016B), 113-016B (Cayman Chemical Item No. 37831, CAS No. 2566523-07-5), SSPalmO-Phe (Cayman Chemical Item No. 37670, CAS No. 2377474-67-2), SSPalmM (Cayman Chemical Item No. 37377, CAS No. 1436860-60-4), Lipid A9 (Cayman Chemical Item No. 37667, CAS No. 2036272-50-9), OF-02 (Cayman Chemical Item No. 37652, CAS No. 1883431-67-1), 113-O12B (Cayman Chemical Item No. 37671, CAS No. 2803699-72-9), ATX-100 (Cayman Chemical Item No. 36935, CAS No. 2230647-37-5), CL4H6 (Cayman Chemical Item No. 37279, CAS No. 2256087-35-9), 9A1P9 (Cayman Chemical Item No. 37276, CAS No. 2760467-57-8), 80-016B (Cayman Chemical Item No. 37564, CAS No. 1624618-02-5), CIN-16645 (Cayman Chemical Item No. 37278, CAS No. 1799316-64-5, also known as LP-01), 306-O12B (Cayman Chemical Item No. 37549, CAS No. 2566523-06-4), 306-O12B-3 (Cayman Chemical Item No. 37096), Lipid C24 (Cayman Chemical Item No. 37122, CAS No. 2767561-52-2), cKK-E12 (Cayman Chemical Item No. 36700, CAS No. 1432494-65-9), NT1-014B (Cayman Chemical Item No. 37095, CAS No. 2739805-64-0), TCL053 (Cayman Chemical Item No. 37045, CAS No. 2361162-70-9), C12-200 (Cayman Chemical Item No. 36699, CAS No. 1220890-25-4), DLin-DMA (Cayman Chemical Item No. 36701, CAS No. 871258-12-7, also known as 1,2-Dilinoleyloxy-N,N-dimethyl-3-aminopropane), YSK05 (Cayman Chemical Item No. 35786, CAS No. 1318793-78-0), 3060i10 (Cayman Chemical Item No. 36698, CAS No. 2322290-93-5), Lipid 29 (Cayman Chemical Item No. 35337, CAS No. 2244716-55-8), L-319 (Cayman Chemical Item No. 35051, CAS No. 1351586-50-9), 93-0170 (Cayman Chemical Item No. 34366, CAS No. 2227214-78-8), ALC-0315 (Cayman Chemical Item No. 34337, CAS No. 2036272-55-4), 93-017S (Cayman Chemical Item No. 34367, CAS No. 2227008-67-3), Lipid 5 (Cayman Chemical Item No. 34372, CAS No. 2089251-33-0), DLin-MC3-DMA (Cayman Chemical Item No. 34364, CAS No. 1224606-06-7, also known as MC3), 1,2-Dioleyloxy-3-dimethylamino-propane (Cayman Chemical Item No. 15109, CAS No. 104162-47-2, also known as DODMA or MBN 305A), 1,2-Dioleoyl-3-dimethylammonium-propane (Cayman Chemical Item No. 25726, CAS No. 127512-29-2, also known as 18:1 DAP or DODAP), SM-102 (Cayman Chemical Item No. 33474, CAS No. 2089251-47-6, also known as Lipid H or LNP-102), DLin-KC2-DMA (Cayman Chemical Item No. 34363, CAS No. 1190197-97-7, also known as KC2), a variant or derivative thereof, or a combination thereof.

### Components of Lipidic Delivery Systems: Structural Lipids

As used herein, the term “structural lipid” refers to sterols and to lipids containing sterol moieties. Incorporation of structural lipids in the lipidic delivery systems of the present disclosure (e.g., a LNP, a liposome, or a combination thereof) can help mitigate aggregation of other lipids in the particle.

In some aspects, the structural lipid is selected from the group consisting of cholesterol, beta-cholesterol, ergosterol, 7-dehydrocholesterol, 24S-hydroxycholesterol, lanosterol, cycloartenol, fucosterol, saringosterol, campesterol, 0-sitosterol, sitostanol, coprostanol, avenasterol, stigmasterol, and any combination thereof. Other sterols suitable for use as structural lipids comprise cholesterol sulfate, desmosterol-d6, lathosterol-d7, desmosterol, dihydrolanosterol, zymosterol, lathosterol, zymosterol-d5, 14-demethyl-lanosterol, 14-demethyl-lanosterol-d6, 8(9)-dehydrocholesterol, 8(14)-dehydrocholesterol, diosgenin, DHEA sulfate, DHEA, lanosterol-d6, dihydrolanosterol-d7, campesterol-d6, lanosterol-95, dihydro FF-MAS-d6, zymostenol-d7, zymostenol, campestanol, 7-dehydrodesmosterol, pregnenolone, sitosterol-d7, dihydro T-MAS, delta 5-avenasterol, brassicasterol, dihydro FF-MAS, 24-methylene cholesterol, cholic acid derivatives, cholesteryl esters, glycosylated sterols, hopanoids, hydroxysteroid, phytosterol, zoosterol, gonane, dexamethasone, and medrogestone. In particular aspects, the LNP of the present disclosure comprise cholesterol.

### Components of Lipidic Delivery Systems: Helper Lipids

The term “helper lipid”, as used herein, refers to lipids other than the cationic or ionizable lipids and stabilizing lipid (generally PEG-conjugated lipids) that can influence the properties of the lipidic delivery systems of the present disclosure (e.g., a LNP, a liposome, or a combination thereof). Helper lipids function to stabilize and improve processing of the lipidic delivery systems of the present disclosure (e.g., a LNP, a liposome, or a combination thereof).

In some aspects, the helper lipid is a phospholipid. A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. Phospholipids can be of a symmetric or an asymmetric type. As used herein, the term “symmetric phospholipid” includes glycerophospholipids having matching fatty acid moieties and sphingolipids in which the variable fatty acid moiety and the hydrocarbon chain of the sphingosine backbone include a comparable number of carbon atoms. As used herein, the term “asymmetric phospholipid” includes lysolipids, glycerophospholipids having different fatty acid moieties (e.g., fatty acid moieties with different numbers of carbon atoms and/or unsaturations (e.g., double bonds)), and sphingolipids in which the variable fatty acid moiety and the hydrocarbon chain of the sphingosine backbone include a dissimilar number of carbon atoms (e.g., the variable fatty acid moiety include at least two more carbon atoms than the hydrocarbon chain or at least two fewer carbon atoms than the hydrocarbon chain). In some aspects, the helper lipid comprises at least one symmetric phospholipid. In some aspects, the symmetric phospholipid comprises or consists of a symmetric phosphocholine. In some aspects, the symmetric phosphocholine is selected from the group consisting of
1,2-dipropionyl-sn-glycero-3-phosphocholine (03:0 PC),
1,2-dibutyryl-sn-glycero-3-phosphocholine (04:0 PC),
1,2-dipentanoyl-sn-glycero-3-phosphocholine (05:0 PC),
1,2-dihexanoyl-sn-glycero-3-phosphocholine (06:0 PC),
1,2-diheptanoyl-sn-glycero-3-phosphocholine (07:0 PC),
1,2-dioctanoyl-sn-glycero-3-phosphocholine (08:0 PC),
1,2-dinonanoyl-sn-glycero-3-phosphocholine (09:0 PC),
1,2-didecanoyl-sn-glycero-3-phosphocholine (10:0 PC),
1,2-diundecanoyl-sn-glycero-3-phosphocholine (11:0 PC, DUPC),
1,2-dilauroyl-sn-glycero-3-phosphocholine (DLPC),
1,2-dilinoleoyl-sn-glycero-3-phosphocholine (DLOPC),
1,2-ditridecanoyl-sn-glycero-3-phosphocholine (13:0 PC),
1,2-dimyristoyl-sn-glycero-3-phosphocholine (14:0 PC, DMPC),
1,2-dipentadecanoyl-sn-glycero-3-phosphocholine (15:0 PC),
1,2-dipalmitoyl-sn-glycero-3-phosphocholine (16:0 PC, DPPC),
1,2-diphytanoyl-sn-glycero-3-phosphocholine (4ME 16:0 PC),
1,2-diheptadecanoyl-sn-glycero-3-phosphocholine (17:0 PC),
1,2-distearoyl-sn-glycero-3-phosphocholine (18:0 PC, DSPC),
1,2-dinonadecanoyl-sn-glycero-3-phosphocholine (19:0 PC),
1,2-diarachidoyl-sn-glycero-3-phosphocholine (20:0 PC),
1,2-dihenarachidoyl-sn-glycero-3-phosphocholine (21:0 PC),
1,2-dibehenoyl-sn-glycero-3-phosphocholine (22:0 PC),
1,2-ditricosanoyl-sn-glycero-3-phosphocholine (23:0 PC),
1,2-dilignoceroyl-sn-glycero-3-phosphocholine (24:0 PC),
1,2-dimyristoleoyl-sn-glycero-3-phosphocholine (14:1 (Δ9-Cis) PC),
1,2-dimyristelaidoyl-sn-glycero-3-phosphocholine (14:1 (Δ9-Trans) PC),
1,2-dipalmitoleoyl-sn-glycero-3-phosphocholine (16:1 (Δ9-Cis) PC),
1,2-dipalmitelaidoyl-sn-glycero-3-phosphocholine (16:1 (Δ9-Trans) PC),
1,2-dipetroselenoyl-sn-glycero-3-phosphocholine (18:1 (Δ6-Cis) PC),
1,2-dioleoyl-sn-glycero-3-phosphocholine (18:1 (Δ9-Cis) PC, DOPC),
1,2-dielaidoyl-sn-glycero-3-phosphocholine (18:1 (Δ9-Trans) PC),
1,2-dilinoleoyl-sn-glycero-3-phosphocholine (18:2 (Cis) PC, DLPC),
1,2-dilinolenoyl-sn-glycero-3-phosphocholine (18:3 (Cis) PC, DLnPC),
1,2-dieicosenoyl-sn-glycero-3-phosphocholine (20:1 (Cis) PC),
1,2-diarachidonoyl-sn-glycero-3-phosphocholine (20:4 (Cis) PC, DAPC),
1,2-dierucoyl-sn-glycero-3-phosphocholine (22:1 (Cis) PC),
1,2-didocosahexaenoyl-sn-glycero-3-phosphocholine (22:6 (Cis) PC, DHAPC),
1,2-dinervonoyl-sn-glycero-3-phosphocholine (24:1 (Cis) PC),
1,2-dierucoyl-sn-glycero-3-phosphocholine (DEPC),
1,2-di-O-octadecenyl-sn-glycero-3-phosphocholine (18:0 diether PC), and combinations thereof.

In some aspects, the symmetric phosphocholine is DSPC and/or DOPC. In some aspects, the symmetric phospholipid comprises or consists of a symmetric phosphoethanolamine (PE). In some aspects, the symmetric phosphoethanolamine is selected from the group consisting of:
1,2-dihexanoyl-sn-glycero-3-phosphoethanolamine (06:0 PE),
1,2-dioctanoyl-sn-glycero-3-phosphoethanolamine (08:0 PE),
1,2-didecanoyl-sn-glycero-3-phosphoethanolamine (10:0 PE),
1,2-dilauroyl-sn-glycero-3-phosphoethanolamine (12:0 PE),
1,2-dimyristoyl-sn-glycero-3-phosphoethanolamine (14:0 PE, DMPE),
1,2-dipentadecanoyl-sn-glycero-3-phosphoethanolamine (15:0 PE),
1,2-dipalmitoyl-sn-glycero-3-phosphoethanolamine (16:0 PE, DPPE),
1,2-diphytanoyl-sn-glycero-3-phosphoethanolamine (4ME 16:0 PE),
1,2-diheptadecanoyl-sn-glycero-3-phosphoethanolamine (17:0 PE),
1,2-distearoyl-sn-glycero-3-phosphoethanolamine (18:0 PE, DSPE),
1,2-dipalmitoleoyl-sn-glycero-3-phosphoethanolamine (16:1 PE),
1,2-dioleoyl-sn-glycero-3-phosphoethanolamine (18:1 (Δ9-Cis) PE, DOPE),
1,2-dielaidoyl-sn-glycero-3-phosphoethanolamine (18:1 (Δ9-Trans) PE),
1,2-dilinoleoyl-sn-glycero-3-phosphoethanolamine (18:2 PE, DLPE),
1,2-dilinolenoyl-sn-glycero-3-phosphoethanolamine (18:3 PE, DLnPE),
1,2-diarachidonoyl-sn-glycero-3-phosphoethanolamine (20:4 PE, DAPE),
1,2-didocosahexaenoyl-sn-glycero-3-phosphoethanolamine (22:6 PE, DHAPE),
1,2-dierucoyl-sn-glycero-3-phosphoethanolamine (DEPE), and any combination thereof.

In some aspects, the symmetric phosphoethanolamine is DSPE and/or DOPE. In some aspects, the helper lipid can comprise an asymmetric phospholipid such as MPPC, MSPC, PMPC, PSPC, POPC, PLPC, SMPC, SPPC, SOPC, OMPC, OPPC, OSPC, POPE, or a combination thereof. In some aspects, the helper lipid can comprise a lysolipid, e.g., a lyso PC or a lyso PE. Lysophospholipids are derivatives of a phospholipid in which one or both fatty acyl chains have been removed by hydrolysis. In some aspects, the helper lipid can comprise a phosphoglycerol (PG), such as DEPG, DLPG, DMPG, DOPG or DPPG; a phosphoserine (PS), such as DEPS, DLPS, DMPS, DOPS or DPPS; a phosphatidic acid (PA), such as DEPA, DLPA, DMPA, DOPA, or DPPA, or a combination thereof.

### Components of Lipidic Delivery Systems: PEG-Lipids

As used herein, the term “PEG-modified lipid” or “PEG-lipid” refers to a lipid-linked (e.g., covalently attached) to at least one PEG polymer chain. In some embodiments, the PEG-lipid described herein comprises a poly(ethylene) glycol chain of up to 5, 10 or 20 kDa in length covalently attached to a lipid with alkyl chain(s) of C6-C20 length.

In some embodiments, the PEG-lipid is reversibly linked to the lipidic delivery system of the present disclosure (e.g., a LNP, a liposome, or a combination thereof) and the PEG moiety is gradually released in blood circulation upon administration. In some aspects, an alternative to a PEG-lipid can be used, for example, a derivatized lipid such as a derivatized ceramide (PEG-CER), including N-Octanoyl-Sphingosine-1-[Succinyl(Methoxy Polyethylene Glycol)-2000](C8 PEG-2000 ceramide). In some embodiments, the PEG-lipid described herein comprises or consists of a PEG-phospholipid and/a PEG-ceramide. In some aspects, the PEG-lipid is a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, or a combination thereof. In some aspects, the PEG-lipid is selected from the group consisting of PEG-c-DOMG, PEG-DMG, PEG-DLPE, PEG-DMPE, PEG-DPPC, PEG-DSPE, and any combination thereof. In some aspects, the PEG-lipid is DMG-PEG2000. In some aspects, the PEG-lipid is DSPE-PEG2000. In some aspects, the PEG-lipid is a ceramide PEG derivatives such as C8 PEG2000 ceramide, C16 PEG2000 ceramide, C8 PEG5000 ceramide, C16 PEG5000 ceramide, C8 PEG750 ceramide, and C16 PEG750 ceramide. In some aspects, the PEG-lipid is a PEG derivative, such as 16:0 PEG5000 PE, 14:0 PEG5000 PE, 18:0 PEG5000 PE, 18:1 PEG5000 PE, 16:0 PEG3000 PE, 14:0 PEG3000 PE, 18:0 PEG3000 PE, 18:1 PEG3000 PE, 16:0 PEG2000 PE, 14:0 PEG2000 PE, 18:0 PEG2000 PE, 18:1 PEG2000 PE, 16:0 PEG1000 PE, 14:0 PEG1000 PE, 18:0 PEG1000 PE, 18:1 PEG1000 PE, 16:0 PEG750 PE, 14:0 PEG750 PE, 18:0 PEG750 PE, 18:1 PEG750 PE, 16:0 PEG550 PE, 14:0 PEG550 PE, 18:0 PEG550 PE, 18:1 PEG550 PE, 16:0 PEG350 PE, 14:0 PEG350 PE, 18:0 PEG350 PE, and 18:1 PEG350. In some aspects, the PEG-lipid is a sterol PEG derivative such as Chol-PEG600. In some aspects, the PEG-lipid is a glycerol PEG derivative such as DMG-PEG5000, DSG-PEG5000, DPG-PEG5000, DMG-PEG3000, DSG-PEG3000, DPG-PEG3000, DMG-PEG2000, DSG-PEG2000, DPG-PEG2000, DMG-PEG1000, DSG-PEG1000, DPG-PEG1000, DMG-PEG750, DSG-PEG750, DPG-PEG750, DMG-PEG550, DSG-PEG550, DPG-PEG550, DMG-PEG350, DSG-PEG350, and DPG-PEG350. In some aspects, the PEG-lipid is a phospholipid PEG derivative such as DSPE-PEG5000, DSPE-PEG2000, DSPE-PEG1000, or DSPE-PEG550.

Contemplated PEG-modified lipids include, but are not limited to, a polyethylene glycol chain of up to 5 kDa in length covalently attached to a lipid with alkyl chain(s) of C6-C20 length. In some specific aspects, the PEG-lipid employed in the compositions and methods of the present disclosure is 1,2-dimyristoyl-sn-glycerol, methoxypolyethylene Glycol (2000 MW PEG) “DMG-PEG2000.”

The addition of PEG-modified lipids to the lipid delivery vehicle can prevent complex aggregation and can also provide a means for increasing circulation lifetime and increasing the delivery of the lipid-polynucleotide composition to the target tissues, (Klibanov et al. (1990) FEBS Letters, 268 (1): 235-237), or they can be selected to rapidly exchange out of the formulation in vivo (see U.S. Pat. No. 5,885,613). Particularly useful exchangeable lipids are PEG-ceramides having shorter acyl chains (e.g., C14 or C18). In some embodiments, the lipid moiety of the PEG-lipids includes those having lengths of from about C14 to about C22, such as from about C14 to about C16. In some embodiments, a PEG moiety, for example an mPEG-NH2, has a size of about 1000, 2000, 5000, 10,000, 15,000 or 20,000 daltons. In some aspects, the PEG-lipid is a non-diffusible PEG conjugates. Non-limiting examples of non-diffusible PEG conjugates include PEG-DSG and PEG-DSPE.

### Components of Lipidic Delivery Systems: Chemically Modified Lipids

In some aspects, the lipidic delivery system of the present disclosure (e.g., a LNP, a liposome, or a combination thereof) comprises a chemically modified lipid. As used herein, the term “chemically modified lipid” refers to a lipid that has been modified to be derivatizable by incorporating a chemically reactive group (e.g., a maleimide group or a sulfhydryl group) that can be used to attach a biologically active moiety (e.g., an antibody) covalently (e.g., via reaction between the maleimide group and a sulfhydryl group) or non-covalently to the lipid.

In some aspects, the lipidic delivery system of the present disclosure (e.g., a LNP, a liposome, or a combination thereof) further comprises a derivatizable lipid, e.g., a PEG lipid comprising a maleimide group such as DSPE-PEG2000-maleimide) wherein the maleimide reactive group is free (i.e., prior to the reaction with an antibody). In some aspects, the derivatizable lipid is conjugated to a targeting molecule (e.g., an antibody, such as a bispecific anti CD3/anti CD28 antibody) thereby anchoring the targeting molecule to the surface of the LNP.

In some aspects, where a certain class of lipids present in a lipidic delivery system of the present disclosure includes both a chemically modified lipid and an unmodified lipid, the chemically modified lipid can be derived from the unmodified lipid. By way of example, the one or more lipids of a lipidic delivery system of the present disclosure can include an unmodified DSPE-PEG2000 lipid and a modified DSPE-PEG2000 lipid that includes a functionalized group capable of forming a covalent bond, e.g., DMG-PEG2000-maleimide or DSPE-PEG2000-maleimide.

In some aspects, a lipidic delivery system of the present disclosure (e.g., a LNP, a liposome, or a combination thereof) can comprise DMG-PEG2000-maleimide or DSPE-PEG2000-maleimide. In some aspects, a lipidic delivery system of the present disclosure (e.g., a LNP, a liposome, or a combination thereof) can comprise DMG-PEG2000-SH or DSPE-PEG2000-SH.

In some aspects, the chemically modified lipid is selected from the group consisting of DSPE-PEG2000-maleimide, DSPE-PEG5000-maleimide, DMG-PEG2000-maleimide, DMG-PEG5000-maleimide, cholesterol-PEG2000-maleimide, cholesterol-PEG5000-maleimide, DSPE-PEG2000-SH, DSPE-PEG5000-SH, DMG-PEG2000-SH, DMG-PEG5000-SH, cholesterol-PEG2000-SH, and cholesterol-PEG5000-SH.

### Components of Lipidic Delivery Systems: Other Lipids Components

In some aspects, a lipidic delivery system of the present disclosure (e.g., a LNP, a liposome, or a combination thereof) can comprise additional components such as fatty acids, lysolipids, or vitamins. In some aspects, the fatty acid is a short-chain, medium-chain, or long-chain fatty acid. In some aspects, the fatty acid is a saturated fatty acid. In some aspects, the fatty acid is an unsaturated fatty acid. In some aspects, the fatty acid is a monounsaturated fatty acid. In some aspects, the fatty acid is a polyunsaturated fatty acid, such as a ω-3 (omega-3) or ω-6 (omega-6) fatty acid.

### Specific Liposome Compositions

In some aspect, an AAV-ITR based gene delivery system of the present disclosure or a component thereof, such as an AAV-ITR vector or Rep vector, is encapsulated in a liposome. These delivery systems can systems can be selected from commercially available systems and/or can be specially designed. For example, in some aspects, the AAV-ITR vector can be encapsulated in a LIPOFECTAMINE 2000™ liposome. In some aspects, the Rep vector can be encapsulated in a LIPOFECTAMINE MESSENGER MAX™ liposome.

LIPOFECTAMINE™ is a cationic liposome formulation, which complexes with negatively charged nucleic acid molecules to allow them to overcome the electrostatic repulsion of the cell membrane. LIPOFECTAMINE™'s cationic lipid molecules are formulated with a neutral co-lipid (helper lipid). The DNA-containing liposomes (with positive charge on their surfaces) can fuse with the negatively charged plasma membrane of living cells, due to the neutral co-lipid mediating fusion of the liposome with the cell membrane, allowing nucleic acid to cross into the cytoplasm and contents to be available to the cell for replication or expression.

LIPOFECTAMINE™ consists of a 3:1 mixture of DOSPA (2,3-dioleoyloxy-N-[2(sperminecarboxamido)ethyl]-N,N-dimethyl-1-propaniminium trifluoroacetate) and DOPE (1,2-dioleoyl-sn-glycero-3-phosphoethanolamine), which complexes with negatively charged nucleic acid molecules to allow them to overcome the electrostatic repulsion of the cell membrane. See Yang et al., Gene Ther 5, 380-387 (1998); Dalby et al. Methods. 33 (2): 95-103 (2004); and U.S. Pat. No. 7,479,573, which are herein incorporated by reference in their entireties.

### Specific LNP Compositions

In some aspects, an AAV-ITR based gene delivery system of the present disclosure or a component thereof, such as an AAV-ITR vector or Rep vector, is encapsulated in a LNP. In some aspects, the LNP contains a molar ratio (mol-%) of the cationic or ionizable lipid or lipidoid (e.g., cKK-E12, ALC-0315, SSOP, SM-102, Lipid 10, or a combination thereof) from about 30% to about 60% mol-%. In some aspects, the molar ratio of the cationic or ionizable lipid or lipidoid (e.g., cKK-E12, ALC-0315, KC2, MC3, SSOP, SM-102, Lipid 10, or a combination thereof) is about 30%, about 35%, about 40%, about 45%, about 50%, about 55%, or about 60%. In some aspects, the molar ratio of the cationic or ionizable lipid or lipidoid (e.g., cKK-E12, ALC-0315, KC2, MC3, SSOP, SM-102, Lipid 10, or a combination thereof) is between about 30% and about 35%, between about 35% and 40%, between about 40% and about 45%, between about 45% and about 50%, between about 50% and about 55%, or between about 55% and about 60%. In some aspects, the molar ratio of the cationic or ionizable lipid or lipidoid (e.g., cKK-E12, ALC-0315, SSOP, SM-102, Lipid 10, or a combination thereof) is between 30% and about 60%, between about 35% and about 55%, or between about 40% and about 50%. In a specific aspect, the molar ratio of the cationic or ionizable lipid or lipidoid (e.g., cKK-E12, ALC-0315, KC2, MC3, SSOP, SM-102, Lipid 10, or a combination thereof) is about 35%.

In some aspects, the LNP contains a molar ratio (mol-%) of the structural lipid (e.g., cholesterol, beta-cholesterol, or a combination thereof) from about 20% to about 60% mol-%. In some aspects, the molar ratio of the structural lipid (e.g., cholesterol) is about 20%, about 25%, about 30%, about 35%, about 40%, about 45%, about 50%, about 55%, or about 60%. In some aspects, the molar ratio of the structural lipid (e.g., cholesterol) is between about 20% and about 25%, about 25% and about 30%, about 30% and about 35%, between about 35% and 40%, between about 40% and about 45%, between about 45% and about 50%, between about 50% and about 55%, or between about 55% and about 60%. In some aspects, the molar ratio of the structural lipid (e.g., cholesterol) is between 20% and about 60%, between about 25% and about 55%, or between about 30% and about 50%, or between about 35% and about 45%. In a specific aspect, the molar ratio of the structural lipid (e.g., cholesterol) is about 46.5%.

In some aspects, the LNP contains a molar ratio (mol-%) of total phospholipid from about 5% to about 30% mol-%. As used herein total phospholipid refers to the total amount of helper lipid (e.g., DSPE, DSPC, DOPE, DOPE, or a combination thereof), PEG lipid (e.g., a DMG-PEG2000, DSPE-PEG2000, or a combination thereof), and chemically modified lipid (e.g., DSPE-PEG2000-maleimide). In some aspects, the molar ratio of total phospholipid is about 5%, about 10%, about 15%, about 20%, about 25%, or about 30%. In some aspects, the molar ratio of total phospholipid is between about 5% and about 10%, about 10% and about 15%, about 15% and about 20%, about 20% and about 25%, or about 25% and about 30%. In some aspects, the molar ratio of total phospholipid is between about 5% and about 30%, about 10% and about 25%, and about 15% and about 20%. In a specific aspect, the molar ratio of total phospholipid is about 18.5% (comprising about 16% of helper lipid, about 2% of PEG-lipid, and about 0.5% of chemically modified lipid).

In some aspects, the LNP comprises:
(1) a molar ratio (mol-%) of the cationic or ionizable lipid or lipidoid (e.g., cKK-E12, ALC-0315, KC2, MC3, SSOP, SM-102, Lipid 10, or a combination thereof) from about 30% to about 60% mol-%;
(2) a molar ratio (mol-%) of the structural lipid (e.g., cholesterol, beta-cholesterol, or a combination thereof) from about 20% to about 60% mol-%; and,
(3) a molar ratio (mol-%) of total phospholipid from about 5% to about 30% mol-%.

In some aspects, the LNP comprises:
(1) a molar ratio of the cationic or ionizable lipid or lipidoid (e.g., cKK-E12, ALC-0315, KC2, MC3, SSOP, SM-102, Lipid 10, or a combination thereof) of about 30%, about 35%, about 40%, about 45%, about 50%, about 55%, or about 60%;
(2) a molar ratio of the structural lipid (e.g., cholesterol) of about 20%, about 25%, about 30%, about 35%, about 40%, about 45%, about 50%, about 55%, or about 60%; and,
(3) a molar ratio of total phospholipid about 5%, about 10%, about 15%, about 20%, about 25%, or about 30%.

In some aspects, the LNP comprises:
(1) a molar ratio of the cationic or ionizable lipid or lipidoid (e.g., cKK-E12, ALC-0315, KC2, MC3, SSOP, SM-102, Lipid 10, or a combination thereof) between about 30% and about 35%, between about 35% and 40%, between about 40% and about 45%, between about 45% and about 50%, between about 50% and about 55%, or between about 55% and about 60%;
(2) a molar ratio of the structural lipid (e.g., cholesterol) between about 20% and about 25%, about 25% and about 30%, about 30% and about 35%, between about 35% and 40%, between about 40% and about 45%, between about 45% and about 50%, between about 50% and about 55%, or between about 55% and about 60%; and,
(3) a molar ratio of total phospholipid between about 5% and about 10%, about 10% and about 15%, about 15% and about 20%, about 20% and about 25%, or about 25% and about 30%.

In some aspects, the LNP comprises:
(1) a molar ratio of the cationic or ionizable lipid or lipidoid (e.g., cKK-E12, ALC-0315, KC2, MC3, SSOP, SM-102, Lipid 10, or a combination thereof) between 30% and about 60%, between about 35% and about 55%, or between about 40% and about 50%;
(2) a molar ratio of the structural lipid (e.g., cholesterol) between 20% and about 60%, between about 25% and about 55%, or between about 30% and about 50%, or between about 35% and about 45%; and,
(3) a molar ratio of total phospholipid between about 5% and about 30%, about 10% and about 25%, and about 15% and about 20%.

In some aspects, the LNP comprises:
(1) a molar ratio of the cationic or ionizable lipid or lipidoid (e.g., cKK-E12, ALC-0315, KC2, MC3, SSOP, SM-102, Lipid 10, or a combination thereof) of about 35%;
(2) a molar ratio of the structural lipid (e.g., cholesterol) of about 46.5%; and,
(3) a molar ratio of total phospholipid of about 18.5% (comprising about 16% of helper lipid, about 2% of PEG-lipid, and about 0.5% of chemically modified lipid).

In some aspects, the LNP comprises:
(a) a cationic or ionizable lipid or lipidoid (e.g., cKK-E12, AIC-0315, SM-102, YK-009, DLin-MC3-DMA, KC2, A6, OF-02, A18-Iso5-2DC18, 98N12-5, 9A1p9, C12-200, 7C1, G0-C14, L319, 304O13, OF-Deg-Lin, 306-O12B, 3060i10, Lipid 10, or FTT5);
(b) a structural lipid (e.g., cholesterol or beta-cholesterol);
(c) a helper lipid (e.g., a phospholipid such as DSPE, DSPC, DOPC or DOPE); and,
(d) a stabilizing lipid (e.g., a PEG lipid such as DMG-PEG1000, DMG-PEG2000, DMG-PEG1000).

In some aspects, the weight ratio (w/w) between the cationic or ionizable lipid or lipidoid (e.g., cKK-E12, ALC-0315, KC2, MC3, SSOP, SM-102, Lipid 10, or a combination thereof) and the payload (e.g., an AAV-ITR vector or a Rep vector) is from about 5% to about 15%. In some aspects, the weight ratio (w/w) between the cationic or ionizable lipid or lipidoid (e.g., cKK-E12, ALC-0315, KC2, MC3, SSOP, SM-102, Lipid 10, or a combination thereof) and the payload (e.g., an AAV-ITR vector or a Rep vector) is about 5%, about 6%, about 7%, about 8%, about 9%, about 10%, about 11%, about 12%, about 13%, about 14%, or about 15%. In some aspects, the weight ratio (w/w) between the cationic or ionizable lipid or lipidoid (e.g., cKK-E12, ALC-0315, KC2, MC3, SSOP, SM-102, Lipid 10, or a combination thereof) and the payload (e.g., an AAV-ITR vector or a Rep vector) is between about 5% and about 6%, about 6% and about 7%, about 7% and about 8%, about 8% and about 9%, about 9% and about 10%, about 10% and about 11%, about 11% and about 12%, about 12% and about 13%, about 13% and about 14%, or about 14% and about 15%. In some aspects, the weight ratio (w/w) between the cationic or ionizable lipid or lipidoid (e.g., cKK-E12, ALC-0315, KC2, MC3, SSOP, SM-102, Lipid 10, or a combination thereof) and the payload (e.g., an AAV-ITR vector or a Rep vector) is between about 5% and about 15%, about 6% and about 14%, about 7% and about 13%, about 8% and about 12%, or about 9% and about 11%. In a specific aspect, the weight ratio (w/w) between the cationic or ionizable lipid or lipidoid (e.g., cKK-E12, ALC-0315, KC2, MC3, SSOP, SM-102, Lipid 10, or a combination thereof) and the payload (e.g., a AAV-ITR vector or a Rep vector) about 18.5% (comprising about 16% of helper lipid, about 2% of PEG-lipid, and about 0.5% of chemically modified lipid).

In some aspects, the LNP comprises a molar ratio (mol-%) of cholesterol of about 46.5%. In some aspects, the LNP comprises a molar ratio (mol-%) of cholesterol of about 40%, about 41%, about 42%, about 43%, about 44%, about 45%, about 46%, about 47%, about 48%, about 49%, about 50%, about 51% or about 52%.

In some aspects, the LNP comprises a molar ratio (mol-%) of DSPE-PEG2000-maleimide of about 0.5%. In some aspects, the LNP comprises a molar ratio (mol-%) of DSPE-PEG2000-maleimide of about 0.1%, about 0.2%, about 0.3%, about 0.4%, about 0.5%, about 0.6%, about 0.7%, about 0.8%, about 0.9%, or about 1%.

In some aspects, the LNP comprises a molar ratio (mol-%) of DOPE of about 16%. In some aspects, the LNP comprises a molar ratio (mol-%) of DOPE of about 10%, about 11%, about 12%, about 13%, about 14%, about 15%, about 16%, about 17%, about 18%, about 19%, about 20%, about 21% or about 22%.

In some aspects, the LNP comprises a molar ratio (mol-%) of cKK-E12 of about 35%. In some aspects, the LNP comprises a molar ratio (mol-%) of cKK-E12 of about 30%, about 31%, about 32%, about 33%, about 34%, about 35%, about 36%, about 37%, about 38%, about 39%, or about 40%.

In some aspects, the LNP comprises a molar ratio (mol-%) of Lipid 10 of about 35%. In some aspects, the LNP comprises a molar ratio (mol-%) of Lipid 10 of about 30%, about 31%, about 32%, about 33%, about 34%, about 35%, about 36%, about 37%, about 38%, about 39%, or about 40%.

In some aspects, the LNP comprises a molar ratio (mol-%) of DMG-PEG2000 of about 2%. In some aspects, the LNP comprises a molar ratio (mol-%) of DMG-PEG2000 of about 1%, about 1.2%, about 1.4%, about 1.6%, about 1.8%, about 2%, about 2.2%, about 2.4%, about 2.6%, about 2.8%, or about 3%.

In some aspects, the LNP comprises:
(1) a molar ratio (mol-%) of cholesterol of about 46.5%;
(2) a molar ratio (mol-%) of DSPE-PEG2000-maleimide of about 0.5%;
(3) a molar ratio (mol-%) of DOPE of about 16%;
(4) a molar ratio (mol-%) of cKK-E12, ALC-0315, SSOP, SM-102, Lipid 10, or a combination thereof of about 35%; and,
(5) a molar ratio (mol-%) of DMG-PEG2000 of about 2%.

In some aspects, the LNP comprises:
(1) a molar ratio (mol-%) of cholesterol of about 38.5%;
(2) a molar ratio (mol-%) of DSPE-PEG2000-maleimide of about 0.2%;
(3) a molar ratio (mol-%) of DOPE, DSPC, or a combination thereof of about 10%;
(4) a molar ratio (mol-%) of cKK-E12, ALC-0315, KC2, MC3, SSOP, SM-102, Lipid 10, or a combination thereof of about 50%; and,
(5) a molar ratio (mol-%) of DMG-PEG2000 of about 1.3%.

In some aspects, the LNP comprises:
(1) a molar ratio (mol-%) of cholesterol of about 40%, about 41%, about 42%, about 43%, about 44%, about 45%, about 46%, about 47%, about 48%, about 49%, about 50%, about 51% or about 52%;
(2) a molar ratio (mol-%) of DSPE-PEG2000-maleimide of about 0.1%, about 0.2%, about 0.3%, about 0.4%, about 0.5%, about 0.6%, about 0.7%, about 0.8%, about 0.9%, or about 1%;
(3) a molar ratio (mol-%) of DOPE of about 10%, about 11%, about 12%, about 13%, about 14%, about 15%, about 16%, about 17%, about 18%, about 19%, about 20%, about 21% or about 22%;
(4) a molar ratio (mol-%) of cKK-E12, AL-0315, KC2, MC3, SSOP, SM-102, Lipid 10, or a combination thereof of about 30%, about 31%, about 32%, about 33%, about 34%, about 35%, about 36%, about 37%, about 38%, about 39%, or about 40%; and,
(5) a molar ratio (mol-%) of DMG-PEG2000 of about 1%, about 1.2%, about 1.4%, about 1.6%, about 1.8%, about 2%, about 2.2%, about 2.4%, about 2.6%, about 2.8%, or about 3%.

In some aspects, the LNP comprises:
(1) a molar ratio (mol-%) of cholesterol of about 46.5±4%;
(2) a molar ratio (mol-%) of DSPE-PEG2000-maleimide of about 0.5±0.1%;
(3) a molar ratio (mol-%) of DOPE of about 16%±2%;
(4) a molar ratio (mol-%) of cKK-E12 (or, e.g., AL-0315, KC2, MC3, SSOP, SM-102, Lipid 10, or a combination thereof) of about 35±4%; and,
(5) a molar ratio (mol-%) of DMG-PEG2000 of about 2±0.2%.

The selection of cationic or ionizable lipids, structural lipids, PEG-lipids, helper lipids, and chemically modified lipids and lipid conjugates which comprise the LNP, as well as the relative molar ratio of such lipids to each other, is based upon the characteristics of the selected lipid(s), the nature of the intended target cells, and the characteristics of the payload (e.g., an AAV-ITR vector or a Rep vector) to be delivered. Additional considerations include, for example, the saturation of the alkyl chain, as well as the size, charge, pH, pKa, fusogenicity and toxicity of the selected lipid(s). Thus, the molar ratios of each individual component can be adjusted accordingly.

In some aspects, the LNP comprises GenVoyILM™. In some aspects, the LNP does not comprise GenVoyILM™. In some aspects, the LNP does not comprise 50% ionizable lipid, 10% DSPC, 37% cholesterol, and 2.5% stabilizing lipid. In some aspects, the LNP does not comprise 50% ionizable lipid. In some aspects, the LNP does not comprise 10% DSPC. In some aspects, the LNP does not comprise 37%% cholesterol. In some aspects, the LNP does not comprise 2.5% stabilizing lipid. See Kitte R, Rabel M, Geczy R, Park S, Fricke S, Kohl U, Tretbar U S, Lipid Nanoparticles (LNPs) outperform Electroporation in mRNA-based CAR T cell Engineering, Molecular Therapy: Methods & Clinical Development (2023), doi.org/10.1016/j.omtm.2023.101139, which is herein incorporated by reference in its entirety.

In some aspects, the LNP does not have an average Mw in g/mol of approximately 630.5 Da. In some aspects, the LNP does not have Z-Avg diameter of 45-75 nm when the payload is siRNA. In some aspects, the LNP does not have a Z-Avg diameter pf 60-120 nm when the payload is mRNA. In some aspects, the LNP is not a LNP composition disclosed in WO2023057979A1, WO2018119514A1, WO2020210901A1, WO2020206231A1, WO2018064755A1, WO2020252589A1, WO2021000041A1, or WO2019210394A1, which are herein incorporated by reference in their entireties.

The lipidic delivery systems for use in the AAV-ITR based gene delivery system of the present disclosure can be prepared by various techniques which are presently known in the art. Nucleic acid-lipid particles and their method of preparation are disclosed in, for example, U.S. Patent Publication Nos. 2004/0142025 and 2007/0042031, the disclosures of which are herein incorporated by reference in their entirety for all purposes. Selection of the appropriate particle size and composition of the lipidic delivery system, e.g., a liposome or LNP must take into consideration, e.g., the target cell and the payload.

### Polymeric Delivery Systems

In aspects, an AAV-ITR based gene delivery system of the present disclosure or one of its components (e.g., the AAV-ITR vector or the Rep vector) can be formulated in a polymeric delivery system. See, e.g., Cai et al. (2023) Pharmaceutics 15(5): 1502, which is incorporated herein by reference in its entirety. In some aspect, the polymeric delivery system comprises a cationic polymer, e.g., a synthetic polymer or a natural polymer.

Synthetic polymers are a class of polymers produced artificially by the chemical polymerization reaction of small molecules or modification of natural polymers, as distinct from naturally occurring polymers. Depending on the reaction monomer and the reaction mechanism, different kinds of synthetic polymers can be obtained. In some aspects, the synthetic polymer is a conventional polymer, e.g., a polymer selected from the group consisting of a DEAE-dextran, a poly-amino acid, a poly(amidoamine) (PAMAM) dendrimer, PPI (poly-(propylenimine)), PEI (polyethylenimine), PDMAEMA (polymethacrylic acid N,N-dimethylaminoethyl ester). DEAE-dextran is a chemically modified analog of dextran. By modification with diethylaminoethylen groups, the amidation of the dextran chains is readily protonated, which allows it to self-assemble into nanoparticles with negatively charged nucleic acids. DEAE-dextran was the first cationic polymer to be used for nucleic acid transfection. Poly-amino acids such as PLL (poly-L-Lysine) can also be used in polymeric delivery systems. PLL is a positively charged poly-amino acid under physiological conditions. PLL can bind to plasmid DNAs (pDNAs) and condense them into compact particles when the chain length is more than 20 residues. The covalent attachment of ligands on PLL significantly enhances endocytosis via receptor-mediated pathways. PLL linked with folic acid or transferrin can be used for the transfection of pDNAs into cancer cells. PLO (poly-L-ornithine) shares the properties of PLL, but achieves a 10-fold increase in transfection efficiency compared to PLL. Poly(amidoamine) (PAMAM) dendrimers are repetitive units of three-dimensional branched macromolecules with plenty of active amine groups on the surface. Both pDNA and siRNA can be encapsulated into condensed forms by PAMAM. PPI (poly-(propylenimine)) is a highly branched spherical dendrimer with primary amino groups at the periphery, and is hydrophobic in the interior. Genes transfected by PPI are able to be expressed in the liver. PEI (polyethylenimine) can perform with high transfection efficiency when forming a complex with pDNA. Its condensation ability and surface charge rose as the molecular weight of the compound grows. Similar to PAMAM, PEI induces the proton sponge effect. Its buffering capacity causes osmotic swelling and endosome rupture, allowing the polyplexes to escape into the cytoplasm. Other factors, in addition to the proton sponge effect, are thought to cause endosome escape. PEI, for example, can cause endosome membrane destabilization by generating hydrophilic pores in the lipid bilayer, resulting in endosome disruption. Two kinds of PEI, branched PEI (BPEI) and linear PEI (LPEI), are commercially accessible. LPEI exhibits better transfection efficiency than BPEI, especially in non-dividing cells. PDMAEMA (polymethacrylic acid N,N-dimethylaminoethyl ester) is responsive to both temperature and pH. PDMAEMA with a molecular weight of more than 300 kDa efficiently condenses DNA into compact nanoparticles, while those less than 300 kDa cannot. Linear, highly branched, and star-shaped structures are three kinds of PDMAEMA. The star-shaped PDMAEMA, according to previous studies, shows better transfection efficiency than the other two.

In some aspects, the synthetic polymer is a polyester. PBAE (poly(O-amino ester)) is a cationic polymer synthesized with acrylates and amines via Michael addition reactions. The surface charge density of PBAE is lower than that of other cationic polymers, which means its binding strength with nucleic acid is lower. As a result, the amount of PBAE required for nucleic acid binding should be significantly higher than that required for PEI, PPI, and other cation polymers. Even so, transfection with PBAEs as vectors is more efficient, due to its reduced cytotoxicity. Endcaps containing secondary amines or other amine-containing groups have been demonstrated to boost the binding capacity of PBAE and the overall transfection efficiency. PHP (Poly(4-hydroxy-L-proline ester)), which is made of hydroxyproline from natural sources such as collagen, gelatin, and other proteins, was the first polyester used as a gene carrier. PHP ester's transfection effectiveness is on par with that of poly-L-lysine. PAGA (poly[α-(4-aminobutyl)-1-glycolic acid]) can strongly form complexes with DNA at charge ratios below 2. PVL poly(6-valerolactone)-based polymers have been used as vectors for delivering genes. The transfection effectiveness was significantly impacted by the monomer arrangement and aliphatic side-chain length. He and colleagues produced new functional PVL copolymers using the ring opening polymerization of tertiary amine- and alkylated valerolactone-containing monomers. They proved that pDNA and functional PVL copolymers can successfully interact to generate pDNA/PVL polyplexes. The transfection effectiveness of the best functional PVL copolymer with the ideal chemical structure can be 2.2 times greater than PEI. Aminated PAHA (aminated poly(α-hydroxy acids)) can also be used in polymeric delivery systems. Poly(1)-g-AET2 (PAET), a newly developed amino PAHA polymer, has low toxicity and outstanding cell penetration and gene delivery characteristics. PPE (polyphosphoester)-based micelle systems can bind siRNA and treat hypoxic tumors and lung cancer. Chemotherapeutic agents, such as paclitaxel, can also be delivered through these micelles, and do not cause carrier-related toxicity nor activate innate immunity.

In some aspects, the synthetic polymer is a polylactide(Poly(lactic acid), PLA). Micelles constructed of polylactides are a dependable mRNA delivery system. PLA itself has only seldom been used as a gene delivery vector, due to the scarcity of cationic compounds on its backbone. To address this issue newly created cationic polyactides (CPLAs) have been shown to be efficient at delivering siRNA and pDNA into cancer cells.

In some aspects, the synthetic polymer is a polycarbonate. Polyethylenimine-grafted polycarbonates can produced high gene transfection at high polymer concentrations while causing moderate cytotoxicity. Biodegradable cationic siRNA carrier for pancreatic cancer therapy have used polycarbonates generated from carbon dioxide (CPCHC). CPCHCs are capable of efficiently preventing siRNA from being destroyed by RNase, and encouraging siRNA to escape from the endosomes for an extended period of time.

In some aspects, the synthetic polymer is a polyurethane (PUs). Cationic polyurethanes can be used as non-viral vectors, since they have positive charges and possible biofeatures. One such non-viral vector for gene delivery is N,N-diethylethylenediamine-polyurethane (DEDA-PU), which contains tertiary amines in the backbone and side chains. DEDA-PU has a transfection effectiveness that is comparable to PDMAEMA and much less cytotoxicity than PDMAEMA, allowing it to deliver plasmid DNA into cells.

In some aspects, the synthetic polymer is a conjugated polymers (CP). CPs are a type of polymer with a delocalized electronic structure and π-conjugated backbones. CPs can be modified with different side chains for use in drug and gene delivery systems for cancer therapy. Some of the most commonly used CPs for gene delivery include poly(p-phenyleneethynylene), polythiophene, poly(fluorine-co-phenylene), and poly(p-phenylenevinylene). These polymers are biocompatible, and can bond to nucleic acids through electrostatic interactions, hydrophobic interactions, or hydrogen bonds.

In some aspects, the polymer is a natural polymer. Naturally occurring polymers synthesized from living organisms are classified as natural polymers. These polymers possess good biocompatibility and are readily available in large yields, making them advantageous for the development of natural polymers for biological applications. In some aspects the natural polymer is a protein. In some aspects, the protein is selected from the group consisting of a histone, gelatin, and protamine. Histones are able to bind negatively charged nucleic acids through electrostatic interactions, and naturally target the nucleus. The histone-mediated transfection of nucleic acids, called histonefection, is effective in transfecting DNA, mRNA, and siRNA, especially into primary cell lines. A non-viral fibroblast-targeting DNA carrier called H2A-YG2 was made by fusing histone H2A with the PDGFR-binding peptide, YG2. The fusion vector increased DNA internalization only in PDGFRP-positive cells. Gelatin, unlike other polymers, n has amino acid sequences in its structure, such as Arg-Gly-Asp (RGD). Gelatin is, therefore, always cationized to enhance the polymer's ability to bind with negatively charged DNA or cellular membranes for effective gene delivery. Polyethyleneimine, cholamine, ethylenediamine (EDA), putrescine, and spermidine are commonly used to introduce amine residues to the carboxyl groups of gelatin, in order to create cationized gelatin. Protamine is a small polycationic peptide consisting of 50-110 amino acids, with a molecular weight of 4000-5000 Da. Due to electrostatic interactions between the positively charged protamine and the negatively charged nucleic acid-phosphate backbone, it binds to DNA. The basic amino acid in monoprotamines is arginine, while diprotamines also include lysine or histidine, and triprotamines have all three. By adjusting the concentration of salt, the ratio of protamine to RNA, and the concentration of the complex, it is possible to generate particles with average diameters specifically ranging from 50 nanometers (nm) to 1000 nm for different delivery needs.

### Payloads

The present disclosure provides AAV-ITR based gene delivery systems comprising an AAV-ITR vector comprising a gene of interest (GOI) and a Rep vector. In some aspects, the AAV-ITR vector and/or Rep vector of an AAV-ITR based gene delivery system of the present disclosure are encapsulated in a lipidic delivery system (e.g., a LNP or a liposome). As used herein, the term “payload” can refer to a “gene of interest” or “GOI,” i.e., a biologically active molecule encoded by a double stranded DNA inserted in the AAV-ITR vector component of an AAV-ITR based gene delivery system of the present disclosure.

The term “payload” also refers to biologically active molecules (e.g., small molecule drugs) that can be co-encapsulated with an AAV-ITR vector and/or Rep vector in their respective lipidic delivery systems. Additionally, the term “payload” also refers to biologically active molecules (e.g., targeting molecules such as an antibodies) that are attached or incorporated to a lipid bilayer of a lipidic delivery system of an AAV-ITR based gene delivery system of the present disclosure.

Non-limiting examples of payloads that can be used in the AAV-ITR based gene delivery systems include therapeutic agents such as, nucleotides (e.g., therapeutic nucleotides or nucleotides comprising a detectable moiety), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety), polypeptides (e.g., enzymes), lipids, carbohydrates, and small molecules (e.g., small molecule drugs and toxins). In certain aspects, a payload comprises an antigen or a nucleic acid (e.g., an mRNA) encoding an antigen. As used herein, the term “antigen” refers to any agent that when introduced into a subject elicits an immune response (cellular or humoral) to itself. In some aspects, the antigen is used to elicit an immune response, i.e., as a vaccine, e.g., in a cancer vaccine. In other aspects, a payload comprises an adjuvant.

The delivery systems of the present disclosure can be used to deliver a variety of payload, e.g., therapeutic agents, detectable labels, and cell penetrating payloads. In some aspects, the payloads are encoded by a polynucleotide encoding a biologically active molecule of interest (i.e., a GOI) such as, e.g., an enzyme, vaccine antigen, antibody, CAR, etc. In some aspects, the payload is encapsulated in a lipidic delivery system, e.g., a liposome encapsulating an AAV-ITR vector, or a liposome encapsulating a Rep vector. In other aspects, the payload can be covalently or non-covalently linked to the external surface or interior of a lipidic delivery system, e.g., to the internal membrane or internal surface of a liposome encapsulating an AAV-ITR vector, or a liposome encapsulating a Rep vector. In some aspect, a payload can be attached to a liposome encapsulating an AAV-ITR vector, or to a liposome encapsulating a Rep vector of the present disclosure via a linker, for example, cleavable linker.

In some aspects of the present disclosure, the payload comprises a polypeptide, a peptide, a polynucleotide, a chemical compound, or any combination thereof. In some aspects, an AAV-ITR based gene delivery system of the present disclosure can comprise a single payload, e.g., a polynucleotide encoding a GOI in the AAV-ITR vector. In other aspects, an AAV-ITR based gene delivery system of the present disclosure can comprise multiple payloads, e.g., one or more co-encapsulated small molecules (e.g., a molecule encapsulated in a liposome that also contains an AAV-ITR vector or a Rep vector), one or more targeting moieties (e.g., one or more targeting antibodies capable of directing the AAV-ITR based gene delivery system to a specific target cell) attached to the surface of a liposome, or a combination thereof.

In some aspects, the payload is a detectable substance. Detectable substances include, but are not limited to, various organic small molecules, inorganic compounds, nanoparticles, enzymes or enzyme substrates, fluorescent materials, luminescent materials, bioluminescent materials, chemiluminescent materials, radioactive materials, and contrast agents. Labels are contemplated by the present disclosure, including, but not limited to, optically detectable labels. Labels can be attached to a vector (e.g., AAV-ITR vector or Rep vector) in an AAV-ITR based gene delivery system of the present disclosure, encoded by a vector (e.g., AAV-ITR vector) in an AAV-ITR based gene delivery system of the present disclosure, attached to another payload of the present disclosure, e.g., targeting molecule on the surface of a liposome, attached to a lipid in a lipidic delivery system component of an AAV-ITR based gene delivery system of the present disclosure, or any combination thereof. In some aspects, the detectable label is attached using standard chemistries such that the label can be removed upon cleavage of a cleavable linker. A detectable label can useful in therapeutic, diagnostic, imaging (e.g., radioimaging), or basic research applications.

In some aspects, the detectable label is a radioactive label. Examples of a radioactive label include, but are not limited to, the isotopes 3H, 14C, 32P, 35S, 36Cl, 51Cr, 57Co, 58Co, 59Fe, 90Y, 121I, 124I, 125I, 131I, 111In, 117Lu, 211At, 198Au, 67Cu, 225Ac, 213Bi, 99Tc, 186Re and 89Zr.

In some aspects, the detectable label is a chemiluminescent label, fluorescent label, enzyme, biotin, or a combination thereof. In some aspects, the detectable label is a peptide tag. In some aspects, the detectable label is a polyhistidine tag, polyarginine tag, glutathione-S-transferase (GST), maltose binding protein (MBP), chitin binding protein (CBP), Strep-tag, thioredoxin (TRX), poly(NANP), FLAG tag, ALFA-tag, V5-tag, Myc-tag, hemagglutinin (HA) tag, Spot tag, T7 tag, NE tag, or green fluorescence protein (GFP), or a combination thereof. In some aspects, the polyhistidine tag consists of from about 4 to about 10 histidine residues. In some aspects, the polyhistidine tag consists of about 4, about 5, about 6, about 7, about 8, about 9, or about 10 histidine residues. Additional examples of detectable labels and methods for introducing detectable labels into a polypeptide or polynucleotide are known and include routine chemical, molecular biology and recombinant DNA techniques. See, e.g., Hnatowich et al., Science, 220(4597):613-615, 1983; Yao et al., Int. J. Mol. Sci., 17(2):194, 2016; Kimple et al., Curr. Protoc. Protein Sci., 73:Unit 9.9, 2013; Sambrook J, Fritsch E F. Molecular Cloning: A Laboratory Manual. Cold Spring Harbor Laboratory Press; Cold Spring Harbor, N.Y.: 1989; Molecular Cell Biology, 4th edition, Section 3.5, Purifying, Detecting and Characterizing Proteins; and Mahmoodi et al., Cogent Biology, 5(1):DOI: 10/1080/23312025.2019.1665406.

In some aspects, the payload comprises a therapeutic small molecule. In some aspects, the small molecule is a proteolysis-targeting chimera (PROTAC). In some aspects, the small molecule is a nucleotide. In some aspects, the nucleotide is a stimulator of interferon genes protein (STING) agonist.

In some aspects, the payload comprises, consists or consists essentially of a polynucleotide. In some aspects, the polynucleotide is a double stranded DNA encoding a GOI, mRNA, an antisense oligonucleotide (ASO), a phosphorodiamidate morpholino oligonucleotide (PMO), a siRNA, a miRNA, a shRNA, a plasmid, or a vector.

I.B.viii Encapsulation Efficacy and Lipid: Vector Ratios

In some aspects, the encapsulation efficiency of a vector (e.g., a dsDNA vector such as an AAV ITR vector or a mRNA vector such as an Rep vector) in a lipidic delivery system of the present disclosure (e.g., a LNP or liposome) is 95±5%. In some aspects, the encapsulation efficiency is about 100%. In some aspects, the encapsulation efficiency is at least about 70%, at least about 75%, at least about 80%, at least about 85%, at least about 90%, at least about 95%, at least about 96%, at least about 97%, at least about 98%, at least about 99%. In some aspects, the encapsulation efficiency is between about 70% and about 75%, between at least about 75% and about 80%, between about 80% and about 85%, between about 85% and about 90%, between about 90% and about 95%, or between about 95% and 100%.

In some aspects, the ratio of dsDNA and/or mRNA to lipid (w/w) in a lipidic delivery system of the present disclosure (e.g., a LNP or liposome) is between 1:1 and about 1:4. In some aspects, the ratio of dsDNA and/or mRNA to lipid (w/w) in a lipidic delivery system of the present disclosure (e.g., a LNP or liposome) is about 1:1. Thus, in some aspects, the ratio of dsDNA and/or mRNA to lipid (w/w) in a lipidic delivery system of the present disclosure (e.g., a LNP or liposome) can be between about 1 and 4, e.g., about 1.5. In some aspects, such w/w ratio about 1, about 1.5, about 2, about 2.5, about 3, about 3.5, or about 4. In some aspects, such w/w ratio is between about 1 and about 1.5, between about 1.5 and about 2, between about 2 and about 2.5, between about 2.5 and about 3, between about 3 and about 3.5, between about 3.5 and about, between about 1.5 and about 3.5, or between about 2 and about 3.

In some aspects, the percentage of dsDNA and/or mRNA with respect to lipid (w/w) in a lipidic delivery system of the present disclosure (e.g., a LNP or liposome) is between 0.1% and 10%.

In some aspects, the percentage of dsDNA and/or mRNA with respect to lipid (w/w) is at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.25%, at least about 1.5%, at least about 1.75%, at least about 2%, at least about 2.25%, at least about 2.5%, at least about 2.75%, at least about 3%, at least about 3.25%, at least about 3.5%, at least about 3.75%, at least about 4%, at least about 4.25%, at least about 4.5%, at least about 4.75%, at least about 5%, at least about 5.25%, at least about 5.5%, at least about 5.75%, at least about 6%, at least about 60.25%, at least about 6.5%, at least about 60.75%, at least about 7%, at least about 7.25%, at least about 7.5%, at least about 7.75%, at least about 8%, at least about 8.25%, at least about 8.5%, at least about 8.75%, at least about 9%, at least about 9.25%, at least about 9.5%, at least about 9.75%, or at least about 10%.

In some aspects, the percentage of dsDNA and/or mRNA with respect to lipid (w/w) is about 0.1%, about 0.2%, about 0.3%, about 0.4%, about 0.5%, about 0.6%, about 0.7%, about 0.8%, about 0.9%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, about 7%, about 7.25%, about 7.5%, about 7.75%, about 8%, about 8.25%, about 8.5%, about 8.75%, about 9%, about 9.25%, about 9.5%, about 9.75%, or about 10%.

Payload: dsDNA for Gene Therapy

In some aspects, the payload of the AAV-ITR based gene delivery system of the present disclosure comprises a dsDNA in the AAV-ITR vector encoding a gene of interest. Upon integration into the genome of a target cell or host cell, the dsDNA is transcribed to an mRNA that encodes a protein of interest, e.g., an antibody or a CAR. In some aspects, the dsDNA encodes a viral, bacterial, or protozoan protein or fragment or variant thereof, e.g., a vaccine to treat, e.g., COVID-19 (SARS-CoV2 infection), HIV infection, influenza, RSV infection, rabies, HPV infection, malaria, EBV infection, tuberculosis, CMV infection, Herpes zoster, Zika virus infection, HBV infection, yellow fever, PIV infection, hMPV infection, rotavirus infection, Nipah virus infection or Chikungunya virus infection. In some aspects, the dsDNA encodes a neutralizing antibody to treat, e.g., COVID-19 (SARS-CoV2 infection), HIV infection, influenza, RSV infection, rabies, HPV infection, malaria, EBV infection, tuberculosis, CMV infection, Herpes zoster, Zika virus infection, HBV infection, yellow fever, PIV infection, hMPV infection, rotavirus infection, Nipah virus infection or Chikungunya virus infection.

In some aspects, the dsDNA encodes one or more components of gene editing system. In some aspects, the dsDNA encodes a vaccine for the treatment of cancer, e.g., melanoma, NSCLC, cervical cancer, breast cancer, ovarian cancer, liver cancer, gastric cancer, pancreatic cancer, colorectal cancer, bladder cancer, prostate cancer, head and neck cancer, adenoidcystic carcinoma, cSCC, basal cell cancer, renal cell cancer, or AML, In some aspects, the vaccine for the treatment of cancer is a personal vaccine.

In some aspects, the dsDNA encodes a CAR (see below). In some aspects, the dsDNA encodes an antibody or antigen-binding portion thereof. In some aspects, the dsDNA encodes an antibody disclosed below or an antigen-binding portion thereof (e.g., the antigen-binding portion of a CAR). In some aspects, the dsDNA encodes a protein for protein replacement therapy. In some aspects, the dsDNA encodes a component of the CRISPR/Cas nuclease system.

In some aspects, the dsDNA encodes a protein for protein replacement therapy in genetic diseases such as cystic fibrosis, propionic academia, methylmalonic academia, CSD1a, phenylketonuria, CN-1, OTC, or hemophilia. In some aspects, the mRNA encodes a protein for protein replacement therapy in autoimmune disorders. In some aspects, the dsDNA encodes a protein for protein replacement therapy in metabolic disorders, e.g., type 2 diabetes. In some aspects, the dsDNA encodes a protein for protein replacement therapy in cardiovascular disease, e.g., hypercholesterolemia or myocardial ischemia. In some aspects, the dsRNA encodes a protein for protein replacement therapy in fibrosis, e.g., hypertropic scarring, liver fibrosis, lung fibrosis, anemia, or primary sclerosing cholangitis. See Qin et al (2022) Signal Transduction and Targeted Therapy 7:166; Huang et al. (2022) Nature Medicine 28:2273-2287; and Liu et al. (2022) Nature Reviews Cancer 23:526-543, which are herein incorporated by reference in their entireties.

In some aspects, the protein for gene replacement therapy is selected from the group consisting of AAT, ABCB4, ACADVL, ARG1, ATP8B1, C2, CFTR, citrin, CNTF, CPS1, CTNS, G6PT1, GALT, GLA, HPRT, JAG1, LPL, MCAD, MCM, PBGD, REP1, RPE65, RS1, and TTR.

TABLE 7
Exemplary genes and diseases for gene replacement therapy/

| | | | | Protein | DNA |
|---|---|---|---|---|---|
| | | | Replacement | sequence | sequence |
| Disease | Target | | protein | SEQ ID NO | SEQ ID NO |
| MMA | MCM | methylmalonyl Coenzyme | I671V | 424 | 425 |
| | | A mutase | | | |
| Argininemia | ARG1 | arginase-1 | wt, isoform 1 | 426 | 427 |
| Argininemia | ARG1 | arginase-1 | wt, isoform 2 | 428 | 429 |
| Porphyria | PBGD | porphobilinogen deaminase | wt, isoform 1 | 430 | 431 |
| Porphyria | PBGD | porphobilinogen deaminase | wt, isoform 2 | 432 | 433 |
| Porphyria | PBGD | porphobilinogen deaminase | wt, isoform 3 | 434 | 435 |
| Porphyria | PBGD | porphobilinogen deaminase | wt, isoform 4 | 436 | 437 |
| Porphyria | PBGD | porphobilinogen deaminase | I291M/N340S | 438 | 439 |
| | GoF | | | | |
| Porphyria | ApoA1- | ApoA1-PBGD fusion | ApoA1 fusion | 440 | 441 |
| | PBGD | | | | |
| PFIC3 | ABCB4 | phosphatidylcholine | wt, isoform 1 | 442 | 443 |
| | | translocator ABCB4 | | | |
| PFIC3 | ABCB4 | phosphatidylcholine | wt, isoform 2 | 444 | 445 |
| | | translocator ABCB4 | | | |
| Galactosemia | GALT | galactose-1-phosphate | wt, isoform 1 | 446 | 447 |
| | | uridylyltransferase | | | |
| Galactosemia | GALT | galactose-1-phosphate | wt, isoform 2 | 448 | 449 |
| | | uridylyltransferase | | | |
| CTLN2 | Citrin | citrin | wt, isoform 1 | 450 | 451 |
| CTLN2 | citrin | citrin | wt, isoform 2 | 452 | 453 |
| CTLN2 | GPD | glycerol-3-phosphate | wt | 454 | 455 |
| | | dehydrogenase | | | |
| Retinoschisis | RS1 | retinoschisin 1 | wt | 456 | 457 |
| Retinoschisis | RS1 | retinoschisin 1 | W96R | 458 | 459 |
| C2 deficiency | C2 | complement component C2 | wt | 460 | 461 |
| Aligille | JAG1 | Jagged-1 | wt | 462 | 463 |
| Aligille | JAG1 | Jagged-1 | 1-1054 | 464 | 465 |
| Aligille | JAG1 | Jagged-1 | 1-1046 | 466 | 467 |
| Aligille | JAG1 | Jagged-1 | peptide + Fc | 468 | 469 |
| | | | fusion | | |
| Aligille | JAG1 | Jagged-1 | Fc + peptide | 470 | 471 |
| | | | fusion | | |
| Cystinosis | CTNS | cystinosin | wt, isoform 1 | 472 | 473 |
| Cystinosis | CTNS | cystinosin | wt, isoform 2 | 474 | 475 |
| FLLD | LPL | lipoprotein lipase | wt | 476 | 477 |
| FLLD | LPL-stop | lipoprotein lipase | S447Stop | 478 | 479 |
| FAP | TTR | transthyretin | T119M | 480 | 481 |
| Lesch Nyhan | HPRT | hypoxanthine | wt | 482 | 483 |
| | | phosphoribosyltransferase 1 | | | |
| AAT deficiency | AAT | alpha1-antitrypsin | wt | 484 | 485 |
| CPS1D | CPS1 | carbamoyl phosphate | wt | 486 | 487 |
| | | synthase 1 | | | |
| MCADD | MCAD | medium-chain specific acyl- | wt, isoform 1 | 488 | 489 |
| | | CoA dehydrogenase | | | |
| MCADD | MCAD | medium-chain specific acyl- | wt, isoform 2 | 490 | 491 |
| | | CoA dehydrogenase | | | |
| VLCAD | ACADVL | very long-chain specific | wt, isoform 1 | 492 | 493 |
| | | acyl-CoA dehydrogenase | | | |
| VLCAD | ACADVL | very long-chain specific | wt, isoform 2 | 494 | 495 |
| | | acyl-CoA dehydrogenase | | | |
| VLCAD | ACADVL | very long-chain specific | wt, isoform 3 | 496 | 497 |
| | | acyl-CoA dehydrogenase | | | |
| Fabry | GLA | alpha-galactosidase A | wt | 498 | 499 |
| Choroideremia | CNTF | ciliary neurotrophic factor | wt | 500 | 501 |
| Retinisis | REP1 | Rab escort protein 1 | wt | 502 | 503 |
| pigmentosa | | | | | |
| Retinisis | RPE65 | retinal pigment epithelium- | wt | 504 | 505 |
| pigmentosa | | specific protein 65 kDa | | | |
| PFIC1 | ATP8B1 | ATPase, | wt | 506 | 507 |
| | | aminophospholipid | | | |
| | | transporter, class I, type 8B, | | | |
| | | member 1 | | | |
| GSD1b | G6PT1 | glucose-6-phosphate | wt, isoform 1 | 508 | 509 |
| | | translocase | | | |
| GSD1b | G6PT1 | glucose-6-phosphate | wt, isoform 2 | 510 | 511 |
| | | translocase | | | |

In some aspects, the dsDNA encodes a gene for the treatment of type 1 diabetes. In some aspects, the dsDNA encodes GLP-1 (glucagon-like peptide 1). In some aspects, the dsDNA encodes GIP (glucose-dependent insulinotropic polypeptide). In some aspects, the dsDNA encodes a modified GLP-1 gene. In some aspects, the modified GLP-1 is a modified GLP-1 (7-37) cDNA, In some aspect, the dsDNA encodes a GLP-1 agonist. In some aspects, the GLP-1 agonist is exendin-4. In some aspects, the GLP-1 is a GLP-1 fusion protein. In some aspects, the GLP-1 fusion protein is a GLP-1/Fc fusion protein. In some aspects, the exendin-4 is an exendin-4 peptide. In some aspects, the exendin-4 is an exendin-4 fusion protein. In some aspects, the exendin-4 fusion protein is an exendin-4/Fc fusion protein. In some aspects, the GLP-1 agonist is selected from the group consisting of exenatide, liraglutide, lixisenative, taspoglutide, albiglutide, dulaglutide, emaglutide, and semaglutide. See A. Palani et al. 7.12—GLP-1 Receptor Agonists for the Treatment of Diabetes and Obesity, Editor: Chackalamannil et al. Comprehensive Medicinal Chemistry III, Elsevier, 2017, pages 481-490; Dowarah et al. Anti-diabetic drugs recent approaches and advancements, Bioorganic & Medicinal Chemistry, 2020, 28(5):115263; and Deng et al. Drug discovery approaches targeting the incretin pathway, Bioorganic Chemistry, 2020, 99: 103810, which are herein incorporated by references in their entireties.

TABLE 8
Sequences of GLP-1 and agonists. The sequences shown correspond
to GLP-1 (SEQ ID NO: 512), exenatide (SEQ ID NO: 513),
liraglutide (SEQ ID NO: 514), lixisenatide (SEQ ID NO: 515),
taspoglutide (SEQ ID NO: 516), albiglutide (SEQ ID NO:517),
dulaglutide (SEQ ID NO: 518), and semaglutide (SEQ ID NO: 519).

| | |
|---|---|
| GLP-1: | H¹AEGT⁵FTSDV¹⁰SSYLE¹⁵GQAAK²⁰EFIAW²⁵LVKGR³⁰ [symbol] NH₂ |
| Exenatide: | 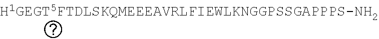 |
| Liraglutide: | 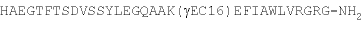 |
| Lixisenatide | 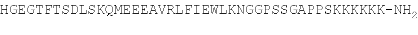 |
| Taspoglutide | HUEGTFTSDVSSYLEGQAAKEFIAWLVKUR-NH₂ |
| Albiglutide | [HGEGTFTSDVSSYLEGQAAKEFIAWLVKGR]₂-Albumin |
| Dulaglutide | [HGEGTFTSDVSSYLEEQAAKEFIAWLVKGG-linker]₂-IgG4 Fc domain |
| Semaglutide | HUEGTFTSDVSSYLEGQAAK(PEG2PEG2γEC18OH)EFIAWLVRGRG |

[symbol]

indicates data missing or illegible when filed

In some aspects, the dsDNA encodes a therapy for type 2 diabetes. Genetic association studies have identified at least 75 independent genetic loci for T2DM and various new therapeutic targets have been determined. For example, three novel mutations in gene KCNJ11 are associated with the development of autosomal dominant, early-onset T2DM. Contrasting with their limited effects on disease incidence and development, genetic loci can have much stronger effects on drug response. A large number of genetic polymorphisms affect the response to oral anti-diabetic drugs. Genes encoding SLC2A2 and organic cation transporters (OCT1, OCT2, and OCT3) are associated with glycemic response to metformin, while SLCO1B3 and KCNQ are associated with sulfonylureas response. There are several genetic loci with potential for T2DM gene therapy. One good example is nucleotide-binding oligomerization domain-like receptor protein 3 (NLRP3). Inhibition of gene NLRP3 attenuates inflammation, protects pancreatic j-cells from apoptosis, and prevents T2DM development in mice. See Yue et al. (2019) Advances and potential of gene therapy for type 2 diabetes mellitus, Biotechnology & Biotechnological Equipment, 33:1, 1150-1157, which is herein incorporated by reference in its entirety.

TABLE 9
Potential targets for type 2 diabetes gene therapy.

| Class | Gene(s) | Main function |
|---|---|---|
| Genes | GLUTs | Re-absorption of filtered glucose from the kidney |
| regulating | | into the bloodstream |
| glucose | SGLTs | Play a fundamental role in the muscle and liver |
| homeostasis | | glucose fluxes |
| | FGFs | Play significant roles in glucose homeostasis |
| | SIRT6 | Associated with increased glycolysis and GLUTs |
| | | expression |
| Genes | GLP-1 and its analogs/agnoists | Increase beta-cell survival, stimulate insulin gene |
| improving | | expression, and secretion |
| insulin | GPGRs and their agonists | Stimulate insulin and GLP-1 secretion |
| secretion | | |
| and/or | CTB-APSL | Promotes insulin secretion and insulin resistance |
| sensitivity | IKK ε, TBK1 | Associated with weight reduction, insulin |
| | | resistance, fatty liver, and inflammation |
| Genes | IL-1β | Associated with inflammation and Î²-cell failure |
| ameliorating | ADPN | Ameliorates diabetic nephropathy |
| diabetic | TGF-α | Plays a role in DKD associated with nephron |
| induced | | reduction |
| complications | NLRP3 | Ameliorates diabetic cardiomypathy |
| | CDKN2A/2B | Associated with T-cell phenotype modulation and |
| | | chronic inflammation |
| | HSP70 | Associated with mitochondrial bioenergetics and |
| | | diabetic sensory neuropathy |
| | MicroRNAs | Involved in regulating the diabetic |
| | | microvasculature |

SGLTs, sodium-glucose co-transporters; GLUTs, glucose transporters; FGFs, fibroblast growth factors; SIRT6, Sirtuin 6; GLP-1, glycogen like peptide 1; GPGRs, G protein-coupled receptors; CTB-APSL, cholera toxin B subunit and active peptide from shark liver; ADPN, adiponectin; TGF-α, transforming growth factor-alpha; NLRP3, nucleotide-binding oligomerization domain-like receptor protein 3; DKD, diabetic kidney disease; HSP70, heat shock protein 70.

In some aspects, the dsDNA encodes a gene for the treatment of lipoprotein lipase deficiency. In some aspects, the dsDNA encodes the GOI in alipogene tiparvovec (GLYBERA™).

In some aspects, the dsDNA encodes a gene for the treatment of metachromatic leukodystrophy. In some aspects, the dsDNA encodes the GOI in atidarsagene autotemcel (LIBMELDY™). In some aspects, the dsDNA encodes a gene for the treatment of B-cell lymphoma. In some aspects, the dsDNA encodes the GOI in axicabtagene ciloleucel (YESCARTA™). In some aspects, the dsDNA encodes a gene for the treatment of wounds or Dystrophic epidermolysis bullosa (DEB). In some aspects, the dsDNA encodes the GOI in beremagene geperpavec (VYJUVEK™). In some aspects, the dsDNA encodes a gene for the treatment beta thalassemia. In some aspects, the dsDNA encodes the GOI in betibeglogene autotemcel (ZYNTEGLO™). In some aspects, the dsDNA encodes a gene for the treatment of mantle cell lymphoma or acute lymphoblastic leukemia. In some aspects, the dsDNA encodes the GOI in brexucabtagene autoleucel (TECARTUS™). In some aspects, the dsDNA encodes a gene for the treatment of vascular endothelial growth factor peripheral artery disease. In some aspects, the dsDNA encodes the GOI in cambiogenplasmid (NEOVASCULGEN™). In some aspects, the dsDNA encodes a gene for the treatment multiple myeloma. In some aspects, the dsDNA encodes the GOI in ciltacabtagene autoleucel (CARVYKTI™). In some aspects, the dsDNA encodes a gene for the treatment of Duchenne muscular dystrophy. In some aspects, the dsDNA encodes the GOI in delandistrogene moxeparvovec (ELEVIDYS™). In some aspects, the dsDNA encodes a gene for the treatment of adrenoleukodystrophy. In some aspects, the dsDNA encodes the GOI in elivaldogene autotemcel (SKYSONA™). In some aspects, the dsDNA encodes a gene for the treatment of hemophilia B. In some aspects, the dsDNA encodes the GOI in etranacogene dezaparvovec (HEMGENIX™). In some aspects, the dsDNA encodes a gene for the treatment of head and neck squamous cell carcinoma. In some aspects, the dsDNA encodes the GOI in gendicine. In some aspects, the dsDNA encodes a gene for the treatment of multiple myeloma. In some aspects, the dsDNA encodes the GOI in idecabtagene vicleucel (ABECMA™). In some aspects, the dsDNA encodes a gene for the treatment of B-cell lymphoma. In some aspects, the dsDNA encodes the GOI in lisocabtagene maraleucel (BREYANZI™). In some aspects, the dsDNA encodes a gene for the treatment of bladder cancer. In some aspects, the dsDNA encodes the GOI in nadofaragene firadenovec (ADSTILADRIN™). In some aspects, the dsDNA encodes a gene for the treatment of spinal muscular atrophy. In some aspects, the dsDNA encodes the GOI in onasemnogene abeparvovec (ZOLGENSMA™). In some aspects, the dsDNA encodes a gene for the treatment of adenosine deaminase deficiency (ADA-SCID). In some aspects, the dsDNA encodes the human enzyme adenosine deaminase (ADA) GOI in strimvelis (autologous CD34+). In some aspects, the dsDNA encodes a gene for the treatment of melanoma in patients who have recurring skin lesions. In some aspects, the dsDNA encodes the GOI in talimogene laherparepvec (IMLYGIC™). In some aspects, the dsDNA encodes a gene for the treatment of B cell lymphoblastic leukemia. In some aspects, the dsDNA encodes the GOI in tisagenlecleucel (KYMRIAH™). In some aspects, the dsDNA encodes a gene for the treatment of hemophilia A. In some aspects, the dsDNA encodes the GOI in valoctocogene roxaparvovec (ROCTAVIAN™) In some aspects, the dsDNA encodes a gene for the treatment of Leber congenital amaurosis. In some aspects, the dsDNA encodes the GOI in voretigene neparvovec (LUXTURNA™).

### Payload: Chimeric Antigen Receptor (CARs)

In some aspects the payload comprises a CAR (e.g., integrated into the lipid bilayer of a lipidic delivery system component of an AAV-ITR based gene delivery system of the present disclosure) or polynucleotide encoding a CAR (e.g., a dsDNA polynucleotide in the AAV-ITR vector).

As used herein, the term “Chimeric Antigen Receptor” or alternatively a “CAR” refers to a recombinant polypeptide construct comprising at least an extracellular antigen binding domain, a transmembrane domain, and a cytoplasmic signaling domain comprising a functional signaling domain derived from a stimulatory molecule. In one aspect, the stimulatory molecule is the zeta chain associated with the T cell receptor complex. In one aspect, the cytoplasmic signaling domain further comprises one or more functional signaling domains derived from at least one costimulatory molecule as defined below. In one aspect, the costimulatory molecule is chosen from 4-1BB (i.e., CD137), CD3, and/or CD28.

In one aspect, the CAR comprises a chimeric fusion protein comprising an extracellular antigen recognition domain, a transmembrane domain, and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule.

In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the scFv domain during cellular processing and localization of the CAR to the cellular membrane.

The portion of the CAR composition comprising an antibody or antibody fragment thereof can exist in a variety of forms where the antigen binding domain is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen-binding domain of a CAR composition disclosed herein comprises an antibody fragment. In one aspect, the CAR comprises an antibody fragment that comprises a scFv.

In some aspects, the CAR comprises an extracellular antigen recognition domain comprising an antibody disclosed herein or an antigen-binding portion thereof. In some aspects, the extracellular antigen recognition domain comprises a scFv derived from an antibody disclosed herein. In some aspects, the extracellular antigen recognition domain comprises an antibody in a format disclosed in the present application.

In some aspects, the CAR is tisagenlecleucel (KYMRIAH®), an anti-CD19 CAR for the treatment of B-cell precursor ALL, diffuse large B-cell lymphoma, or follicular lymphoma. In some aspects, the CAR is axicabtagene ciloleucel (YESCARTA®), an anti-CD19 CAR for the treatment of diffuse large B-cell lymphoma and follicular lymphoma. In some aspects, the CAR is brexucabtagene autoleucel (TECARTUS®) an anti-CD19 CAR for the treatment of mantle cell lymphoma and B-cell precursor ALL. In some aspects, the CAR is lisocabtagene maraleucel (BREYANZI®) an anti-CD19 CAR for the treatment of diffuse large B-cell lymphoma. In some aspects, the CAR is idecabtagene vicleucel (ABECMA®) an anti-BCMA CAR for the treatment of multiple myeloma. In some aspects, the CAR is ciltacabtagene autoleucel (CARVYKTI®) an anti-BCMA CAR for the treatment of multiple myeloma.

In some aspects the payload comprises a CAR or polynucleotide encoding a CAR wherein the CAR's binding portion is an anti-CD19, anti-BCMA, anti-HER2, anti-CD20, anti-CD22, anti-IL13Ra2, anti-GPC3, or combination thereof. In some aspects the payload comprises a CAR or polynucleotide encoding a CAR wherein the CAR's binding portion comprises a scFv derived from a therapeutic antibody disclosed herein, e.g., a therapeutic antibody disclosed herein targeting an antigen expressed on the surface of T cells. In some aspects, the CAR is a monospecific CAR. In some aspects, the CAR is a bispecific CAR.

In some aspects, the payload comprises a polynucleotide encoding a CAR disclosed in the following table. Thus, in some aspects, the AAV-ITR vector comprises a coding sequence encoding a CAR disclosed in the following table.

TABLE 10
Exemplary CAR sequences. When two chains are present the first SEQ ID
NO correspondence to the top protein chain and the second SEQ ID NO
corresponds to the bottom protein chain in the protein sequence cell.

| SEQ ID | |
|---|---|
| NO | CAR name |
| 520 | acmucabtagene autoleucel |
| 521 | anbalcabtagene autoleucel |
| 522 | axicabtagene ciloleucel YESCARTA ® |
| 523 | azercabtagene zapreleucel |
| 524 | brexucabtagene autoleucel TECARTUS ™ |
| 525 | cemacabtagene ansegedleucel |
| 526 | evoncabtagene pazurgedleucel |
| 527 | inaticabtagene autoleucel |
| 528 | lisocabtagene maraleucel BREYANZI ™ |
| 529 | obecabtagene autoleucel |
| 530 | anitocabtagene autoleucel |
| 531 | ciltacabtagene autoleucel CARVYKTI ™ |
| 532 | durcabtagene autoleucel |
| 533 | equecabtagene autoleucel |
| 534 | fencabtagene autoleucel |
| 535 | firicabtagene autoleucel |
| 536 | gavocabtagene autoleucel |
| 537 | idecabtagene vicleucel |
| 538 | itezocabtagene autoleucel |
| 539 | motacabtagene lurevgedleucel |
| 540 | obecabtagene autoleucel |
| 541 | orvacabtagene autoleucel |
| 542 | plixacabtagene autoleucel |
| 543 | pomlucabtagene autoleucel |
| 544 | prizloncabtagene autoleucel |
| 545 | rapcabtagene autoleucel |
| 546 | relmacabtagene autoleucel |
| 547 | ribrecabtagene autoleucel |
| 548 | satricabtagene autoleucel |
| 549 | tebrocabtagene autoleucel |
| 550 | tinocabtagene autoleucel |
| 551/552 | trovocabtagene autoleucel. Comprises two chains that can be in separate AAV-ITR |
| | vectors or in an bicistronic AAV-ITR vector. 1:1 ratio |
| 553/554 | vadacabtagene leraleucel. Comprises two chains that can be in separate AAV-ITR |
| | vectors or in an bicistronic AAV-ITR vector. 1:1 ratio |
| 555/556 | varnimcabtagene autoleucel. Comprises two chains that can be in separate AAV- |
| | ITR vectors or in an bicistronic AAV-ITR vector. 1:1 ratio |
| 557/558 | volamcabtagene durzigedleucel. Comprises two chains that can be in separate AAV- |
| | ITR vectors or in an bicistronic AAV-ITR vector. 1:1 ratio |
| 559/560 | zamtocabtagene autoleucel. Comprises two chains that can be in separate AAV-ITR |
| | vectors or in an bicistronic AAV-ITR vector. 1:1 ratio |
| 561/562 | zevorcabtagene autoleucel. Comprises two chains that can be in separate AAV-ITR |
| | vectors or in an bicistronic AAV-ITR vector. 1:1 ratio |
| 563 | afamitresgene autoleucel |
| 564 | besvatresgene autoleucel |
| 565 | letetresgene autoleucel |
| 566 | mipetresgene autoleucel |
| 567 | olitresgene autoleucel |
| 568 | suvutresgene autoleucel |
| 569 | tacatresgene autoleucel |
| 570 | tisagenlecleucel |

In some aspects, the CAR comprises a bispecific antibody covalently linked to the spacer-TM (spacer and transmembrane domain) and intracellular domain portion of a CAR in the table above. In some aspects, the CAR comprises a MSTAR antibody covalently linked to the spacer-TM (spacer and transmembrane domain) and intracellular domain portion of a CAR in the table above.

### Payload: Therapeutic Antibodies

In some aspects, the payload comprises a polynucleotide in the AAV-ITR vector encoding a therapeutic antibody or an antigen-binding portion thereof, e.g., a therapeutic antibody disclosed below or an antigen-binding portion thereof. In some aspects, the payload comprises a therapeutic antibody or an antigen-binding portion thereof, e.g., a therapeutic antibody disclosed below or an antigen-binding portion thereof, e.g., covalently attached to the surface of a liposome. In some aspects the payload can comprise a polynucleotide in the AAV-ITR vector encoding a fusion protein comprising a therapeutic antibody disclosed below or an antigen-binding portion thereof.

It is to be noted that the used of the therapeutic antibodies and antigen-binding portions thereof disclose below is not limited to serve as payloads in the delivery systems of the present disclosure, e.g., a polynucleotide encoding an antibody as the GOI is an AAV-ITR vector. Thus, in some aspects, a therapeutic antibody disclosed below or an antigen-binding portion thereof can be or can be part of a surface anchored targeting molecule in the lipidic delivery system of an AAV-ITR based gene delivery system of the present disclosure (e.g., a liposome encapsulating the AAV-ITR vector and/or a liposome encapsulating the Rep vector), and direct the AAV-ITR based gene delivery system of the present disclosure to a cell or tissue expressing the molecule to which an antibody disclosed below binds specifically.

In some aspects, VH and/or VL domains of antibodies disclosed below can be used as part of the targeting portion of a CAR. In some aspects, a CAR used as payload in a LNP delivery system of the present disclosure can comprise a scFv comprising VH and VL domains from any of the therapeutic antibodies disclosed below.

In some aspects, the payload comprises an antibody or antigen-binding portion thereof attached to a lipidic delivery system of an AAV-ITR based gene delivery system of the present disclosure, or a polynucleotide in the AAV-ITR encoding an antibody or antigen-binding portion thereof, wherein the antibody is selected from the group consisting of 3F8 (anti-GD2 ganglioside), abagovomab (anti-CA-125), abciximab (anti-CD41, integrin alpha-IIb), abituzumab (anti-CD51), abrezekimab (anti-IL-13), abrilumab (anti-integrin α4β7), actoxumab (anti-Clostridium difficile), adalimumab (anti-TNF-α), adecatumumab (anti-EpCAM), aducanumab (anti-Amyloid beta), afasevikumab (anti-IL-17A, IL-17F), afelimomab (anti-TNF-α), alacizumab pegol VEGFR2), alemtuzumab (anti-CD52), alirocumab (anti-PCSK9), altumomab pentetate (anti-carcinoembryonic antigen (CEA)), amatuximab[(anti-mesothelin), amivantamab (anti-epidermal growth factor receptor (EGFR), cMet), anatumomab mafenatox (anti-tumor-associated glycoprotein 72 (TAG-72)), andecaliximab (anti-gelatinase B), anetumab ravtansine (anti-mesothelin (MSLN)), anifrolumab (anti-IFN-α/β receptor), ansuvimab (anti-Ebola virus glycoprotein), anrukinzumab (anti-IL-13), apolizumab[(anti-HLA-DR), aprutumab ixadotin (anti-FGFR2), arcitumomab (anti-Carcinoembryonic antigen (CEA)), ascrinvacumab (anti-activin receptor-like kinase 1), aselizumab (anti-L-selectin (CD62L)), atezolizumab (anti-PD-L1), atidortoxumab (anti-Staphylococcus aureus alpha toxin), atinumab (anti-RTN4), atorolimumab (anti-Rhesus factor), avelumab (anti-PD-L1), azintuxizumab vedotin (anti-CD319), bamlanivimab (anti-spike protein receptor binding domain (RBD) of SARS-CoV-2), bapineuzumab (anti-β-amyloid), basiliximab (anti-CD25 (α chain of IL-2 receptor)), bavituximab (anti-phosphatidylserine), BCD-100 (anti-PD-1), bebtelovimab (anti-spike protein receptor binding domain (RBD) of SARS-CoV-2), bectumomab (anti-CD22), bedinvetmab (anti-nerve growth factor (NGF)), begelomab (anti-DPP4), belantamab mafodotin (anti-B-cell maturation antigen (BCMA)), belimumab (anti-B-cell activating factor (BAFF)), bemarituzumab (anti-FGFR2), benralizumab[(anti-CD125), berlimatoxumab (anti-Staphylococcus aureus bi-component leucocidin), bermekimab (anti-IL-la), bersanlimab (anti-ICAM-1), bertilimumab (anti-CCL11 (eotaxin-1)), besilesomab (anti-carcinoembryonic antigen (CEA)-related antigen), bevacizumab (anti-VEGF-A), bezlotoxumab[(anti-Clostridium difficile), biciromab (anti-beta chain), bimagrumab (anti-ACVR2B), bimekizumab (anti-IL-17A, IL-17F, IL-17AF), birtamimab (anti-serum amyloid A protein), bivatuzumab (anti-CD44 v6), bleselumab (anti-CD40), blinatumomab (anti-CD19), blontuvetmab (anti-CD20), blosozumab (anti-SOST), bococizumab (anti-PCSK9), brazikumab (anti-IL-23), brentuximab vedotin (anti-CD30 (TNFRSF8)), briakinumab (anti-IL-12, IL-23), brodalumab (anti-IL-17), brolucizumab (anti-vascular endothelial growth factor A (VEGFA)), brontictuzumab (anti-Notch 1), burosumab (anti-FGF 23), cabiralizumab (anti-CSF1R), camidanlumab tesirine (anti-CD25 (α chain of IL-2 receptor), camrelizumab (anti-PD-1), canakinumab (anti-IL-1), cantuzumab mertansine (anti-CanAg (a glycoform of MUC1)), cantuzumab ravtansine (anti-CanAg (α glycoform of MUC1)), caplacizumab (anti-VWF), casirivimab (anti-spike protein receptor binding domain (RBD) of SARS-CoV-2), capromab (anti-Glutamate carboxypeptidase II), carlumab (anti-MCP-1), carotuximab (anti-endoglin), catumaxomab (anti-EpCAM, CD3), cBR96-doxorubicin immunoconjugate (anti-Lewis-Y antigen), cedelizumab (anti-CD4), cemiplimab (anti-PD-1), cergutuzumab amunaleukin (anti-IL-2), certolizumab pegol (anti-TNF-α), cetrelimab (anti-PD-1), cetuximab (anti-epidermal growth factor receptor (EGFR)), cibisatamab (anti-CEACAM5), cilgavimab (anti-spike protein receptor binding domain (RBD) of SARS-CoV-2), cirmtuzumab (anti-ROR1), citatuzumab bogatox (anti-EpCAM), cixutumumab (anti-IGF-1 receptor (CD221)), clazakizumab (anti-IL-6), clenoliximab (anti-CD4), clivatuzumab tetraxetan (anti-MUC1), codrituzumab (anti-glypican 3), cofetuzumab pelidotin (anti-PTK7), coltuximab ravtansine (anti-CD19), conatumumab (anti-TRAIL-R2), concizumab (anti-tissue factor pathway inhibitor (TFPI)), cosfroviximab (anti-ebolavirus glycoprotein), crenezumab (anti-β-amyloid (1-40 and 1-42)), crizanlizumab (anti-selectin P), crotedumab (anti-glucagon receptor (GCGR)), CR6261 (anti-Hemagglutinin (influenza)), cusatuzumab (anti-CD70), dacetuzumab (anti-CD40), daclizumab (anti-CD25 (α chain of IL-2 receptor), dalotuzumab (anti-IGF-1 receptor (CD221)), dapirolizumab pegol (anti-CD154 (CD40L)), daratumumab (anti-CD38), dectrekumab (anti-IL-13), demcizumab (anti-DLL4), denintuzumab mafodotin (anti-CD19), denosumab (anti-RANKL), depatuxizumab mafodotin (anti-EGFR), derlotuximab biotin (anti-histone complex), detumomab (anti-B-lymphoma cell), dezamizumab (anti-serum amyloid P component), dinutuximab (anti-GD2 ganglioside), dinutuximab beta (anti-GD2 ganglioside), diridavumab (anti-Hemagglutinin (influenza)), domagrozumab (anti-GDF-8), donanemab (anti-Amyloid beta), dostarlimab (anti-PCDP1), drozitumab (anti-DR5), DS-8201 (anti-HER2), duligotuzumab (anti-ERBB3 (HER3)), dupilumab (anti-IL-4Ra), durvalumab (anti-PD-L1), dusigitumab (anti-IGF-2), duvortuxizumab (anti-CD19, CD3E), ecromeximab (anti-GD3 ganglioside), eculizumab (anti-C5), edobacomab (anti-endotoxin), edrecolomab (anti-EpCAM), efalizumab (anti-LFA-1 (CD11a)), efungumab (anti-Hsp90), eldelumab (anti-CXCL10 (IP-10)), elezanumab (anti-repulsive guidance molecule A (RGMA)), elgemtumab (anti-ERBB3 (HER3)), elotuzumab (anti-SLAMF7), elsilimomab (anti-IL-6), emactuzumab (anti-CSF1R), emapalumab (anti-IFN-7), emibetuzumab (anti-HGFR), emicizumab (anti-activated F9, F10), enapotamab vedotin (anti-AXL), enavatuzumab (anti-TWEAK receptor), enfortumab vedotin (anti-nectin-4), enlimomab pegol (anti-ICAM-1 (CD54)), enoblituzumab (anti-CD276), enokizumab (anti-IL-9), enoticumab (anti-DLL4), ensituximab (anti-MUCSAC), epcoritamab (anti-CD3, CD20), epitumomab cituxetan (anti-episialin), epratuzumab (anti-CD22), eptinezumab (anti-calcitonin gene-related peptide), erenumab (anti-calcitonin gene-related peptide receptor (CGRP)), erlizumab (anti-ITGB2 (CD18)), ertumaxomab (anti-HER2/neu, CD3), etaracizumab (anti-integrin avP3), etesevimab (anti-spike protein receptor binding domain (RBD) of SARS-CoV-2), etigilimab (anti-TIGIT), etrolizumab (anti-integrin 07), evinacumab (anti-angiopoietin 3), evolocumab (anti-PCSK9), exbivirumab (anti-hepatitis B surface antigen), fanolesomab (anti-CD15), faralimomab (anti-IFN receptor), faricimab (anti-VEGF-A and Ang-2), farletuzumab (anti-folate receptor 1), fasinumab (anti-nerve growth factor (NGF)), FBTA05 (anti-CD20), felvizumab (anti-respiratory syncytial virus), fezakinumab (anti-IL-22), fibatuzumab (anti-ephrin receptor A3), ficlatuzumab (anti-Hepatocyte growth factor (HGF)), figitumumab (anti-IGF-1 receptor (CD221)), firivumab (anti-Hemagglutinin (influenza)), flanvotumab (anti-TYRP1 (glycoprotein 75)), fletikumab (anti-IL-20), flotetuzumab (anti-IL-3 receptor), fontolizumab (anti-IFN-7), foralumab (anti-CD3E), foravirumab (anti-rabies virus glycoprotein), fremanezumab (anti-calcitonin gene-related peptide alpha and beta), fresolimumab (anti-TGF-β), frovocimab (anti-PCSK9), frunevetmab (anti-nerve growth factor (NGF)), fulranumab (anti-nerve growth factor (NGF)), futuximab (anti-Epidermal growth factor receptor (EGFR)), galcanezumab (anti-calcitonin), galiximab (anti-CD80), gancotamab (anti-HER2/neu), ganitumab (anti-IGF-1 receptor (CD221)), gantenerumab (anti-β-amyloid (1-40 and 1-42)), gatipotuzumab (anti-MUC1), gavilimomab (anti-CD147 (basigin)), gedivumab (anti-Hemagglutinin (influenza)), gemtuzumab ozogamicin (anti-CD33), gevokizumab (anti-IL-1β), gilvetmab (anti-PCDC1), gimsilumab (anti-CSF2), girentuximab (anti-carbonic anhydrase 9 (CA-IX)), glembatumumab vedotin (anti-GPNMB), glofitamab (anti-CD20, CD3), golimumab (anti-TNF-α), gomiliximab (anti-CD23 (IgE receptor), gosuranemab (anti-tau protein), guselkumab (anti-IL-23), ianalumab (anti-BAFF-R), ibalizumab (anti-CD4), sintilimab (anti-PD-1), ibritumomab tiuxetan (anti-CD20), icrucumab (anti-VEGFR-1), idarucizumab (anti-dabigatran), ifabotuzumab (anti-EPHA3), igovomab (anti-CA-125), iladatuzumab vedotin (anti-CD79B), imalumab (anti-macrophage migration inhibitory factor (MIF)), imaprelimab (anti-melanoma cell adhesion molecule (MCAM)), imciromab (anti-cardiac myosin), imdevimab (anti-spike protein receptor binding domain (RBD) of SARS-CoV-2), imgatuzumab (anti-Epidermal growth factor receptor (EGFR)), inclacumab (anti-selectin P), indatuximab ravtansine (anti-SDC1), indusatumab vedotin (anti-GUCY2C), inebilizumab (anti-CD19), infliximab (anti-TNF-α), intetumumab (anti-CD51), inolimomab (anti-CD25 (α chain of IL-2 receptor)), inotuzumab ozogamicin (anti-CD22), ipilimumab (anti-CD152), iomab-B (anti-CD45), iratumumab (anti-CD30 (TNFRSF8)), isatuximab (anti-CD38), iscalimab (anti-CD40), istiratumab (anti-IGF-1 receptor (CD221)), itolizumab (anti-CD6), ixekizumab (anti-IL-17A), keliximab (anti-CD4), labetuzumab (anti-Carcinoembryonic antigen (CEA)), lacnotuzumab (anti-CSF1, macrophage colony stimulating factor (MCSF)), ladiratuzumab vedotin (anti-LIV-1), lampalizumab (anti-Complement factor D (CFD)), lanadelumab (anti-kallikrein), landogrozumab (anti-GDF-8), laprituximab emtansine (anti-epidermal growth factor receptor (EGFR)), larcaviximab (anti-ebolavirus glycoprotein), lebrikizumab (anti-IL-13), lecanemab (anti-β-amyloid), lemalesomab (anti-NCA-90 (granulocyte antigen)), lendalizumab (anti-C5), lenvervimab (anti-hepatitis B surfage antigen), lenzilumab (anti-CSF2), lerdelimumab (anti-TGF-β2), leronlimab (anti-CCR5), lesofavumab (anti-Hemagglutinin (influenza)), letolizumab (anti-tumor necrosis factor related activation protein (TRAP)), lexatumumab (anti-TRAIL-R2), libivirumab (anti-hepatitis B surface antigen), lifastuzumab vedotin (anti-phosphate-sodium co-transporter), ligelizumab (anti-IGHE), loncastuximab tesirine (anti-CD19), losatuxizumab vedotin (anti-EGRF, ERBB1 HER1), lilotomab satetraxetan (anti-CD37), lintuzumab (anti-CD33), lirilumab (anti-KIR2D), lodelcizumab (anti-PCSK9), lorvotuzumab mertansine (anti-CD56), lucatumumab (anti-CD40), lulizumab pegol (anti-CD28), lumiliximab (anti-CD23 (IgE receptor)), lumretuzumab (anti-ERBB3 (HER3)), lupartumab amadotin (anti-LYPD3), lutikizumab (anti-IL-la), mapatumumab (anti-TRAIL-R1), margetuximab (anti-HER2), marstacimab (anti-tissue factor pathway inhibitor (TFPI)), maslimomab (anti-T-cell receptor), mavrilimumab (anti-GMCSF receptor α-chain), matuzumab (anti-epidermal growth factor receptor (EGFR)), mepolizumab (anti-IL-5), metelimumab (anti-TGF-β1), milatuzumab (anti-CD74), minretumomab (anti-TAG-72), mirikizumab (anti-IL-23), mirvetuximab soravtansine (anti-folate receptor alpha), mitumomab (anti-GD3 ganglioside), modotuximab (anti-EGFR extracellular domain III), mogamulizumab (anti-CCR4), monalizumab (anti-NKG2A), morolimumab (anti-Rhesus factor), mosunetuzumab (anti-CD3E, MS4A1, CD20), motavizumab (anti-respiratory syncytial virus), moxetumomab pasudotox (anti-CD22), muromonab-CD3 (anti-CD3), nacolomab tafenatox (anti-C242 antigen), namilumab (anti-CSF2), naptumomab estafenatox (anti-5T4)), naratuximab emtansine (anti-CD37)), narnatumab (anti-MST1R (aka RON)), natalizumab (anti-integrin α4), navicixizumab (anti-DLL4 and VEGFA), navivumab (anti-Hemagglutinin (influenza)), naxitamab (anti-c-Met), nebacumab (anti-endotoxin), necitumumab (anti-epidermal growth factor receptor (EGFR)), nemolizumab (anti-IL-31 receptor A), NEOD001 (anti-amyloid), nerelimomab (anti-TNF-α, nesvacumab (anti-angiopoietin 2), netakimab (anti-IL-17A), nimotuzumab (anti-epidermal growth factor receptor (EGFR)), nirsevimab (anti-RSV fusion glycoprotein), nivolumab (anti-PD-1), nofetumomab merpentan (pancarcinoma murine antibody NR-LU-10 linked with gamma-emitting radioisotope technetium 99m (Tc 99m)), obiltoxaximab (anti-Bacillus anthracis anthrax), obinutuzumab anti-CD20, ocaratuzumab (anti-CD20), ocrelizumab (anti-CD20). atoltivimab/maftivimab/odesivimab (INMAZEB®, REGN-EB3) (anti-Zaire ebolavirus glycoprotein combination therapy), odulimomab (anti-LFA-1 (CD11a)), ofatumumab (anti-CD20), olaratumab (anti-PDGFRA), oleclumab (anti-5′-nucleotidase), olendalizumab (anti-complement C5a), olokizumab (anti-IL-6), omalizumab (anti-IgE Fc region), omburtamab (anti-CD276), oMS721 (anti-MASP-2), onartuzumab (anti-human scatter factor receptor kinase), ontuxizumab (anti-TEM1), onvatilimab (anti-VISTA (protein) (VSIR)); opicinumab (anti-LINGO-1), oportuzumab monatox (anti-EpCAM), oregovomab (anti-CA-125), orticumab (anti-oxLDL), otelixizumab (anti-CD3), otilimab (anti-GMCSF), otlertuzumab (anti-CD37), oxelumab (anti-OX-40), ozanezumab (anti-NOGO-A), ozoralizumab (anti-TNF-α), pagibaximab (anti-lipoteichoic acid), palivizumab (anti-F protein of respiratory syncytial virus), pamrevlumab (anti-connective tissue growth factor (CTGF)), panitumumab (anti-epidermal growth factor receptor (EGFR)), pankomab (anti-tumor specific glycosylation of MUC1), panobacumab (anti-Pseudomonas aeruginosa), parsatuzumab (anti-EGFL7), pascolizumab (anti-IL-4), pasotuxizumab (anti-folate hydrolase), pateclizumab (anti-lymphotoxin alpha (LTA)), patritumab (anti-ERBB3 (HER3)), PDR001 (anti-PD-1), pembrolizumab (anti-PD-1), pemtumomab (anti-MUC1), perakizumab (anti-IL-17A), pertuzumab (anti-HER2/neu), pexelizumab (anti-C5), pidilizumab (anti-PD-1), pinatuzumab vedotin (anti-CD22), pintumomab (anti-adenocarcinoma antigen), placulumab (anti-TNF), pozelimab (anti-C5), prezalumab (anti-TNF), plozalizumab (anti-CCR2), pogalizumab (anti-tumor necrosis factor receptor (TNFR) superfamily member 4), polatuzumab vedotin (anti-CD79B), ponezumab (anti-β-amyloid), porgaviximab (anti-Zaire ebolavirus glycoprotein), prasinezumab (anti-Alpha-synuclein), prezalizumab (anti-inducible T-cell co-stimulatory ligand (ICOSL)), priliximab (anti-CD4), pritoxaximab (anti-E. coli shiga toxin type-1), pritumumab (anti-vimentin), PRO 140 (anti-CCR5), quilizumab (anti-IGHE), racotumomab (anti-NGNA ganglioside), radretumab (anti-fibronectin extra domain-B), rafivirumab (anti-rabies virus glycoprotein), ralpancizumab (anti-PCSK9), ramucirumab (anti-VEGFR2), ranevetmab (anti-NGF), ranibizumab (anti-VEGF-A), raxibacumab (anti-anthrax toxin protective antigen), ravagalimab (anti-CD40), ravulizumab (anti-C5), refanezumab (anti-myelin-associated glycoprotein), regavirumab (anti-cytomegalovirus glycoprotein B), regdanvimab (anti-spike protein receptor binding domain (RBD) of SARS-CoV-2), relatlimab (anti-LAG3), remtolumab (anti-IL-17A, TNF), reslizumab (anti-IL-5), retifanlimab (anti-PD-1), rilotumumab (anti-hepatocyte growth factor (HGF)), rinucumab (anti-PDGFRB), risankizumab (anti-IL-23A), rituximab (anti-CD20), rivabazumab pegol (anti-Pseudomonas aeruginosa type III secretion system), robatumumab (anti-IGF-1 receptor (CD221)), rmab (anti-rabies virus G glycoprotein), roledumab (anti-RHD (gene) (RHD)), romilkimab (anti-IL-13), romosozumab (anti-sclerostin), rontalizumab (anti-IFN-α), rosmantuzumab (anti-root plate-specific spondin 3), rovalpituzumab tesirine (anti-DLL3), rovelizumab (anti-CD11, anti-CD18), rozanolixizumab (anti-FCGRT), ruplizumab (anti-CD154 (CD40L)), SA237 (anti-IL-6 receptor), sacituzumab govitecan (anti-TROP-2), samalizumab (anti-CD200), samrotamab vedotin (anti-LRRC15), sarilumab (anti-IL-6), satralizumab (anti-IL-6 receptor), satumomab pendetide (anti-TAG-72), secukinumab (anti-IL-17A), selicrelumab (anti-CD40), seribantumab (anti-ERBB3 (HER3)), setoxaximab (anti-E. coli shiga toxin type-2), setrusumab (anti-sclerostin (SOST)), sevirumab (anti-cytomegalovirus), sibrotuzumab (anti-FAP (gene) (FAP)), SGN-CD19A (anti-CD19), SHP647 (anti-mucosal addressin cell adhesion molecule), sifalimumab (anti-IFN-α), siltuximab (anti-IL-6), simtuzumab (anti-LOXL2), siplizumab (anti-CD2), sirtratumab vedotin (anti-SLITRK6), sirukumab (anti-IL-6), sofituzumab vedotin (anti-CA-125), solanezumab (anti-β-amyloid), solitomab (anti-EpCAM), sonepcizumab (anti-sphingosine-1-phosphate), sontuzumab (anti-episialin), sotrovimab (anti-spike protein receptor binding domain (RBD) of SARS-CoV-2), spartalizumab (anti-PD-1), spesolimab (anti-Interleukin 36 receptor (IL1RL2/IL1RAP)), stamulumab (anti-myostatin), sulesomab (anti-NCA-90 (granulocyte antigen)), suptavumab (anti-RSVFR), sutimlimab (anti-complement component is (Cis)), suvizumab (anti-HIV-1), suvratoxumab (anti-Staphylococcus aureus alpha toxin), tabalumab (anti-B-cell activating factor (BAFF)), tacatuzumab tetraxetan (anti-alpha-fetoprotein), tadocizumab (anti-integrin aIbp3), tafasitamab (anti-CD19), talacotuzumab (anti-CD123), talizumab (anti-IgE), talquetamab (anti-GPRCSD, anti-CD3), tamtuvetmab (anti-CD52), tanezumab (anti-nerve growth factor (NGF)), taplitumomab paptox (anti-CD19), tarextumab (anti-Notch receptor), tavolimab (anti-CD134), teclistamab (anti-B-cell maturation antigen (BCMA), CD3), tefibazumab (anti-clumping factor A), telimomab aritox (anti-CD5), telisotuzumab (anti-HGFR), telisotuzumab vedotin (anti-HGFR), tenatumomab (anti-tenascin C), teneliximab (anti-CD40), teplizumab (anti-CD3), tepoditamab (anti-dendritic cell-associated lectin 2), teprotumumab (anti-IGF-1 receptor (CD221)), tesidolumab (anti-C5), tetulomab (anti-CD37), tezepelumab (anti-thymic stromal lymphopoietin (TSLP)), TGN1412 (anti-CD28), tibulizumab (anti-B-cell activating factor (BAFF)), tildrakizumab (anti-IL-23), tigatuzumab (anti-TRAIL-R2), timigutuzumab (anti-HER2), timolumab (anti-AOC3), tiragotumab (anti-TIGIT), tislelizumab (anti-PCDC1, anti-CD279), tisotumab vedotin (anti-coagulation factor III), tixagevimab (anti-spike protein receptor binding domain (RBD) of SARS-CoV-2), TNX-650 (anti-IL-13), tocilizumab (anti-IL-6 receptor), tomuzotuximab (anti-Epidermal growth factor receptor (EGFR), anti-HER1), toralizumab (anti-CD154 (CD40L)), tosatoxumab (anti-Staphylococcus aureus), tositumomab (anti-CD20), tovetumab (anti-PDGFRA), tralokinumab (anti-IL-13), trastuzumab (anti-HER2/neu), trastuzumab duocarmazine (anti-HER2/neu), trastuzumab emtansine (anti-HER2/neu), TRBS07 (anti-GD2 ganglioside), tregalizumab (anti-CD4), tremelimumab (anti-CTLA-4), trevogrumab (anti-growth differentiation factor 8), tucotuzumab celmoleukin (anti-EpCAM), tuvirumab (anti-hepatitis B virus), ublituximab (anti-CD20), ulocuplumab (anti-CXCR4 (CD184)), urelumab (anti-4-1BB (CD137)), urtoxazumab (anti-Escherichia coli), ustekinumab (anti-IL-12, anti-IL-23), utomilumab (anti-4-1BB (CD137)), vadastuximab talirine (anti-CD33), vanalimab (anti-CD40), vandortuzumab vedotin (anti-STEAP1), vantictumab (anti-Frizzled receptor), vanucizumab (anti-angiopoietin 2), vapaliximab (anti-AOC3/VAP-1), varisacumab (anti-VEGF-A), varlilumab (anti-CD27), vatelizumab (anti-ITGA2/CD49b), vedolizumab (anti-integrin α4β7), veltuzumab (anti-CD20), vepalimomab (anti-AOC3, a.k.a., VAP-1), vesencumab (anti-NRP1), vilobelimab (anti-C5a receptor; C5a), visilizumab (anti-CD3), vobarilizumab (anti-IL-6 receptor), volociximab (anti-integrin α5β1), vonlerolizumab (anti-CD134), vopratelimab (anti-CD278, a.k.a. ICOS), vorsetuzumab mafodotin (anti-CD70), votumumab (anti-tumor antigen CTAA16.88), vunakizumab (anti-IL-17A), xentuzumab (anti-IGF-1, anti-IGF-2), XMAB-5574 (anti-CD19), zalutumumab (anti-Epidermal growth factor receptor, EGFR), zanolimumab (anti-CD4), zatuximab (an anti-HER1), zenocutuzumab (anti-ERBB3/HER3), ziralimumab (anti-CD147/basigin), zolbetuximab (anti-claudin 18 isoform 2), and zolimomab aritox (anti-CD5).

In some aspects, the payload comprises an antibody or antigen-binding portion thereof targets an apoptosis regulator, e.g., attached to the surface of a liposome encapsulating the AAV-ITR vector or the Rep vector. In some aspects, the payload comprises a dsDNA polynucleotide in the AAV-ITR vector encoding an antibody or antigen-binding portion thereof that targets an apoptosis regulator. In some aspects, the apoptosis regulator is pro-apoptotic gene product, e.g, FasL (Fas ligand), BAX, BID, BAK or BAD. In some aspects, the apoptosis regulator is an anti-apoptotic gene product, e.g, Bcl-XI, a IAPs (e.g., XIAP), or Bcl-2. In some aspects, the apoptosis regulator is a prosurvival factor such as cFLIP, BNIP3, FADD, Akt, or NF-κB.

### Payloads: Gene Editing System Components

In some aspects, the payload of AAV-ITR based gene delivery system of the present disclosure comprises a polynucleotide comprising one or more components of a gene editing system. In some aspects, the payload comprises a gRNA or a dsDNA encoding a gRNA. In some aspects, the payload comprises a dsDNA encoding a nuclease. In some aspects, the payload comprises a gRNA or a dsDNA encoding a gRNA and a dsDNA encoding a nuclease. In some aspects, the AAV-ITR vector comprises a dsDNA encoding a gRNA and a dsDNA encoding a nuclease. In some aspects, the AAV-ITR vector comprises a dsDNA encoding a nuclease.

CRISPR/Cas: In some aspects, the gene editing system is a CRISPR/Cas system. In some aspects, the payload of an AAV-ITR based gene delivery system of the present disclosure comprises a polynucleotide encoding a CRISPR Cas nuclease, e.g., a dsRNA encoding a Cas9 nuclease. In some aspects, the CRISPR/Cas nuclease is codon-optimized for the desired cell type in which it is to be expressed. In some aspects, the CRIPS/Cas gene editing system can also employ a guide RNA (gRNA) that comprises two separate molecules. An exemplary two-molecule gRNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA” or “scaffold”) molecule. A crRNA comprises both the DNA-targeting segment (single stranded) of the gRNA and a stretch of nucleotides that forms one-half of a double stranded RNA (dsRNA) duplex of the protein-binding segment of the gRNA. A corresponding tracrRNA (activator-RNA) comprises a stretch of nucleotides that forms the other half of the dsRNA duplex of the protein-binding segment of the gRNA. Thus, a stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. The crRNA additionally provides the single stranded DNA-targeting segment. Accordingly, a gRNA comprises a sequence that hybridizes to a target sequence, and a tracrRNA. Thus, a crRNA and a tracrRNA (as a corresponding pair) hybridize to form a gRNA. If used for modification within a cell, the exact sequence and/or length of a given crRNA or tracrRNA molecule can be designed to be specific to the species in which the RNA molecules will be used.

In some aspects, the CRISPR/Cas gene editing system can employ a fused crRNA-tracrRNA construct (i.e., a single transcript) that functions with the codon-optimized Cas9. This single RNA is often referred to as a guide RNA or gRNA. Within a gRNA, the crRNA portion is identified as the “target sequence” for the given recognition site and the tracrRNA is often referred to as the “scaffold.” To generate a gRNA, a short DNA fragment containing the target sequence is inserted into a guide RNA expression nucleic acid. The gRNA expression nucleic acid comprises the target sequence (in some aspects around 20 nucleotides), a form of the tracrRNA sequence (the scaffold) as well as a suitable promoter that is active in the cell and necessary elements for proper processing in eukaryotic cells.

In some aspects, the payload of AAV-ITR based gene delivery system of the present disclosure encodes a gRNA comprising the target sequence (in some aspects around 20 nucleotides), a form of the tracrRNA sequence (the scaffold) as well as a suitable promoter. In some aspects, custom, complementary oligonucleotides are annealed to form a double stranded DNA and are then cloned into the gRNA expression nucleic acid, which is included as payload in the AAV-ITR based gene delivery system of the present disclosure. In some aspects, the payload comprises a two-molecule gRNA or a fused crRNA-tracrRNA construct. In some aspects, the payload comprises a dsDNA encoding a Cas9 nuclease and a dsDNA encoding a gRNA. In some aspects, the gRNA can be provided in the form of separate crRNA and tracrRNA molecules, or separate dsDNA molecules encoding the crRNA and tracrRNA, respectively.

In some aspects, the gRNA comprises a third nucleic acid sequence encoding a Clustered Regularly Interspaced Short Palindromic Repeats (CRISPR) RNA (crRNA) and a trans-activating CRISPR RNA (tracrRNA). In some aspects, the Cas protein to be used with the AAV-ITR based gene delivery system of the present disclosure is a type I Cas protein. In some aspects, the Cas protein is a type II Cas protein. In some aspects, the type II Cas protein is Cas9. In some aspects, the type II Cas, e.g., Cas9 protein, is a human codon-optimized Cas.

In certain aspects, the Cas protein is a “nickase” that can create single strand breaks (i.e., “nicks”) at the target site without cutting both strands of double stranded DNA (dsDNA). Cas9, for example, comprises two nuclease domains—a RuvC-like nuclease domain and an HNH-like nuclease domain—which are responsible for cleavage of opposite DNA strands. Mutation in either of these domains can create a nickase. Examples of mutations creating nickases can be found, for example, WO/2013/176772A1 and WO/2013/142578A1, each of which is herein incorporated by reference.

In certain aspects, two separate Cas proteins (e.g., nickases) specific for a target site on each strand of dsDNA can create overhanging sequences complementary to overhanging sequences on another nucleic acid, or a separate region on the same nucleic acid. The overhanging ends created by contacting a nucleic acid with two nickases specific for target sites on both strands of dsDNA can be either 5′ or 3′ overhanging ends. For example, a first nickase can create a single strand break on the first strand of dsDNA, while a second nickase can create a single strand break on the second strand of dsDNA such that overhanging sequences are created. The target sites of each nickase creating the single strand break can be selected such that the overhanging end sequences created are complementary to overhanging end sequences on a different nucleic acid molecule. The complementary overhanging ends of the two different nucleic acid molecules can be annealed by the methods disclosed herein. In some aspects, the target site of the nickase on the first strand is different from the target site of the nickase on the second strand.

In some aspects, the first nucleic acid comprises a mutation that disrupts at least one amino acid residue of nuclease active sites in the Cas protein, wherein the mutant Cas protein generates a break in only one strand of the target DNA region, and wherein the mutation diminishes non-homologous recombination in the target DNA region. In some aspects, the first nucleic acid that encodes the Cas protein further comprises a nuclear localization signal (NLS). In some aspects, the nuclear localization signal is a SV40 nuclear localization signal.

Talen: In some aspects, the AAV-ITR based gene delivery system of the present disclosure can comprise a polynucleotide encoding a TALEN system. TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI.

The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi:10.1093/nar/gkg704; and Miller et al. (2011) Nature Biotechnology 29:143-148; all of which are herein incorporated by reference.

Examples of suitable TAL nucleases, and methods for preparing suitable TAL nucleases, are disclosed, e.g., in US Patent Application No. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1 (each hereby incorporated by reference).

In various aspects, TAL effector nucleases are engineered that cut in or near a target nucleic acid sequence in, e.g., a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified in a target cell using the AAV-ITR based gene delivery system of the present disclosure.

In some aspects, each monomer of the TALEN comprises 12-25 TAL repeats, wherein each TAL repeat binds a 1 bp subsite. In some aspects, the nuclease agent is a chimeric protein comprising a TAL repeat-based DNA binding domain operably linked to an independent nuclease. In some aspects, the independent nuclease is a FokI endonuclease. In some aspects, the nuclease agent comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by about 6 bp to about 40 bp cleavage site, and wherein the FokI nucleases dimerize and make a double strand break at a target sequence.

In some aspects, the nuclease agent comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by a 5 bp or 6 bp cleavage site, and wherein the FokI nucleases dimerize and make a double strand break.

Zinc-finger nucleases: In some aspects, the AAV-ITR based gene delivery system of the present disclosure can comprise a polynucleotide encoding a zinc-finger nuclease (ZFN) system. In some aspects, each monomer of the ZFN comprises 3 or more zinc finger-based DNA binding domains, wherein each zinc finger-based DNA binding domain binds to a 3 bp subsite. In other aspects, the ZFN is a chimeric protein comprising a zinc finger-based DNA binding domain operably linked to an independent nuclease. In some aspects, the independent endonuclease is a FokI endonuclease. In some aspects, the nuclease agent comprises a first ZFN and a second ZFN, wherein each of the first ZFN and the second ZFN is operably linked to a FokI nuclease, wherein the first and the second ZFN recognize two contiguous target DNA sequences in each strand of the target DNA sequence separated by about 6 bp to about 40 bp cleavage site or about a 5 bp to about 6 bp cleavage site, and wherein the FokI nucleases dimerize and make a double strand break. See, for example, US20060246567; US20080182332; US20020081614; US20030021776; WO/2002/057308A2; US20130123484; US20100291048; and, WO/2011/017293A2, each of which is herein incorporated by reference.

Meganucleases: In some aspects, the AAV-ITR based gene delivery system of the present disclosure can comprise a polynucleotide encoding a meganuclease system. Meganucleases (or homing endonucleases or HEases) have been classified into four families based on conserved sequence motifs, the families are the “LAGLIDADG,” “GIY-YIG,” “H-N-H,” and “His-Cys box” families. These motifs participate in the coordination of metal ions and hydrolysis of phosphodiester bonds.

HEases are notable for their long recognition sites, and for tolerating some sequence polymorphisms in their DNA substrates. Meganuclease domains, structure and function are known, see for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764.

In some aspects, the meganuclease can be a naturally occurring variant or an engineered derivative. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346.

Any meganuclease can be used with the AAV-ITR based gene delivery system of the present disclosure, including, but not limited to, I-SceI, I-SceII, I-SceIII, I-SceIV, I-SceV, I-SecVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, PI-PspI, F-SceI, F-SceII, F-SuvI, F-TevI, F-TevII, I-AmaI, I-Anil, I-ChuI, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NcIIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorIIP, I-PbpIP, I-SpBetaIP, I-ScaI, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MtuI, PI-MtuHIP, PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, PI-TliII, or any active variants or fragments thereof.

In some aspects, the meganuclease recognizes double-stranded DNA sequences of 12 to 40 base pairs. In some aspects, the meganuclease recognizes one perfectly matched target sequence in one of the heterologous plasmids described herein. In some aspects, the meganuclease is a homing nuclease. In some aspects, the homing nuclease is a “LAGLIDADG” family of homing nuclease. In some aspects, the “LAGLIDADG” family of homing nuclease is selected from I-SceI, I-CreI, and I-Dmol.

Restriction endonucleases: In some aspects, the AAV-ITR based gene delivery system of the present disclosure can comprise a polynucleotide encoding a restriction endonuclease, which includes Type I, Type II, Type III, and Type IV endonucleases. Type I and Type III restriction endonucleases recognize specific recognition sites, but typically cleave at a variable position from the nuclease-binding site, which can be hundreds of base pairs away from the cleavage site (recognition site). In Type II systems the restriction activity is independent of any methylase activity, and cleavage typically occurs at specific sites within or near to the binding site. Most Type II enzymes cut palindromic sequences, however Type IIa enzymes recognize non-palindromic recognition sites and cleave outside of the recognition site, Type IIb enzymes cut sequences twice with both sites outside of the recognition site, and Type IIs enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res 31:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.).

### Payload: Therapeutic Oligonucleotides.

Nucleic acid active agents suitable for use in the AAV-ITR based gene delivery system of the present disclosure include all types of RNA and all types of DNA, including also oligonucleotides such as probes and primers used in the polymerase chain reaction (PCR), hybridizations, or DNA sequencing. In some aspects, the nucleic acid comprises dsDNA, mRNA, miRNA, miRNA sponge, tough decoy miRNA (TD), antimir (antagomir), small RNA, rRNA, siRNA, shRNA, gDNA, cDNA, pDNA, PNA, BNA, antisense oligonucleotide (ASO), aptamer, cyclic dinucleotide, or any combination thereof.

In some aspects, the payload is a nucleic acid, e.g., a dsDNA, in the AAV-ITR vector of a AAV-ITR based gene delivery system of the present disclosure that encodes a therapeutic oligonucleotide disclosed below or combination thereof (e.g., several concatenated oligonucleotides). In some aspects, the payload is a therapeutic oligonucleotide disclosed below or combination thereof coencapsulated into the lipidic delivery system (e.g., liposome) encapsulating the AAV-ITR vector. In some aspects, the payload is a therapeutic oligonucleotide disclosed below or combination thereof coencapsulated into the lipidic delivery system (e.g., liposome) encapsulating the Rep vector. In some aspects, the payload is a therapeutic oligonucleotide disclosed below or combination thereof attached to the surface of the lipidic delivery system (e.g., liposome) encapsulating the AAV-ITR vector. In some aspects, the payload is a therapeutic oligonucleotide disclosed below or combination thereof attached to the surface the lipidic delivery system (e.g., liposome) encapsulating the Rep vector.

In some aspects, the payload comprises a short interfering RNA (siRNA), which is a double-stranded RNA that can induce sequence-specific post-transcriptional gene silencing, thereby decreasing or even inhibiting gene expression. For example, siRNAs can trigger the specific degradation of homologous RNA molecules, such as mRNAs, within the region of sequence identity between both the siRNA and the target RNA. Non-limiting exemplary siRNAs are disclosed in WO 02/44321, which is incorporated by reference in its entirety.

In some aspects, the payload comprises a short hairpin RNAs (shRNAs). In some aspects, the biologically active molecule comprises a miRNA or a miRNA inhibitor (antimiR). In some aspects, the payload can be 10-30 nucleotides in length, for example from 14-25 nucleotides in length. In some aspects, the biologically active molecule (payload) has a length of 16-30 nucleotides, 18-25 nucleotides, particularly 18, 19, 20, 21, 22, 23, 24, or 25 nucleotides.

Sequences for miRNAs are available publicly, for example, through the miRBase registry (Griffiths-Jones, et al., Nucleic Acids Res., 36(Database Issue):D154-D158 (2008); Griffiths-Jones, et al., Nucleic Acids Res., 36(Database Issue):D140-D144 (2008); Griffiths-Jones, et al., Nucleic Acids Res., 36(Database Issue):D109-D111 (2008)) and other publically accessible databases.

In some aspects, the miRNA inhibitors are oligomers or polymers of ribonucleic acid (RNA) or deoxyribonucleic acid (DNA) or modifications thereof. In some aspects, the miRNA antagonists are antimir. Antimirs are a specific class of miRNA inhibitors that are described, for example, in US2007/0213292 to Stoffel et al. Antimirs are RNA-like oligonucleotides that contain various modifications for RNase protection and pharmacologic properties such as enhanced tissue and cellular uptake. Antimirs differ from normal RNA by having complete 2′-O-methylation of sugar, phosphorothioate backbone and a cholesterol-moiety at 3′-end.

Non-limiting examples of antimirs and other miRNA inhibitors are described in WO2009/020771, WO2008/091703, WO2008/046911, WO2008/074328, WO2007/090073, WO2007/027775, WO2007/027894, WO2007/021896, WO2006/093526, WO2006/112872, WO2007/112753, WO2007/112754, WO2005/023986, or WO2005/013901, all of which are hereby incorporated by reference.

In some aspects, the nucleic acids are phosphodiester antisense oligonucleotides, and any oligonucleotides where the sugar-phosphate “backbone” has been derivatized or replaced with “backbone analogues” such as with phosphorothioate, phosphorodithioate, phosphoroamidate, alkyl phosphotriester, or methylphosphonate linkages. In some aspects, the nucleic acids active agents are antisense oligonucleotides, and any oligonucleotides or oligodeoxynucleotides with non-phosphorous backbone analogues such as sulfamate, 3′-thioformacetal, methylene(methylimino) (MMI), 3′-N-carbamate, or morpholino carbamate.

In some aspects, the payload is an antimir. As used herein, the terms “antimir,” “anti microRNA,” “anti miRNA,” and variants thereof refer to molecules (e.g., synthetically generated molecules) that are used to neutralize microRNA (miRNA) function in cells for desired responses. miRNA are complementary sequences (approx. 20-22 bp) to mRNA that are involved in the cleavage of RNA or the suppression of the translation. By controlling the miRNA that regulate mRNAs in cells, antimirs (also called anti-miRNA oligonucleotides, AMOs, or antagomirs) can be used as further regulation as well as for therapeutic for certain cellular disorders. This regulation can occur through a steric blocking mechanism as well as hybridization to miRNA.

These interactions within the body between antimirs and a miRNA can be for therapeutics in disorders in which over/under expression occurs or aberrations in miRNA lead to coding issues. Some of the miRNA-linked disorders that are encountered in the humans include cancers, muscular diseases, autoimmune disorders, and viruses.

Various components of antimirs can be manipulated to affect the binding affinity and potency of the antimir. The 2′-sugar of the antimirs can be modified to be substituted with fluorine and various methyl groups, almost all with an increase in binding affinity. However, some of these modified 2′-sugar antimirs lead to negative effects on cell growth. Modifying the 5′-3′ phosphodiester backbone linkage to a phosphorothioate (P-S) backbone linkage is also known to have an effect on target affinity. Using the P-S mutation was shown to decrease the Tm of the oligonucleotide, which leads to a lower target affinity. A final requirement for antimirs is mismatch specificity and length restrictions. Due to miRNAs in the same families sharing “seed” (shared) sequences and differ by only a couple of additional nucleotides; one antimir can potentially target multiple miRNA sequences.

In some aspects, the payload comprises a therapeutic oligonucleotide or a combination thereof. In some aspects, the payload is a therapeutic oligonucleotide disclosed below.

In some aspects the therapeutic oligonucleotide is selected from the group consisting of 1018 ISS, AB-729, abetimus, AEG35156 (GEM640), afovirsen, aganirsen, agatolimod, alicaforsen, ALNAAT-02, amlivirsen, anivamersen, apatorsen, aprinocarsen, APTA-16, AR-177 (ZINTEVIR™), ARC19499 (BAX-499), archexin, AROANG-3, AROAPOC-3, ARO-HSD, AS1411 (AGRO100), ASM-8, asvasiran, atesidorsen, ATL-1102, ATU-027, avacincaptad pegol (ZIMURA™), AVI-4126 (Resten-MP™), AVI-7288, AVI-7537, AVT-02, AZD-8233, AZD-8701, baliforsen, bamosiran, bazlitoran, BC007, beclanorsen, belcesiran, bepirovirsen, bevasiranib, BIIB-080, BMN 044, BMN 053, brivoligide, casimersen, cavrotolimod, cemdisiran, cenersen, cepadacursen (CIVI 008), cimdelirsen, cobitolimod, cobomarsen, CODA-001 (NEXAGON™), cofirasersen, cosdosiran, CpG 7909, CPG-8954, cupabimod, custirsen, danvatirsen, daplusiran, defibrotide (DEFITELIO™), dematirsen, donidalorsen, drisapersen (KYNDRISA™), DYN-101, edifoligide, egaptivon pegol, EIF-4E, eluforsen, emapticap pegol, eplontersen, eteplirsen (EXONDYS 51™), fazisiran, fesomersen, fitusiran, fomivirsen (VITRAVENE™), frenlosirsen, gataparsen, givosiran (GIVLAARI™), GNKG-168 (CPG-685), golodirsen (SRP-4053, VYONDYS 53™), GPI-2A, GTI-2040 (LOR-2040), GTI-2501, GTX-102, HBVAXPRO, imetelstat, IMT-504, inclisiran, inotersen (TEGSEDI™), ION-224, ION-253, ION-363, ION-464, ION-541, ION-859, IONIS-AGTLRx, IONIS-APO(a)-Rx, IONISAR-2.5Rx, IONIS-C9Rx, IONIS-DNM2-2.5Rx, IONISENAC-2.5Rx, IONIS-FB-LRx, IONIS-FXILRx, IONIS-FXIRx, IONIS-GCGRRx, IONIS-HBVLRX, IONIS-MAPTRx, IONIS-PKKRx, IONISTMPRSS-6LRx, IONIS-TTRRx, ISIS EIF4E Rx, ISIS-104838, ISIS-1082, ISIS-113715, ISIS-2503, ISIS-333611, ISIS-426115, ISIS-449884, ISIS-463588, ISIS-5132, ISIS-702843, ISIS-757456, ISIS-863633, ISTH-0036, JNJ-3989, lademirsen, lexanersen (WVE-120102), lexaptepid pegol (NOX-H94), litenimod, LSP-GR3, lumasiran, mipomersen (KYNAMRO™), miravirsen, monarsen, mongersen, MT-5745, MTL-CEBPA, ND-L02-s0201 (BMS-986263), nedosiran, NS-089, nusinersen (SPINRAZA™), oblimersen (SPC2996, GENASENSE™), olaptesed pegol (NOX-A12), olezarsen, olpasiran, OLX-101, patisiran (ONPATTRO™), pegaptanib (MACUGEN™), PEGnivacogin, pegpleranib (FOVISTA™), pelacarsen, prexigebersen, PUL-042, QPI-1007, QR-1123, QRX-421a, radavirsen, remlarsen, renadirsen, revusiran, RG-012, RG-101, RG-6346, RGLS-4326, rimigorsen, rosomidnar, rovanersen (WVE-120101), sapablursen, SBO10, sepofarsen, siG-12D-LODER, SLN124, SR-063, SRP-5051, STK-001, STP-705, suvodirsen, tadnersen, temavirsen, teprasiran, tilsotolimod, tivanisiran (SYLENTIS™), tofersen, tominersen, tomligisiran, TOP-1731, trabedersen (AP-12009), trecovirsen, varodarsen, VEGLIN 3, vidutolimod, viltolarsen (VILTEPSO™), VIR-2218, volanesorsen (WAYLIVRA™) vupanorsen, vutrisiran, WVE-003, WVE-004, WVEN-531, zilebesiran, and zilganersen.

In some aspects, the payload comprises an antisense oligonucleotide that targets an apoptosis regulator. In some aspects, the apoptosis regulator is pro-apoptotic gene product, e.g., FasL (Fas ligand), BAX, BID, BAK or BAD. In some aspects, the apoptosis regulator is an anti-apoptotic gene product, e.g, Bcl-XI, an IAPs (e.g., XIAP), or Bcl-2. In some aspects, the apoptosis regulator is a prosurvival factor such as cFLIP, BNIP3, FADD, Akt, or NF-κB.

In some aspects, the nucleic acid therapeutic agent is 1018 ISS, also known as ISS-1018, which is a CpG oligonucleotide which functions as an immunostimulant. The oligonucleotide sequence of 1018 ISS is set forth in SEQ ID NO:571. In some aspects, the nucleic acid therapeutic agent is AEG35156, an antisense oligonucleotide for the treatment of hepatocellular carcinoma, acute myeloid leukemia, B-cell lymphoma, chronic lymphocytic leukemia, multiple sclerosis, non-small cell lung cancer, or pancreatic cancer that targets X-Linked Inhibitor of Apoptosis (XIAP). The oligonucleotide sequence of AEG35156 is set forth in SEQ ID NO:572. In some aspects, the nucleic acid therapeutic agent is AB-729, an anti-miRNA (antimir) for the treatment of hepatitis B infection that targets hepatitis virus B's HBsAg.

In some aspects, the nucleic acid therapeutic agent is abetimus, an immunosuppressant oligonucleotide for the treatment of lupus nephritis. The oligonucleotide sequences of abetimus comprises a subunit 1 of SEQ ID NO:573, a subunit 2 of SEQ ID NO:574, a subunit 3 of SEQ ID NO:575, a subunit 4 of SEQ ID NO:576, a subunit 5 of SEQ ID NO:577, a subunit 6 of SEQ ID NO:578, a subunit 7 of SEQ ID NO:579, and a subunit 8 of SEQ ID NO:580. In some aspects, the nucleic acid therapeutic agent is afovirsen, an antisense oligonucleotide for the treatment of human papillomavirus infection that targets the mRNA of human papillomavirus. The oligonucleotide sequence of afovirsen is set forth in SEQ ID NO:581.

In some aspects, the nucleic acid therapeutic agent is aganirsen, also known as GS101, which is an antisense oligonucleotide used for the inhibition of corneal neovascularization, a major risk factor of corneal graft rejection that targets insulin receptor substrate-1 (IRS1). The oligonucleotide sequence of aganirsen is set forth in SEQ ID NO:582. In some aspects, the nucleic acid therapeutic agent is agatolimod, also known as CPG-7909, AMA1-C1 or PF-3512676, which is a CpG oligodeoxynucleotide for the treatment of cancers such as basal cell cancer, non-Hodgkin's lymphoma, breast cancer, metastatic or recurrent malignancies, non-small cell lung cancer, infectious diseases, allergies, and asthma that acts as a toll-like receptor 9 (TLR9) agonist. The oligonucleotide sequence of agatolimod is set forth in SEQ ID NO:583.

In some aspects, the nucleic acid therapeutic agent is alicaforsen, an antisense oligonucleotide for the treatment of acute distress flares in moderate to severe inflammatory bowel disease that targets ICAM-1. The oligonucleotide sequence of alicaforsen is set forth in SEQ ID NO:584. In some aspects, the nucleic acid therapeutic agent is AGRO100, also known as AS1411, which is an aptamer used for the treatment of acute myeloid leukemia, advanced solid tumors, metastatic renal cell carcinoma, and myeloid leukemia that targets IKBKG. The oligonucleotide sequence of AGRO100 is set forth in SEQ ID NO:585. In some aspects, the nucleic acid therapeutic agent is amlivirsen, an antiviral antisense oligonucleotide. The oligonucleotide sequence of amlivirsen is set forth in SEQ ID NO:586.

In some aspects, the nucleic acid therapeutic agent is anivamersen and/or pegnivacogin. Anivamersen and pegnivacogin are components of the REG1 anticoagulation system. Pegnivacogin is an RNA aptamer inhibitor of coagulation factor IXa and anivamersen is a complementary sequence reversal oligonucleotide. The oligonucleotide sequence of pegnivacogin is set forth in SEQ ID NO:587. The oligonucleotide sequence of anivamersen is set forth in SEQ ID NO:588. In some aspects, the nucleic acid therapeutic agent is apatorsen, also known as OGX-427, which is an antisense oligonucleotide used for the treatment of advanced squamous cell lung cancers that targets Hsp27. The oligonucleotide sequence of apatorsen is set forth in SEQ ID NO:589. In some aspects, the nucleic acid therapeutic agent is aprinocarsen, an antisense oligonucleotide for the treatment of cancer that targets PKC-c. The oligonucleotide sequence of aprinocarsen is set forth in SEQ ID NO:590.

In some aspects, the nucleic acid therapeutic agent is APTA-16, an aptamer for the treatment of acute myeloid leukemia, myelodysplastic syndromes, or liver cancer, that targets histone methyltransferase. In some aspects, the nucleic acid therapeutic agent is AR-177, also known as ZINTEVIR™, which is an oligonucleotide analogue that functions as an integrase inhibitor and can be used for the treatment of HIV-1 infection. The oligonucleotide sequence of AR-177 is set forth in SEQ ID NO:591. In some aspects, the nucleic acid therapeutic agent is ARC19499, also known as BAX-499, which is an RNA aptamer for the treatment of hemophilia that targets TFPI. The oligonucleotide sequence of ARC19499 is set forth in SEQ ID NO:592.

In some aspects, the nucleic acid therapeutic agent is archexin, also known as RX-201, which is an antisense oligonucleotide for the treatment of metastatic renal cancer, ovarian cancer, renal cell carcinoma, glioblastoma, stomach cancer, pancreatic cancer, lung cancer, or cervical carcinomas that targets the AKT-1 protein kinase. The oligonucleotide sequence of archexin is set forth in SEQ ID NO:593. In some aspects, the nucleic acid therapeutic agent is asvasiran, a siRNA for the treatment of respiratory syncytial virus infection that targets the RSV N gene. The oligonucleotide sequence of asvasiran is a duplex RNA comprising the antisense sequence of SEQ ID NO:594 and the sense sequence of SEQ ID NO:595. In some aspects, the nucleic acid therapeutic agent is atesidorsen, also known as ATL1103, which is an antisense oligonucleotide for the treatment of acromegaly, cancer, or diabetic retinopathy that targets somatotropin receptors. The oligonucleotide sequence of atesidorsen is set forth in SEQ ID NO:596. In some aspects, the nucleic acid therapeutic agent is ATU-027. ATU-027 is a siRNA targeting protein kinase N3 that inhibits cancer progression, e.g., in prostate and pancreatic cancer. The oligonucleotide sequence of ATU-027 is a RNA duple comprising the antisense sequence set forth in SEQ ID NO:597 and the sense sequence set forth in SEQ ID NO:598. In some aspects, the nucleic acid therapeutic agent is AVT-02 developed by Avontec GmbH. AVT-02 is a short, double stranded oligonucleotide decoy for the treatment of psoriasis vulgaris that targets STAT-1. In some aspects, the nucleic acid therapeutic agent is avacincaptad pegol (ZIMURA™). Avacincaptad pegol is PEG-conjugated oligonucleotide for the treatment of polyploidal choroidal vasculopathy, Stargardt disease, or wet age-related macular degeneration that functions as a complement C5 inhibitor. The oligonucleotide sequence of avacincaptad pegol is set forth in SEQ ID NO:599.

In some aspects, the nucleic acid therapeutic agent is AVI-7537. AVI-7537 is a morpholino antisense oligonucleotide that targets the VP24 gene of Ebola virus. The oligonucleotide sequence of AVI-7537 is set forth in SEQ ID NO:600. In some aspects, the nucleic acid therapeutic agent is AVI-7288. AVI-7288 is a morpholino antisense oligonucleotide that targets Marburg virus nucleoprotein (NP). In some aspects, the nucleic acid therapeutic agent is baliforsen. Baliforsen, also known as IONIS-598769, is an antisense oligonucleotide for the treatment of myotonic dystrophy. The oligonucleotide sequence of baliforsen is set forth in SEQ ID NO:601. In some aspects, the nucleic acid therapeutic agent is bamosiran. Bamosiran, also known as SYL-040012, is a siRNA for the treatment of glaucoma or ocular hypertension that targets beta 2 adrenergic receptors. The oligonucleotide sequence of bamosiran is a duplex RNA comprising an antisense strand of SEQ ID NO:602 and a sense strand of SEQ ID NO:603. In some aspects, the nucleic acid therapeutic agent is bazlitoran. Bazlitoran, also known as IMO-8400, is a DNA oligonucleotide for the treatment of Waldenstrom's macroglobulinemia that targets toll-like receptors TLR7, TLR8 and TLR9. The oligonucleotide sequence of bazlitoran is set forth in SEQ ID NO:604. In some aspects, the nucleic acid therapeutic agent is BC007. BC007 is a non-modified DNA aptamer out of a family of aptamers that bind to and lead to the neutralization of autoantibodies that are directed against G-protein-coupled receptors (GPCR-AABs). BC007 binds to 131-adrenergic-receptor-autoantibodies. BC007 can be used for the treatment of dilated cardiomyopathy or chronic fatigue syndrome.

In some aspects, the nucleic acid therapeutic agent is beclanorsen. Beclanorsen, also known as SPC-2996, is an antisense oligonucleotide for the treatment of lymphoid leukemias that targets Bcl-2. The oligonucleotide sequence of beclanorsen is set forth in SEQ ID NO:605. In some aspects, the nucleic acid therapeutic agent is bepirovirsen. Bepirovirsen, also known as ISIS-505358, ISIS-GSK3RX, GSK-3228836 or IONIS HBVRX, is an antisense oligonucleotide for the treatment of hepatitis B. The oligonucleotide sequence of bepirovirsen is set forth in SEQ ID NO:606. In some aspects, the nucleic acid therapeutic agent is bevasiranib. Bevasiranib is a siRNA for the treatment of exudative age-related macular degeneration that targets VEGF. The oligonucleotide sequence of bevasiranib is an RNA duplex comprising an antisense strand of SEQ ID NO:607 and a sense strand of SEQ ID NO:608. In some aspects, the nucleic acid therapeutic agent is BMN 044. BMN 044, also known as PR044, is an antisense oligonucleotide for the treatment of Duchenne muscular dystrophy that targets mRNA encoding dystrophin. In some aspects, the nucleic acid therapeutic agent is BMN 053. BMN 053, also known as PR053, is an antisense oligonucleotide for the treatment of Duchenne muscular dystrophy that targets dystrophin. In some aspects, the nucleic acid therapeutic agent is brivoligide. Brivoligide is a 23 bp decoy DNA that functions an early growth response protein 1 inhibitor. Brivoligide can be used to treat pain, e.g., postoperative pain. The oligonucleotide sequence of brivoligide is a duplex DNA comprising SEQ ID NO:609 and SEQ ID NO:610. In some aspects, the nucleic acid therapeutic agent is casimersen. Casimersen is a morpholino antisense oligonucleotide for the treatment of Duchenne muscular dystrophy that targets dystrophin's exon 45. The oligonucleotide sequence of casimersen is set forth in SEQ ID NO:611.

In some aspects, the nucleic acid therapeutic agent is cavrotolimod. Cavrotolimod is an immunostimulant oligonucleotide that functions as a TLR9 agonists and can be used for the treatment of hematological malignancies, Merkel cell carcinoma, solid tumors, or squamous cell cancer. The oligonucleotide sequence of cavrotolimod is set forth in SEQ ID NO:612. In some aspects, the nucleic acid therapeutic agent is cemdisiran. Cemdisiran, also known as AD062643, is a siRNA for the treatment of hemolytic uremic syndrome, IgA nephropathy, paroxysmal nocturnal hemoglobinuria, or myasthenia gravis that targets complement C5. The oligonucleotide sequence of cemdisiran is an RNA duplex comprising an antisense strand of SEQ ID NO:613 and a sense strand of SEQ ID NO:614. In some aspects, the nucleic acid therapeutic agent is cenersen. Cenersen is an antisense oligonucleotide for the treatment of myelodysplastic syndromes, acute myeloid leukemia, or chronic lymphocytic leukemia that targets p53. The oligonucleotide sequence of cenersen is set forth in SEQ ID NO:615. In some aspects, the nucleic acid therapeutic agent is cobitolimod. Cobitolimod is an oligodeoxyribonucleotide for the treatment of ulcerative colitis or brain edema that is an agonist of Toll-like 9 receptors. The oligonucleotide sequence of cobitolimod is set forth in SEQ ID NO:616. In some aspects, the nucleic acid therapeutic agent is cobomarsen. Cobomarsen, also known as MRG-106 or M11667, is an anti-miRNA (antimir) for the treatment of cutaneous T cell lymphoma, adult T-cell leukemia-lymphoma, chronic lymphocytic leukemia, diffuse large B cell lymphoma, or amyotrophic lateral sclerosis that targets miR-155. In some aspects, the nucleic acid therapeutic agent is CODA-001. CODA-001, also known as NEXAGON™, is an antisense oligonucleotide for the treatment of wounds, leg ulcers, diabetic foot ulcers, or corneal injuries that targets gap junctions. NEXAGON™ is a natural, unmodified oligonucleotide (30-mer) that downregulates expression of the key gap junction protein Cx43. The oligonucleotide sequence of CODA-001 is set forth in SEQ ID NO:617.

In some aspects, the nucleic acid therapeutic agent is cofirasersen. Cofirasersen, also known as is an IONIS-ENACRX and ION-827359, is an antisense oligonucleotide for the treatment of pulmonary disease, chronic bronchitis, or cystic fibrosis that targets ENaC. The oligonucleotide sequence of cofirasersen is set forth in SEQ ID NO:618. In some aspects, the nucleic acid therapeutic agent is cosdosiran. Cosdosiran, also known as QPI-1007, is a neuroprotective siRNA for the treatment of nonarteritic anterior ischemic optic neuropathy that inhibits caspase 2 synthesis. The oligonucleotide sequence of cosdosiran is an RNA duplex comprising an antisense strand of SEQ ID NO:619 and a sense strand of sequence SEQ ID NO:620. In some aspects, the nucleic acid therapeutic agent is CPG-8954. CPG-8954 is a CpG oligonucleotide for the treatment of cancer and viral infections. The oligonucleotide sequence of CPG-8954 is set forth in SEQ ID NO:621. In some aspects, the nucleic acid therapeutic agent is cupabimod. Cupabimod, also known as AMG-0103, is an oligonucleotide for the treatment of pain, e.g., chronic discogenic lumbar back pain. The oligonucleotide sequence of cupabimod is a double stranded DNA comprising the sequences set forth in SEQ ID NO:622 and SEQ ID NO:623. In some aspects, the nucleic acid therapeutic agent is custirsen. Custirsen, also known as OGX-011 and ISIS-112989, is an antisense oligonucleotide for the treatment of metastatic castrate resistant prostate cancer that targets clusterin. The oligonucleotide sequence of custirsen is set forth in SEQ ID NO:624. In some aspects, the nucleic acid therapeutic agent is danvatirsen. Danvatirsen, also known as AZD 9150 and ISIS-481464, is an antisense oligonucleotide for the treatment of bladder cancer, colorectal cancer, head and neck cancer, malignant ascites, non-small cell lung cancer, pancreatic cancer, solid tumors, liver cancer, non-Hodgkin's lymphoma, or diffuse large B cell lymphoma, and targets the STAT3 transcription factor. The oligonucleotide sequence of danvatirsen is set forth in SEQ ID NO:625.

In some aspects, the nucleic acid therapeutic agent is daplusiran. Daplusiran is an antiviral siRNA. The oligonucleotide sequence of daplusiran is an RNA duplex comprising an antisense strand of SEQ ID NO:626 and a sense strand of SEQ ID NO:627. In some aspects, the nucleic acid therapeutic agent is defibrotide (DEFITELIO™). Defibrotide, also known as DASOVAS™, NORAVID™, or PROCICLIDE™, is a heparanase inhibitor that functions as an angiogenesis and platelet aggregation inhibitor. Defibrotide is a mixture of single-stranded oligonucleotides that can be used for the treatment of veno-occlusive disorders, graft-versus-host disease, neurological disorders, thrombotic microangiopathies, deep vein thrombosis, thrombosis, diabetic nephropathies, or multiple myeloma. In some aspects, the nucleic acid therapeutic agent is the antisense oligonucleotide dematirsen. The oligonucleotide sequence of dematirsen is set forth in SEQ ID NO:628. In some aspects, the nucleic acid therapeutic agent is the antisense oligonucleotide donidalorsen. Donidalorsen, also known as ISIS-721744, is a plasma kallikrein inhibitor that can be used for the treatment of COVID 2019 infections, hereditary angioedema, or acute respiratory disease. The oligonucleotide sequence of donidalorsen is set forth in SEQ ID NO:629. In some aspects, the nucleic acid therapeutic agent is drisapersen (KYNDRISA™) Drisapersen, also known as GSK 2402968A, is an antisense oligonucleotide for the treatment of Duchenne muscular dystrophy that targets mRNA encoding dystrophin. The oligonucleotide sequence of drisapersen is set forth in SEQ ID NO:630. In some aspects, the nucleic acid therapeutic agent is edifoligide. Edifoligide is a 14 bp decoy DNA that functions as a CDC2 kinase inhibitor and can be used for the treatment of coronary artery restenosis or vascular graft occlusion. The oligonucleotide sequence of edifoligide is a double stranded DNA comprising the sequences set forth in SEQ ID NO:631 and SEQ ID NO:632.

In some aspects, the nucleic acid therapeutic agent is egaptivon pegol. Egaptivon pegol, also known as ARC1779, is an aptamer for the treatment of intracranial embolism, cerebral thromboembolism, carotid stenosis, von Willebrand disease, thrombotic thrombocytopenic purpura, thrombotic microangiopathy, thrombosis, or acute myocardial infarction that targets VWF GP1BA. The oligonucleotide sequence of egaptivon pegol is set forth in SEQ ID NO:633. In some aspects, the nucleic acid therapeutic agent is EIF-4E ASO. EIF-4E ASO is an antisense oligonucleotide for the treatment of prostate cancer. The oligonucleotide sequence of EIF-4E ASO is set forth in SEQ ID NO:634. In some aspects, the nucleic acid therapeutic agent is eluforsen. Eluforsen, also known as QR-010, is an oligonucleotide partly complementary to Phe508del-CFTR RNA. Eluforsen, also known as QR-010 is designed to repair CFTR-encoded mRNA. The oligonucleotide sequence of eluforsen is set forth in SEQ ID NO:635. In some aspects, the nucleic acid therapeutic agent is emapticap pegol. Emapticap pegol, also known as NOX-E36, is an aptamer for the treatment of systemic lupus erythematosus type 2, diabetes mellitus, chronic inflammatory diseases, albuminuria, and renal impairment that targets CCL2. The oligonucleotide sequence of emapticap pegol is set forth in SEQ ID NO:636. In some aspects, the nucleic acid therapeutic agent is eplontersen. Eplontersen, also known as ION-TTR-LRX or AKCEA-TTR-LRX, is an antisense oligonucleotide that functions as a prealbumin expression inhibitor and can be used to treat amyloidosis or transthyretin-related hereditary amyloidosis. The oligonucleotide sequence of eplontersen is set forth in SEQ ID NO:637. In some aspects, the nucleic acid therapeutic agent is eteplirsen (EXONDYS 51™). Eteplirsen, also known, AVI-4658, is an antisense oligonucleotide for the treatment of Duchenne muscular dystrophy that targets DMD exon 51. The oligonucleotide sequence of eteplirsen is set forth in SEQ ID NO:638.

In some aspects, the nucleic acid therapeutic agent is the antisense oligonucleotide fesomersen. The oligonucleotide sequence of fesomersen is set forth in SEQ ID NO:639. In some aspects, the nucleic acid therapeutic agent is fitusiran. Fitusiran, also known as ALN-57213, is a siRNA for the treatment of hemophilia A and B that targets SERPINC1. The oligonucleotide sequence of fitusiran is a RNA duplex comprising an antisense strand of SEQ ID NO:640 and a sense strand of SEQ ID NO:641. In some aspects, the nucleic acid therapeutic agent is fomivirsen (VITRAVENE™). Fomivirsen is an antisense oligonucleotide for the treatment of cytomegalovirus-induced retinitis and HIV infections that targets cytomegalovirus mRNA. The oligonucleotide sequence of fomivirsen is set forth in SEQ ID NO:642. In some aspects, the nucleic acid therapeutic agent is gataparsen. Gataparsen is an antisense oligonucleotide for the treatment of acute myeloid leukemia, non-small cell lung cancer, or prostate cancer that targets BIRC5. The oligonucleotide sequence of gataparsen is set forth in SEQ ID NO:643.

In some aspects, the nucleic acid therapeutic agent is givosiran (GIVLAARI™). Givosiran is a siRNA for the treatment of acute hepatic porphyria that targets 5-aminolevulinate synthetase (ALAS1). The oligonucleotide sequence of givosiran comprises the antisense sequence set forth in SEQ ID NO:644 and the sense sequence set forth in SEQ ID NO:645. In some aspects, the nucleic acid therapeutic agent is GNKG-168. GNKG-168, also known as CPG-685, is an oligonucleotide that functions as a TLR9 agonist. GNKG-168 can be used for the treatment of chronic lymphocytic leukemia. The oligonucleotide sequence of GNKG-168 is set forth in SEQ ID NO:646. In some aspects, the nucleic acid therapeutic agent is golodirsen (VYONDYS 53™) Golodirsen, also known as SRP-4053 and VYONDYS 53™, is an antisense oligonucleotide used to treat Duchenne muscular dystrophy via splicing modulation that targets DMD exon 53. The oligonucleotide sequence of golodirsen is set forth in SEQ ID NO:647. In some aspects, the nucleic acid therapeutic agent is GPI-2A. GPI-2A is an antisense oligonucleotide for the treatment of HIV that inhibits the expression of human immunodeficiency virus type 1 capsid. The oligonucleotide sequence of GPI-2A is set forth in SEQ ID NO:648. In some aspects, the nucleic acid therapeutic agent is GTI-2040. GTI-2040, also known as LOR-2040, is an antisense oligonucleotide for the treatment of renal cell carcinoma that functions as a DNA synthesis inhibitor. GTI-2040 can also be used for the treatment of acute myeloid leukemia, bladder cancer, breast cancer, chronic myeloid leukemia, colorectal cancer, myelodysplastic syndromes, non-small cell lung cancer, or prostate cancer. The oligonucleotide sequence of GTI-2040 is set forth in SEQ ID NO:649. In some aspects, the nucleic acid therapeutic agent is GTI-2501. GTI-2501 is an antisense oligonucleotide for the treatment of renal cell carcinoma that functions as a DNA synthesis inhibitor by targeting the ribonucleoside-diphosphate reductase large subunit. GTI-2501 can also be used for the treatment of acute myeloid leukemia, bladder cancer, breast cancer, chronic myeloid leukemia, colorectal cancer, myelodysplastic syndromes, non-small cell lung cancer, or prostate cancer. The oligonucleotide sequence of GTI-2501 is set forth in SEQ ID NO:650.

In some aspects, the nucleic acid therapeutic agent is HBVAXPRO. HBVAXPRO is a decoy for the treatment of Hepatitis B that targets HBV. In some aspects, the nucleic acid therapeutic agent is IMT-504. IMT-504 is a B cell immunostimulant oligonucleotide for the treatment of diabetes mellitus, rabies, breast cancer, chronic lymphocytic leukemia, hepatitis B, influenza virus infections, neuropathic pain, osteoporosis, or sepsis. The oligonucleotide sequence of IMT-504 is set forth in SEQ ID NO:651. In some aspects, the nucleic acid therapeutic agent is inclisiran. Inclisiran, also known as ALN-60212, is a siRNA for the treatment of hypercholesterolemia that targets PCSK9. The oligonucleotide sequence of inclisiran is an RNA duplex comprising an antisense strand of SEQ ID NO:652 and a sense strand of SEQ ID NO:653.

In some aspects, the nucleic acid therapeutic agent is inotersen (TEGSEDI™) Inotersen is an antisense oligonucleotide for the treatment of hereditary transthyretin-mediated amyloidosis or polyneuropathy that targets TTR. The oligonucleotide sequence of inotersen is set forth in SEQ ID NO:654. In some aspects, the nucleic acid therapeutic agent is imetelstat. Imetelstat is a telomerase inhibitor oligonucleotide for the treatment of myelodysplastic syndromes, myelofibrosis, multiple myeloma, acute myeloid leukemia, chronic myeloid leukemia, breast cancer, essential thrombocythaemia, lymphoproliferative disorders, non-small cell lung cancer, polycythaemia vera, or solid tumors. The oligonucleotide sequence of imetelstat is set forth in SEQ ID NO:655. In some aspects, the nucleic acid therapeutic agent is IONIS-APO(a)-Rx. IONIS-APO(a)-Rx is an antisense oligonucleotide for the treatment of high lipoprotein levels that targets apolipoprotein A. In some aspects, the nucleic acid therapeutic agent is IONIS-C9Rx. IONIS-C9Rx is an antisense oligonucleotide for the treatment of ALS that targets C90RF72. In some aspects, the nucleic acid therapeutic agent is IONIS-DNM2-2.5Rx. IONIS-DNM2-2.5Rx is antisense oligonucleotide for the treatment of centronuclear myopathy that targets DNM2. In some aspects, the nucleic acid therapeutic agent is IONIS-FXIRx. IONIS-FXIRx is an antisense oligonucleotide for the treatment of total knee arthroplasty that targets Factor XI. In some aspects, the nucleic acid therapeutic agent is IONIS-GCGRRx. IONIS-GCGRRx is an antisense oligonucleotide for the treatment of type 2 diabetes that targets glucagon receptor. In some aspects, the nucleic acid therapeutic agent is IONIS-MAPTRx. IONIS-MAPTRx is an antisense oligonucleotide for the treatment of Alzheimer disease that targets MAPT. In some aspects, the nucleic acid therapeutic agent is IONIS-TTRRx. IONIS-TTRRx is an antisense oligonucleotide for the treatment of familial amyloid polyneuropathy (FAP) that targets transthyretin. In some aspects, the nucleic acid therapeutic agent is ISIS EIF4E Rx. ISIS EIF4E Rx is an antisense oligonucleotide for the treatment of castrate-resistant prostate cancer that targets eIF-4E. In some aspects, the nucleic acid therapeutic agent is ISIS-104838, ISIS-1082, ISIS-2503, ISIS-333611, ISIS-113715, ISIS-426115, ISIS-449884, ISIS-463588, ISIS-5132, ISIS-702843, or ISIS-757456.

In some aspects, the nucleic acid therapeutic agent is lademirsen. Lademirsen is an antisense oligonucleotide that targets miR-21. The oligonucleotide sequence of lademirsen is set forth in SEQ ID NO:656. In some aspects, the nucleic acid therapeutic agent is lexaptepid pegol. Lexaptepid pegol, also known as NOX-H94, is an aptamer for the treatment of anemia, end stage renal disease, anemia of chronic disease, chronic diseases, or inflammation that targets hepcidin. The oligonucleotide sequence of lexaptepid pegol is set forth in SEQ ID NO:657. In some aspects, the nucleic acid therapeutic agent is litenimod. Litenimod is a 26-mer modified oligodeoxynucleotides (ODN) that functions as a TLR9 agonist. The oligonucleotide sequence of litenimod is set forth in SEQ ID NO:658. In some aspects, the nucleic acid therapeutic agent is lumasiran. Lumasiran is a siRNA for the treatment of primary hyperoxaluria type 1 that targets HAO1. The oligonucleotide sequence of lumasiran is a double stranded RNA (dsRNA) comprising an antisense strand of SEQ ID NO:659 and a sense strand of SEQ ID NO:660. In some aspects, the nucleic acid therapeutic agent is mipomersen (KYNAMRO™). Mipomersen is antisense oligonucleotide for the treatment of familial hypercholesterolemia that targets APOB. The oligonucleotide sequence of mipomersen is set forth in SEQ ID NO:661. In some aspects, the nucleic acid therapeutic agent is miravirsen. Miravirsen, also known as SPC3649, is an antisense oligonucleotide for the treatment of chronic hepatitis C (CHC) infection that targets miRNA-122. The oligonucleotide sequence of miravirsen is set forth in SEQ ID NO:662.

In some aspects, the nucleic acid therapeutic agent is mongersen. Mongersen, also known as GED-0301, is an antisense oligonucleotide for the treatment of Crohn's disease that targets SMAD7. The oligonucleotide sequence of mongersen is set forth in SEQ ID NO:663. In some aspects, the nucleic acid therapeutic agent is MTL-CEBPA. MTL-CEBPA is a small activating RNA (saRNA) designed to reduce immune suppression of myeloid cells by restoring C/EBP-α protein to normal levels using the RNA activation mechanism. MTL-CEBPA can be used for the treatment of liver cancer, solid tumors, colorectal cancer, hepatocellular carcinoma, or liver disorders. In some aspects, the nucleic acid therapeutic agent is ND-L02-s0201. ND-L02-s0201, also known as BMS-986263, is a siRNA for the treatment of extensive hepatic fibrosis that targets HSP47. In some aspects, the nucleic acid therapeutic agent is nedosiran. Nedosiran is a siRNA for the treatment of primary hyperoxaluria that targets LDHA. The oligonucleotide sequence of nedosiran is a double stranded RNA (dsRNA) comprising an antisense strand of SEQ ID NO:664 and a sense strand of SEQ ID NO:665. In some aspects, the nucleic acid therapeutic agent is nusinersen (SPINRAZA™). Nusinersen is an antisense oligonucleotide (splice modulator) for the treatment of infantile-onset spinal muscular atrophy that targets exon 7 of the Survival of Motor Neuron 2 (SMN2) splicing modulator. The oligonucleotide sequence of nusinersen is set forth in SEQ ID NO:666. In some aspects, the nucleic acid therapeutic agent is oblimersen (GENASENSE™). Oblimersen is an antisense oligonucleotide for the treatment of melanoma that targets Bcl-2. The oligonucleotide sequence of oblimersen is set forth in SEQ ID NO:667. In some aspects, the nucleic acid therapeutic agent is olaptesed pegol. Olaptesed pegol, also known as NOX-A12, is an aptamer for the treatment of chronic lymphocytic leukemia, multiple myeloma, hematopoietic stem cell transplantation, autologous stem cell transplantation, glioblastoma, metastatic colorectal cancer, or metastatic pancreatic cancer that targets CXCL12. The oligonucleotide sequence of olaptesed pegol is set forth in SEQ ID NO:668.

In some aspects, the nucleic acid therapeutic agent is olpasiran. Olpasiran is a siRNA for the treatment of cardiovascular disorders that targets lipoprotein(a) (Lp(a). The oligonucleotide sequence of olpasiran is a double stranded RNA (dsRNA) comprising an antisense strand of SEQ ID NO:669 and a sense strand of SEQ ID NO:670. In some aspects, the nucleic acid therapeutic agent is patisiran (ONPATTRO™). Patisiran is a siRNA for the treatment of hereditary transthyretin-mediated amyloidosis and neuropathy that targets transthyretin. The oligonucleotide sequence of patisiran is a double stranded RNA (dsRNA) comprising an antisense strand of SEQ ID NO:671 and a sense strand of SEQ ID NO:672. In some aspects, the nucleic acid therapeutic agent is pegaptanib (MACUGEN™). Pegaptanib is an aptamer for the treatment of wet macular degeneration neovascular age-related macular degeneration that targets VEGF. The oligonucleotide sequence of pegaptanib is set forth in SEQ ID NO:673. In some aspects, the nucleic acid therapeutic agent is pegpleranib (FOVISTA™). Pegpleranib is an aptamer for the treatment of subfoveal neovascular age-related macular degeneration that targets PDGF-B. The oligonucleotide sequence of pegpleranib is set forth in SEQ ID NO:674. In some aspects, the nucleic acid therapeutic agent is pelacarsen. Pelacarsen, also known as IONIC-APO(a)-LRX and ISIS-681257, is an antisense oligonucleotide form the treatment of hyperlipoproteinemia that targets apolipoprotein A. The oligonucleotide sequence of pelacarsen is set forth in SEQ ID NO:675. In some aspects, the nucleic acid therapeutic agent is prexigebersen. Prexigebersen is an antisense oligonucleotide for the treatment of acute myeloid leukemia, myelodysplastic syndromes, chronic myeloid leukemia, precursor cell lymphoblastic leukemia-lymphoma, colorectal cancer, head and neck cancer, lymphoma, solid tumors, thyroid cancer, or breast cancer that targets GRB2. The oligonucleotide sequence of prexigebersen is set forth in SEQ ID NO:676.

In some aspects, the nucleic acid therapeutic agent is radavirsen. Radavirsen, also known as AVI-7100, is an antisense oligonucleotide for the treatment of influenza A virus infections. The oligonucleotide sequence of radavirsen is set forth in SEQ ID NO:677. In some aspects, the nucleic acid therapeutic agent is remlarsen. Remlarsen is a miRNA mimic for the treatment of cutaneous fibrosis. Remlarsen mimics miR-29. The oligonucleotide sequence of remlarsen is a double stranded RNA (dsRNA) comprising an antisense strand of SEQ ID NO:678 and a sense strand of SEQ ID NO:679. In some aspects, the nucleic acid therapeutic agent is renadirsen. Renadirsen, also known as renapersen, is an antisense oligonucleotide for the treatment of Duchenne muscular dystrophy that functions by stimulating the expression of dystrophin. The oligonucleotide sequence of renadirsen is set forth in SEQ ID NO:680. In some aspects, the nucleic acid therapeutic agent is Resten-MP™. Resten-MP™, also known as AVI-4126, is an antisense oligonucleotide for the treatment of de novo native coronary artery lesions that targets c-myc The oligonucleotide sequence of AVI-4126 is set forth in SEQ ID NO:681. In some aspects, the nucleic acid therapeutic agent is revusiran. Revusiran, also known as AD-51547, is a siRNA for the treatment of hereditary amyloidosis that targets TTR. The oligonucleotide sequence of revusiran is a double stranded RNA (dsRNA) comprising an antisense strand of SEQ ID NO:682 and a sense strand of SEQ ID NO:683. In some aspects, the nucleic acid therapeutic agent is RGLS-4326. RGLS-4326 is antisense oligonucleotide antimir for the treatment of autosomal dominant polycystic kidney disease that targets miR-17. The oligonucleotide sequence of RGLS-4326 is set forth in SEQ ID NO:684.

In some aspects, the nucleic acid therapeutic agent is rimigorsen. Rimigorsen is an antisense oligonucleotide for the treatment of Duchenne muscular dystrophy that promotes the synthesis of functional dystrophin. The oligonucleotide sequence of rimigorsen is set forth in SEQ ID NO:685. In some aspects, the nucleic acid therapeutic agent is rosomidnar. Rosomidnar, also known as PNT-100, is an oligonucleotide inhibitor of apoptosis regulator Bcl2 that can be used for the treatment of diffuse large B cell lymphoma, Richter's syndrome, non-Hodgkin's lymphoma, or solid tumors. The oligonucleotide sequence of rosomidnar is set forth in SEQ ID NO:686. In some aspects, the nucleic acid therapeutic agent is SB010. SB010 is an antisense oligonucleotide for the treatment of mild allergic asthma that targets GATA-3. In some aspects, the nucleic acid therapeutic agent is SLN124. SLN124 is a siRNA for the treatment of 0-thalassemia that targets TMPRSS6. In some aspects, the nucleic acid therapeutic agent is SRP-5051. SRP-5051 is a PPMO antisense oligonucleotide for the treatment of Duchenne muscular dystrophy that target DMD exon 51. SRP-5051 is next generation eteplirsen, in that it targets the same population, those amenable to exon 51 skipping, but the compound is “charged”, meaning that its cell-penetrating capacity is increased. In some aspects, the nucleic acid therapeutic agent is RG-012. RG-012 is an antisense oligonucleotide antimir for the treatment of Aport syndrome that target miR-21. In some aspects, the nucleic acid therapeutic agent is suvodirsen. Suvodirsen, also known as WVE-210201, is an antisense oligonucleotide for the treatment of Duchenne muscular dystrophy that targets DMD exon 51. The oligonucleotide sequence of suvodirsen is set forth in SEQ ID NO:687. In some aspects, the nucleic acid therapeutic agent is temavirsen. Temavirsen, also known as RG-101 and RG-2459, is an antiviral antisense oligonucleotide that targets the hepatitis C virus. The oligonucleotide sequence of temavirsen is set forth in SEQ ID NO:688. In some aspects, the nucleic acid therapeutic agent is teprasiran. Teprasiran, also known as QPI-1002, is a siRNA inhibitor of tumor suppressor protein p53. Teprasiran can be used for the treatment of acute kidney injury or delayed graft function. The oligonucleotide sequence of teprasiran is a double stranded RNA (dsRNA) comprising an antisense strand of SEQ ID NO:689 and a sense strand of SEQ ID NO:690.

In some aspects, the nucleic acid therapeutic agent is tivanisiran (SYLENTIS™) Tivanisiran, also known as SYL-1001, is a siRNA that targets the Transient Receptor Potential Vanilloid-1 (TRPV1) channel family. Tivanisiran can be used to treat ocular pain. The oligonucleotide sequence of tivanisiran is a double stranded RNA (dsRNA) comprising an antisense strand of SEQ ID NO:691 and a sense strand of SEQ ID NO:692. In some aspects, the nucleic acid therapeutic agent is tofersen. Tofersen, also known as IONIS-SOD1Rx and BIIB-067, is an antisense oligonucleotide for the treatment of Amyotrophic Lateral Sclerosis (ALS) that targets SOD1. The oligonucleotide sequence of tofersen is set forth in SEQ ID NO:693. In some aspects, the nucleic acid therapeutic agent is tominersen. Tominersen, also known as IONIS-HTTRx, RG-6042 and ISIS-443139, is an antisense oligonucleotide for the treatment of Huntington's disease that targets HTT. The oligonucleotide sequence of tominersen is set forth in SEQ ID NO:694. In some aspects, the nucleic acid therapeutic agent is trabedersen. Trabedersen, also known as AP-12009 and A-12009, is an antisense oligonucleotide which is an inhibitor of transforming growth factor beta 2. Trabedersen can be used to treat glioblastoma, malignant melanoma, pancreatic cancer, COVID 2019 infections, COVID-19 pneumonia, ovarian cancer, colorectal cancer, or anaplastic astrocytoma. The oligonucleotide sequence of trabedersen is set forth in SEQ ID NO:695. In some aspects, the nucleic acid therapeutic agent is trecovirsen. Trecovirsen is an antisense oligonucleotide for the treatment of AIDS that targets GAG. The oligonucleotide sequence of trecovirsen is set forth in SEQ ID NO:696. In some aspects, the nucleic acid therapeutic is vidutolimod. Vidutolimod, also known as CMP-001 is an immunostimulant oligonucleotide for the treatment of malignant melanoma, head and neck cancer, lymphoma, solid tumors, squamous cell cancer, colorectal cancer, non-small cell lung cancer, allergic asthma, atopic dermatitis, hepatitis B, perennial allergic rhinitis, or seasonal allergic rhinitis. The oligonucleotide sequence of vidutolimod is set forth in SEQ ID NO:697.

In some aspects, the nucleic acid therapeutic agent is viltolarsen (VILTEPSO™) (SEQ ID NO:698). Viltolarsen is an antisense oligonucleotide for the treatment of Duchenne's muscular dystrophy that targets DMD exon 53. In some aspects, the nucleic acid therapeutic agent is volanesorsen (WAYLIVRA™) (SEQ ID NO:699). Volanesorsen, also known as ISIS-304801, is an antisense oligonucleotide for the treatment of hypertriglyceridemia, familial chylomicronemia syndrome, or familial partial lipodystrophy that targets ApoC-III. In some aspects, the nucleic acid therapeutic agent is vupanorsen (SEQ ID NO:700). Vupanorsen, also known as IONIS-ANGPTL3-LRx, AKCEA-ANGPTL3-LRx and ISIS-703802, is an antisense oligonucleotide conjugate (GalNAc3) for the treatment of cardiovascular disease and reduce triglyceride and cholesterol levels, that targets angiopoietin-like 3 (ANGPTL3). In some aspects, the nucleic acid therapeutic agent is vutrisiran. Vutrisiran, also known as ALN-TTRsc02 and ALN-65492, is a siRNA for the treatment of hereditary amyloidosis that targets TTR. The oligonucleotide sequence of vutrisiran is a double stranded RNA (dsRNA) comprising an antisense strand of SEQ ID NO:701 and a sense strand of SEQ ID NO:702. In some aspects, the nucleic acid therapeutic agent is WVE-120101 (rovanersen, also known as WV-1092) or WVE-120102 (lexanersen, also known as WV-2603). WVE-120101 and WVE-120102 are antisense oligonucleotides for the treatment of Huntington's disease that target mutant HTT. WVE-120101 and WVE-120102 interfere with the mutant mRNA copy of the HTT gene. The oligonucleotide sequence of WVE-120101 (rovanersen) is set forth in SEQ ID NO:703. The oligonucleotide sequence of WVE-120102 (lexanersen) is set forth in SEQ ID NO:704. In some aspects, the nucleic acid therapeutic agent is CIVI008/cepadacursen (SEQ ID NO:705). In some aspects, cepadacursen can be used to treat diseases or conditions caused by abnormal expression levels and/or activity of PCSK9 selected from the group consisting of atherosclerosis, hypercholesterolemia (e.g., familiar hypercholesterolemia or statin resistant hypercholesterolemia), HDL/LDL cholesterol imbalance, dyslipidemia (e.g., familial hyperlipidemia (FCHL) or acquired hyperlipidemia), coronary artery disease (CAD), and coronary heart disease (CHD).

In some aspects, the nucleic acid therapeutic agent is ISIS-863633, which has the sequence set forth in SEQ ID NO:706. In some aspects, the nucleic acid therapeutic agent is ALNAAT-02, which targets SERPINA1. In some aspects, the nucleic acid therapeutic agent is AROANG-3, which targets ANGPTL3. In some aspects, the nucleic acid therapeutic agent is AROAPOC-3, which targets APOC3. In some aspects, the nucleic acid therapeutic agent is ARO-HSD, which targets HSD17B13. In some aspects, the nucleic acid therapeutic agent is AS1411, which targets nucleolin and has the sequence set forth in SEQ ID NO:707. In some aspects, the nucleic acid therapeutic agent is ASM-8, which targets CCR4 and CSF2RB. In some aspects, the nucleic acid therapeutic agent is ATL-1102, which targets ITGA4. In some aspects, the nucleic acid therapeutic agent is AZD-8233, which targets PCSK9. In some aspects, the nucleic acid therapeutic agent is AZD-8701, which targets FOXP3. In some aspects, the nucleic acid therapeutic agent is belcesiran, which targets SERPINA1 and has the sequences set forth in SEQ ID NO:708 and SEQ ID NO:709. In some aspects, the nucleic acid therapeutic agent is BIIB-080, which targets MAPT. In some aspects, the nucleic acid therapeutic agent is cimderlirsen, which targets GHR and has the sequence set forth in SEQ ID NO:710. In some aspects, the nucleic acid therapeutic agent is CpG 7909, which targets TLR9. In some aspects, the nucleic acid therapeutic agent is DYN-101, which targets DYN2. In some aspects, the nucleic acid therapeutic agent is fazisiran, which targets SERPINA1. In some aspects, the nucleic acid therapeutic agent is frenlosirsen, which targets IRF4 and has the sequence set forth in SEQ ID NO:711. In some aspects, the nucleic acid therapeutic agent is GTX-102, which targets UBE2A. In some aspects, the nucleic acid therapeutic agent is ION-224, which targets DGAT2. In some aspects, the nucleic acid therapeutic agent is ION-253. In some aspects, the nucleic acid therapeutic agent is ION-363, which targets FUS. In some aspects, the nucleic acid therapeutic agent is ION-464, which targets SNCA. In some aspects, the nucleic acid therapeutic agent is ION-541, which targets ATXN2. In some aspects, the nucleic acid therapeutic agent is ION-859, which targets LRRK2. In some aspects, the nucleic acid therapeutic agent is IONIS-AGTLRx, which targets AGT. In some aspects, the nucleic acid therapeutic agent is IONISAR-2.5Rx, which targets AR. In some aspects, the nucleic acid therapeutic agent is IONISENAC-2.5Rx, which targets SCNN1A. In some aspects, the nucleic acid therapeutic agent is IONIS-FB-LRx, which targets CFB. In some aspects, the nucleic acid therapeutic agent is IONIS-FXILRx, which targets F11. In some aspects, the nucleic acid therapeutic agent is IONIS-HBVLRX, which targets HBV. In some aspects, the nucleic acid therapeutic agent is IONIS-PKKRx, which targets KLKB1. In some aspects, the nucleic acid therapeutic agent is IONISTMPRSS-6LRx, which targets TMPRSS6. In some aspects, the nucleic acid therapeutic agent is ISTH-0036, which targets TGFB2. In some aspects, the nucleic acid therapeutic agent is JNJ-3989, which targets HBV. In some aspects, the nucleic acid therapeutic agent is LSP-GR3, which targets GR3 and has the sequence set forth in SEQ ID NO:712. In some aspects, the nucleic acid therapeutic agent is monarsen, which targets AchE and has the sequence set forth in SEQ ID NO:713. In some aspects, the nucleic acid therapeutic agent is MT-5745, which targets CHST15. In some aspects, the nucleic acid therapeutic agent is NS-089, which targets DMD. In some aspects, the nucleic acid therapeutic agent is olezarsen, which targets APOC3 and has the sequence set forth in SEQ ID NO:714.

In some aspects, the nucleic acid therapeutic agent is OLX-101, which targets CTGF. In some aspects, the nucleic acid therapeutic agent is PUL-042, which targets TLR2, TLR6, and TLR9 and has the sequence set forth in SEQ ID NO:715. In some aspects, the nucleic acid therapeutic agent is QPI-1007, which targets CASP2. In some aspects, the nucleic acid therapeutic agent is QR-1123, which targets RHO. In some aspects, the nucleic acid therapeutic agent is QRX-421a, which targets USH2A. In some aspects, the nucleic acid therapeutic agent is RG-101, which targets miR-122. In some aspects, the nucleic acid therapeutic agent is RG-6346, which targets HBsAg. In some aspects, the nucleic acid therapeutic agent is sapablursen, which targets TMPRSS6 and has the sequence set forth in SEQ ID NO:716. In some aspects, the nucleic acid therapeutic agent is sepofarsen, which targets CEP290 and has the sequence set forth in SEQ ID NO:717. In some aspects, the nucleic acid therapeutic agent is siG-12D-LODER, which targets KRAS. In some aspects, the nucleic acid therapeutic agent is SR-063, which targets AR. In some aspects, the nucleic acid therapeutic agent is STK-001, which targets SVN1A. In some aspects, the nucleic acid therapeutic agent is STP-705, which targets PTGS2/TGFB1. In some aspects, the nucleic acid therapeutic agent is tadnersen, which targets C9orf72 and has the sequence set forth in SEQ ID NO:718. In some aspects, the nucleic acid therapeutic agent is tilsotolimod, which targets TLR9 and has the sequences set forth in SEQ ID NO:719 and SEQ ID NO:720. In some aspects, the nucleic acid therapeutic agent is tomligisiran, which has the sequences set forth in SEQ ID NO:721 and SEQ ID NO:722. In some aspects, the nucleic acid therapeutic agent is TOP-1731, which has the sequence set forth in SEQ ID NO:723. In some aspects, the nucleic acid therapeutic agent is varodarsen, which has the sequence set forth in SEQ ID NO:724. In some aspects, the nucleic acid therapeutic agent is VEGLIN 3, which targets VEGF and has the sequence set forth in SEQ ID NO:725. In some aspects, the nucleic acid therapeutic agent is VIR-2218, which targets HBsAg. In some aspects, the nucleic acid therapeutic agent is WVE-003, which targets HTT. In some aspects, the nucleic acid therapeutic agent is WVE-004, which targets C9orf72. In some aspects, the nucleic acid therapeutic agent is WVEN-531, which targets DMD. In some aspects, the nucleic acid therapeutic agent is zilebesiran, which targets AGT and has the sequences set forth in SEQ ID NO:726 and SEQ ID NO:727. In some aspects, the nucleic acid therapeutic agent is zilganersen, which targets GFAP.

### Payload: Chemotherapy Agents

In some aspects, the payload comprises a chemotherapy agent. In some aspects, the chemotherapy agent comprises an alkylating agent, antimetabolite, anti-microtubule, topoisomerase inhibitor, cytotoxic antibiotic, or a combination thereof. In some aspects, the chemotherapy agent comprises, e.g., cyclophosphamide, methotrexate, 5-fluorouracil, doxorubicin, docetaxel, vinblastine, vincristine, prednisolone, bleomycin, etoposide, cisplatin, epirubicin, capecitabine, ifosfamide, folinic acid, oxaliplatin, vinorelbine, procarbazine, mistune, dacarbazine, or a combination thereof. In some aspects, the payload comprises a nucleoside analog selected from the group consisting of azacitidine, capecitabine, carmofur, cladribine, clofarabine, cytarabine, decitabine, floxuridine, fludarabine, fluorouracil, gemcitabine, mercaptopurine, nelarabine, pentostatin, tegafur, and tioguanine. In some aspects the payload comprises an antifolate selected from the group consisting of methotrexate, pemetrexed, and raltitrexed. In some aspects, the payload comprises a topisomerase I inhibitor such as irinotecan or topotecan. In some aspects, the payload comprises an anthracycline such as daunorubicin, doxorubicin, epirubicin, idarubicin, mitoxantrone or valrubicine. In some aspects, the payload comprises a podophyllotoxin such as etoposide or teniposide. In some aspects, the payload comprises a taxane such a cabazitaxel, docetaxel, or paclitaxel. In some aspects, the payload comprises a vinca alkaloid selected from the group consisting of vinblastine, vincristine, vindesine, vinflunine, and vinrelbine. In some aspects, the payload comprises an alkylating agent selected from the group consisting of bendamustine, busulfan, carmustine, chlorambucil, chormethine, cyclophosphamide, dacarbazine, folemustine, ifosfamide, lomustine, melphalan, streptozotocin, and temozolomide. In some aspects, the payload comprises a platinum compound selected from the group consisting of carboplatin, cisplatin, nedaplatin, and oxaliplatin.

In some aspects, the payload comprises a targeted antineoplastic therapeutic agent. In some aspects, the targeted antineoplastic therapeutic agent comprises an antibody, e.g., a monoclonal antibody, or an antigen-binding portion thereof. In some aspects, the monoclonal antibodsy comprises alemtuzumab (anti-CD52), bevacizumab (anti-VEGF), cetuximab (anti-EGFR), denosumab (antri-RNAKL), gemtuzumab ozogamicin (anti-CD33), ibritumomab tiuxetan (anti-CD20), ipilimumab (anti-CTLA4), nivolumab (anti-PD1), ofatumumab (anti-CD20), panitumumab (anti-EGFR), pembrolizumab (anti-PD1), pentuzimab (anti-HER2), rituximab (anti-CD20), tositumomab (anti-CD20), trastuzumab (anti-HER2) or an antigen binding portion thereof.

In some aspects, the payload comprises a small molecules tyrosine kinase inhibitor. In some aspects, the tyrosine kinase inhibitor is selected from the group consisting of afatinib (EGFR, HER2 and HER4 inhibitor), aflibercept (VEGF and PGF inhibitor), axitinib (multikinase inhibitor), bosutinib (Bcr-Abl and SRc kinase inhibitor), crizotinib (ALK, HGFR, and RON inhibitor), dasatinib (BCR-ABL, SRC family, c-Kit, EPHA2 and PDGFR-β kinase inhibitor), erlotinib (EGFR inhibitor), gefitinib (EGFR inhibitor), imatinib (Bcr-Abl kinase inhibitor), lapatinib (HER2 inhibitor), nilotinib (Bcr-Abl kinase inhibitor), pazopanib (Multikinase inhibitor, including c-KIT, FGFR, PDGFR and VEGFR), ponatinib (Multikinase inhibitor (BEGFR, PDGFR, FGFR, EPH receptors and SRC families of kinases, and KIT, RET, TIE2 and FLT3), that also inhibits T135I Bcr-Abl kinase), regorafenib (Multikinase inhibitor for RET, VEGFR1, VEGFR2, VEGFR3, KIT, PDGFR-alpha, PDGFR-beta, FGFR1, FGFR2, TIE2, DDR2, Trk2A, Eph2A, RAF-1, BRAF, BRAFV600E, SAPK2, PTK5, and Bcr-Abl.), ruxolitinib (JAK1 and JAK2 inhibitor), sorafenib (Multikinase inhibitor, including VEGF and PDGF receptor kinases), sunitinib (Multikinase inhibitor, including VEGF & PDGF receptor tyrosine kinases), and vandelanic (Tyrosine kinase inhibitor (TKI) with selective activity against RET, VEGFR-2 and EGFR).

In some aspects, the payload comprises an mTOR inhibitor such as everolimus or temsirolimus. In some aspects, the payload comprises a retinoid selected from the group consisting of bexarotene (RXR agonist), isotretinoin (RXR and RAR agonist), tamibarotene (RAR agonist), and tretinoin (RXR and RAR agonist). In some aspects, the payload comprises an immunomodulatory agent (IMID) such as lenalidomide, thalidomide, or pomalidomide. In some aspects, the payload comprises a histone deacetylase inhibitor such as romidepsin, valproate, or vorinostat.

### Bioavailability and Biodistribution Modifying Agents

In some aspects, an AAV-ITR based gene delivery system of the present disclosure can comprise a bio-distribution modifying agent. As used herein, the term a “bio-distribution modifying agent,” which refers to an agent that can modify the distribution of an AAV-ITR based gene delivery system of the present disclosure in vivo or in vitro to direct it to a specific cell type or tissue, e.g., liver tissues, lung tissue, muscle tissue; to a specific physiological compartment, e.g., the central nervous system; or across a physiological barrier, e.g., the blood-brain barrier.

In some aspects, the term “targeting moiety” can be used interchangeably with the term bio-distribution modifying agent. In some aspects, the targeting moiety alters the tropism of the components of an AAV-ITR based gene delivery system of the present disclosure (“tropism moiety”). As used herein, the term “tropism moiety” refers to a targeting moiety that alters and/or enhances the natural movement of the AAV-ITR based gene delivery system of the present disclosure. In that respect, an antibody or combination thereof attached to a lipidic delivery system (e.g., a LNP or liposome) component of an AAV-ITR based gene delivery system of the present disclosure, wherein the antibody or combination thereof targets CD3 and/or CD28, can directs an AAV-ITR based gene delivery system of the present disclosure to T-cells. Thus unless indicated otherwise, the term “targeting moiety” or “targeting molecule” as used herein, encompasses tropism moieties.

Pharmacokinetics, biodistribution, and in particular tropism and retention in the desired tissue or anatomical location can also be accomplish by selecting the appropriate administration route (e.g., intrathecal administration or intraocular administration to improve tropism to the central nervous system).

In principle, an AAV-ITR based gene delivery system of the present disclosure comprising at least one tropism moiety that can direct the AAV-ITR based gene delivery system to a specific target cell or tissue (e.g., T cells) can be administered using any suitable administration method known in the art (e.g., intravenous injection or infusion) since the presence of the tropism moiety (alone or in combination with the presence of an antiphagocytic signal and the use of a specific administration route) will induce a tropism of the AAV-ITR based gene delivery system towards the desired target cell or tissue.

In some aspects, the AAV-ITR based gene delivery system comprises a surface ligand covalently attached to the surface of the lipidic delivery system component (e.g., a LNP or liposome) of the AAV-ITR based gene delivery system, wherein the surface ligand can increase permeation through the blood-brain barrier. In some aspects, the surface ligand is a transferrin receptor.

In some aspects, the AAV-ITR based gene delivery system comprises a surface ligand covalently attached to the surface of the lipidic delivery system component (e.g., a LNP or liposome) of the AAV-ITR based gene delivery system, wherein the surface ligand is a tissue or cell-specific target ligand that increases tropism to a tissue or physiological compartment.

In some aspects, the AAV-ITR based gene delivery system comprises a surface ligand covalently attached to the surface of the lipidic delivery system component (e.g., a LNP or liposome) of the AAV-ITR based gene delivery system, wherein the surface ligand is a surface anchored anti-phagocytic signal. In some aspects, the anti-phagocytic signal comprises CD47, CD24, a fragment thereof, and any combination thereof.

### MSTAR Antibody Architectures

In some aspects, an AAV-ITR vector of the present disclosure can comprise a polynucleotide encoding an MSTAR antibody. In some aspects, the polynucleotides encoding each of the polypeptide components of an MSTAR antibody are concatenated in the AAV-ITR vector. Thus, in some aspects, the AAV-ITR vector encoding an MSTAR antibody can be bicistronic or multicistronic. In some aspects, a targeting antibody directing an AAV-ITR based gene delivery system of the present disclosure to a specific target cell (e.g., a T cell) or tissue, can be covalently attached to the surface of an lipidic delivery system (e.g., a liposome or LNP) disclosed herein. MSTAR antibody formats and methods to conjugate such antibodies to a lipidic delivery system are disclosed in U.S. Appl. Publ. Nos. US20230227553A1, US20230235092A1, US20230203199A1, and PCT Publ. Nos. WO2024007012, WO2023056312, WO2023056313, WO2023056314, and U.S. Provisional Patent Application No. Appl. No. 63/594,875, which are herein incorporated by reference in their entireties.

### Vectors and Cells

The present disclosure provides vectors such as the components of the AAV-ITR based gene delivery system of the present disclosure, i.e., AAV-ITR vectors and Rep vectors, and combinations thereof. The disclosure also provides cells, e.g., for an ex vivo treatment or for recombinant expression, that comprise, e.g., a gene of interest integrated in its genome using an AAV-ITR based gene delivery system of the present disclosure. The methods to construct the vectors components of the AAV-ITR based gene delivery system of the present disclosure are well known to those skilled in the art, and can be used, e.g., to assemble the different components of the AAV-ITR vectors and Rep vectors disclosed herein. These methods include, for example, in vitro recombinant DNA techniques, synthetic techniques, and in vivo genetic recombination.

In some aspects, an AAV-ITR based gene delivery system of the present disclosure can be use to transfect a host cell by conventional techniques and the resulting cell can then be cultured by conventional techniques to produce a therapeutic protein, peptide, or nucleic acid, e.g., an antibody (that, e.g., can be secreted), an enzyme (e.g., for a gene replacement therapy), or a peptide (e.g., a hormone, growth factor, cytokine, etc.).

In some aspects, the present disclosure provides a host cell containing a polynucleotide integrated into its genome by using an AAV-ITR based gene delivery system of the present disclosure, wherein the polynucleotide encoding an antigen binding polypeptide or polypeptide complex comprising, e.g., comprising six CDRs, VH, VL, VH and VL, heavy chain, light chain, or heavy and light chain, or a domain thereof (e.g., one or more CDRs, VH, VL, VH and VL, heavy chain, or light chain). Such host-vector systems represent vehicles by which the coding sequences of interest can be produced and subsequently purified, but also represent cells which can, when transformed or transfected with the appropriate nucleotide coding sequences, express a polypeptide or polypeptide complex provided herein in situ.

In some aspects, the polynucleotide integrated into the genome of a host cell by using an AAV-ITR based gene delivery system of the present disclosure encodes both heavy and light chains, or a an antigen binding portion of an antibody. In some aspects, the antibody is a monospecific, bispecific, trispecific or tetraspecific antibody. In some aspects, the antibody is an MSTAR antibody. In some aspects, the polynucleotide integrated into the genome of a host cell by using an AAV-ITR based gene delivery system of the present disclosure encodes a CAR. In some aspects, the polynucleotide integrated into the genome of a host cell by using an AAV-ITR based gene delivery system of the present disclosure encodes a component of a gene editing systems or a combination thereof (e.g., a nuclease such as Cas, and a gRNA).

In some aspects, the AAV-ITR and/or Rep vector of the present disclosure can be produced in a variety of systems. These systems include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing AAV ITR_GOI sequences; or mammalian cell systems (e.g., COS (e.g., COS1 or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40, YB/20, and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In some aspects, the AAV-ITR based gene delivery systems of the present disclosure cells can be used for the recombinant production of a gene of interest (e.g., a polypeptide or polypeptide complex) described herein in CHO cells, for example CHO cells from the CHO GS System™ (Lonza). In some aspects, cells for expressing the polypeptides or polypeptide complexes provided herein are human cells, e.g., human cell lines. In some aspects, a mammalian expression vector is pOptiVEC™ or pcDNA3.3. In some aspects, eukaryotic cells (e.g., mammalian cells) are used for the expression of recombinant polypeptides. For example, mammalian cells such as Chinese hamster ovary (CHO) cells in conjunction with a vector such as the major intermediate early gene promoter element from human cytomegalovirus is an effective expression system for polypeptides (Foecking M K & Hofstetter H (1986) Gene 45: 101-105; and Cockett M I et al., (1990) Biotechnology 8: 662-667). In some aspects, the polypeptides or polypeptide complexes provided herein are produced by HEK-293T cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells that possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

Once a polypeptide or polypeptide complex provided herein has been produced by recombinant expression, it can be purified by any method known in the art for purification of a protein or immunoglobulin molecule, for example, by chromatography (e.g., ion exchange, affinity, particularly by affinity for the specific antigen after Protein A, and size exclusion chromatography), centrifugation, differential solubility, or by any other standard technique for the purification of proteins. Further, the polypeptides or polypeptide complexes provided herein can be fused to heterologous polypeptide sequences provided herein (e.g., peptide tags) or otherwise known in the art to facilitate purification.

In some aspects, a polypeptide or polypeptide complex provided herein can be isolated or purified. Generally, an isolated polypeptide or polypeptide complex is one that is substantially free of other polypeptides or polypeptide complexes with different antigenic specificities. For example, in some aspects, a preparation of a polypeptide or polypeptide complex described herein can be substantially free of cellular material and/or chemical precursors.

### Pharmaceutical Composition

The present disclosure provides pharmaceutical compositions comprising AAV-ITR based gene delivery system of the present disclosure. In some aspects, the pharmaceutical composition comprises a pharmaceutically acceptable carrier. In some aspects, provided herein is a pharmaceutical composition comprising a double stranded polynucleotide encoding a gene of Interest (GOI) within an AAV-ITR vector of the present disclosure. In some aspects, the pharmaceutical composition further comprises a Rep vector of the present disclosure. In some aspects, the pharmaceutical composition in encapsulated in a lipidic delivery system (e.g., a LNP or liposome) or combination thereof

In a specific aspect, a pharmaceutical composition provided herein comprises (1) an AAV-ITR vector encoding a GOI; (2) a Rep68 vector or Rep78 vector; and (3) a pharmaceutically acceptable carrier. In a specific aspect, a pharmaceutical composition provided herein comprises (1) an AAV-ITR vector encoding a CAR; (2) a Rep68 vector or Rep78 vector; and (3) a pharmaceutically acceptable carrier. In a specific aspect, a pharmaceutical composition provided herein comprises (1) an AAV-ITR vector encoding an antibody, e.g., a MSTAR antibody; (2) a Rep68 vector or Rep78 vector; and (3) a pharmaceutically acceptable carrier.

In some aspects, the AAV-ITR vector is encapsulated in a lipidic delivery system (e.g., a LNP or liposome). In some aspects, the Rep68 vector or Rep78 vector is encapsulated in a lipidic delivery system (e.g., a LNP or liposome). In some aspects, the AAV-ITR vector is encapsulated in a first lipidic delivery system (e.g., a LNP or liposome) and the Rep68 vector or Rep78 vector is encapsulated in a second lipidic delivery system (e.g., a LNP or liposome). In some aspects, the first and second lipidic delivery systems are the same. In some aspects, the first and second lipidic delivery systems are different.

Thus, is some aspects, the pharmaceutical composition of the present disclosure comprises (1) an AAV-ITR vector of the present disclosure encapsulated in a first lipidic delivery system (e.g., a LNP or liposome) and (2) a Rep vector (e.g., Rep78 vector) of the present disclosure encapsulated in a second lipidic delivery system (e.g., a LNP or liposome).

The term “pharmaceutically acceptable carrier” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is known in the art. Except insofar as any conventional media or agent is incompatible with the active ingredient, its use in the therapeutic formulations is contemplated. Supplementary active ingredients can also be incorporated into the pharmaceutical compositions provided herein. In addition, various excipients, such as are commonly used in the art, can be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies. In some aspects, the pharmaceutical composition is for parenteral, intravenous or subcutaneous administration.

Once a pharmaceutical composition has been formulated, it can be stored in sterile vials as a solution, suspension, gel, emulsion, solid, crystal, or as a dehydrated or lyophilized powder. Such formulations can be stored either in a ready-to-use form or in a form (e.g., lyophilized) that is reconstituted prior to administration.

### Kits

The present disclosure provides a kit comprising (1) an AAV-ITR based gene delivery system of the present disclosure, or one or more of its components, or a pharmaceutical compositions disclosed herein, and (2) optionally instructions for use, e.g., instruction to replace a GOI integrated in an AAV-ITR vector included in the kit with a GOI of interest to the user of the kit. In some aspects, the kit comprises an AAV-ITR based gene delivery system of the present disclosure. In some aspects, the kit comprises an AAV-ITR vector disclosed herein. In some aspects, the kit comprises a Rep vector disclosed herein. In some aspects, the kit comprises an AAV-ITR and a Rep vector disclosed herein, wherein the AAV-ITR vector and/or Rep vector are encapsulated in a lipidic delivery system (e.g., a LNP or liposome).

In some aspects, the kit comprises (1) an AAV-ITR and/or a Rep vector disclosed herein and (2) optionally instructions for the encapsulation of the AAV-ITR and/or Rep vector.

### METHODS OF USE

The present disclosure provides method of treating or preventing a disease or disorder in a subject in need thereof comprising administering to the subject an AAV-ITR based gene delivery system of the present disclosure. In some aspects, the present disclosure provides a gene therapy method comprising administering an AAV-ITR based gene delivery system of the present disclosure to a subject in need thereof. In some aspects, the gene therapy is long-term gene therapy. In some aspects, the gene is short-term gene therapy. In some aspects, the gene therapy is in vivo. In some aspects, the gene therapy is ex vivo.

In some aspects, the AAV-ITR vector comprises a double-stranded DNA encoding a gene of interest, e.g., a protein (e.g., an enzyme, a growth factor, a hormone, a CAR, or an antibody, or a component of a gene editing system) or a nucleic acid (e.g., a miRNA, an antisense oligonucleotide, or a gRNA). In some aspects, the AAV-ITR vector comprises a double stranded DNA encoding an mRNA, a miRNA, a gRNA, or a combination thereof.

The present disclosure also provides a CAR-T cell therapy method comprising administering a AAV-ITR based gene delivery system of the present disclosure to the subject, wherein the AAV-ITR vector comprising a double stranded polynucleotide encoding a CAR protein. In some aspects, the AAV-ITR based gene delivery system of the present disclosure comprises a lipidic delivery system (e.g., a LNP or liposome) that has a neuron targeting protein integrated in the lipidic delivery system (e.g., a LNP or liposome) or covalently linked to the lipidic delivery system (e.g., a LNP or liposome). In some aspects, the AAV-ITR based gene delivery system of the present disclosure comprises a lipidic delivery system (e.g., a LNP or liposome) that has a T cell targeting protein integrated in the lipidic delivery system (e.g., a LNP or liposome) or covalently linked to the lipidic delivery system (e.g., a LNP or liposome). In some aspects, the AAV-ITR based gene delivery system of the present disclosure comprises a lipidic delivery system (e.g., a LNP or liposome) that has a B cell targeting protein integrated in the lipidic delivery system (e.g., a LNP or liposome) or covalently linked to the lipidic delivery system (e.g., a LNP or liposome). In some aspects, the AAV-ITR based gene delivery system of the present disclosure comprises a lipidic delivery system (e.g., a LNP or liposome) that has a lipid cell targeting protein integrated in the lipidic delivery system (e.g., a LNP or liposome) or covalently linked to the lipidic delivery system (e.g., a LNP or liposome). In some aspects, the AAV-ITR based gene delivery system of the present disclosure comprises a lipidic delivery system (e.g., a LNP or liposome) that has a muscle cell targeting protein integrated in the lipidic delivery system (e.g., a LNP or liposome) or covalently linked to the lipidic delivery system (e.g., a LNP or liposome). In some aspects, the AAV-ITR based gene delivery system of the present disclosure comprises a lipidic delivery system (e.g., a LNP or liposome) that has a lung cell targeting protein integrated in the lipidic delivery system (e.g., a LNP or liposome) or covalently linked to the lipidic delivery system (e.g., a LNP or liposome).

The present disclosure provides a method to target at least one gene of interest to a target cell or tissue comprising (i) encapsulating an AAV-ITR vector comprising a double stranded polynucleotide encoding at least one gene of interest (GOI) into a first lipidic delivery system (e.g., a LNP or liposome); (ii) encapsulating a Rep vector (e.g., encoding a Rep68 or Rep78 protein) in a second lipidic delivery system (e.g., a LNP or liposome); (iii) attaching a targeting molecule to the surface or the first and second lipidic delivery systems wherein the targeting molecule specifically binds to a surface protein (e.g., a receptor) on the surface of the target cell or tissue; and (iv) optionally attaching another payload to the surface of the first and/or second lipidic delivery system, and/or encapsulating another payload into the first and/or second lipidic delivery system.

In some aspects, a payload is attached to the inner surface of a lipidic delivery system (e.g., a LNP or liposome), attached to the outer surface or the lipidic delivery system (e.g., a LNP or liposome), transverses the lipid layer of the lipidic delivery system (e.g., a LNP or liposome), or, a combination thereof.

The present disclosure further provides a method of replicating a nucleic acid encoding a therapeutic polypeptide in a subject in need of treatment for a disease or condition. In some aspects, the method of replicating a nucleic acid encoding a therapeutic polypeptide in a patient in need of treatment for a disease or condition comprises administering to the subject a composition comprising an AAV-ITR vector comprising a polynucleotide encoding a GOI, wherein expression of the GOI improves at least one symptom of the disease or condition in the subject.

In some aspects, the method further comprises administering to the subject a composition comprising a Rep vector. In some aspects, the method comprises administering Rep vector, wherein the Rep vector comprises a Rep68 vector. In some aspects, the method comprises administering a Rep vector, wherein the Rep vector comprises a Rep78 vector. In some aspects, the method comprises administering an mRNA encoding Rep68. In some aspects, the method comprises administering an mRNA encoding Rep78. In some aspects, the method comprises administering to the subject a composition comprising an AAV-ITR vector comprising a double stranded polynucleotide sequence encoding a gene of interest, and a Rep vector comprising an mRNA encoding a Rep68 protein or a Rep78 protein.

In some aspects, the method of replicating a nucleic acid encoding a therapeutic polypeptide in a patient in need of treatment of a disease or condition comprises administering a combination of an AAV-ITR vector and a Rep vector (e.g., a Rep68 vector or Rep78 vector). In some aspects, the method comprises administering a combination of an AAV-ITR vector and a Rep68 vector. In some aspects, the method comprises administering a combination of an AAV-ITR vector and a Rep78 vector. In some aspects, the method comprises administering a combination of an AAV-ITR vector and a Rep68 mRNA.

In some aspects, the method of replicating a nucleic acid encoding a therapeutic polypeptide in a patient in need of treatment of a disease or condition comprises administering a combination of a composition comprising an AAV-ITR vector and a composition comprising a Rep68 vector and/or Rep 78 vector. In some aspects, the method comprises administering a combination of a composition comprising an AAV-ITR vector and a composition comprising a Rep68 vector. In some aspects, the method comprises administering a combination of a composition comprising an AAV-ITR vector and a composition comprising a Rep78 vector. In some aspects, the method comprises administering a combination of a composition comprising an AAV-ITR vector and a composition comprising a Rep68 mRNA. In some aspects, the method comprises administering a combination of an AAV-ITR vector and a composition comprising a Rep78 mRNA.

In some aspects, the method of replicating a nucleic acid encoding a therapeutic polypeptide in a patient in need of treatment of a disease or condition comprises administering a composition comprising (i) an AAV-ITR vector and (ii) a Rep vector (e.g., Rep68 vector or Rep78 vector). In some aspects, the method comprises administering a composition comprising an AAV-ITR vector and a Rep68 vector. In some aspects, the method comprises administering a composition comprising an AAV-ITR vector and a Rep78 vector. In some aspects, the method comprises administering a composition comprising an AAV-ITR vector and a composition comprising a Rep68 mRNA. In some aspects, the method comprises administering a composition comprising an AAV-ITR vector and a Rep78 mRNA. In some aspects, the method comprises administering a combination of a composition comprising an AAV-ITR vector and a composition comprising a Rep vector, wherein the composition comprising the AAV-ITR vector and the composition comprising the Rep vector are administered at the same time. In some aspects, the composition comprising the AAV-ITR vector and the composition comprising the Rep IVT vector are administered consecutively. In some aspects, the composition comprising the AAV-ITR vector is administered before the composition comprising the Rep vector. In some aspects, the composition comprising the Rep vector is administered before the composition comprising the AAV-ITR vector. In some aspects, the composition comprising an AAV-ITR and the composition comprising a Rep vector are administered within a time period of 1 minute, 2 minutes, 5 minutes, 10 minutes, 20 minutes, 30 minutes, 45 minutes, 1 hour, 1.5 hours, 2 hours, 2.5 hours, 3 hours, 3.5 hours, 4 hours, 4.5 hours, 5 hours, 5.5 hours, or 6 hours.

In some aspects, the method of replicating a nucleic acid encoding a therapeutic polypeptide in a patient in need of treatment of a disease or condition comprises administering a combination of a composition comprising an AAV-ITR vector and a composition comprising a Rep vector, wherein both compositions are present within a delivery system. In some aspects, the delivery system is a lipid nanoparticle (LNP). In some aspects, the delivery system is a liposome. In some aspects, the delivery system is a cationic lipid liposome. In some aspects, the delivery system is a polymeric delivery platform. In some aspects, the method of replicating a nucleic acid encoding a therapeutic polypeptide in a patient in need of treatment of a disease or condition comprises administering a combination of a composition comprising an AAV-ITR vector and a composition comprising a Rep vector, e.g., a Rep68 vector or a Rep78 vector, wherein the two compositions are present within different delivery systems. In some aspects, a composition comprising an AAV-ITR vector is present in a LNP delivery system or a liposome delivery system. In some aspects, a composition comprising a Rep vector is present in a LNP delivery system or a liposome delivery system.

In some aspects, an AAV-ITR vector is administered to a subject in need thereof in an LNP. In some aspects, a Rep vector is administered to a subject in need thereof in an LNP. In some aspects, an AAV-ITR vector is administered to a subject in need thereof in a liposome. In some aspects, a Rep vector is administered to a subject in need thereof in a liposome. In some aspects, an AAV-ITR vector is administered to a subject in need thereof in a cationic liposome. In some aspects, a Rep vector is administered to a subject in need thereof in a cationic liposome. In some aspects, an AAV-ITR is administered to a subject in need thereof in a polymeric delivery system. In some aspects, a Rep vector is administered to a subject in need thereof in a polymeric delivery system. In some aspects, an AAV-ITR vector is administered to a subject in need thereof in an LNP and a Rep vector is administered to the subject in a liposome. In some aspects, an AAV-ITR vector is administered to a subject in need thereof in a liposome and a Rep vector is administered to the subject in an LNP. In some aspects, an AAV-ITR vector is administered to a subject in need thereof in an LNP and a Rep vector is administered to the subject in a cationic liposome. In some aspects, an AAV-ITR vector is administered to a subject in need thereof in a cationic liposome and a Rep vector is administered to the subject in an LNP. In some aspects, an AAV-ITR vector is administered to a subject in need thereof in an LNP and a Rep vector is administered to the subject in a polymeric delivery system. In some aspects, an AAV-ITR vector is administered to a subject in need thereof in a polymeric delivery system and a Rep vector is administered to the subject in an LNP.

As used herein, the terms “prevent” or “preventing” refer to the prevention of the onset, recurrence or spread, in whole or in part, of a disease or condition provided herein, or a symptom thereof.

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, “administering” is meant a method of giving a dosage of AAV-ITR based gene delivery system of the present disclosure comprising one or more therapeutic agents (e.g., DNA and/or mRNA) to a subject in need thereof (e.g., a patient). Administering can be by any suitable means, including parenteral, intrapulmonary or intranasal. Parenteral infusions include, for example, intramuscular, intravenous, intraarterial, intraperitoneal or subcutaneous administration. Dosing can be by any suitable route, e.g., by injection, such as intravenous or subcutaneous injection. Various dosing schedules including, but not limited to, single or multiple administrations over various time-points, bolus administration, and pulse infusion are contemplated herein.

As used herein, a “therapeutically effective amount” is an amount of an AAV-ITR based gene delivery system of the present disclosure comprising one or more therapeutic agents (e.g., an mRNA encoding a gene of interest) that is sufficient to achieve the desired effect and can vary according to the nature and severity of the condition, and the potency of the AAV-ITR based gene delivery system of the present disclosure comprising one or more therapeutic agents (e.g., DNA and/or mRNA).

A therapeutic effect is the relief, to at least some extent, of one or more symptoms of the disease or disorder, and can include curing a disease or disorder. “Curing” means that the symptoms of active disease are eliminated. However, certain long-term or permanent effects of a disease or disorder can exist even after a cure is obtained.

As used herein, the term “subject” means a human or a non-human mammal, e.g., a dog, cat, mouse, rat, cow, sheep, pig, goat, non-human primate or bird, e.g., chicken, as well as any other vertebrate or invertebrate. In some aspects, the subject is a human. In some aspects, the subject is a veterinary animal. In some aspects, the subject is a mammal. In some aspects, the terms subject and patient are used interchangeably.

In some aspects, the present disclosure provides a method of treating cancer. In some aspects, the cancer is a hematologic cancer. In some aspects, the hematologic cancer is a lymphoma, myeloma, or leukemia. In some aspects, the lymphoma is a Hodgkin lymphoma or Non-Hodgkin lymphoma. In some aspects, the myeloma is a multiple myeloma, solitary plasmocytoma, or extamedullary plasmocytoma. In some aspects, the leukemia is an Acute Lynphocytic Leukemia (ALL), Chronic Lymphocytic Leukemia (CLL), Acute Myeloid Leukemia (AML), Chronic Myeloid Leukemia (CML), Hairy Cell Leukemia, Childhood Acute Lymphoblastic Leukemia, or Childhood Acute Myeloid Leukemia. In some aspects, the cancer is a solid cancer. In some aspects, the solid cancer is a breast cancer, colon cancer, kidney cancer, lung cancer, liver cancer, skin cancer, brain cancer, bladder cancer, prostate cancer, endometrial cancer, pancreatic cancer, or thyroid cancer.

In some aspects, the present disclosure provides a method for treating an immune disease. In some aspects, the immune disease is artherosclosis, Lyme disease, or fibromyalgia. In some aspects, the immune disease is an autoimmune disease. In some aspects, the autoimmune disease is Addison disease, rheumatoid arthritis, inflammatory bowel disease, celiac disease, Hashimoto thyroiditis, Graves disease, Myasthenia gravis, multiple sclerosis, dermatomyositis, systemic lupus erythematosus, type I diabetes, Sjögren syndrome, Giant cell arteritis, reactive arthritis, pernicious anemia, alopecia areata, ankylosing spondylitis, antiphospholipid syndrome, Guillain-Barre syndrome, or psoriasis. In one aspect, the disease or disorder is a cancer, an inflammatory disease, a neurodegenerative disorder, a central nervous disease or a metabolic disease. In some aspects, a disease or disorder that can be treated with the present methods comprises a cancer, graft-versus-host disease (GvHD), autoimmune disease, infectious diseases, or fibrotic diseases. In some aspects, the disease or disorder is a cancer. When administered to a subject with a cancer, in certain aspects, a composition of the present disclosure can up-regulate an immune response and enhance the tumor targeting of the subject's immune system. In some aspects, the cancer being treated is characterized by infiltration of leukocytes (T-cells, B-cells, macrophages, dendritic cells, monocytes) into the tumor microenvironment, or so-called “hot tumors” or “inflammatory tumors.” In some aspects, the cancer being treated is characterized by low levels or undetectable levels of leukocyte infiltration into the tumor microenvironment, or so-called “cold tumors” or “non-inflammatory tumors.” In some aspects, the composition of the present disclosure is administered in an amount and for a time sufficient to convert a “cold tumor” into a “hot tumor,” i.e., said administering results in the infiltration of leukocytes (such as T-cells) into the tumor microenvironment. In certain aspects, cancer comprises bladder cancer, cervical cancer, renal cell cancer, testicular cancer, colorectal cancer, lung cancer, head and neck cancer, and ovarian, lymphoma, liver cancer, glioblastoma, melanoma, myeloma, leukemia, pancreatic cancers, or combinations thereof. In other aspects, the terms “distal tumor” or “distant tumor” refer to a tumor that has spread from the original (or primary) tumor to distant organs or distant tissues, e.g., lymph nodes. In some aspects, the composition of the present disclosure can treat a tumor after the metastatic spread.

In some aspects, the disease or disorder is a graft-versus-host disease (GvHD). In some aspects, the disease or disorder that can be treated with the present disclosure is an autoimmune disease. Non-limiting examples of autoimmune diseases include: multiple sclerosis, peripheral neuritis, Sjogren's syndrome, rheumatoid arthritis, alopecia, autoimmune pancreatitis, Behcet's disease, bullous pemphigoid, celiac disease, Devic's disease (neuromyelitis optica), glomerulonephritis, IgA nephropathy, assorted vasculitides, scleroderma, diabetes, arteritis, vitiligo, ulcerative colitis, irritable bowel syndrome, psoriasis, uveitis, systemic lupus erythematosus, and combinations thereof.

In some aspects, a disease or disorder that can be treated with the present methods comprises a Pompe disease, Gaucher, a lysosomal storage disorder, mucovicidosis, cystic fibrosis, Duchenne and Becker muscular dystrophy, transthyretin amyloidosis, hemophilia A, hemophilia B, adenosine-deaminase deficiency, Leber's congenital amaurosis, X-linked adrenoleukodystrophy, metachromatic leukodystrophy, OTC deficiency, glycogen storage disease 1A, Criggler-Najjar syndrome, primary hyperoxaluria type 1, acute intermittent porphyria, phenylketonuria, familial hypercholesterolemia, mucopolysaccharidosis type VI, al antitrypsin deficiency, Retts Syndrome, Dravet Syndrome, Angelman Syndrome, DM1 disease, Fragile X disease, Huntingtons Disease, Friedreichs ataxia, CMT disease (also known as Charcot-Marie-Tooth disease, hereditary motor and sensory neuropathy (HMSN), or peroneal muscular atrophy), CMT1X disease, catecholaminergic polymorphic ventricular tachycardia, spinocerebellar ataxia type 3 (SCA3) disease, limb-girdle muscular dystrophy, or a hypercholesterolemia. In some aspects, the treatment is prophylactic.

In some aspects, the disease or disorder is a neurodegenerative disease. In some aspects, the neurodegenerative disease is selected from Alzheimer's disease, Parkinson's disease, prion disease, motor neuron disease, Huntington's disease, spinocerebellar ataxia, spinal muscular atrophy, and any combination thereof.

In certain aspects, the disease or disorder comprises a muscular dystrophy. In some aspects, the muscular dystrophy is selected from Duchenne type muscular dystrophy (DMD), myotonic muscular dystrophy, facioscapulohumeral muscular dystrophy (FSHD), congenital muscular dystrophy, limb-girdle muscular dystrophy (including, but not limited to, LGMD2B, LGMD2D, LGNMD2L, LGMD2C, LGMD2E and LGMD2A), and any combination thereof.

In some aspects, the disease or disorder is selected from AADC deficiency (CNS), ADA-SCID, Alpha-1 antitrypsin deficiency, β-thalassemia (severe sickle cell), Cancer (head and neck squamous cell), Niemman-Pick Type C Disease, Cerebral ALD, Choroideremia, Congestive heart failure, Cystic Fibrosis, Duchenne muscular dystrophy (DMD), Fabry disease, Glaucoma, Glioma (cancer), Hemophilia A, Hemophilia B, HoFH (hypercholesterolemia), Huntington's Disease, Lipoprotein lipase deficiency, Leber hereditary optic neuropathy (LHON), Metachromatic leukodystrophy, MPS I (Hurler syndrome), MPS II (Hunter's syndrome), MPS III (Sanfilippo Syndrome), Parkinson's disease, Pompe Disease, Recessive Dystrophic Epidermolysis Bullosa, RPE65 deficiency (vision loss), Spinal Muscular Atrophy (SMA I), Wet AMD (retinal disease), Wiskott Aldrich syndrome (WAS), Mucopolysaccharidosis type IIIA (MPS IIIA), X-linked myotubular myopathy, X-linked retinitis pigmentosa, and any combination thereof.

### EMBODIMENTS

In the text below the term “Ex,” wherein x is an integer, refers to an embodiment of the present invention. Thus, e.g., E1 represents Embodiment 1.

E1 comprises an AAV-ITR based gene delivery system comprising a composition, optionally capsid-free, comprising (i) a linear dsDNA AAV-ITR vector comprising 5′ and 3′ AAV ITR flanking a polynucleotide sequence encoding a therapeutic polypeptide, a therapeutic polynucleotide, or a combination thereof, and, (ii) a mRNA Rep vector encoding a Rep protein. E2 comprises the AAV-ITR based gene delivery system of embodiment E1, wherein the ITR comprises an ITR sequence from AAV1, AAV2, AAV3, AAV3B, AAV4, AAV5, AAV6, AAV7, AAV8, AAV9, AAV10, AAV11, AAV12, AAV13, an ortholog parvoviral ITR, or a functional fragment of functional variant thereof. E3 comprises the AAV-ITR based gene delivery system of embodiment E1 or E2, wherein the AAV-ITR vector comprises a regulatory element or a combination thereof. E4 comprises the AAV-ITR based gene delivery system of embodiment E2, wherein the regulatory element comprises a promoter. E5 comprises the AAV-ITR based gene delivery system of embodiment E4, wherein the promoter is selected from the group consisting of a CMV promoter, a SV40 promoter, an AdMLP, a polyoma promoter, a β-globin promoter, an SRa promoter, a U6 promoter, a CBA promoter, a CAG promoter, an UBC promoter, a β glucuronidase promoter, an NSE promoter, a Synapsin promoter, a MeCP2 promoter, an EF 1a promoter, and a GFAP promoter.

E6 comprises the AAV-ITR based gene delivery system of embodiment E4, wherein the promoter is a tissue-specific promoter. E7 comprises the AAV-ITR based gene delivery system of embodiment E6, wherein the tissue-specific promoter is selected from the group consisting of a muscle-specific promoter, a B cell promoter, a monocyte promoter, a leukocyte promoter, a macrophage promoter, a pancreatic acinar cell promoter, an endothelial cell promoter, a lung tissue promoter, an astrocyte promoter, and a nervous system promoter. E8 comprises the AAV-ITR based gene delivery system of embodiment E7, wherein the muscle-specific promoter is selected from the group consisting of an MCK promoter, a DES promoter, a TNNI2 promoter, an hAAT promoter, a TBG promoter, and an ASKA promoter. E9 comprises the AAV-ITR based gene delivery system of embodiment E7, wherein the tissue-specific promoter is a nervous system promoter selected from the group consisting of a NSE promoter, a PDGF promoter, a PDGF-β promoter, a Syn promoter, a MeCP2 promoter, a CaMKII promoter, a mGluR2 promoter, a NFL or NFH promoter, a flP2 promoter, a PPE promoter, an Enk promoter, an EAAT2 promoter, a GFAP promoter and a MBP promoter.

E10 comprises the AAV-ITR based gene delivery system of embodiment E4, wherein the promoter is a CMV promoter. E11 comprises the AAV-ITR based gene delivery system of embodiment E3, wherein the regulatory element comprises an enhancer. E12 comprises the AAV-ITR based gene delivery system of embodiment E11, wherein the enhancer is selected from the group consisting of a CMV enhancer, an SV40 enhancer, an AdMLP enhancer, and a polyoma enhancer. E13 comprises the AAV-ITR based gene delivery system of embodiment E11, wherein the enhancer is a CMV enhancer. E14 comprises the AAV-ITR based gene delivery system of embodiment E3, wherein the regulatory element comprises an intron. E15 comprises the AAV-ITR based gene delivery system of embodiment E14, wherein the intron is selected from the group consisting of an MVM intron, an FIX truncated intron 1, a β-globin SD/immunoglobulin heavy chain splice acceptor, an adenovirus splice donor/immunoglobin splice acceptor, a SV40 late splice donor/splice acceptor (19S/16S), and a hybrid adenovirus splice donor/IgG splice acceptor.

E16 comprises the AAV-ITR based gene delivery system of embodiment E3, wherein the regulatory element comprises a polyadenylation (Poly A) tail. E17 comprises the AAV-ITR based gene delivery system of embodiment E16, wherein the Poly A tail is selected from the group consisting of a Poly A of bovine growth hormone (BGH) mRNA, a rabbit beta-globin Poly A, and an SV40 Poly A. E18 comprises the AAV-ITR based gene delivery system of embodiment E17, wherein the Poly A tail is a BGH Poly A tail. E19 comprises the AAV-ITR based gene delivery system of embodiment E1, wherein the topology of the AAV-ITR vector corresponds to Schema I: [ITRL]-[E]-[P]-[I]-[GOI]-[P(A) signal]-[ITRR] wherein: ITRL and ITRR are ITRs, and ITRR is the reverse complement if ITRL; E is an enhancer; P is a promoter; I is an intron; GOI is a polynucleotide sequence encoding a therapeutic polypeptide, a therapeutic polynucleotide, or a combination thereof, and, P(A) signal is a polyadenylation signal. E20 comprises the AAV-ITR based gene delivery system of embodiment E1, wherein the Rep vector comprises a promoter.

E21 comprises the AAV-ITR based gene delivery system of embodiment E20, wherein the promoter is a T7 promoter. E22 comprises the AAV-ITR based gene delivery system of embodiment E1, wherein the Rep vector comprises a 5′ UTR. E23 comprises the AAV-ITR based gene delivery system of embodiment E22, wherein the 5′ UTR is selected from the 5′ UTR sequences of SEQ ID NOS: 388 to 405. E24 comprises the AAV-ITR based gene delivery system of embodiment E1, wherein the Rep vector comprises a 3′ UTR. E25 comprises the AAV-ITR based gene delivery system of embodiment E24, wherein the 3′ UTR is selected from the group consisting of 3′ UTR sequences of SEQ ID NOS: 406 to 423. E26 comprises the AAV-ITR based gene delivery system of embodiment E1, wherein the Rep vector comprises a Poly A tail. E27 comprises the AAV-ITR based gene delivery system of embodiment E26, wherein the Poly A tail is about 80 to about 250 nucleotides long. E28 comprises the AAV-ITR based gene delivery system of embodiment E26, wherein the Poly A tail is about 80, about 90, about 100, about 110, about 120, about 130, about 140, about 150, about 160, about 170, about 180, about 190, about 200, about 210, about 220, about 230, about 240 or about 250 nucleotides long.

E29 comprises the AAV-ITR based gene delivery system of embodiment E1, wherein the Rep vector comprises an mRNA encoding an AAV Rep protein. E30 comprises the AAV-ITR based gene delivery system of embodiment E29, wherein the AAV Rep protein comprises a Rep78 protein, a Rep68 protein, a variant thereof, or a functional fragment thereof. E31 comprises the AAV-ITR based gene delivery system of embodiment E30, wherein the AAV Rep protein is an AAV Rep78 protein from AAV1, AAV2, AAV3, AAV3B, AAV4, AAV5, AAV6, AAV7, AAV8, AAV9, AAV10, AAV 11, AAV12, or AAV13, a variant thereof, or a functional fragment thereof. E32 comprises the AAV-ITR based gene delivery system of embodiment E30, wherein the AAV Rep protein is an AAV Rep58 protein from AAV1, AAV2, AAV3, AAV3B, AAV4, AAV5, AAV6, AAV7, AAV8, AAV9, AAV10, AAV11, AAV12, or AAV13, a variant thereof, or a functional fragment thereof. E33 comprises the AAV-ITR based gene delivery system of embodiment E1, wherein the topology of the Rep vector corresponds to Schema II. [P]-[5′ UTR]-[Rep78/68]-[3′ UTR]-[P(A) tail] wherein: P is a promoter; 5′ UTR is a 5′ untranslated region; 3′ UTR is a 3′ untranslated region; Rep78/68 is a polynucleotide sequence encoding a Rep78 or Rep68 protein; and, P(A) tail is a polyadenylation tail.

E34 comprises the AAV-ITR based gene delivery system of embodiment E1, wherein the polynucleotide sequence encoding a therapeutic polypeptide, a therapeutic polynucleotide, or a combination thereof encodes a therapeutic polypeptide. E35 comprises the AAV-ITR based gene delivery system of embodiment E1, wherein the polynucleotide sequence encoding a therapeutic polypeptide, a therapeutic polynucleotide, or a combination thereof encodes a therapeutic polynucleotide. E36 comprises the AAV-ITR based gene delivery system of embodiment E1, wherein the polynucleotide sequence encoding a therapeutic polypeptide, a therapeutic polynucleotide, or a combination thereof encodes a therapeutic polypeptide and a therapeutic polynucleotide. E37 comprises the AAV-ITR based gene delivery system of embodiment E1, wherein the polynucleotide sequence encoding a therapeutic polypeptide, a therapeutic polynucleotide, or a combination thereof comprises at least two cistrons.

E38 comprises the AAV-ITR based gene delivery system of embodiment E37, wherein the polynucleotide sequence comprises an IRES element, a 2A element, or a combination thereof. E39 comprises the AAV-ITR based gene delivery system of embodiment E38, wherein the polynucleotide sequence comprises an IRES element interposed between two cistrons. E40 comprises the AAV-ITR based gene delivery system of embodiment E38, wherein the polynucleotide sequence comprises a 2A element interposed between two cistrons. E41 comprises the AAV-ITR based gene delivery system of embodiment E40, wherein the 2A element comprises a P2A element (porcine teschovirus-1 2A), a F2A element (foot-and-mouth disease virus), E2A element (equine rhinitis A virus), or T2A element (thosea asigna virus 2A). E42 comprises the AAV-ITR based gene delivery system of embodiment E38, wherein the polynucleotide sequence comprises a 2A element that is a tandem P2A-T2A element.

E43 comprises the AAV-ITR based gene delivery system of embodiment E38, wherein the polynucleotide sequence comprises a 2A element that is a tandem 2A-T2A-E2A element. E44 comprises the AAV-ITR based gene delivery system of embodiment E1, wherein the polynucleotide sequence encodes an antibody, an enzyme, a receptor, an ion channel, a vaccine antigen, a CAR, a hormone, a cytokine, a growth factor, or an apoptosis regulator. E45 comprises the AAV-ITR based gene delivery system of embodiment E44, wherein the polynucleotide sequence encodes an antibody. E46 comprises the AAV-ITR based gene delivery system of embodiment E45, wherein the antibody is monospecific, bispecific, trispecific, or tetraspecific. E47 comprises the AAV-ITR based gene delivery system of embodiment E45, wherein the antibody is an MSTAR antibody. E48 comprises the AAV-ITR based gene delivery system of embodiment E45, wherein the antibody is a neutralizing SARS-CoV2, influenza virus, or HIV virus antibody. E49 comprises the AAV-ITR based gene delivery system of embodiment E1, wherein the polynucleotide sequence encodes GLP-1 and/or a GLP-1 agonist. E50 comprises the AAV-ITR based gene delivery system of embodiment E49, wherein the GLP-1 is GLP-1 (7-37).

E51 comprises the AAV-ITR based gene delivery system of embodiment E49, wherein the GLP-1 agonist is selected from exenatide, liraglutide, lixisenative, taspoglutide, albiglutide, dulaglutide, emaglutide, and semaglutide. E52 comprises the AAV-ITR based gene delivery system of embodiment E1, wherein the wherein the polynucleotide sequence encodes a GIP agonist. E53 comprises the AAV-ITR based gene delivery system of embodiment E1, wherein the polynucleotide sequence encodes a vaccine antigen. E54 comprises the AAV-ITR based gene delivery system of embodiment E53, wherein the vaccine antigen is a viral, bacterial, or protozoan protein or fragment or variant thereof to treat COVID-19 (SARS-CoV2 infection), HIV infection, influenza, RSV infection, rabies, HPV infection, malaria, EBV infection, tuberculosis, CMV infection, Herpes zoster, Zika virus infection, HBV infection, yellow fever, PIV infection, hMPV infection, rotavirus infection, Nipah virus infection or Chikungunya virus infection. In some aspects, the dsDNA encodes a neutralizing antibody to treat, e.g., COVID-19 (SARS-CoV2 infection), HIV infection, influenza, RSV infection, rabies, HPV infection, malaria, EBV infection, tuberculosis, CMV infection, Herpes zoster, Zika virus infection, HBV infection, yellow fever, PIV infection, hMPV infection, rotavirus infection, Nipah virus infection or Chikungunya virus infection. E55 comprises the AAV-ITR based gene delivery system of embodiment E1, wherein the polynucleotide sequence encodes a gene product for replacement therapy selected from the group consisting of AAT, ABCB4, ACADVL, ARG1, ATP8B1, C2, CFTR, citrin, CNTF, CPS1, CTNS, G6PT1, GALT, GLA, HPRT, JAG1, LPL, MCAD, MCM, PBGD, REP1, RPE65, RS1, and TTR. E56 comprises the AAV-ITR based gene delivery system of embodiment E1, wherein the polynucleotide sequence encodes the gene of interest in a gene therapy agent selected from the group consisting of alipogene tiparvovec (GLYBERA™), atidarsagene autotemcel (LIBMELDY™), axicabtagene ciloleucel (YESCARTA™), beremagene geperpavec (VYJUVEK™), betibeglogene autotemcel (ZYNTEGLO™), brexucabtagene autoleucel (TECARTUS™), cambiogenplasmid (NEOVASCULGEN™), ciltacabtagene autoleucel (CARVYKTI™), delandistrogene moxeparvovec (ELEVIDYS™), elivaldogene autotemcel (SKYSONA™), etranacogene dezaparvovec (HEMGENIX™), gendicine, vicleucel (ABECMA™), lisocabtagene maraleucel (BREYANZI™), nadofaragene firadenovec (ADSTILADRIN™), onasemnogene abeparvovec (ZOLGENSMA™), strimvelis, talimogene laherparepvec (IMLYGIC™), tisagenlecleucel (KYMRIAH™), valoctocogene roxaparvovec (ROCTAVIAN™), and voretigene neparvovec (LUXTURNA™).

E57 comprises the AAV-ITR based gene delivery system of any one of embodiments E1 to E56, wherein the AAV-ITR vector and Rep vector are encapsulated in a lipidic delivery system, polymeric delivery system, or a combination thereof. E58 comprises the AAV-ITR based gene delivery system of embodiment E57, wherein the lipidic delivery system comprises a cationic or ionizable lipid or lipidoid; a structural lipid; a helper lipid; a stabilizing lipid; or, a combination thereof. E59 comprises the AAV-ITR based gene delivery system of embodiment E58, wherein the ionizable lipid or lipidoid is selected from the group consisting of cKK-E12, AIC-0315, SM-102, YK-009, DLin-MC3-DMA (MC3), DLin-KC2-DMA (KC2), A6, OF-02, A18-Iso5-2DC18, 98N12-5, 9A1p9, C12-200, 7C1, G0-C14, L319, 304O13, OF-Deg-Lin, 306-O12B, 306O110, FTT5, Lipid 10, and combinations thereof. E60 comprises the AAV-ITR based gene delivery system of embodiment E58, wherein the cationic lipid is DOTAP or DOTMA. E61 comprises the AAV-ITR based gene delivery system of embodiment E58, wherein the structural lipid is a sterol.

E62 comprises the AAV-ITR based gene delivery system of embodiment E61, wherein the sterol is selected from the group consisting of cholesterol, beta-cholesterol, ergosterol, 7-dehydrocholesterol, 24S-hydroxycholesterol, lanosterol, cycloartenol, fucosterol, saringosterol, campesterol, 0-sitosterol, sitostanol, coprostanol, avenasterol, stigmasterol, and any combination thereof. E63 comprises the AAV-ITR based gene delivery system of embodiment E61, wherein the sterol is cholesterol. E64 comprises the AAV-ITR based gene delivery system of embodiment E58, wherein the helper lipid comprises at least one symmetric phospholipid, asymmetric lipid, lysolipid, fatty acid, or a combination thereof. E65 comprises the AAV-ITR based gene delivery system of embodiment E64, wherein the symmetric phospholipid comprises a phosphocholine (PC). E66 comprises the AAV-ITR based gene delivery system of embodiment E65, wherein the PC is selected from the group consisting of DSPC, DOPC, DLPV, DMPC, and any combination thereof. E67 comprises the AAV-ITR based gene delivery system of embodiment E64, wherein the symmetric phospholipid comprises a phosphoethanolamine (PE).

E68 comprises the AAV-ITR based gene delivery system of embodiment E67, wherein the PE is the selected from the group consisting of DSPE, DOPE, DLPE, DMPE, and a combination thereof. E69 comprises the AAV-ITR based gene delivery system of embodiment E58, where the stabilizing lipid comprises a PEG-lipid. E70 comprises the AAV-ITR based gene delivery system of embodiment E69, where the PEG-lipid is a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, or a combination thereof. E71 comprises the AAV-ITR based gene delivery system of embodiment E69, wherein the PEG-lipid is selected from the group consisting of PEG-c-DOMG, PEG-DMG, PEG-DLPE, PEG-DMPE, PEG-DPPC, PEG-DSPE, and any combination thereof. E72 comprises the AAV-ITR based gene delivery system of embodiment E69, wherein PEG-lipid is selected from the group consisting of DMG-PEG2000, DSPE-PEG2000, and a combination thereof. E73 comprises the AAV-ITR based gene delivery system of any one of embodiments E58 to E72, wherein the lipidic delivery system comprises a lipid nanoparticle, a liposome, or a combination thereof. E74 comprises the AAV-ITR based gene delivery system of embodiment E73, wherein the liposome comprises LIPOFECTAMINE™ 2000, LIPOFECTAMINE™ MESSENGERMAX™, LIPOFECTAMINE™ 3000, LIPOFECTAMINE™ RNAiMAX, LIPOFECTINE™, LIPOFECTAMINE™ LTX, LIPOFECTAMINE™ Stem Transfection Reagent, LIPOFECTAMINE™ CRISPRMAX™ Cas9 Transfection Reagent, VIAFECT™ TRANSTAST™, INVIVOFECTAMINE™ 3.0, MIRUS BIO™ TRANSIT™-2020, or GIBCO™ EXPIFECTAMINE™.

E75 comprises the AAV-ITR based gene delivery system of any one of embodiments E1 to E74, wherein the AAV-ITR vector is encapsulated in a liposome or lipid nanoparticle. E76 comprises the AAV-ITR based gene delivery system of any one of embodiments E1 to E75, wherein the Rep vector is encapsulated in a liposome or lipid nanoparticle. E77 comprises the AAV-ITR based gene delivery system of any one of embodiments E1 to E76, wherein the AAV-ITR vector is encapsulated in a first liposome and the Rep vector is encapsulated in a second liposome. E78 comprises the AAV-ITR based gene delivery system of any one of embodiments E1 to E74, wherein the AAV-ITR vector is encapsulated in a first LNP and the Rep vector is encapsulated in a second LNP. E79 comprises the AAV-ITR based gene delivery system of embodiment E77 or E78, wherein the first liposome or LNP and the second liposome or LNP have the same composition. E80 comprises the AAV-ITR based gene delivery system of embodiment E77 or E78, wherein the first liposome or LNP and the second liposome or LNP have different compositions.

E81 comprises the AAV-ITR based gene delivery system of any one of embodiments E1 to E80, wherein the AAV-ITR vector is encapsulated in a LNP and the Rep vector is encapsulated in a liposome. E82 comprises the AAV-ITR based gene delivery system any one of embodiments E1 to E80, wherein the AAV-ITR vector is encapsulated in a liposome and the Rep vector is encapsulated in a LNP. E83 comprises the AAV-ITR based gene delivery system of embodiment E81 or E82, wherein the liposome and LNP have the same composition. E84 comprises the AAV-ITR based gene delivery system of embodiment E81 or E82, wherein the liposome and LNP have different compositions. E85 comprises the AAV-ITR based gene delivery system of any one of embodiments E1 to E84, wherein the AAV-ITR vector and Rep vector are encapsulated in the same liposome or LNP. E86 comprises the AAV-ITR based gene delivery system of any one of embodiments E1 to E85, wherein the delivery system is targeted to bone marrow cells, hematopoietic stem cells (HSCs), hematopoietic stem and progenitor cells (HSPCs), peripheral blood mononuclear cells (PBMCs), myeloid progenitor cells, lymphoid progenitor cells, T-cells, B-cells, NKT cells, NK cells, dendritic cells, monocytes, granulocytes, erythrocytes, megakaryocytes, mast cells, basophils, eosinophils, neutrophils, macrophages, erythroid progenitor cells (e.g., HUDEP cells), megakaryocyte-erythroid progenitor cells (MEPs), common myeloid progenitor cells (CMPs), multipotent progenitor cells (MPPs), hematopoietic stem cells (HSCs), short term HSCs (ST-HSCs), IT-HSCs, long term HSCs (LT-HSCs), endothelial cells, neurons, astrocytes, pancreatic cells, pancreatic 3-islet cells, liver cells, muscle cells, skeletal muscle cells, cardiac muscle cells, hepatic cells, fat cells, intestinal cells, cells of the colon, or cells of the stomach.

E87 comprises the AAV-ITR based gene delivery system of embodiment E86, wherein the delivery system is targeted using a targeting molecule. E88 comprises the AAV-ITR based gene delivery system of embodiment E87, wherein the targeting molecule is an antibody. E89 comprises the AAV-ITR based gene delivery system of embodiment E88, wherein the antibody is an M-STAR antibody. E90 comprises the AAV-ITR based gene delivery system of embodiment E99, wherein the targeting molecule is covalently attached to a lipidic delivery system or polymeric delivery system co-encapsulating the AAV-ITR vector and Rep vector. E91 comprises a method of replicating a polynucleotide encoding a therapeutic polypeptide, therapeutic polynucleotide or combination thereof in vivo in a patient in need of treatment for a disease or condition, comprising co-delivering the AAV-ITR based gene delivery system of any one of embodiments E1 to E90 to the patient to transiently express the Rep protein encoded by the Rep vector to replicate and amplify the polynucleotide encoding the therapeutic polypeptide, therapeutic polynucleotide or combination encoded by the AAV-ITR vector.

E92 comprises a method of method of delivering a therapeutic polypeptide, therapeutic polynucleotide or combination thereof in vivo to a patient in need of treatment for a disease or condition, comprising co-delivering the AAV-ITR based gene delivery system of any one of embodiments E1 to E90 to the patient, wherein the Rep protein encoded by the Rep vector is expressed transiently and replicates and amplifies the polynucleotide encoding the therapeutic polypeptide, therapeutic polynucleotide or combination encoded by the AAV-ITR vector, and wherein the expression of the therapeutic polypeptide, therapeutic polynucleotide or combination thereof treats the disease or condition in the patient. E93 comprises a method the treat a disease or condition in a subject in need thereof, comprising co-delivering the AAV-ITR based gene delivery system of any one of embodiments E1 to E90 to the patient, wherein the Rep protein encoded by the Rep vector is expressed transiently and replicates and amplifies the polynucleotide encoding the therapeutic polypeptide, therapeutic polynucleotide or combination encoded by the AAV-ITR vector, and wherein the expression of the therapeutic polypeptide, therapeutic polynucleotide or combination thereof treats the disease or condition in the patient.

E94 comprises a therapeutic polypeptide, therapeutic polynucleotide or combination produced in vivo or ex vivo by co-delivering the AAV-ITR based gene delivery system of any one of embodiments E1 to E90 to a host cell, wherein the Rep protein encoded by the Rep vector is expressed transiently and replicates and amplifies the polynucleotide encoding the therapeutic polypeptide, therapeutic polynucleotide or combination encoded by the AAV-ITR vector, and wherein the cell expresses the therapeutic polypeptide, therapeutic polynucleotide or combination thereof. E95 comprises a set of vectors comprising (i) a linear dsDNA AAV-ITR vector comprising 5′ and 3′ AAV ITR flanking a polynucleotide sequence encoding a therapeutic polypeptide, a therapeutic polynucleotide, or a combination thereof, and, (ii) a linear mRNA Rep vector encoding a Rep protein. E96 comprises the set of vectors of embodiment E95, wherein the vectors are co-encapsulated in a lipidic or polymeric delivery system. E97 comprises the set of vectors of embodiment E96, wherein the lipidic or polymeric delivery system is targets to a specific cell or tissue. E98 comprises a host cell comprising (i) a linear dsDNA AAV-ITR vector comprising 5′ and 3′ AAV ITR flanking a polynucleotide sequence encoding a therapeutic polypeptide, a therapeutic polynucleotide, or a combination thereof, and, (ii) a linear mRNA Rep vector encoding a Rep protein. E99 comprises a pharmaceutical composition comprising the AAV-ITR based gene delivery system of any one of embodiments E1 to E90 and a pharmaceutically acceptable excipient. E100 comprises a pharmaceutical composition comprising the therapeutic polypeptide, therapeutic polynucleotide or combination thereof of embodiment E94, set of vectors of embodiments E95 to E97, or host cell of embodiment E98, and a pharmaceutically acceptable excipient. E101 comprises a lipid complex mixture suitable for co-encapsulation of an AAV-ITR vector and a Rep vector of any one of embodiments E95 to E97. E102 comprises a kit comprising (i) a linear dsDNA AAV-ITR vector comprising 5′ and 3′ AAV ITR flanking a polynucleotide sequence encoding a marker; and, (ii) a linear mRNA Rep vector encoding a Rep protein, and instructions to replace the marker with a polynucleotide encoding a therapeutic polypeptide, a therapeutic polynucleotide, or a combination thereof. E103 comprises a kit comprising (i) lipid complex mixture suitable for co-encapsulation of an AAV-ITR vector and a Rep vector; and (ii) instructions to co-encapsulate the vectors.

### Examples

### Materials and Methods

Cell Culture and In Vitro Assay. C2C12 cells were cultured in Dulbecco's Modified Eagle Medium (DMEM) supplemented with 10% fetal bovine serum (FBS) (Optima, Cat #512450H). C2C12 cell line was purchased from ATCC and used at passage under 20. Transfection was performed using Lipofectamine 2000 (Invitrogen, Cat #11668019) for AAV2 ITR-EGFP DNA and Lipofectamine MessengerMax (Invitrogen, Cat #LMRNA003) for Rep78 according to the User's manual.

Briefly, AAV2 ITR-EGFP linear DNA fragment was mixed with Lipofectamine 2000 reagent at 1:1 ratio in serum free medium. Rep78 mRNA was mixed with Lipofectamine MessengerMax reagent at 1:1 ratio in serum free medium. After 5 minutes incubation, complexed AAV ITR-EGFP and complexed Rep78 mRNA were mixed with different ratio, then added to C2C12 cells accordingly. DNA/mRNA lipid complexes were removed 24 hours post transfection and transfected cells were maintained as usual with regular split at confluence. EGFP expression was measured using flow cytometry and qPCR at indicated time points.

DNA, RNA Isolation and qPCR. Genomic DNA was isolated with the PureLink Genomic DNA kit (Invitrogen, Cat #K1820-00) according to the manufacturer's manual. Episomal DNA was isolated by using Hirt Supernatant DNA preparation protocol. Total RNA was isolated with the ReliaPrep™ RNA Miniprep Systems Kit (Promega, Cat #Z6011) according to the manufacturer's manual, followed by cDNA synthesis using Verso cDNA Synthesis Kit (Thermo Scientific, Cat #AB-1453). For qPCR, PowerUp Syber Green Master Mix (Applied Biosystems, Cat #A25742) was used according to the manufacturer's protocol. The PCR program used for amplification was 1) 95° C. for 10 min, 2) 95° C. for 10 s, 3) 60° C. for 30 s, 4) 72° C. for 30 s. Steps 2 to 4 were repeated for 40 cycles to amplify EGFP and GAPDH. The primers for EGFP were forward primer, 5′-cactacctgagcacccagtc-3′ (SEQ ID NO: 728), and reverse primer, 5′-tacagctcatccatgccgag-3′ (SEQ ID NO: 729). The primers for GAPDH were forward primer, 5′-ggagtcaacggatttggtcg-3′ (SEQ ID NO: 730), and reverse primer, 5′-gacggtgccatggaatttgc-3′ (SEQ ID NO: 731). One-way ANOVA was used to determine statistical significance in qPCR analysis. P<0.05 was considered as statistically significant

Western blot analysis. 100 ng of complexed AAV ITR-EGFP was added into each well of the 96-well-dish with indicated amount of Rep78 mRNA. The level of Rep78 protein was measured in AAV ITR-EGFP, AAV ITR-EGFP with Rep78 mRNA transfected C2C12 cells. whole-cell lysates were harvested by 2×SDS sample buffer. Proteins were separated in 7-12% SDS-polyacrylamide gel, transferred to a nitrocellulose membrane, probed with indicated antibodies, followed by detection with enhanced chemiluminescence and visualized by iBright.

Single Cell Sorting and Whole Genome Sequencing. Week 8 cultures from AAV ITR-EGFP lipid complexes alone transfected C2C12 cells and AAV ITR-EGFP with 3.5% of Rep78 mRNA lipid complexes mixture transfected C2C12 cells were chosen to performed single cell sorting. The samples were prepared for samples according to the recommended procedure (Sony, model LE-MA900BP). Briefly, the sorting chamber was wiped down with DI water. Following the directions of the Sony MA-900 software, a new 100 uM chip was placed, followed by startup and then autocalibration using the automatic setup beads. The 96 well plate sorting was calibrated to ensure droplets end up in the corresponding wells. The cells were washed with PBS, placed into a 15 mL tube, and loaded into the chamber. The cells were acquired and then gated on the main population by FSC-A vs BSC-A to gate out debris and then on EGFP positive cells. EGFP positive cells were sorted into one cell/well in a 96 well plate which contained pre-conditioned media. Sorted single cells were cultured in Dulbecco's Modified Eagle Medium (DMEM) supplemented with 10% fetal bovine serum (FBS) (Optima, Cat #S12450H). 3 individual clones from each group were chosen and performed whole genome sequencing. The whole genome sequencing was performed by Azenta (Burlington, MA). The genomic DNAs were prepared from frozen cell pellets and fragmented for sequencing. The FASTQ data sets from sequencing were aligned against Genome data base and annotated variants. In addition, EGFP sequence from the ITR-EGFP vector was aligned against the genome sequences. No EGFP sequences were found in any genomic DNA fragments.

### Example 1

Design of AAV ITR_GOI Vector DNA and Rep68/78 mRNA

A linear AAV-ITR vector (AAV ITR_GOI DNA) containing a left side AAV2 ITR/Rep78 binding site (Ori)/terminal resolution site (TRS), a CMV IE enhancer/promoter/intron (as splicing donor) and a HIV-1 R region (as splicing acceptor), a gene of interest (GOI) with a polylinker, a bovine growth hormone polyA (Tbgh), followed by a right AAV2 ITR was synthesized and cloned into a pUC57 vector. The AAV ITR_GOI DNA was propagated in E. coli SURE2 strain. The plasmid DNA sequence was confirmed by sequencing. For in vitro and in vivo experiments, the AAV ITR_GOI dsDNA plasmid was digested using PvuII.

In vitro transcribed (IVT) vectors expressing AAV2 Rep68 or Rep78 mRNAs were made by cloning the synthesized AAV2 Rep68 and Rep78 genes into the standard IVT plasmid using SwaI/SpeI restriction sites. Linearized IVT vector was used for in vitro transcription using standard procedure and mRNA generated was purified using the MEGAclear Transcription Clean-Up Kit. The purified mRNA then was aliquoted and stored at −80° C. for future use.

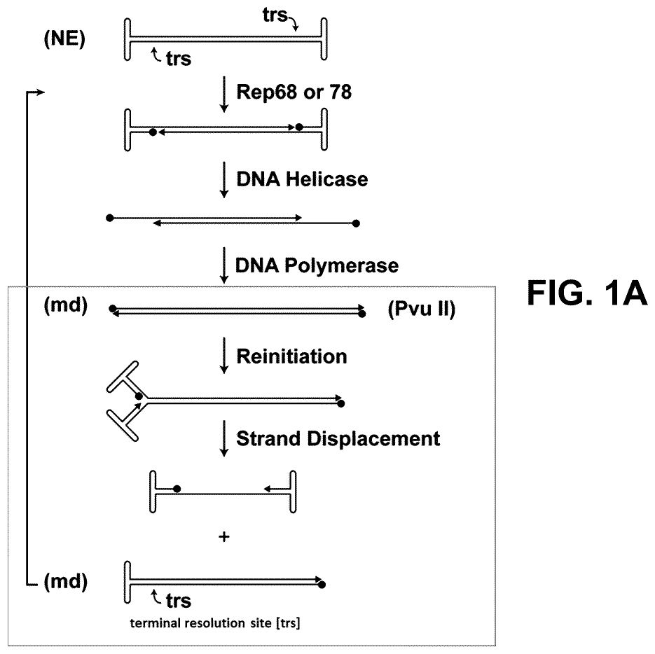

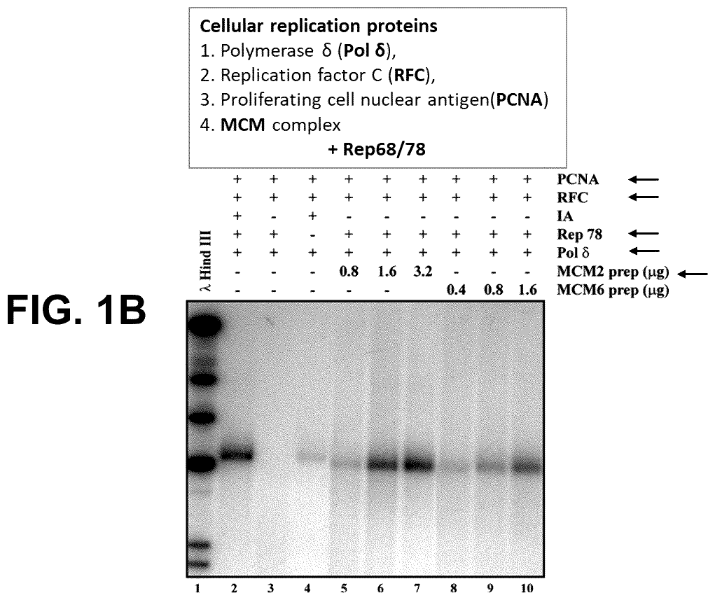

AAV replication is initiated by Rep68/78 protein (FIG. 1A). Replication assays confirmed that AAV ITR constructs can be replicated when providing Rep68/78 mRNA to dividing cells expressing Pol δ, RFC, PCNA, and MCM complex (FIG. 1B).

FIG. 2A shows an AAV-ITR linear DNA vector comprising a nucleic acid encoding a gene of interest and FIG. 2B shows a Rep78 IVT mRNA vector. The AAV-ITR linear DNA vector contains the following elements
(1) Left side AAV2 ITR/Rep78 binding site (Ori)/terminal resolution site (TRS);
(2) CMV IE enhancer/promoter/intron (as splicing donor) and HIV-1 R region (as splicing acceptor);
(3) Gene of Interest (GOI) with polylinker;
(4) Bovine growth hormone polyA (Tbgh),
(5) Right side AAV2 ITR.

The AAV-ITR linear DNA vector was synthesized and cloned into pUC57 vector, followed by propagation in E. coli SURE2 strain. Plasmid DNA sequence was further confirmed by sequencing. The dsDNA plasmid was digested using PvuII to releasing the AAV ITR dsDNA fragment containing the gene of interest for in vitro and in vivo experiments. IVT vectors for making AAV2 Rep68 or Rep78 mRNAs were made by cloning synthesized AAV2 Rep68 and Rep78 genes into the standard IVT plasmid using SwaI/SpeI. Linearized IVT vector was used for in vitro transcription using standard procedure and purified using the MEGAclear Transcription Clean-Up Kit. The purified mRNA then was aliquoted and stored at −80° C. for future use.

### Example 2

### Selection of in Vitro Delivering Vehicle: Liposome or LNPs

Current LNPs have not been shown to be good delivery vehicles for intramuscular (IM) administration due to their intrinsic EpoE binding properties and LDLR mediated entry into LDLR expressing cells. Muscle cells lack LDLR expression resulting in low entry delivered by LNPs. Antibody mediated targeting to muscle cells can be a viable approach but requires identification of muscle cell surface proteins as targets. Cationic lipid based liposomes and polymer-based nanoparticles have a long history for transfection of various cell lines in vitro with great efficiency. However, their application in vivo is hampered by toxicity and induction of immune responses against a transgene they carry due to the proinflammatory nature of the lipids and polymers. Furthermore, the presence of viral Rep proteins in cells can contribute to a proinflammatory response. Therefore, provided herein are approaches to limit proinflammatory responses induced by viral Rep proteins. In one approach, the time period during which Rep 68/78 protein is present in a target cell is limited by providing Rep 68/78 encoding nucleic acids as RNAs.

### Example 3

### In Vitro Transgene Expression

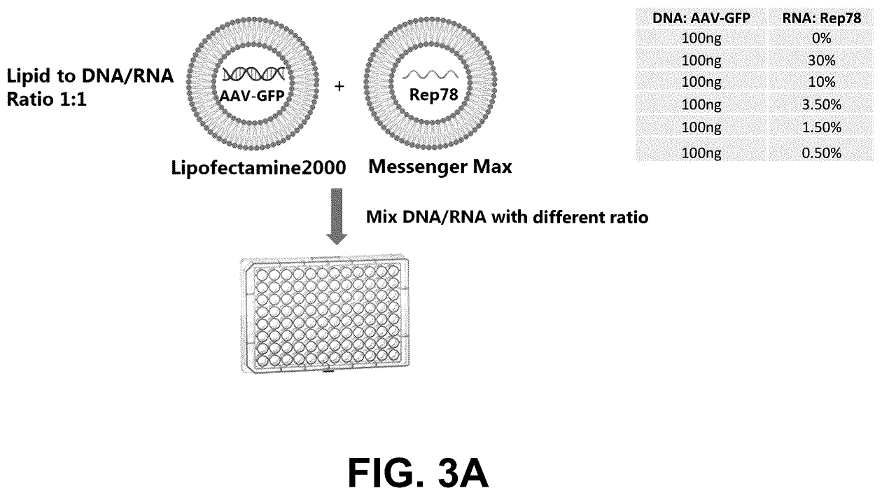

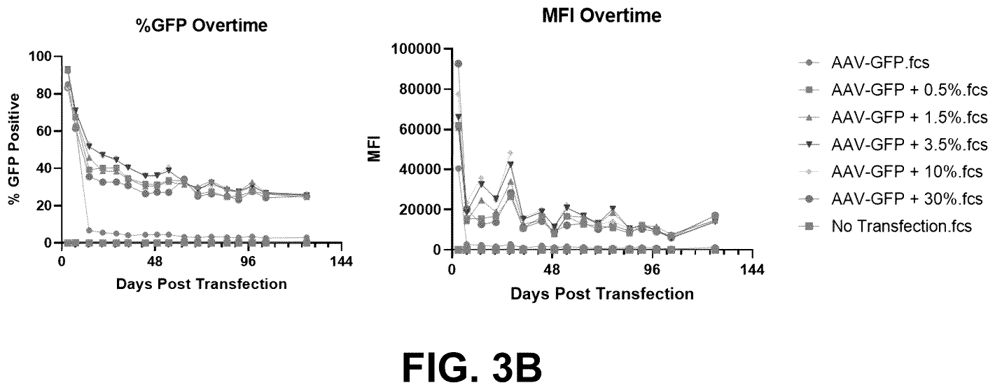

FIG. 3A is schematic diagram indicating how the AAV ITR-EGFP lipid complexes and Rep78 mRNA lipid complexes were formed and transfected to C2C12 cells with different ratios. 100 ng of complexed AAV ITR-EGFP was added into each well of the 96-well-dish with indicated amount of Rep78 mRNA.

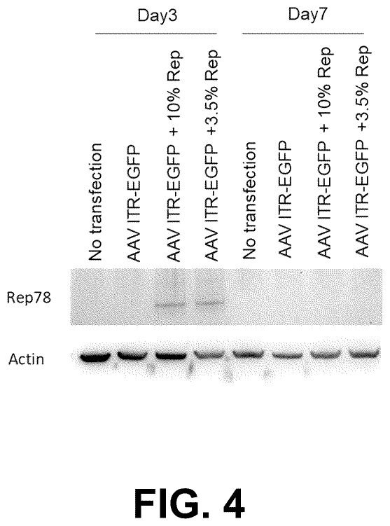

EGFP expression was measured using flow cytometry at the indicated time points. Percentage of GFP+ cells was plotted both as percentage of GFP+ cells over time and medium fluorescent intensity (MFI) over time. ITR_EGFP dsDNA co-transfected with Rep78 mRNA results in sustained GFP protein expression in C2C12 mouse myoblast cells (FIG. 3B). ITR_EGFP dsDNA co-transfected with Rep78 mRNA resulted in sustained GFP protein expression in 3T3 embryonic fibroblasts cells (FIG. 4). 100 ng of complexed AAV ITR-EGFP was added into each well of the 96-well-dish with indicated amount of Rep78 mRNA. The level of Rep78 protein was measured in AAV ITR-EGFP, AAV ITR-EGFP with Rep78 mRNA transfected C2C12 cells. whole-cell lysates were harvested by 2×SDS sample buffer. Proteins were separated in 7-12% SDS-polyacrylamide gel, transferred to a nitrocellulose membrane, probed with indicated antibodies, followed by detection with enhanced chemiluminescence and visualized by iBright.

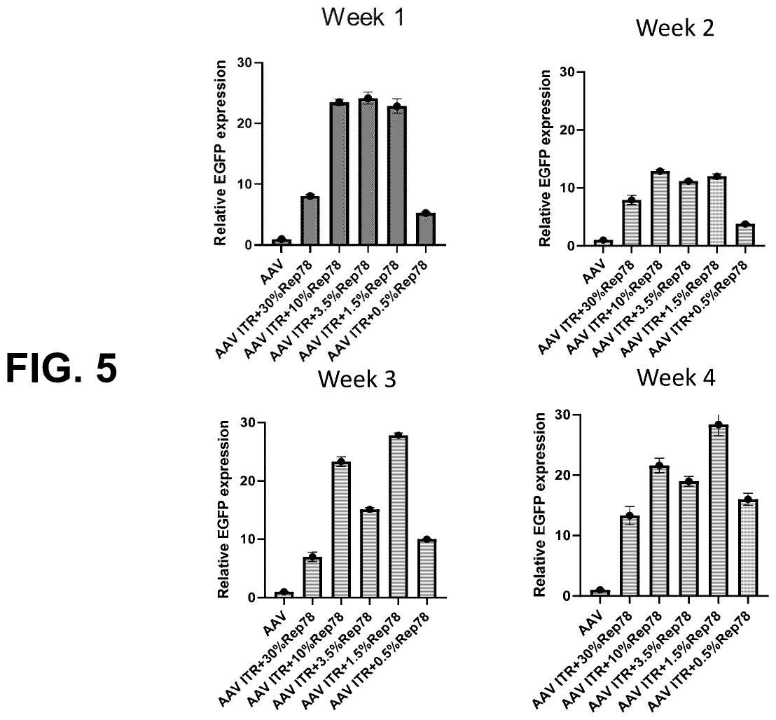

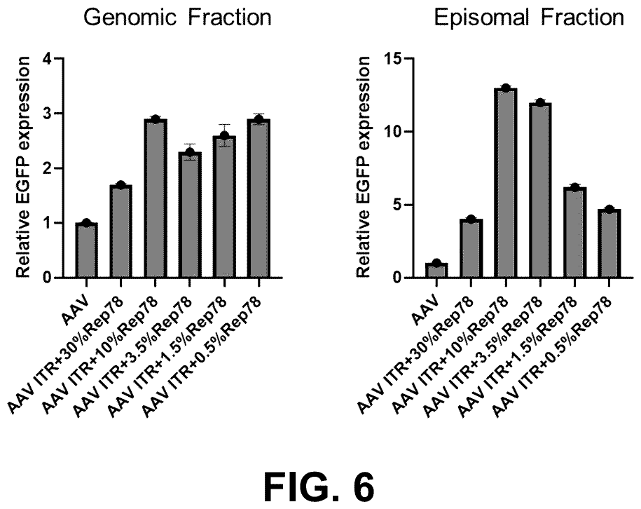

Quantitative analysis of EGFP mRNA level and ITR-EGFP DNA level from C2C12 cells co-transfected with ITR-EGFP dsDNA and Rep78 mRNA is shown in FIG. 5 and FIG. 6. C2C12 cells were co-transfected using AAV ITR-EGFP lipid complexes and Rep78 mRNA lipid complexes as previous described at different ratios. Total mRNA, genomic DNA and episomal fraction of DNA were prepared as described in the Material and Methods section. En FIG. 5, the level of EGFP RNA was measured using qPCR to detect EGFP mRNA as described in the Material and Methods section. In FIG. 6 genomic and episomal DNA fractions were also quantitated using qPCR to detect the EGFP gene.

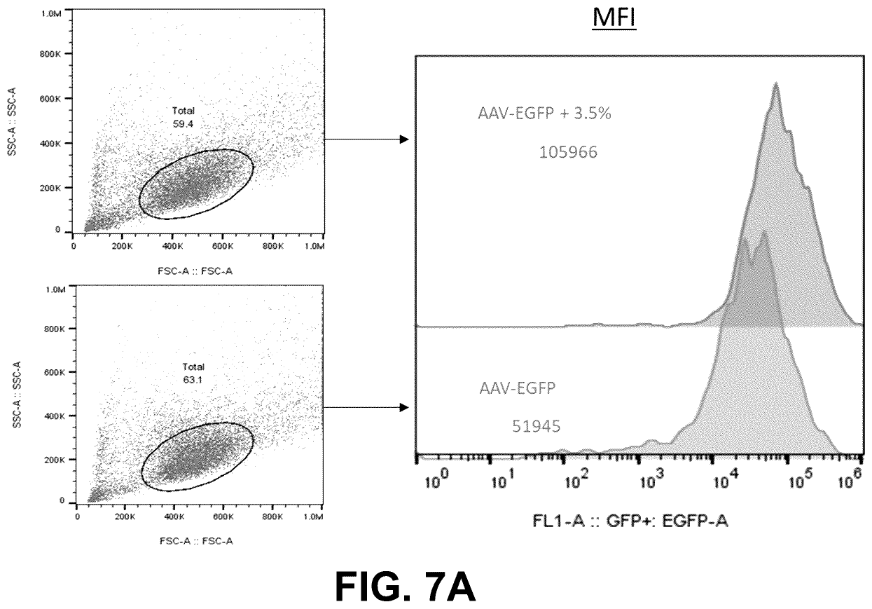

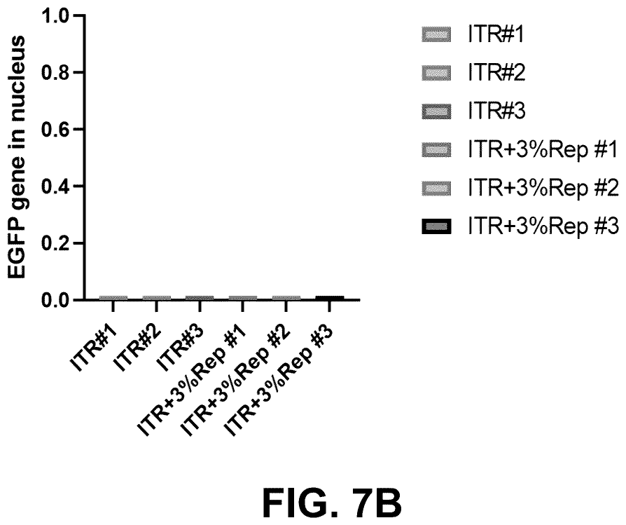

Integration of ITR-EGFP DNA into the host cell genome was analyzed using whole genome sequencing (FIG. 7A and FIG. 7B). FIG. 7A shows AAV ITR-EGFP+lipid complexes and different ratios of Rep78 mRNA+lipid complexes co-transfected into C2C12 cells. EGFP positive cells were selected and single cell sorting was performed. Three individual clones from each group were chosen used to conduct whole genome sequencing.

FIG. 7B shows whole genome sequencing results indicating that in none of the individual clones the EGFP gene was located in the cell nucleus fraction. Thus, AAV ITR was not integrated into a chromosome.

In summary, AAV Rep78 expressed from mRNA had a short time of expression, which is important to avoid immune response when applied in vivo to an animal model or to a human subject. But transient expression of Rep78 induced significant level of linear ITR_GOI vector replication, helping establish 10-fold higher persistent transgene expression through episomal maintenance or increased chromosomal integration compared to linear ITR_GOI vector DNA alone. Higher persistent transgene expression resulted in higher total recombinant therapeutic protein expression in vitro. When different ratios of AAV ITR_GFP DNA to Rep78 RNA were used, highest transduction efficiencies were observed when ratios of Rep78 vector to AAV ITR_GFP DNA vector between 1.5% and 10% was present in DNA/RNA lipid preparations. Thus, co-delivery of AAV Rep78 mRNA with linear ITR_GOI vector can be a robust gene delivery system for expression of therapeutic protein in vivo through ex vivo or in vivo delivery.

### Example 4

### In Vivo Transgene Expression

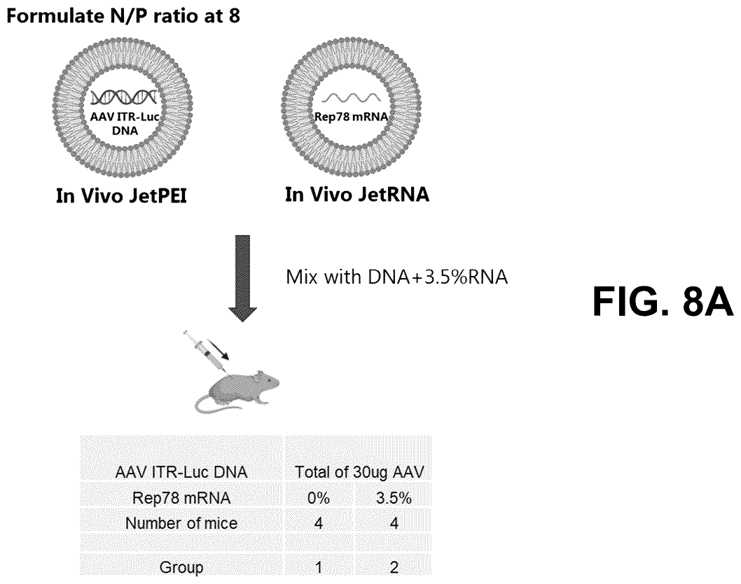

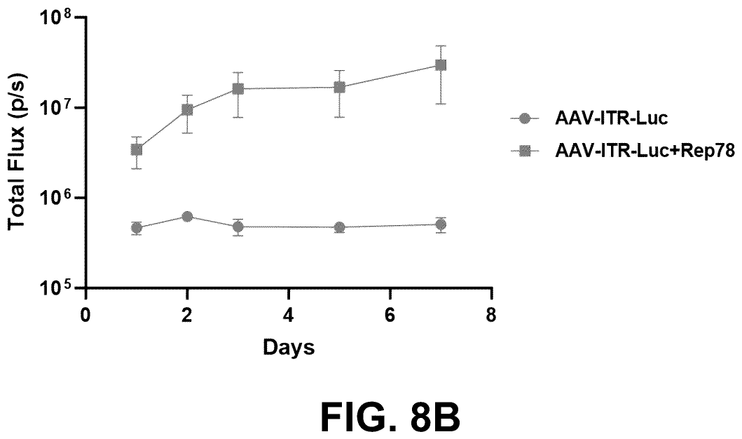

In vivo delivery was performed using in vivo-jetPEI (Polyplus, Cat #101000040) for AAV2 ITR-luciferase DNA and in vivo-jetRNA (Polyplus, Cat #101000122) for Rep78 according to the User's manual. Briefly, AAV2 ITR-luciferase linear DNA fragment and in vivo-jetPEI reagent were diluted in 5% glucose separately, then mixed AAV2 ITR-luciferase linear DNA fragment and in vivo-jetPEI® reagent together at N/P ratio 8. Rep78 mRNA was mixed in vivo-jetRNA® reagent at 1:2 ratio in mRNA buffer. After 15 minutes incubation at RT, 3.5% of complexed Rep78 were mixed with complexed AAV ITR-luciferase and injected to 6-week-old BALB/c mice (Charles River) mice through intramuscular route (IM). 50 ul of DNA/(mRNA)/jetPEI complex containing 15 ug DNA was injected into each leg. Each mouse received total of 30 ug DNA with or w/o 3.5% (w/w) of Rep78 mRNA/jetPEI complex. See FIG. 8A. Following the IM injection of the test material, mice were imaged in the IVIS system in a timely dependent manner. Briefly, mice were administered D-luciferin at 150 mg/kg intraperitoneally and were imaged 10 minutes post-injection. All images were processed for total flux and each animal with whole body ROI. All animals and user protocols were conducted in compliance with CRL IACUC under IACUC No. 1035. See FIG. 8B.

Results indicated robust Luciferase expression when liposomes containing an AAV2-ITR vector comprising the luciferase gene were co-administered with liposomes containing a Rep78 vector comprising an mRNA encoding AAV2 Rep78. Thus, the in vivo data conformed that co-delivery of AAV Rep78 mRNA with linear a AAV-ITR vector encoding a GOI can be a robust gene delivery system for expression of therapeutic protein in vivo through ex vivo or in vivo delivery.

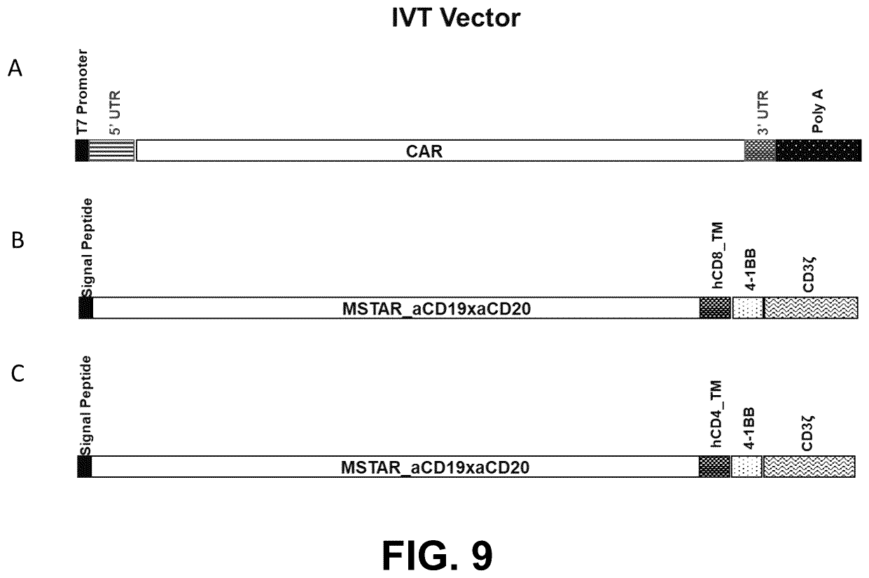

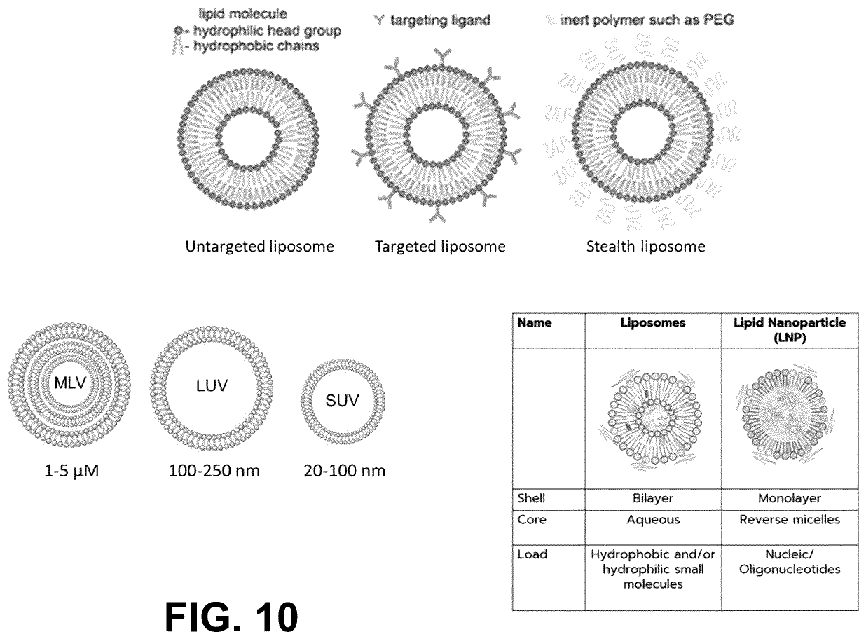

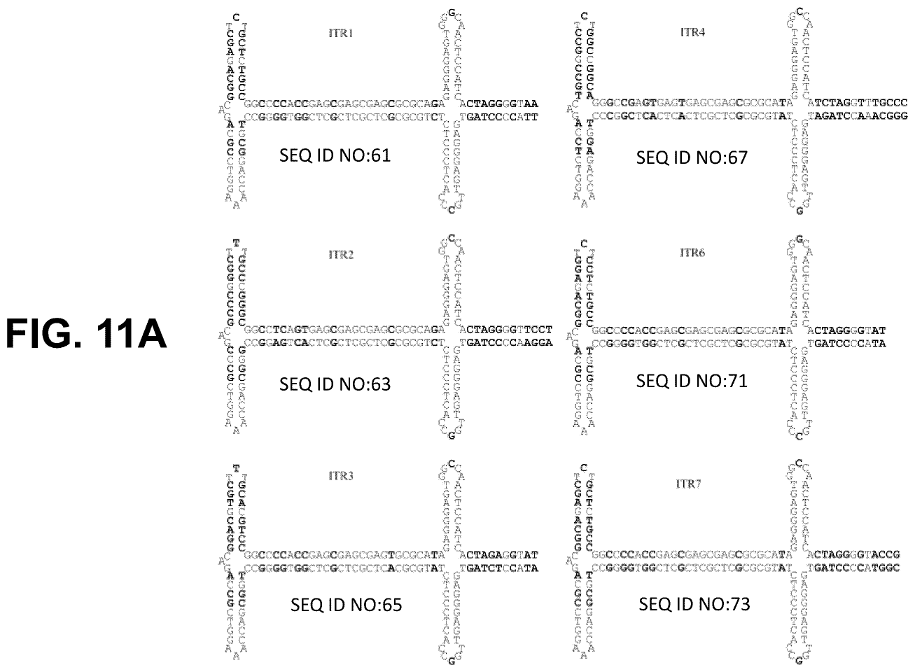

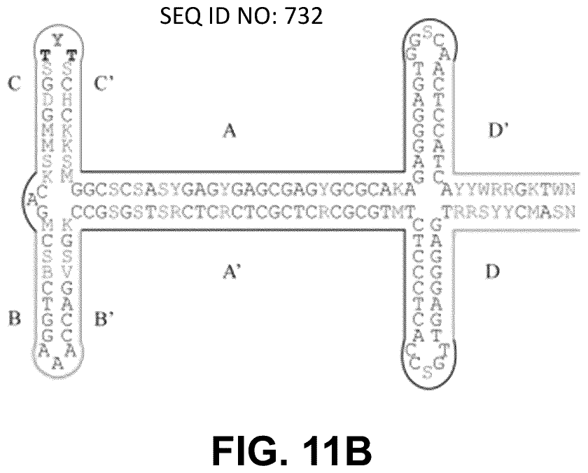

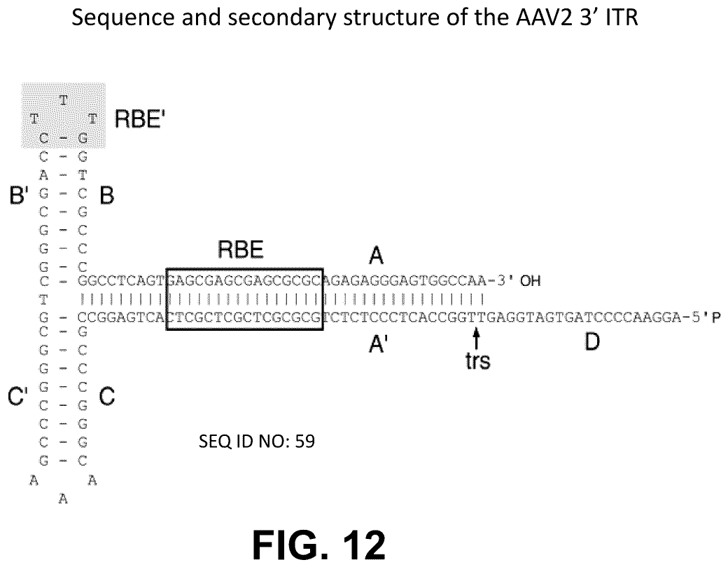

TABLE 11
Exemplary construct sequences.

| | Molecular design / Protein name | Polypeptide | Encoding |
|---|---|---|---|
| Construct | Description | Chains | Polynucleotides |
| MX1954 | STAR_ahCD19_hB43xahCD20_ofa_CD8BBZ | SEQ ID | SEQ ID |
| | See FIG. 9, structure B | NO: 1 | NO: 18 |
| | Construct encoding a bispecific aCD19/aCD20 | | |
| | CAR comprising a CD8-derived spacer and TM | | |
| | domain, and an IC domain comprising 4-1BB | | |
| | and CD3ζ. | | |
| MX1955 | STAR_ahCD19_hB43xahCD20_ofa_CD4TM_BBZ | SEQ ID | SEQ ID |
| | See FIG. 9, structure C | NO: 2 | NO: 19 |
| | Construct encoding a bispecific aCD19/aCD20 | | |
| | CAR comprising a CD4-derived spacer and TM | | |
| | domain, , and an IC domain comprising 4-1BB | | |
| | and CD3ζ. | | |
| MX1956 | STAR_ahCD19_hB43xahCD20_ofa(altL1)_CD8TM_BBZ | SEQ ID | SEQ ID |
| | | NO: 3 | NO: 20 |
| MX1957 | STAR_ahCD19_hB43xahCD20_ofa(altL2)_CD8TM_BBZ | SEQ ID | SEQ ID |
| | | NO: 4 | NO: 21 |
| MX1958 | EGFP | SEQ ID | SEQ ID |
| | Enhanced Green Fluorescent Protein (marker) | NO: 5 | NO: 22 |
| MX1959 | hEPO | SEQ ID | SEQ ID |
| | Human Erythropoietin | NO: 6 | NO: 23 |
| MX1560 | mCherry | SEQ ID | SEQ ID |
| | mCherry Red Fluorescente Protein (marker) | NO: 7 | NO: 24 |
| MX1561 | mEPO | SEQ ID | SEQ ID |
| | Human Erythropoietin | NO: 8 | NO: 25 |
| MX1962 | Rep78 | SEQ ID | SEQ ID |
| | AAV2 Rep78 protein (Genbank QDH44117.1) | NO: 9 | NO: 26 |
| | from Adeno-associated virus isolate | | |
| | CHC442_AAV.FL.linear isolate (Genbank | | |
| | MK139249.1) | | |
| MX717 | VRC01.23/PGDM1400XePGT121.18_DQ | SEQ ID | SEQ ID |
| | | NO: 10 | NO: 27 |
| | | SEQ ID | SEQ ID |
| | | NO: 11 | NO: 28 |
| | | SEQ ID | SEQ ID |
| | | NO: 12 | NO: 29 |
| | | SEQ ID | SEQ ID |
| | | NO: 13 | NO: 30 |
| MX718 | VRC01.23/PGDM1400XePGT121.18_LS | SEQ ID | SEQ ID |
| | | NO: 14 | NO: 31 |
| | | SEQ ID | SEQ ID |
| | | NO: 15 | NO: 32 |
| | | SEQ ID | SEQ ID |
| | | NO: 16 | NO: 33 |
| | | SEQ ID | SEQ ID |
| | | NO: 17 | NO: 34 |

### Example 5

### Identification of 5′ and 3′ ITR and Rep Sequences in Parvoviral Genomes

Identification of 5′ and 3′ ITR and Rep sequences in Parvoviral genomes can be conducted routinely by a person of ordinary skill in the art using publicly available tools and without undue experimentation as disclosed in detail in U.S. Provisional Application No. 63/552,575, filed on Feb. 12, 2024, which is herein incorporated by reference in its entirety. Namely, the ITRs are identified by pairwise alignment between the genomic sequence and its reverse complement, wherein the ITRs correspond to areas of approximately 10000 sequence complementarity. The reverse complement of the genomic sequence of the parvovorus can be generated using, for example, the Reverse Complement tool available at www.bioinformatics.org/sms/rev_comp.html. The alignment of the original genomic sequence and its reverse complement can be conducted using, for example, Clustal Omega, available at www.ebi. ac.uk/jdispatcher/msa/clustalo. The ITR sequences identified using this alignment method disclosed above can be used to identify the parvoviral ITRs corresponding to any of the parvirus genomes disclosed in the present application to generate the AAV-ITR vectors of the present disclosure.

To identify the sequence encoding the Rep78 protein in the same parvoviral genome in which the ITRs were identified as described above, the parvoviral genomic sequence can be translated to the corresponding amino acid sequence, e.g., using the Translate tool available at web.expasy.org/translate/. The identified protein sequence can be extracted in fasta format as disclosed in detail in U.S. Provisional Application No. 63/552,575, and used to map the amino acid sequence encoding the Rep protein onto the genomic sequence, yielding the Rep-encoding polynucleotide sequence that can be used to generate a Rep vector of the present disclosure.

In summary, 5′ ITR, 3′ ITR, and AAV-Rep78 ortholog sequences can be identified for each one of the parvovirus genomes disclosed in the present application and available in public databases by a person of skill in the art using the methods disclosed above without undue experimentation.

### INCORPORATION BY REFERENCE

The contents of all cited references (including literature references, patents, patent applications, and websites) that can be cited throughout this application are hereby expressly incorporated by reference in their entirety for any purpose, as are the references cited therein, in the versions publicly available on the date the present application was filed Protein and nucleic acid sequences identified by database accession number and other information contained in the subject database entries (e.g., non-sequence related content in database entries corresponding to specific Genbank accession numbers) are incorporated by reference, and correspond to the corresponding database release publicly available on the date the present application was filed.

### EQUIVALENTS

While various specific aspects have been illustrated and described, the above specification is not restrictive. It will be appreciated that various changes can be made without departing from the spirit and scope of the invention(s). Many variations will become apparent to those skilled in the art upon review of this specification.

It is to be appreciated that the Detailed Description section, and not the Summary and Abstract sections, is intended to be used to interpret the claims. The Summary and Abstract sections can set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

1. An AAV-ITR based gene delivery system comprising
(i) a linear double-stranded DNA (dsDNA) AAV-ITR vector comprising 5′ and 3′ AAV internal terminal repeats (ITR) flanking a polynucleotide sequence encoding a therapeutic polypeptide, a therapeutic polynucleotide, or a combination thereof (“AAV-ITR vector”); and,
(ii) a mRNA vector encoding a AAV Rep protein (“AAV Rep vector”),
wherein the AAV-ITR vector is encapsulated in a first liposome or lipid nanoparticle (LNP) and the Rep vector is encapsulated in a second liposome or LNP, and wherein the delivery system is targeted to a cell or tissue using at least one targeting molecule.

2. (canceled)

3. The AAV-ITR based gene delivery system of claim 1_, wherein the ITR comprises an ITR sequence from AAV1, AAV2, AAV3, AAV3B, AAV4, AAV5, AAV6, AAV7, AAV8, AAV9, AAV10, AAV 11, AAV12, AAV13, an ortholog parvoviral ITR, or a functional fragment of functional variant thereof.

4. (canceled)

5. The AAV-ITR based gene delivery system of claim 1, wherein the AAV Rep protein comprises a Rep78 protein, a Rep68 protein, a variant thereof, or a functional fragment thereof.

6. The AAV-ITR based gene delivery system of claim 3, wherein the AAV Rep78 protein is from AAV1, AAV2, AAV3, AAV3B, AAV4, AAV5, AAV6, AAV7, AAV8, AAV9, AAV10, AAV 11, AAV12, or AAV13, a variant thereof, or a functional fragment thereof.

7. The AAV-ITR based gene delivery system of claim 1, wherein the topology of the AAV-ITR vector corresponds to Schema I
[ITR_{L}]-[E]-[P]-[I]-[GOI]-[P(A) signal]-[ITR_{R}]
wherein:
ITR_{L} and ITR_{R} are ITRs, and ITR_{R} is the reverse complement if ITR_{L};
E is an enhancer;
P is a promoter;
I is an intron;
GOI is a polynucleotide sequence encoding a therapeutic polypeptide, a therapeutic polynucleotide, or a combination thereof, and,
P(A) signal is a polyadenylation signal.

8. The AAV-ITR based gene delivery system of claim 1, wherein the topology of the AAV Rep vector corresponds to Schema II
[P]-[5′ UTR]-[Rep78/68]-[3′ UTR]-[P(A) tail]
wherein:
P is a promoter;
5′ UTR is a 5′ untranslated region;
3′ UTR is a 3′ untranslated region;
Rep78/68 is a polynucleotide sequence encoding a Rep78 or Rep68 protein; and,
P(A) tail is a polyadenylation tail.

9. The AAV-ITR based gene delivery system of claim 1, wherein the polynucleotide sequence encodes an antibody, an enzyme, a receptor, an ion channel, a vaccine antigen, a chimeric antigen receptor (CAR), a hormone, a cytokine, a growth factor, or an apoptosis regulator.

10. The AAV-ITR based gene delivery system of claim 1, wherein the liposome and/or LNP comprise
(i) a cationic or ionizable lipid or lipidoid;
(ii) a structural lipid;
(iii) a helper lipid;
(iv) a stabilizing lipid; or,
(v) a combination thereof.

11. The AAV-ITR based gene delivery system of claim 10, wherein the ionizable lipid or lipidoid is selected from the group consisting of cKK-E12, AIC-0315, SM-102, YK-009, DLin-MC3-DMA (MC3), DLin-KC2-DMA (KC2), A6, OF-02, A18-Iso5-2DC18, 98N₁₂-5, 9A1p9, C12-200, 7C1, G0-C14, L319, 304O₁₃, OF-Deg-Lin, 306-O12B, 306O110, FTT5, Lipid 10, and combinations thereof.

12. (canceled)

13. The AAV-ITR based gene delivery system of claim 1, wherein the at least one targeting molecule is an antibody or a combination thereof.

14. The AAV-ITR based gene delivery system of claim 13, wherein the antibody is an M-STAR antibody or a combination thereof.

15. A method of replicating a polynucleotide encoding a therapeutic polypeptide, therapeutic polynucleotide or combination thereof in vivo in a patient in need of treatment for a disease or condition, comprising delivering the AAV-ITR based gene delivery system of claim 1 to the patient to transiently express the AAV Rep protein encoded by the AAV Rep vector to replicate and amplify the polynucleotide encoding the therapeutic polypeptide, therapeutic polynucleotide or combination encoded by the AAV-ITR vector.

16. A method of method of delivering a therapeutic polypeptide, therapeutic polynucleotide or combination thereof in vivo to a patient in need of treatment for a disease or condition, comprising delivering the AAV-ITR based gene delivery system of claim 1 to the patient, wherein the AAV Rep protein encoded by the AAV Rep vector is expressed transiently and replicates and amplifies the polynucleotide encoding the therapeutic polypeptide, therapeutic polynucleotide or combination encoded by the AAV-ITR vector, and wherein the expression of the therapeutic polypeptide, therapeutic polynucleotide or combination thereof treats the disease or condition in the patient.

17. A therapeutic polypeptide, therapeutic polynucleotide or combination produced in vivo or ex vivo by delivering the AAV-ITR based gene delivery system of claim 1 to a host cell, wherein the AAV Rep protein encoded by the AAV Rep vector is expressed transiently and replicates and amplifies the polynucleotide encoding the therapeutic polypeptide, therapeutic polynucleotide or combination encoded by the AAV-ITR vector, and wherein the cell expresses the therapeutic polypeptide, therapeutic polynucleotide or combination thereof.

18. (canceled)

19. A host cell comprising (i) a linear double-stranded DNA (dsDNA) AAV-ITR vector comprising 5′ and 3′ AAV internal terminal repeats (ITR) flanking a polynucleotide sequence encoding a therapeutic polypeptide, a therapeutic polynucleotide, or a combination thereof, and, (ii) a linear mRNA AVV Rep vector encoding an AAV Rep protein.

20. A kit comprising (i) a linear double-stranded DNA (dsDNA) AAV-ITR vector comprising 5′ and 3′ AAV internal terminal repeats (ITR) flanking a polynucleotide sequence encoding a marker; and, (ii) a linear mRNA AAV Rep vector encoding an AAV Rep protein, and instructions to replace the marker with a polynucleotide encoding a therapeutic polypeptide, a therapeutic polynucleotide, or a combination thereof.

21. The kit of claim 20, further comprising lipids to generate at least one liposome or LNP to encapsulate the AAV-ITR vector and the AAV Rep vector, and instructions to generate the at least one liposome or LNP.

22. The kit of claim 21, further comprising a targeting molecule and/or targeting reagents to conjugate the targeting molecule to at least one liposome or LNP, and instructions to conjugate the targeting molecule to at least one liposome or LNP.

23. The AAV-ITR based gene delivery system of claim 1, wherein the first and second liposome or LNP are the same liposome or LNP, and wherein the AAV-ITR vector and the AAV Rep vector are co-delivered in the same liposome or LNP.

24. The AAV-ITR based gene delivery system of claim 9, wherein the CAR is a bispecific anti-CD19/anti-CD20 CAR.
